(19) United States
(12) Patent Application Publication
(10) Pub. No.: US 2025/0075190 A1
(43) Pub. Date: Mar. 6, 2025

(54) DE NOVO DESIGNED LUCIFERASE

(71) Applicant: UNIVERSITY OF WASHINGTON, Seattle, WA (US)

(72) Inventors: David BAKER, Seattle, WA (US); Hsien-Wei YEH, Seattle, WA (US); Christopher NORN, Seattle, WA (US); Yakov KIPNIS, Seattle, WA (US)

(21) Appl. No.: 18/727,868

(22) PCT Filed: Jan. 13, 2023
(86) PCT No.: PCT/US2023/060615

§ 371 Date: Jul. 10, 2024

Related U.S. Application Data
(60) Provisional application No. 63/381,922, filed on Nov. 1, 2022.
(60) Provisional application No. 63/300,171, filed on Jan. 17, 2022.

(51) Int. Cl. C12N 9/02; C12N 15/52; C12N 15/62; C12Q 1/66

(52) U.S. Cl. CPC C12N 9/0069; C12N 15/52; C12N 15/62; C12Q 1/66; C12Y 113/12007; C12N 2510/00

(57) ABSTRACT

Proteins having luciferse activity are disclosed, having the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain; wherein: (a) the H1 domain is at least 18 or 19 amino acids in length; residue 14 of the H1 domain is Y, D, or E, and residue 18 of the H1 domain is D or E: (b) the E3 domain is at least 6, 7, 8, 9, or 10 amino acids in length and residue 2 of the E3 domain is R; and (c) the E5 domain is at least 10, 11, 12, 13, or 14 amino acids in length and residue 9 of the E5 domain is H or M.

### REFERENCE TO RELATED APPLICATIONS

This application claims priority to U.S. Provisional Patent Application Serial Nos. 63/300,171 filed Jan. 17, 2022 and 63/381,922 filed Nov. 1, 2022, each incorporated by reference herein in their entirety.

### FEDERAL FUNDS STATEMENT

This invention was made with government support under Grant No. K99EB031913, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jan. 8, 2023 having the file name “21-1622-WO.xml” and is 638 kb in size.

### BACKGROUND OF THE INVENTION

Bioluminescent light produced by the enzymatic oxidation of a luciferin substrate is widely used for bioassays and imaging in biomedical research. Because no excitation light source is needed, luminescent photons are produced in the dark which results in higher sensitivity than fluorescence imaging in live animal models and in biological samples where autofluorescence or phototoxicity is a concern. However, the development of luciferases as molecular probes has lagged behind that of well-developed fluorescent protein toolkits for a number of reasons: (i) very few native luciferases have been identified; (ii) many of those that have been identified require multiple disulfide bonds to stabilize the structure and are therefore prone to misfolding in mammalian cells; (iii) most native luciferases do not recognize synthetic luciferins with more desirable photophysical properties; and (iv) multiplexed imaging to follow multiple processes in parallel using mutually orthogonal luciferase-luciferin pairs has been limited by the low substrate specificity of native luciferases.

### SUMMARY OF THE INVENTION

In one aspect, the disclosure provides proteins having luciferase activity, comprising the secondary structure arrangement H1-L1-H2-L2-E1-L3-E2-L4-H3-L5-E3-L6-E4-L7-E5-L8-E6, wherein “H” is a helical domain. “L” is a loop domain, and “E” is a beta strand domain; wherein:
(a) the H1 domain is at least 18 or 19 amino acids in length; residue 14 of the H1 domain is Y, D, or E, and residue 18 of the H1 domain is D or E: (b) the E3 domain is at least 6, 7, 8, 9, or 10 amino acids in length and residue 2 of the E3 domain is R; and
(c) the E5 domain is at least 10, 11, 12, 13, or 14 amino acids in length and residue 9 of the E5 domain is H or N.

In various embodiments, 7 of the E5 domain is M; the E6 domain is at least 9, 10, 11, 12, or 13 amino acids in length and wherein residue 5 of the E6 domain is V; residue 1 of the L5 domain is S; residue 7 of the E5 domain is M and residue 5 of the E6 domain is V; and/or residue 7 of the E5 domain is M, residue 5 of the E6 domain is V, and residue 1 of the L5 domain is S.

In other embodiments, the H2 domain is at least 5, 6, or 7 amino acids in length, the H3 domain is at least 9, 10, 11, 12, 13, or 14 amino acids in length, the E1 domain is at least 3 or 4 amino acids in length, the E2 domain is at least 3 or 4 amino acids in length, and/or the E4 domain is at least 8, 9, 10, 11, or 12 amino acids in length.

In one embodiment, 1, 2, 3, 4, or all 5 of the following is true:
(a) residue 13 of domain H1 is F;
(b) residue 1 of domain L3 is W;
(c) residue 5 of domain E5 is V or another hydrophobic residue;
(d) residue 8 of domain E5 is A or L or another hydrophobic residue; and/or
(e) residue 11 of domain E5 is W.

In another embodiment, 1, 2, 3, 4, 5, or all 6 of the following are true:
(a) residue 2 of domain E1 is I or another hydrophobic residue;
(b) residue 4 of domain H3 is F;
(c) residue 6 of domain E4 is V or another hydrophobic residue;
(d) residue 8 of domain E4 is L or another hydrophobic residue;
(e) residue 5 of domain E6 is M or V or another hydrophobic residue; and/or
(f) residue 7 of domain E6 is V or another hydrophobic residue.

In a further embodiment, the protein comprises an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-181, or SEQ ID NO:1-3. In another embodiment, the protein comprises the amino acid sequence of SEQ ID NO:4.

In one aspect, the disclosure provides proteins having luciferase activity, and comprising an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1, wherein:

Residue 14 is Y, D, or E and residue 98 is H or N; and

Residue 18 is D or E and residue 65 is R.

In various embodiments, the protein comprises one or both of A96M and M110V substitutions relative to SEQ ID NO:1; both of A96M and M110V substitutions relative to SEQ ID NO:1; and/or an R60S substitution relative to SEQ ID NO:1; R60S, A96M, and M110V substitutions relative to SEQ ID NO:1. In other embodiments, the protein comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-3, or 1-181.

In another aspect, the disclosure provides a protein comprising the formula X1-Z1-X2-Z2-X3-Z3-X4-Z4-X5-Z5-X6-Z6-X7-Z7-X8-Z8, wherein:
X1 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of MSEEQIRQFL RRFYEALD (SEQ ID NO: 182), wherein residue 14 is Y, D, or E and residue 18 is D or E;
X2 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of ADTAASLF (SEQ ID NO: 183);
X3 has an amino acid sequence at least 50%, 75%, or 100% identical to the amino acid sequence of TIHL (SEQ ID NO: 184);
X4 has an amino acid sequence at least 33%, 66%, or 100% identical to the amino acid sequence of VTF;
X5 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of EEFR EWFERLFST (SEQ ID NO: 185);
X6 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of QREIKSL EVR (SEQ ID NO: 186), wherein residue 2 is R;
X7 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of VEVH VQLHATH (SEQ ID NO: 187);
X8 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of KHTVDATHHW HFR (SEQ ID NO: 188), wherein residue 8 is H or N;
X9 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of VTEM RVHINPTG (SEQ ID NO: 189); and
wherein Z1, Z2, Z3, Z4, Z5, Z6, Z7, and Z8 are independently present or absent, and when present may comprise any amino acid sequence.

In another embodiment, the disclosure provides self-complementing multipartite protein shaving luciferase activity, comprising at least a first polypeptide component and a 20 second polypeptide component, wherein the at least first polypeptide component and the second polypeptide component are not covalently linked, wherein in total the at least first polypeptide component and the second polypeptide component comprise domains X1-Z1-X2-Z2-X3-Z3-X4-Z4-X5-Z5-X6-Z6-X7-Z7-X8-Z8-X9, wherein each domain is as defined herein:
wherein (a) each X domain is fully present within one polypeptide component of the at least first polypeptide component and the second polypeptide component, and (b) none of the at least first polypeptide component and the second polypeptide component include each of X1, X2, X3, X4, X5, X6, X7, X8, and X9.

In a further embodiment, the disclosure provides self-complementing multipartite protein having luciferase activity, comprising at least a first polypeptide component and a second polypeptide component, wherein the at least first polypeptide component and the second polypeptide component are not covalently linked, wherein in total the at least first polypeptide component and the second polypeptide component comprise the secondary structure arrangement H1-L1-H2-L2-E1-L3-E2-L4-H3-L5-E3-L6-E4-L7-E5-L8-E6, wherein each domain is as defined herein

In a further embodiment, the disclosure provides proteins having luciferase activity, comprising the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain; wherein:
(a) the H1 domain is at least 18 or 19 amino acids in length; residue 14 of the H1 domain is Y. D. or E, and residue 18 of the H1 domain is D or E;
(b) the E3 domain is at least 6, 7, 8, 9, or 10 amino acids in length and residue 2 of the E3 domain is R; and
(c) the E5 domain is at least 10, 11, 12, 13, or 14 amino acids in length and residue 9 of the E5 domain is H or N.

The disclosure also provides fusion proteins comprising:
(a) the protein or polypeptide component of any embodiment; and
(b) one or more additional functional domains.

The disclosure further provides nucleic acids encoding the protein, polypeptide component, or fusion proteins of the disclosure, expression vector comprising the nucleic acids operatively linked to a suitable control element, host cells comprising a protein, polypeptide component, fusion protein, nucleic acid, and/or expression vector of the disclosure; and kits comprising a protein, polypeptide component, fusion protein, nucleic acid, expression vector, and/or host cell of the disclosure; and instructions for their use. The disclosure also provides methods for use of a protein, polypeptide component, fusion protein, nucleic acid, expression vector, host cell, and/or kit of the disclosure.

### BRIEF DESCRIPTION OF THE FIGURES

FIG. 1: Generation of idealized scaffolds and computational design of de novo luciferases. (a) Family-wide hallucination. Sequences encoding proteins with the desired topology are optimized by Monte Carlo sampling with a multicomponent loss function. Structurally conserved regions are evaluated based on consistency with input residue-residue distance and orientation distributions obtained from 85 experimental structures of NTF2-like proteins, while variable non-ideal regions are evaluated based on the confidence of predicted inter-residue geometries calculated as the KL-divergence between network predictions and the background distribution. The sequence-space MCMC-sampling incorporates both sequence changes and insertions/deletions (see Methods) to guide the hallucinated sequence towards encoding structures with the desired folds. Hydrogen-bonding networks are incorporated into the designed structures to increase structural specificity. (b-d) The design of luciferase active sites. (b) Generation of DTZ conformers using AIMNet. (c) Generation of a rotamer interaction field (RIF) to stabilize the anionic DTZ and form hydrophobic packing interactions around the DTZ conformers. (d) Docking of the RIF into the hallucinated scaffolds, and optimization of substrate-scaffold interactions using position-specific score matrices (PSSM)-biased sequence design. (e) Selection of the NTF2 topology. The RIF was docked into 4000 native small molecule binding proteins, excluding proteins that bind the luciferin substrate using more than 5 loop residues. Most of the top hits were from the NTF2-like protein superfamily. Using the family-wide hallucination scaffold generation protocol, we generated 1615 scaffolds and found that these yielded better predicted RIF binding energies than the native proteins. (f) Scaffolds generated with family-wide hallucination sample more within the space of the native structures than previous blueprint generated scaffolds, and (g) have stronger sequence to structure relationships than native or blueprint de novo NTF2 scaffolds.

FIG. 2. Biophysical characterization of LuxSit. (a) Coomassie-stained SDS-PAGE of purified recombinant LuxSit from E. coli. (b) Size-exclusion chromatography of purified LuxSit suggested monodispersed and monomeric properties. (c) Far-ultraviolet CD spectra at 25° C., 95° C., and cooled back to 25° C. Insert: CD melting curve of LuxSit at 220 nm. (d) Luminescence emission spectra of DTZ in the presence and absence of LuxSit. (e) Structural alignment of the design model and AlphaFold2 predicted model, which are in close agreement at both backbone (left) and sidechain level (right). (f-i) Site saturation mutagenesis of substrate interacting residues. Zoomed-in views (left) of design and AlphaFold2 models at sidechain level illustrated the designed enzyme-substrate interactions of (f), Tyr14-His98 core HBNets, (g), Asp18-Arg65 dyad, (h), π-stacking, and i, hydrophobic packing residues. Sequence profiles (right) are scaled by the activities of different sequence variants: (activity for the indicated amino acid)/(the sum of activities over all tested amino acids at the position). Substitutions with increased activity (Ala96 and Met110) are highlighted.

FIG. 3. Characterization of luciferase activity in vitro and in human cells. (a) Substrate concentration dependence of LuxSit. LuxSit-f, and LuxSit-i activity. Numbers indicate the signal-to-background ratio at Vₘₐₓ. (bFluorescence and luminescence imaging of live HEK293T cells transiently expressing LuxSit-i-mTagBFP2; LuxSit-i activity can be detected at single-cell resolution. Left: fluorescence channel representing mTagBFP2 signal. Right: total luminescence photons were collected during a course of 10 s exposure. Inserts: negative control, untransfected cells with DTZ. The luminescence images were acquired immediately after adding 25 μM DTZ without excitation light. Scale bar: 20 μm. 40×.

FIG. 4. High substrate specificity of designed luciferases allows multiplexed bioassay. (a) Chemical structures of Coelenterazine substrate analogs. (b) Activity of LuxSit-i on selected luciferin substrates. Luminescence image (top) and signal quantification (bottom) of the indicated substrate in the presence of 100 nM LuxSit-i. LuxSit-i has high specificity for the design target substrate, DTZ. (c) Heatmap visualization of the substrate specificity of LuxSit-i. Renilla luciferase (RLuc), Gaussia luciferase (GLuc), engineered NLuc from Oplophorus luciferase. The heatmap shows luminescence for each enzyme on each substrate; values are normalized on a per-enzyme basis to the highest signal for that enzyme over all substrates. (d) Luminescence emission spectrum of LuxSit-i/DTZ and RLuc/PP-CTZ can be spectrally resolved by 528/20 and 390/35 filters (shown in dashed bars) and only recognize the cognate substrate. (e) Schematic of the multiplex luciferase assay. HEK293T cells transiently transfected with CRE-RLuc, NFkB-LuxSit-i, and CMV-CyOFP plasmids were treated with either Forskolin or human tumor necrosis factor alpha (TNFα) to induce the expression of labeled luciferases. (f-g) Luminescence signals from cells can be measured under either substrate-resolved or spectrally resolved methods by a plate reader. (f) For the substrate-resolved method, luminescence intensity was recorded without a filter after adding either PP-CTZ or DTZ. (g) For the spectrally resolved method, both PP-CTZ and DTZ were added, and the signals were acquired using 528/20 and 390/35 filters simultaneously. In (f) and (g), the lower panel indicates the addition of Forskolin or TNFα. Luminescence signals were acquired from the lysate of 15,000 cells in CelLytic™ M reagent while CyOFP fluorescence signal was used to normalize cell numbers and transfection efficiencies. All data were normalized to the corresponding non-stimulated control. Data are presented as mean±SD (n=3).

FIG. 5. Proposed catalytic mechanism of coelenterazine-utilizing luciferases. Density-functional theory (DFT) calculation suggested that the formation of an anionic state is the essential electron source for the activation of triplet oxygen (³O₂). Supported by both theoretical^{26,27} and experimental evidence^{28,29}, the next oxygenation process is likely through a single electron transfer (SET) mechanism in which the surrounding reaction field could highly influence the change of Gibbs free energy (ΔGₛₑₜ). Finally, the thermolysis of a dioxetane light emitter intermediate can produce photons via the mechanism of gradually reversible charge-transfer-induced luminescence (GRCTIL), which is generally exergonic. Since all the historical pieces of evidence are based on calculations in the virtual solvents or chemiluminescence in ideal organic solvents. The detailed mechanism of a luciferase-catalyzed luminescence reaction has remained unclear. We proposed that the key step of the enzyme is to promote the formation of an anionic state and create a suitable environment to facilitate efficient SET. Hence, the goal of this study is to design an enzyme reaction field surrounding the substrate to stabilize the anionic substrate state and alter the local proton activity, solvent polarity, and hydrophobicity for the efficient activation of ³O₂.

FIG. 6. Schematic representative of colony-based luciferase screening. Computationally designed DNA sequences were provided in an oligo array, where the fragments were amplified by PCR, assembled, and ligated into a pBAD bacterial expression vector. The plasmid library was used to transform DH10B cells. Each colony grown on the LB agar plate represented one luciferase design. The plates were sprayed with DTZ solution and imaged to identify active colonies using a ChemiDoc™ imager. All active colonies were inoculated in 96-well plates, expressed, and purified to confirm individual luciferase activity. Selected plasmids can then be sequenced to point out active design models that provide insights into the design principle and enzyme functions or can be subjected to random mutagenesis for further evolution. Insert: three luciferases were identified from this screening. We refer to the most active and DTZ-specific luciferase as “LuxSit”.

FIG. 7. Expression, purification, and structural characterization of LuxSit variants. (a-c) The recombinant expression of (a) LuxSit, (b) LuxSit-i, and (c) LuxSit-f in E. coli. Annotations for each lane are the following—1: Pre-IPTG; 2: Post-IPTG: 3: Soluble lysate; 4: Flow-through; 5: Wash; 6: Elusion: 7: Post-TEV cleavage; 8: Post-SEC. (d-f) Size-exclusion chromatography of the purified (d) LuxSit; (e) LuxSit-i; and (f) LuxSit-f monomer. (g-i) Deconvoluted mass spectrum of (g) LuxSit, (h) LuxSit-i, and (i) LuxSit-f. (j-k) Far-ultraviolet circular dichroism (CD) spectra (Left panel) of (j) LuxSit-i; and (k) LuxSit-f at 25° C. 95° C., and cooled back to 25° C. CD melting curve at 220 nm (Right panel). (l) Dimeric SEC peak was observed when LuxSit-i was concentrated to high concentration (˜50 μM) in Tris pH 8.0 buffer. Both dimeric and monomeric SEC fractions showed the expected size on SDS PAGE and both peaks were catalytically active to emit luminescence in the presence of 25 μM DTZ.

FIG. 8. Screening of a randomized NNK library at 60, 96, and 110 positions and sequence alignment between LuxSit and its variants. We generated a fully randomized library at 60, 96, and 110 positions to exhaustively screen all possible combinations. After the colony-based screening, we identified many colonies with strong luciferase activities with DTZ. Each colony was expressed individually in each well of 96-well plates (1 mL culture) and purified accordingly (see Methods). (a) Individual luminescence activity of each selected mutant was plotted and compared to the parent LuxSit. Luminescence activities were measured in the presence of 25 μM DTZ. Luminescence activity (RLU) was shown as the integrated signal over the first 15 min. Statistical analysis of the amino acid frequency versus the luciferase activity at residue (b) 60, (c) 96, and (d) 110. Among all selected mutants, Arg60 is confirmed to be mutable as Arg60 may be structurally less well defined as it emanates from a loop and has no hydrogen-bonding partner. Ala96 prefers larger sidechain (Leu, Ile, Met, and Cys), and Met110 favors hydrophobic residues (Val, Ile, and Ala). A newly discovered variant (R60S/A96L/M110V) with more than 100-fold higher photon flux over LuxSit was assigned LuxSit-i for its high brightness.

FIG. 9. Sequence alignment of Lux-Sit (SEQ ID NO: 1), Lux-Sit-i (SEQ ID NO: 2), and Lux-Sit-f (SEQ ID NO: 3). In the sequence alignment, mutations are highlighted. The conserved catalytic dyads of Asp18-Arg65 and Tyr14-His98 are shown.

FIG. 10. Additional characterization of LuxSit variants. (a) Normalized emission kinetics of 15,000 intact HeLa cells expressing LuxSit-i, 100 nM purified LuxSit-i, or 100 nM purified LuxSit-f in the presence of 50 μM DTZ. The more extended emission kinetics in HeLa cells is likely due to the diffusion rate of DTZ across cell membranes. (b) Normalized luminescence decay curves of LuxSit-i in various pH buffers revealed a pH-dependent catalytic mechanism. (c) Luminescent quantum yield was estimated from the integrated luminescence signal until completely converting 125 pmol substrates to photons in the presence of 50 nM corresponding luciferase (see Methods). All data points were plotted as the average of triplicate measurements.

FIG. 11. Expression, localization, and luminescence activity of LuxSit-i in live HEK293T and HeLa cells. (a-b) Fluorescence imaging of live (a) HEK293T and (b) HeLa cells expressing LuxSit-i-mTagBFP2, which is untargeted or localized to the nucleus (Histone2B), plasma membrane (KRasCAAX), or mitochondria (DAKAP) cellular compartments. Scale bar: 10 μm. (c-d) Luminescence signals were measured with 15,000 intact (c) HEK293T or (d) HeLa cells in the presence of 25 μM DTZ in DPBS. Transfection efficiencies range from 60-70% for HEK293T cells and 5-10% for HeLa cells. (e) Luminescence emission spectra acquired from LuxSit-i expressing HEK293T cells is consistent with the emission spectra of recombinant LuxSit-i purified from E. coli. (f-g) Luminescence signals were measured with 15,000 (f) intact LuxSit-i expressing HEK293T cells or (g) cell lysate in the presence of 25 μM indicated substrate in DPBS. Luminescence intensities were normalized to DTZ signal, showing high DTZ specificity over other substrates in cell-based assays. Data were shown as total luminescence signal over the first 20 min and were done in technical triplicates. (h) Normalized luminescence intensity profile of lines traversing across different cells (n=10) of main FIG. 3b luminescence image; lines represent untransfected cells. Error bars represent±SEM.

FIG. 12. Substrate specificity of LuxSit-i and spectrally resolved luciferase-luciferin pairs allow multiplexed bioassay. (a) The orthogonality relationship between LuxSit-i-DTZ and RLuc-PP-CTZ (Prolume Purple, methoxy e-Coelenterazine) luminescent pairs. Indicated amounts of each luciferase were mixed at different ratios totaling 100%. (b) After the addition of both 25 μM DTZ and PP-CTZ substrates, filtered light from 528/20 and 390/35 were measured simultaneously. Data are presented as mean±SD (n=3). Heatmap shows the luminescence signal for individual luciferase (100 nM) or 1:1 mixture in the presence of the cognate or non-cognate (DTZ or PP-CTZ or both) substrates. Response signals were acquired by a Neo2T™ plate reader with 528/20 and 390/35 filters simultaneously. (c) Multiplex luciferase assay in live HEK293T after co-transfection of CRE-RLuc, NFκB-LuxSit-i, and CMV-CyOFP plasmids and stimulation by Forskolin (FSK) or human tumor necrosis factor alpha (TNFα). (d,e) 15,000 intact cells were assayed (see Methods) by either (d) substrate-resolved or (e) spectrally resolved modes after adding DTZ, PP-CTZ, or both DTZ and PP-CTZ in DPBS without cell lysis. Area-scanning of CyOFP fluorescence signal was used to estimate cell numbers and transfection efficiency. The reported unit was RLU/a.u.; relative light units/fluorescence intensity measurements at Ex./Em.=480/580 nm. All data were normalized to the corresponding non-stimulated control. Data are presented as mean±SD (n=3).

FIG. 13. Secondary structure is shown mapped onto an exemplary protein of the disclosure (SEQ ID NO: 1).

### DETAILED DESCRIPTION

All references cited are herein incorporated by reference in their entirety.

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be deleted).

All embodiments of any aspect of the disclosure can be used in combination, unless the context clearly dictates otherwise.

Unless the context clearly requires otherwise, throughout the description and the claims, the words ‘comprise’. ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein.” “above,” and “below” and words of similar import, when used in this application, shall refer to this application as a whole and not to any particular portions of the application

In a first aspect, the disclosure provides proteins having luciferase activity, comprising the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain; wherein:
(a) the H1 domain is at least 18 or 19 amino acids in length; residue 14 of the H1 domain is Y, D. or E, and residue 18 of the H1 domain is D or E;
(b) the E3 domain is at least 6, 7, 8, 9, or 10 amino acids in length and residue 2 of the E3 domain is R; and
(c) the E5 domain is at least 10, 11, 12, 13, or 14 amino acids in length and residue 9 of the E5 domain is H or N.

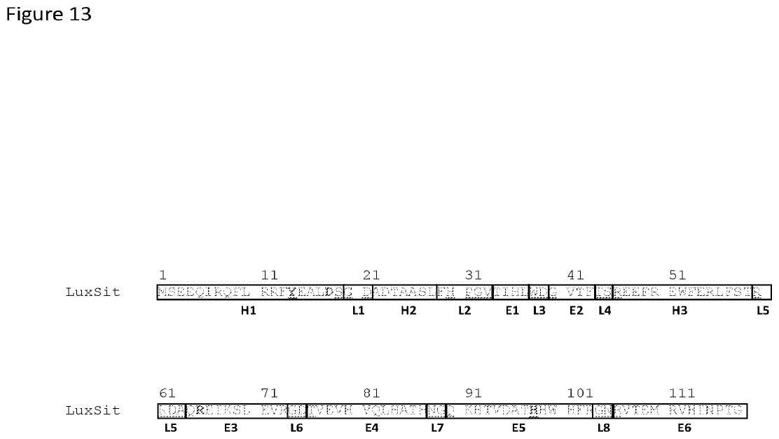

As disclosed in the examples that follow, the proteins of the disclosure are non-naturally occurring, have luciferase activity and share this recited secondary structure arrangement. The arrangement is shown with respect to the amino acid sequence of SEQ ID NO:1 in FIG. 13. The inventors have conducted extensive studies to assess key residues in the polypeptides for retaining luciferase activity and made a large number of modified versions of the polypeptides as detailed in SEQ ID NO:4 and the examples that follow. The required amino acids noted above are those involved in the catalytic dyads, as described below and in the examples.

Except as noted, the different domains may be any suitable length.

In one embodiment, residue 7 of the E5 domain is M. In another embodiment, the E6 domain is at least 9, 10, 11, 12, or 13 amino acids in length and residue 5 of the E6 domain is V. In a further embodiment, residue 1 of the L5 domain is S. In one embodiment, residue 7 of the E5 domain is M and residue 5 of the E6 domain is V. In a further domain, residue 7 of the E5 domain is M, residue 5 of the E6 domain is V, and residue 1 of the L5 domain is S. In another embodiment, the H2 domain is at least 5, 6, or 7 amino acids in length, the H3 domain is at least 9, 10, 11, 12, 13, or 14 amino acids in length, the E1 domain is at least 3 or 4 amino acids in length, the E2 domain is at least 3 or 4 amino acids in length, and the E4 domain is at least 8, 9, 10, 11, or 12 amino acids in length. In all these embodiments, one or more of the recited domains may independently include amino acid residues. In one embodiment, one or more of the domains may independently include an additional 1, 2, 3, 4, or 5 residues.

In one embodiment:
the H1 domain is 19 amino acids in length;
the H2 domain is 7 amino acids in length;
the E1 domain is 4 amino acids in length;
the E2 domain is 4 amino acids in length;
the H3 domain is 14 amino acids in length;
the E3 domain is 10 amino acids in length;
the E4 domain is 12 amino acids in length;
the E5 domain is 14 amino acids in length; and
the E6 domain is 12 or 13 amino acids in length.

The loop domains may be of any length and may include insertions, relative to the sequences exemplified herein, of any residues or functional domains as deemed appropriate, including but not limited to metal binding domains, drug binding domains, GPCR receptors, protein switches, and small molecule binding domains.

In another embodiment, the proteins comprise an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-3, wherein residues in parentheses are optional and may be present or may be deleted.

SEQ ID NO:1 is the Lux-Sit construct disclosed herein. FIG. 13 shows the domain structure mapped onto the SEQ ID NO:1 amino acid sequence.

(SEQ ID NO: 1)
(M) SEEQIRQEL RRFYEALDSG DADTAASLFH PGVTIHLWDG
VTFTSREEFR EWFERLFSTR KDAQREIKSL EVRGDTVEVH
VQLHATHNGQ KHTVDATHHW HERGNRVTEM RVHINPT (G)

SEQ ID NO:2 is the Lux-Sit-i construct disclosed herein.

(SEQ ID NO: 2)
(M) SEEQIRQFL RRFYEALDSG DADTAASLFH PGVTIHLWDG
VTFTSREEFR EWFERLFSTS KDAQREIKSL EVRGDTVEVH
VQLHATHNGQ KHTVDLTHHW HERGNRVTEV RVHINPT (G)

SEQ ID NO:3 is the Lux-Sit-f construct disclosed herein.

(SEQ ID NO: 3)
(M) SEEQIRQFL RRFYEALDSG DADTAASLFH PGVTIHLWDG
VTFTSREEFR EWFERLFSTR KDAQREIKSL EVRGDTVEVH
VQLHATHNGQ KHTVDMTHHW HERGNRVTEV RVHINPT (G).

In each of the annotated sequences shown for SEQ ID NO:1-3:
(a) Bold and underlined and increased font size positions are Dyad 1 (catalytic residues) Y14 (H1 domain residue 14)+H98 (E5 domain residue 9);
(b) Bold and increased font size positions are Dyad 2 (catalytic residues) D18 (H1 domain residue 9)+R65 (E3 domain residue 2);
(c) Increased font and not bolded positions are core packing (recognition residues) F13 (residue 13 of domain H1), 135 (residue 2 of domain E1), W38 (residue 1 of domain L3), F49 (residue 4 of domain H3), V81 (residue 6 of domain E4), L83 (residue 8 of domain E4), V94 (residue 5 of domain E5). A/L 97 (residue 8 of domain E5), W100 (residue 11 of domain E5), M/V110 (residue 5 of domain E6), V112 (residue 7 of domain E6); and
(d) Underlined and not bolded positions are regions (loop domains or immediately adjacent) for splitting the enzyme or inserting other functional domains.

In some embodiments of the proteins, 1, 2, 3, 4, or all 5 of the following is true:
(a) residue 13 of domain H1 is F;
(b) residue 1 of domain L3 is W;
(c) residue 5 of domain E5 is V or another hydrophobic residue;
(d) residue 8 of domain E5 is A or L or another hydrophobic residue; and/or
(e) residue 11 of domain E5 is W.

In other embodiments of the proteins, the H2 domain is 7 amino acids in length, the H3 domain is 14 amino acids in length, the E1 domain is 4 amino acids in length, the E2 domain is 4 amino acids in length, and/or the E4 domain is 12 amino acids in length. In further embodiments, 1, 2, 3, 4, 5, or all 6 of the following are true:
(a) residue 2 of domain E1 is I or another hydrophobic residue;
(b) residue 4 of domain H3 is F;
(c) residue 6 of domain E4 is V or another hydrophobic residue;
(d) residue 8 of domain E4 is L or another hydrophobic residue;
(e) residue 5 of domain E6 is M or V or another hydrophobic residue; and/or
(f) residue 7 of domain E6 is V or another hydrophobic residue.

In another embodiment, the protein comprises the amino acid sequence of SEQ ID NO:4.

SEQ ID NO: 4

position 1: M, or absent
position 2: S, T
position 3: A, E, K, S
position 4: A, D, E, K, Q, R, S, T
position 5: A, D, E, Q
position 6: I, Q
position 7: E, K, R
position 8: A, D, E, K, N, Q, R
position 9: F
position 10: C, N, V, W, Y, L
position 11: A, D, E, K, Q, R
position 12: K, Q, R, F
position 13: F
position 14: Y
position 15: A, D, E, K, L, N, Q, R, S
position 16: A
position 17: L
position 18: D
position 19: A, D, S
position 20: G
position 21: D
position 22: A
position 23: D, E, N, V
position 24: T
position 25: A
position 26: A, S
position 27: A, S
position 28: L
position 29: F
position 30: K, P, R, H
position 31: D, P
position 32: G
position 33: T, V
position 34: E, I, K, L, N, Q, R, V, T
position 35: I
position 36: D, E, H, K, Y
position 37: L
position 38: W
position 39: D
position 40: G
position 41: I, K, R, T, V
position 42: E, I, T, V
position 43: F
position 44: E, F, H, K, N, R, S, T, Y
position 45: K, T, S
position 46: K, Q, R
position 47: A, E
position 48: E, Q
position 49: F
position 50: K, Q, R
position 51: A, D, E, K, Q, S
position 52: W
position 53: F
position 54: E, K, R, V
position 55: A, D, E, K, Q, R. T
position 56: L
position 57: F, H, K, R, Y
position 58: A, S
position 59: E, K, L, Q, R, T
position 60: S, R
position 61: A, D, E, K, N, Q, S, T
position 62: A, D, E, G, N, Q
position 63: A
position 64: A, K, R, S, Q
position 65: R.
position 66: D, E, H, K, R, S
position 67: I, V
position 68: E, I, T, V, K
position 69: A, D, E, K, N, Q, R, S
position 70: F, L, M
position 71: D, E, K, Q, R, S, T, V
position 72: V
position 73: A, D, E, H, I, N, R
position 74: G
position 75: D, N
position 76: D, E, F, I, K, R, T, V
position 77: A, S, V
position 78: D, E, F, H, K, L, N, R, T, Y
position 79: I, V
position 80: E, I, K, N, R, S, T, V, H
position 81: V
position 82: 1, V, Q
position 83: L
position 84: D, E, H, K, R, V
position 85: A
position 86: D, E, F, I, K, N, R, S, T, V, Y
position 87: E, F, H, I, K, V, Y
position 88: A, D, E, K, N, Q, R
position 89: G
position 90: E, K, Q, R. T
position 91: A, D, E, H, K, P, Q
position 92: E, H, K, L, R, V
position 93: E, I, K, R, T, V
position 94: V
position 95: A, E, F, G, H, K, L, N, R, S, D
position 96: L, A, M
position 97: E, H, K, N, R, T, V, A, L
position 98: H
position 99: E, F, I, K, L, N, Q, R, T, V, W, Y, H
position 100: A, F, T, Y, W
position 101: E, F, H, K, L, Q, R, V, Y
position 102: F, W
position 103: D, E, K, R
position 104: G
position 105: D, S, N
position 106: E, H, K, Q, R
position 107: L, V
position 108: K, R, T. V
position 109: E, K, R.
position 110: V, M
position 111: D, E, F, H, K, N, R, S, T, W, Y
position 112: V
position 113: A, D, E, H, K, R, S, T, V
position 114: I
position 115: D, E, F, H, I, K, N, Q, R, S, T, V, Y
position 116: P
position 117: D, L, M, V, T
Position 118: G or is absent

In another embodiment, the proteins comprise an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid selected from the group consisting of SEQ ID NO:1-181, as shown in Table 1. SEQ ID NO:5-181 in Table 1 are re-designed amino acid sequences based on LuxSit-i (SEQ ID NO:2), with their luciferase activities shown.

TABLE 1

| | SEQ | | | SEQ | |
|---|---|---|---|---|---|
| | ID | | | ID | |
| | NO: | Amino acid | | NO: | |
| Name | (AA) | Sequence | Activity | (NT) | Nucleotide sequence |
| LuxSit | 1 | (M)SEEQIRQFLRRFY | 75.3 | 200 | >LuxSit (codon optimized for E. |
| | | EALDSGDADTAASLFH | | | coli expression) |
| | | PGVTIHLWDGVTFTSR | | | (ATG)AGCGAAGAACAGATTCGTCAGTTTCTGC |
| | | EEFREWFERLFSTRKD | | | GTCGTTTTTATGAAGCGCTGGATAGCGGCGATG |
| | | AQREIKSLEVRGDTVE | | | CGGATACCGCTGCGAGCCTGTTTCATCCGGGCG |
| | | VHVQLHATHNGQKHTV | | | TGACAATTCATCTGTGGGATGGCGTTACCTTTA |
| | | DATHHWHFRGNRVTEM | | | CCAGCCGTGAAGAATTTCGTGAATGGTTTGAAC |
| | | RVHINPT(G) | | | GTCTGTTTAGCACCCGTAAAGATGCGCAGCGTG |
| | | | | | AAATTAAGAGCCTGGAAGTACGTGGCGATACCG |
| | | | | | TGGAAGTGCATGTGCAGTTGCACGCGACCCATA |
| | | | | | ATGGCCAGAAACATACCGTAGATGCAACCCATC |
| | | | | | ATTGGCATTTTCGTGGCAATCGTGTGACCGAAA |
| | | | | | TGCGTGTGCATATCAATCCGACC(GGCTAA) |
| LuxSit-i | 2 | (M)SEEQIRQFERREY | 763.6 | 201 | >LuxSit-i (codon optimized for E. |
| | | EALDSGDADTAASLEH | | | coli expression) |
| | | PGVTIHLWDGVTFTSR | | | (ATG)AGCGAAGAACAGATTCGTCAGTTTCTGC |
| | | EEFREWFERLESTSKD | | | GTCGTTTTTATGAAGCGCTGGATAGCGGCGATG |
| | | AQREIKSLEVRGDIVE | | | CGGATACCGCTGCGAGCCTGTTTCATCCGGGCG |
| | | VHVQLHATHNGQKHTV | | | TGACAATTCATCTGTGGGATGGCGTTACCTTTA |
| | | DETHHWHERGNRVTEV | | | CCAGCCGTGAAGAATTTCGTGAATGGTTTGAAC |
| | | RVHINPT(G) | | | GTCTGTTTAGCACCAGTAAAGATGCGCAGCGTG |
| | | | | | AAATTAAGAGCCTGGAAGTACGTGGCGATACCG |
| | | | | | TGGAAGTGCATGTGCAGTTGCACGCGACCCATA |
| | | | | | ATGGCCAGAAACATACCGTAGATTTGACCCATC |
| | | | | | ATTGGCATTTTCGTGGCAATCGTGTGACCGAAG |
| | | | | | TTCGTGTGCATATCAATCCGACC(GGCTAA) |
| | | | | 202 | >LuxSit-i (codon optimized for |
| | | | | | human cell expression) |
| | | | | | (ATG)AGCGAGGAGCAGATCAGACAGTTCCTGA |
| | | | | | GGAGATTCTACGAGGCCCTGGATAGCGGAGACG |
| | | | | | CCGACACAGCTGCCAGCCTTTTCCATCCTGGCG |
| | | | | | TGACCATCCACCTGTGGGACGGCGTCACCTTCA |
| | | | | | CTAGCAGGGAGGAGTTCAGGGAGTGGTTCGAGA |
| | | | | | GACTGTTCAGCACCAGCAAGGACGCCCAGAGAG |
| | | | | | AGATCAAGAGCCTGGAAGTTAGAGGCGACACCG |
| | | | | | TGGAAGTGCACGTGCAGCTGCACGCCACACACA |
| | | | | | ACGGACAGAAGCACACCGTCGACCTGACCCACC |
| | | | | | ACTGGCACTTCAGAGGCAACAGAGTGACCGAGG |
| | | | | | TGAGAGTGCACATCAATCCCACC(GGCTAA) |
| LuxSit-f | 3 | (M)SEEQIRQFERRFY | 534.5 | 203 | >LuxSit-f (codon optimized for E. |
| | | EALDSGDADTAASLEH | | | coli expression) |
| | | PGVTIHLWDGVTFTSR | | | (ATG)AGCGAAGAACAGATTCGTCAGTTTCTGC |
| | | EEFREWFERLESTRKD | | | GTCGTTTTTATGAAGCGCTGGATAGCGGCGATG |
| | | AQREIKSLEVRGDIVE | | | CGGATACCGCTGCGAGCCTGTTTCATCCGGGCG |
| | | VHVQLHATHNGQKHTV | | | TGACAATTCATCTGTGGGATGGCGTTACCTTTA |
| | | DMTHHWHFRGNRVTEV | | | CCAGCCGTGAAGAATTTCGTGAATGGTTTGAAC |
| | | RVHINPT(G) | | | GTCTGTTTAGCACCCGCAAAGATGCGCAGCGTG |
| | | | | | AAATTAAGAGCCTGGAAGTACGTGGCGATACCG |
| | | | | | TGGAAGTGCATGTGCAGTTGCACGCGACCCATA |
| | | | | | ATGGCCAGAAACATACCGTAGATATGACCCATC |
| | | | | | ATTGGCATTTTCGTGGCAATCGTGTGACCGAAG |
| | | | | | TGCGTGTGCATATCAATCCGACC(GGCTAA) |
| luxsiti_ | 181 | MSEQAQRDFVKKEYEA | 152. | 204 | (ATG)AGCGAACAGGCGCAGCGCGATTTTGTGA |
| 0.3_ | | LDAGDAETASALFPDG | 4478231 | | AGAAATTTTATGAAGCCCTGGATGCGGGCGATG |
| 386 | | TQIYLWDGQTFTTQEQ | | | CCGAAACTGCAAGCGCACTGTTTCCGGATGGCA |
| | | FRAWFVELRSTSKDAK | | | CCCAGATTTATCTGTGGGATGGCCAGACCTTTA |
| | | REVVSFKVDGNNAEVE | | | CCACCCAGGAACAGTTTCGCGCGTGGTTTGTGG |
| | | VVLHAVIDGEERTVKL | | | AACTGCGCAGCACCAGCAAAGATGCGAAACGCG |
| | | KHYFQWEGDQLVKVTV | | | AAGTGGTGAGCTTTAAAGTGGATGGTAACAACG |
| | | SIEPL | | | CGGAAGTGGAAGTTGTGCTGCATGCGGTGATTG |
| | | | | | ATGGCGAAGAACGCACCGTGAAACTGAAACATT |
| | | | | | ATTTTCAGTGGGAAGGCGATCAGCTGGTGAAAG |
| | | | | | TGACCGTGAGCATTGAACCGCTGGG(TAA) |
| luxsiti_ | 5 | MSEEQIREFCKRFYEA | 421. | 205 | (ATG)TCTGAAGAACAAATTCGCGAATTTTGCA |
| 0.3_ | | LDAGDAVTASALEPNG | 3061507 | | AACGCTTTTATGAAGCGCTGGATGCGGGCGATG |
| 389 | | TRIHLWDGITFTTQEE | | | CGGTGACTGCTAGCGCGTTGTTTCCGAATGGCA |
| | | FREWFERLYSQSEDAK | | | CCCGCATTCATCTGTGGGATGGCATTACCTTTA |
| | | REIVSLEVEGNVAYVE | | | CCACCCAGGAAGAATTTCGTGAATGGTTTGAAC |
| | | VILHASFRGEEKTVRL | | | GCCTGTATAGCCAGAGCGAAGATGCGAAACGCG |
| | | RHVFQFEGDKLVEVTV | | | AAATTGTGAGCCTGGAAGTGGAAGGCAACGTGG |
| | | EIEPL | | | CGTATGTGGAAGTTATTCTGCATGCGAGCTTTC |
| | | | | | GCGGTGAAGAGAAAACCGTGCGCCTGCGCCATG |
| | | | | | TTTTTCAGTTTGAAGGCGATAAACTGGTCGAAG |
| | | | | | TGACCGTGGAAATTGAACCGCTGGG(TAA) |
| luxsiti_ | 6 | MSEEAQREFWKRFYEA | 2. | 206 | (ATG)AGCGAAGAAGCGCAGCGCGAATTTTGGA |
| 0.3_ | | LDAGDAETAAALFPDG | 03040774 | | AACGCTTTTATGAAGCGCTGGATGCGGGCGATG |
| 392 | | TEIHLWDGTTERTQAE | | | CCGAAACTGCAGCGGCGTTGTTTCCTGATGGCA |
| | | FRAWFEELYSTSQNAK | | | CCGAAATCCATCTGTGGGATGGTACCACCTTTC |
| | | REIVEFRVDGNKSTVT | | | GCACCCAGGCCGAATTTCGCGCGTGGTTTGAAG |
| | | VVLHAIYEGEEKKVLL | | | AACTGTATTCGACCAGCCAGAACGCGAAACGTG |
| | | EHFTEWEGDRLVRVEV | | | AAATTGTGGAATTCCGCGTGGATGGCAACAAAA |
| | | SIVPL | | | GCACCGTGACCGTGGTGCTGCATGCGATTTACG |
| | | | | | AAGGTGAAGAAAAGAAAGTGCTGCTGGAACATT |
| | | | | | TTACCGAATGGGAAGGCGATCGCCTGGTGCGCG |
| | | | | | TTGAAGTGAGCATCGTGCCGCTGGG(TAA) |
| luxsiti_ | 7 | MSEEEQREFVRRFYEA | 5. | 207 | (ATG)TCCGAAGAAGAACAGCGTGAATTTGTGC |
| 0.3_ | | LDAGDADTASALFPDG | 154803041 | | GCCGCTTTTATGAAGCGCTGGATGCGGGTGATG |
| 395 | | TVIELWDGTTERTRAE | | | CGGATACCGCGAGCGCACTGTTTCCAGATGGCA |
| | | FKAWFEALHSKSENAV | | | CCGTGATTGAACTGTGGGATGGTACCACCTTTC |
| | | RHVVEFEVEGNKAKVR | | | GCACCCGCGCGGAATTTAAAGCGTGGTTTGAAG |
| | | VVLHADYKGKKRVVEL | | | CCCTGCATAGCAAAAGCGAAAACGCGGTGCGCC |
| | | EHEFEFEGDRLVKVSV | | | ATGTGGTGGAATTTGAAGTGGAAGGCAACAAAG |
| | | DIHPI | | | CCAAAGTGCGCGTGGTGCTGCATGCTGATTATA |
| | | | | | AAGGCAAGAAACGCGTTGTGGAACTGGAACATG |
| | | | | | AATTCGAGTTCGAAGGCGATCGCCTGGTGAAAG |
| | | | | | TGTCTGTGGATATTCACCCGATTGG(TAA) |
| luxsiti_ | 8 | MSEEEIRKFCERFYAA | 95. | 208 | (ATG)AGCGAAGAAGAAATTCGCAAATTTTGCG |
| 0.3_ | | LDAGDAETASSLEPDG | 78507256 | | AACGCTTTTATGCGGCGCTGGATGCGGGCGATG |
| 402 | | TEIHLWDGKTERTQAE | | | CGGAAACTGCAAGCAGCCTGTTTCCTGATGGCA |
| | | FREWFVRLRSTSDDAR | | | CCGAAATTCATCTGTGGGATGGCAAAACCTTTC |
| | | RHITKLKVDGNVAEVE | | | GCACCCAGGCGGAATTTCGCGAATGGTTTGTTC |
| | | VVLRASYDGEEKVVAL | | | GTCTGCGCAGCACCAGCGATGATGCGCGCCGTC |
| | | KHVFKFEGDKLVEVKV | | | ATATTACCAAACTGAAAGTGGATGGTAACGTGG |
| | | EITPL | | | CGGAAGTGGAAGTTGTGCTGCGCGCGAGCTATG |
| | | | | | ATGGTGAAGAAAAAGTGGTGGCGCTGAAACATG |
| | | | | | TGTTTAAATTTGAAGGCGATAAACTGGTTGAAG |
| | | | | | TGAAAGTTGAAATTACCCCGCTGGG(TAA) |
| luxsiti_ | | MSEEAQREFVRRFYEA | 119. | 209 | (ATG)AGCGAAGAAGCCCAGCGTGAATTTGTGC |
| 0.3_ | | LDAGDAETASALFPDG | 6136835 | | GCCGCTTTTATGAAGCGCTGGATGCGGGCGATG |
| 405 | | TQIYLWDGKTETTREE | | | CGGAAACCGCAAGCGCACTGTTTCCAGATGGCA |
| | | FRSWFVKLHSTSDNAK | | | CCCAGATTTATCTGTGGGATGGCAAAACCTTTA |
| | | RRVVEFRVEGNKAKVK | | | CCACCCGTGAAGAATTTCGCAGCTGGTTTGTGA |
| | | VVLKASINGKERTVLL | | | AATTGCATAGCACCAGCGATAACGCGAAACGCC |
| | | EHFFEFEGDKLVKVEV | | | GCGTGGTTGAATTTAGAGTGGAAGGCAACAAAG |
| | | RIRPL | | | CGAAAGTAAAAGTGGTGCTGAAAGCGAGCATTA |
| | | | | | ACGGTAAAGAACGCACCGTGCTGCTGGAACATT |
| | | | | | TCTTTGAATTCGAAGGCGATAAACTGGTTAAAG |
| | | | | | TAGAAGTGCGCATTCGCCCGCTGGG(TAA) |
| luxsiti_ | 10 | MSEEEQREFWRRFYEA | 45. | 210 | (ATG)TCTGAAGAAGAACAGCGCGAATTTTGGC |
| 0.3_ | | LDAGDAETASALEPDG | 88113338 | | GTCGCTTTTATGAAGCGCTGGATGCGGGCGATG |
| 443 | | TEIYLWDGRVERTQAE | | | CCGAAACCGCAAGCGCGTTGTTTCCTGATGGCA |
| | | FRAWFVELRSKSDDAR | | | CCGAAATTTATCTGTGGGATGGCCGCGTGTTTC |
| | | REVTSERVDGNKADVR | | | GCACCCAGGCGGAATTTCGCGCATGGTTTGTGG |
| | | VVLHANYKGEKKVVEL | | | AACTGCGCAGCAAAAGCGATGATGCGCGCCGCG |
| | | RHVYEFEGDRLVRVEV | | | AAGTGACCAGCTTTCGCGTGGATGGCAACAAAG |
| | | TINPL | | | CGGATGTGCGCGTGGTGCTGCATGCGAACTATA |
| | | | | | AAGGCGAAAAGAAAGTGGTCGAACTGAGACATG |
| | | | | | TGTATGAATTTGAAGGCGATCGCCTGGTGCGTG |
| | | | | | TGGAAGTTACCATTAACCCGCTGGG(TAA) |
| luxsiti_ | 11 | MSEEEIREFVKRFYEA | 520. | 211 | (ATG)AGCGAAGAAGAAATTCGCGAATTTGTGA |
| 0.3_ | | LDAGDAETASALEPDG | 3628196 | | AACGCTTTTATGAAGCGCTGGATGCAGGCGATG |
| 454 | | TKIHLWDGTVFTTREE | | | CGGAAACCGCGAGCGCATTGTTTCCGGATGGCA |
| | | FKSWFVELRSTSDDAK | | | CCAAAATTCATCTGTGGGATGGTACCGTGTTTA |
| | | REVVDLKVEGNKAYVE | | | CCACCCGTGAAGAATTTAAAAGCTGGTTTGTGG |
| | | VVLRAIVNGEDKVVKL | | | AACTGCGCAGCACCAGCGATGATGCGAAACGCG |
| | | RHVFKFEGDKLVEVHV | | | AAGTGGTGGATCTGAAAGTGGAAGGCAACAAAG |
| | | EIDPL | | | CGTATGTGGAAGTTGTGCTGCGCGCGATCGTGA |
| | | | | | ACGGCGAAGATAAAGTGGTTAAACTGCGTCATG |
| | | | | | TGTTTAAATTTGAAGGCGATAAACTGGTCGAAG |
| | | | | | TGCATGTTGAAATTGATCCGCTGGG(TAA) |
| luxsiti_ | 12 | MSKEEQRKEVERFYKA | 65. | 212 | (ATG)AGCAAAGAAGAACAGCGCAAATTTGTGG |
| 0.3_ | | LDAGDAETASALFPDG | 78991016 | | AACGCTTTTATAAAGCGCTGGATGCGGGCGATG |
| 462 | | TKIHLWDGTTEHTRAE | | | CGGAAACTGCGAGCGCATTGTTTCCGGATGGCA |
| | | FRAWFVDLRSKSENAK | | | CCAAAATTCATCTGTGGGATGGTACCACCTTTC |
| | | REVVAFEVDGNKAKVV | | | ATACCCGCGCGGAATTTCGCGCGTGGTTCGTGG |
| | | VVLKANYKGEERTVKL | | | ATCTGCGCAGCAAAAGCGAAAACGCGAAACGTG |
| | | EHEFYFEGDHLVEVNV | | | AAGTGGTGGCGTTTGAAGTTGATGGCAATAAAG |
| | | KIEPV | | | CCAAAGTGGTTGTGGTGCTGAAAGCAAACTATA |
| | | | | | AAGGCGAAGAACGCACCGTGAAACTGGAACATG |
| | | | | | AATTTTATTTTGAAGGCGATCATCTGGTGGAAG |
| | | | | | TGAACGTTAAAATTGAACCAGTGGG(TAA) |
| luxsiti_ | 13 | MSEEEQREFWKRFYEA | 165. | 213 | (ATG)AGCGAAGAAGAACAGCGCGAATTTTGGA |
| 0.3_ | | LDAGDAETASALFPDG | 0207326 | | AACGCTTTTATGAAGCGCTGGATGCGGGCGATG |
| 468 | | TEIHLWDGKTFHTREE | | | CCGAAACTGCCAGCGCACTGTTTCCAGATGGCA |
| | | FRAWFVDLYSKSKDAS | | | CCGAAATTCATCTGTGGGATGGCAAAACCTTTC |
| | | REITKFEVEGNRAFVE | | | ATACCCGTGAAGAATTTCGCGCGTGGTTTGTGG |
| | | VVLRASYDGEDRTVKL | | | ATCTGTATTCGAAAAGCAAAGATGCGAGCCGCG |
| | | RHIFEFEGDALVEVYV | | | AAATTACCAAATTTGAAGTGGAAGGCAACCGCG |
| | | EIEPL | | | CGTTTGTTGAAGTTGTGCTGCGCGCGAGCTATG |
| | | | | | ATGGCGAAGATCGCACCGTGAAACTGAGACATA |
| | | | | | TTTTTGAATTCGAAGGCGATCATCTGGTGGAAG |
| | | | | | TGTATGTGGAAATTGAACCGCTGGG(TAA) |
| luxsiti_ | 14 | MSAEQIREFVARFYAA | 79. | 214 | (ATG)AGCGCGGAACAAATTCGCGAATTTGTGG |
| 0.3_ | | LDAGDADTASALFPDG | 91015895 | | CGCGCTTTTATGCGGCCTTGGATGCCGGTGATG |
| 483 | | TEIHLWDGRTERTQAE | | | CGGATACGGCAAGCGCGTTGTTTCCGGATGGCA |
| | | FRAWFETLRAQSADAR | | | CCGAAATTCATCTGTGGGATGGCCGCACCTTTC |
| | | RHVVALEVTGNTADVE | | | GCACCCAGGCGGAATTTCGCGCATGGTTTGAAA |
| | | VVLHASVDGEERTVAL | | | CCCTGCGCGCGCAGAGCGCAGATGCGCGTAGAC |
| | | RHRFQFEGDRLVRVTV | | | ATGTGGTGGCATTGGAAGTGACCGGCAACACCG |
| | | EITPL | | | CGGATGTGGAAGTTGTGCTGCATGCGAGCGTGG |
| | | | | | ATGGCGAAGAACGCACCGTGGCGCTGAGACATC |
| | | | | | GCTTTCAGTTTGAAGGCGATCGCCTGGTGCGCG |
| | | | | | TGACCGTTGAAATTACCCCGCTGGG(TAA) |
| luxsiti_ | 15 | MSEEEQKEFVKRFYEA | 75. | 215 | (ATG)TCGGAAGAAGAACAGAAAGAATTTGTGA |
| 0.3_ | | LDAGDAETASALFKDG | 79958535 | | AACGCTTTTATGAAGCGCTGGATGCGGGCGATG |
| 485 | | TEIYLWDGTTERTRAE | | | CGGAAACCGCGAGCGCATTGTTTAAAGATGGCA |
| | | FRAWFRRLKSTSEEAR | | | CCGAAATTTATCTGTGGGATGGTACCACCTTTC |
| | | RSVVSFKVDGNVADVE | | | GTACCCGCGCGGAATTTCGCGCGTGGTTTCGCC |
| | | VVLRARWKGEERVVKL | | | GTCTGAAAAGCACCAGCGAAGAAGCCCGCCGTA |
| | | RHRFVEEGDEVVRVEV | | | GCGTGGTGAGCTTTAAAGTGGATGGCAATGTGG |
| | | EIRPL | | | CGGATGTGGAAGTGGTGCTGCGTGCGCGTTGGA |
| | | | | | AAGGTGAAGAACGCGTAGTGAAACTGCGCCATC |
| | | | | | GCTTTGTGTTTGAAGGCGATGAAGTTGTACGCG |
| | | | | | TTGAAGTGGAAATTCGCCCGCTGGG(TAA) |
| luxsiti_ | 16 | MSAEEQREFVKRFYEA | 6. | 216 | (ATG)AGCGCGGAAGAACAGCGCGAATTTGTGA |
| 0.3_ | | LDAGDAETASALFPDG | 145818936 | | AACGTTTTTATGAAGCGCTGGATGCCGGCGATG |
| 494 | | TKIHLWDGTVENTQEE | | | CGGAAACTGCAAGCGCACTGTTTCCGGATGGCA |
| | | FKAWFVKLRSTSENAK | | | CCAAAATTCATCTGTGGGATGGTACCGTGTTTA |
| | | REVTHFEVDGDKAVVE | | | ACACCCAGGAAGAATTTAAAGCGTGGTTTGTTA |
| | | VVLHANIKGKEKTVRL | | | AACTGCGCAGCACCAGCGAAAACGCGAAACGTG |
| | | RHEFLFEGDKIVEVNV | | | AAGTGACCCATTTTGAAGTGGATGGCGATAAAG |
| | | EIEPL | | | CGGTGGTGGAAGTGGTGCTGCATGCGAACATTA |
| | | | | | AAGGCAAAGAAAAGACCGTGCGCCTGCGCCATG |
| | | | | | AATTTCTGTTTGAAGGCGACAAAATTGTTGAAG |
| | | | | | TTAATGTGGAAATTGAACCGCTGGG(TAA) |
| luxsiti_ | 17 | MSAEAQREFVKKFYDA | 8. | 217 | (ATG)AGCGCGGAAGCGCAGCGCGAATTTGTGA |
| 0.3_ | | LDAGDAETASALFPDG | 460262612 | | AGAAATTTTATGATGCGCTGGATGCGGGCGATG |
| 500 | | TEIYLWDGKVFKTQAE | | | CGGAAACTGCAAGCGCGTTGTTTCCGGATGGAA |
| | | FRAWFVELYSKSDNAQ | | | CCGAAATTTATCTGTGGGATGGCAAAGTGTTTA |
| | | RSVVRFEVDGNVAYVE | | | AAACCCAGGCGGAATTTCGCGCGTGGTTTGTGG |
| | | VVLRANFDGEEKTVRL | | | AACTGTATAGCAAAAGCGATAACGCGCAGAGAA |
| | | RHIFYFEGDSLVKVEV | | | GCGTGGTGCGTTTTGAAGTGGATGGTAACGTGG |
| | | TIEPL | | | CGTATGTGGAAGTGGTGCTGCGCGCGAACTTTG |
| | | | | | ATGGCGAAGAAAAGACCGTGCGCCTGCGCCATA |
| | | | | | TTTTCTACTTTGAAGGTGATAGCCTGGTGAAAG |
| | | | | | TTGAAGTTACCATTGAACCGCTGGG(TAA) |
| luxsiti_ | 18 | MSEEEIKEFWRRFYQA | 49. | 218 | (ATG)AGCGAAGAAGAAATTAAAGAATTTTGGC |
| 0.3_ | | LDAGDAETASALFPDG | 79820318 | | GCCGCTTTTATCAGGCGCTGGATGCGGGTGATG |
| 506 | | TEIYLWDGTTERTRAE | | | CCGAAACCGCAAGCGCATTGTTTCCGGATGGCA |
| | | FRAWFEALRATSDAAS | | | CCGAAATTTATCTGTGGGATGGTACCACCTTTC |
| | | RHIVKLEVKGNRAEVE | | | GCACCCGCGCGGAATTTCGTGCATGGTTTGAAG |
| | | VVLRARVDGEERVVRL | | | CGCTGCGTGCAACCAGCGATGCGGCGTCTAGAC |
| | | RHIFEFEGDRLKRVEV | | | ATATTGTGAAACTGGAAGTGAAAGGCAACCGCG |
| | | EIEPL | | | CCGAAGTGGAAGTTGTGCTGCGCGCGAGAGTTG |
| | | | | | ATGGTGAAGAACGCGTTGTGCGCCTGCGCCATA |
| | | | | | TTTTTGAATTTGAAGGCGATCGTCTGAAACGCG |
| | | | | | TCGAAGTTGAAATTGAACCGCTGGG(TAA) |
| luxsiti_ | 19 | MSEEEIREFWRREYEA | 2. | 219 | (ATG)AGCGAAGAAGAAATTCGTGAATTTTGGC |
| 0.3_ | | LDAGDAETASALFPDG | 348306842 | | GCCGCTTTTATGAAGCGCTGGATGCGGGCGATG |
| 515 | | TKIYLWDGIVESTKEE | | | CGGAAACCGCAAGCGCACTGTTTCCAGATGGTA |
| | | FRSWFVELKSKSDNAK | | | CCAAAATTTATCTGTGGGATGGCATTGTGTTTA |
| | | RHVVSLRVDGNVANVK | | | GCACCAAAGAAGAGTTTCGCAGCTGGTTTGTGG |
| | | VVLEAEINGEKKVVEL | | | AACTGAAAAGCAAAAGCGATAATGCGAAACGCC |
| | | THTTKFEGDKLVEVNV | | | ATGTGGTGAGCCTGCGCGTGGATGGTAACGTGG |
| | | DINPL | | | CCAACGTGAAAGTGGTGCTGGAAGCGGAAATCA |
| | | | | | ACGGCGAAAAGAAAGTTGTGGAGCTGACCCATA |
| | | | | | CCACCAAATTTGAAGGCGATAAACTGGTGGAAG |
| | | | | | TGAACGTGGATATTAACCCGCTGGG(TAA) |
| luxsiti_ | 20 | MSEEEQRKFWEKFYEA | 26. | 220 | (ATG)AGCGAAGAAGAACAGCGCAAATTTTGGG |
| 0.3_ | | LDAGDAETASALEKDG | 05459572 | | AAAAATTTTATGAAGCGCTGGATGCGGGCGATG |
| 517 | | TKIYLWDGTVFETQEE | | | CCGAAACCGCAAGCGCATTGTTTAAAGATGGCA |
| | | FRAWFEKLYSTSDNAQ | | | CCAAAATTTATCTGTGGGATGGTACCGTTTTTG |
| | | RHIVSFKVDGNISYVE | | | AAACCCAGGAAGAATTTCGCGCGTGGTTTGAAA |
| | | VVLHANVNGEEKTVKL | | | AACTGTATAGCACCAGCGATAACGCGCAGCGCC |
| | | THLFKFEGDHVVEVKV | | | ATATTGTGAGCTTTAAAGTGGATGGCAACATTA |
| | | KIEPL | | | GCTATGTGGAAGTGGTGCTGCATGCGAACGTGA |
| | | | | | ACGGTGAAGAAAAGACCGTGAAACTGACCCACC |
| | | | | | TGTTCAAATTTGAAGGCGATCATGTGGTTGAGG |
| | | | | | TGAAAGTGAAAATTGAACCGCTGGG{TAA) |
| luxsiti_ | 21 | MSEEEQKEFWKKFYDA | 2. | 221 | (ATG)AGCGAAGAAGAACAGAAAGAATTTTGGA |
| 0.3_ | | LDAGDAETASALFPDG | 204561161 | | AAAAGTTTTACGATGCGCTGGATGCGGGCGATG |
| 519 | | TKIHLWDGKTFTTQAE | | | CAGAAACTGCGAGCGCACTGTTTCCGGATGGCA |
| | | FKAWFVDLKSKSDDAK | | | CCAAAATTCATCTGTGGGATGGTAAAACCTTTA |
| | | RYITKFKVDGNKAYIE | | | CCACCCAGGCCGAATTTAAAGCGTGGTTTGTGG |
| | | VVLKASVKGKKKTVKL | | | ATCTGAAAAGCAAAAGCGATGATGCGAAACGCT |
| | | KHTAQFEGDKLVEVDV | | | ATATTACCAAATTCAAAGTGGATGGAAACAAAG |
| | | TINPL | | | CGTATATTGAAGTGGTGCTGAAAGCGAGCGTGA |
| | | | | | AAGGCAAGAAAAAGACCGTGAAACTGAAACATA |
| | | | | | CCGCGCAGTTTGAAGGCGATAAACTGGTGGAAG |
| | | | | | TGGACGTGACCATTAACCCGCTGGG(TAA) |
| luxsiti_ | 22 | MSEEAIREFVRRFYEA | 1647. | 222 | (ATG)AGCGAAGAAGCGATTCGCGAATTTGTGC |
| 0.3_ | | LDAGDAETASALFPDG | 027643 | | GCCGCTTTTATGAAGCGCTGGATGCAGGCGATG |
| 521 | | TRIHLWDGTTERTRAE | | | CGGAAACTGCGAGCGCACTGTTTCCGGATGGCA |
| | | FRAWEVELHSKSDNAQ | | | CCCGCATTCATCTGTGGGATGGTACCACCTTTC |
| | | REVVRLEVEGNRARVE | | | GTACCCGTGCGGAATTTCGCGCGTGGTTTGTGG |
| | | VVLKANYKGKEKTVRL | | | AACTGCATAGCAAAAGCGATAACGCGCAACGCG |
| | | RHEFLFEGDALVEVRV | | | AAGTGGTGCGCCTGGAAGTGGAAGGCAACCGTG |
| | | EIEPL | | | CAAGAGTTGAAGTTGTGCTGAAAGCGAACTATA |
| | | | | | AAGGCAAAGAAAAGACCGTGCGTCTGCGCCATG |
| | | | | | AATTTCTGTTTGAAGGCGACGCGCTGGTCGAAG |
| | | | | | TGCGCGTTGAAATTGAACCGCTGGG(TAA) |
| luxsiti_ | 23 | MSAEAIREFWRRFYEA | 2. | 223 | (ATG)AGCGCGGAAGCGATCCGCGAATTTTGGC |
| 0.3_ | | LDAGDAETASALFPDG | 97650311 | | GCCGCTTTTATGAAGCGCTGGATGCAGGCGATG |
| 541 | | TEIHLWDGTTERTREE | | | CGGAAACCGCAAGCGCTCTGTTTCCGGATGGCA |
| | | FRAWFVELYSTSDNAK | | | CCGAAATTCATCTGTGGGATGGTACCACCTTTC |
| | | RKIVSLRVDGDRALVE | | | GTACGCGCGAAGAATTTCGCGCGTGGTTTGTGG |
| | | VVLRASVGGEEKTVKL | | | AACTGTATAGCACCAGCGATAACGCGAAACGCA |
| | | KHEALFEGDRLVEVRV | | | AAATTGTGAGCCTGCGCGTTGATGGCGATCGCG |
| | | QIEPL | | | CGCTGGTTGAAGTGGTGCTGAGAGCAAGCGTGG |
| | | | | | GTGGTGAAGAAAAGACCGTGAAACTGAAACATG |
| | | | | | AAGCCCTGTTTGAAGGAGATCGCCTGGTGGAAG |
| | | | | | TGCGCGTGCAGATTGAACCGCTGGG(TAA) |
| luxsiti_ | 24 | MSEEAQRKFVERFYKA | 90. | 224 | (ATG)AGCGAAGAAGCGCAGCGTAAATTTGTGG |
| 0.3_ | | LDAGDADTASALEPDG | 77332412 | | AACGCTTTTACAAAGCGCTGGATGCGGGTGATG |
| 543 | | TKIYLWDGKVETTQEE | | | CGGATACCGCAAGCGCGCTGTTTCCTGATGGCA |
| | | FRAWEVELYSKSENAK | | | CCAAAATTTATCTGTGGGATGGCAAAGTGTTTA |
| | | REVVSFNVDGDVAEVE | | | CCACCCAGGAAGAATTTCGCGCGTGGTTTGTTG |
| | | VVLHANVRGELKTVKL | | | AACTGTATAGCAAAAGCGAAAACGCGAAACGTG |
| | | KHTFKFEGDKLVEVNV | | | AAGTGGTGTCCTTTAACGTGGATGGCGATGTGG |
| | | TIKPL | | | CGGAAGTGGAAGTTGTGCTGCATGCGAACGTGC |
| | | | | | GCGGCGAACTGAAAACCGTGAAATTGAAACATA |
| | | | | | CCTTTAAATTCGAAGGCGATAAACTGGTTGAAG |
| | | | | | TGAACGTGACCATTAAACCGCTGGG(TAA) |
| luxsiti_ | 25 | MSAEAQREFWRRFYAA | 398. | 225 | (ATG)AGCGCGGAAGCGCAGCGCGAATTTTGGC |
| 0.3_ | | LDAGDAETASALFPDG | 2017968 | | GCCGCTTTTATGCGGCGTTGGATGCGGGCGATG |
| 564 | | TKIHLWDGKTFTTRAE | | | CGGAAACTGCAAGCGCGCTGTTTCCAGATGGCA |
| | | FREWEEKLYSTSDNAK | | | CCAAAATTCATCTGTGGGATGGCAAAACCTTTA |
| | | REITELKVDGDKSYVK | | | CCACCCGCGCCGAATTTCGCGAATGGTTTGAAA |
| | | VVLKANINGEEKTVNE | | | AACTGTATAGCACCTCTGATAACGCGAAACGTG |
| | | THEFQFEGDKLVEVNV | | | AAATTACCGAACTGAAAGTGGATGGCGATAAAA |
| | | TINPL | | | GCTATGTGAAAGTTGTGCTGAAAGCGAACATTA |
| | | | | | ACGGCGAAGAAAAGACCGTGAACCTGACCCATG |
| | | | | | AATTTCAGTTTGAAGGCGACAAACTGGTGGAAG |
| | | | | | TGAACGTGACCATTAACCCGCTGGG(TAA) |
| luxsiti_ | 26 | MSEQAIREFWKRFYNA | 1. | 226 | (ATG)AGCGAACAGGCGATTCGCGAATTTTGGA |
| 0.3_ | | LDAGDADTASALFPDG | 360055287 | | AACGCTTTTATAACGCGCTGGATGCTGGTGATG |
| 568 | | TVIHLWDGKTFHTQAE | | | CGGATACCGCGAGCGCGCTGTTTCCTGATGGTA |
| | | FRKWFVDLYSRSDNAK | | | CCGTGATTCATCTGTGGGATGGCAAAACCTTTC |
| | | REIVSLEVDGNHAKIV | | | ATACCCAGGCGGAATTTCGCAAATGGTTTGTGG |
| | | VVLNAIINGEKKTVLL | | | ATCTGTATTCCCGCAGCGATAACGCCAAACGCG |
| | | THETLFEGDHLVEVFV | | | AAATTGTGAGCCTGGAAGTGGATGGTAACCATG |
| | | EIKPL | | | CGAAAATTGTTGTGGTGCTGAATGCCATTATTA |
| | | | | | ACGGCGAAAAGAAAACCGTGCTGCTGACCCATG |
| | | | | | AAACCCTGTTTGAAGGCGATCATCTGGTGGAAG |
| | | | | | TTTTTGTTGAAATTAAACCGCTGGG(TAA) |
| luxsiti_ | 27 | MSEEEQREFWKRFYEA | 146. | 227 | (ATG)AGCGAAGAAGAACAGCGCGAATTTTGGA |
| 0.3_ | | LDAGDAETASALEPDG | 1630961 | | AACGCTTTTATGAAGCGCTGGATGCGGGCGATG |
| 582 | | TKIHLWDGTTFTTQAE | | | CGGAAACTGCAAGCGCACTGTTTCCGGATGGCA |
| | | FRAWFVRLHSTSENAS | | | CCAAAATTCATCTGTGGGATGGTACCACCTTTA |
| | | RSIVSFKVEGNKALVE | | | CCACCCAGGCGGAATTTCGCGCGTGGTTTGTGC |
| | | VVLRASVKGEEKTVKL | | | GCCTGCATAGCACCAGCGAAAACGCCAGCCGTA |
| | | THEFQFEGDKLVEVNV | | | GTATTGTGAGCTTTAAAGTGGAAGGCAACAAAG |
| | | DIKPL | | | CGTTGGTGGAAGTGGTGCTGCGCGCGAGCGTGA |
| | | | | | AAGGTGAAGAAAAGACCGTGAAACTGACCCATG |
| | | | | | AATTTCAGTTTGAAGGCGATAAACTGGTTGAAG |
| | | | | | TGAACGTGGATATTAAACCGCTGGG(TAA) |
| luxsiti_ | 28 | MSEEDIRREVERFYAA | 2. | 228 | (ATG)AGCGAAGAAGATATTCGCCGCTTTGTGG |
| 0.3_ | | LDAGDAETASALFPDG | 874222529 | | AACGCTTTTATGCAGCGCTGGATGCGGGCGATG |
| 584 | | TRIHLWDGTTFTTREE | | | CGGAAACTGCGAGCGCATTGTTTCCGGATGGCA |
| | | FRAWFEKLHATSENAR | | | CCCGCATTCATCTGTGGGATGGAACCACCTTTA |
| | | RHVVKLKVEGNKAYVE | | | CCACCCGTGAAGAATTTCGCGCGTGGTTTGAAA |
| | | VILHASFKGKEKTVKL | | | AACTGCACGCGACCAGCGAAAACGCGCGCCGCC |
| | | THVFLFEDDRIVEVYV | | | ATGTGGTGAAACTGAAAGTGGAAGGTAACAAAG |
| | | KIEPL | | | CGTATGTGGAAGTGATTCTGCATGCCAGCTTTA |
| | | | | | AAGGCAAAGAAAAGACCGTTAAACTGACCCATG |
| | | | | | TGTTTCTGTTTGAAGATGATCGCCTGGTTGAAG |
| | | | | | TGTATGTGAAAATTGAACCGCTGGG(TAA) |
| luxsiti_ | 29 | MSEEEQREFWEKFYNA | 1756. | 229 | (ATG)AGCGAAGAAGAACAGCGCGAATTTTGGG |
| 0.3_ | | LDAGDAETASSLFPDG | 351071 | | AAAAATTTTATAACGCGCTGGATGCGGGTGATG |
| 596 | | TEIYLWDGTVEKTREE | | | CCGAAACCGCAAGTAGCCTGTTTCCGGATGGCA |
| | | FRAWFEKLHSTSENAK | | | CCGAAATTTATCTGTGGGATGGTACCGTGTTTA |
| | | RHIVSFEVDGNKSKVK | | | AAACCCGTGAAGAATTTCGCGCGTGGTTTGAAA |
| | | VVLHAKINGEEVTVEL | | | AACTGCATAGCACCAGCGAAAACGCGAAACGCC |
| | | EHVYEFEGDKLKKVEV | | | ATATTGTGAGCTTTGAAGTGGACGGCAACAAAA |
| | | EIKPL | | | GCAAAGTGAAAGTGGTGCTGCATGCGAAAATTA |
| | | | | | ACGGAGAAGAAGTGACCGTGGAACTGGAACATG |
| | | | | | TGTATGAATTTGAAGGCGATAAACTGAAAAAGG |
| | | | | | TGGAAGTTGAAATTAAACCGCTGGG{TAA) |
| luxsiti_ | 30 | MSEIAQREFWRRFYAA | 2. | 230 | (ATG)AGCGAAATTGCGCAGCGCGAATTTTGGC |
| 0.4_ | | LDAGDAETASALFPDG | 523151348 | | GCCGCTTTTATGCGGCGCTGGATGCGGGTGATG |
| 600 | | TEIYLWDGRTFHTREE | | | CGGAAACTGCAAGCGCGCTGTTTCCAGATGGCA |
| | | FEAWFRELYSKSENAR | | | CGGAAATTTATCTGTGGGATGGCCGCACCTTTC |
| | | REITSFTVIGDIAEIR | | | ATACCCGCGAAGAATTTGAAGCGTGGTTTCGCG |
| | | VVLRATIDGEKRTVSL | | | AACTGTATAGCAAAAGCGAAAACGCGCGTCGCG |
| | | NHVTHFEGDQLVRVEV | | | AAATCACCTCTTTTACCGTGACCGGCGATATTG |
| | | SIFPL | | | CCGAAATTCGCGTGGTGCTGCGCGCGACCATTG |
| | | | | | ATGGCGAAAAACGCACCGTGAGCCTGAACCATG |
| | | | | | TGACCCATTTTGAAGGCGATCAGCTGGTGCGCG |
| | | | | | TGGAAGTGAGCATTTTTCCGCTGGG(TAA) |
| luxsiti_ | 31 | MSEEEQKEFWKRFYEA | 1. | 231 | (ATG)AGCGAAGAAGAACAGAAAGAATTTTGGA |
| 0.4_ | | LDSGDADTASALEPDG | 472011057 | | AACGCTTTTATGAAGCGCTGGATAGCGGCGATG |
| 601 | | TKIYLWDGTVFETKAQ | | | CGGATACAGCGTCCGCTCTGTTTCCGGATGGCA |
| | | FKAWFVELYSRSDNAQ | | | CCAAAATTTATCTGTGGGACGGCACCGTGTTTG |
| | | RSITKFEVDGNTSRVV | | | AAACCAAAGCGCAGTTTAAAGCGTGGTTTGTGG |
| | | VVLKATVRGKEKVVEL | | | AACTGTATAGCCGCAGCGATAACGCGCAGCGCA |
| | | EHVFEFEGDELKEVKV | | | GCATTACCAAATTTGAAGTGGATGGTAACACCA |
| | | EIHPL | | | GCCGCGTGGTGGTTGTGCTGAAAGCGACCGTGC |
| | | | | | GCGGCAAAGAAAAAGTGGTTGAACTGGAACATG |
| | | | | | TGTTCGAATTCGAAGGCGATGAACTGAAAGAAG |
| | | | | | TGAAAGTTGAAATTCATCCGCTGGG(TAA) |
| luxsiti_ | 32 | MSEEAIREFVKRFYDA | 409. | 232 | (ATG)AGCGAAGAAGCGATTCGCGAATTTGTGA |
| 0.4_ | | LDAGDADTASALEPDG | 7712509 | | AACGCTTTTATGATGCCCTGGATGCGGGCGATG |
| 605 | | TLIHLWDGKTERTQAE | | | CGGATACCGCCTCTGCCTTGTTTCCAGATGGCA |
| | | FRAWFEELYSKSENAK | | | CCCTGATTCATCTGTGGGATGGCAAAACCTTTC |
| | | RHVVKLQVDGDRAQVE | | | GTACCCAGGCGGAATTTCGCGCCTGGTTTGAAG |
| | | VVLHANIDGQEVVVRL | | | AACTGTATAGCAAAAGCGAAAACGCGAAACGCC |
| | | RHEFLFEGDRIVEVYV | | | ATGTGGTGAAACTGCAGGTGGATGGCGATCGCG |
| | | QIQPL | | | CGCAGGTCGAAGTGGTGCTGCATGCGAACATTG |
| | | | | | ATGGCCAGGAAGTTGTGGTGCGCCTGCGCCATG |
| | | | | | AATTTCTGTTCGAAGGCGATAGACTGGTGGAAG |
| | | | | | TGTATGTGCAGATTCAGCCGCTGGG(TAA) |
| luxsiti_ | 33 | MTAEAQRAFWDRFYRA | 293. | 233 | (ATG)ACCGCCGAAGCGCAGCGCGCGTTTTGGG |
| 0.4_ | | LDAGDAETASSLEPDG | 3476158 | | ATCGCTTTTATCGCGCGCTTGATGCGGGCGATG |
| 606 | | TEIKLWDGKTFHTREE | | | CGGAAACCGCAAGCAGCCTGTTTCCAGATGGCA |
| | | FRQWFENLRSKSENAR | | | CCGAAATTCATCTGTGGGATGGTAAAACCTTTC |
| | | RHIVDERVDGDRADVR | | | ATACCCGCGAAGAATTTCGCCAGTGGTTTGAAA |
| | | VVLRANYQGEERVVQL | | | ACCTGCGCAGCAAAAGCGAAAACGCGCGCCGCC |
| | | HHEFLFEGDQLVRVEV | | | ATATTGTGGATTTTCGCGTGGATGGCGATCGCG |
| | | RIIPE | | | CAGATGTGCGCGTGGTGCTGCGTGCAAACTATC |
| | | | | | AGGGTGAAGAACGCGTTGTGCAGCTGCATCATG |
| | | | | | AATTTCTGTTTGAAGGCGATCAGCTGGTGCGTG |
| | | | | | TGGAAGTGCGCATTATTCCGGAAGG(TAA) |
| luxsiti_ | 34 | MSEKEQREFCARFYAA | 2. | 234 | (ATG)AGCGAAAAAGAACAGCGCGAATTTTGCG |
| 0.4_ | | LDAGDAETASALFPDG | 787836904 | | CGCGCTTTTATGCGGCCCTGGATGCGGGTGATG |
| 610 | | TRIHLWDGRTETTQAE | | | CGGAAACTGCAAGCGCGTTGTTTCCGGATGGCA |
| | | FGAWERRLRSTSDNAR | | | CCCGCATTCATCTGTGGGATGGCCGCACCTTTA |
| | | RHIVDERVDGDVAKVR | | | CCACCCAGGCGGAATTTGGCGCGTGGTTTCGTC |
| | | VVLHASVGGRERTVQL | | | GTCTGCGTAGCACAAGCGATAACGCGCGCCGTC |
| | | EHVFIFEGDRLVEVHV | | | ATATTGTGGATTTTCGCGTGGATGGCGATGTGG |
| | | SIQPE | | | CGAAAGTGCGCGTAGTGTTGCATGCGAGCGTGG |
| | | | | | GTGGTAGAGAACGCACCGTGCAGCTGGAACATG |
| | | | | | TGTTTATTTTTGAAGGCGATCGCCTGGTGGAAG |
| | | | | | TGCATGTGAGCATTCAGCCGGAAGG(TAA) |
| luxsiti_ | 35 | MSEEEQRKFWEKFYNA | 5. | 235 | (ATG)AGCGAAGAAGAACAGCGCAAATTTTGGG |
| 0.4_ | | LDAGDAETASALFPDG | 106427091 | | AAAAATTTTATAACGCGCTTGATGCGGGCGATG |
| 611 | | TEIYLWDGKVERTRAE | | | CGGAAACCGCAAGCGCACTGTTTCCGGATGGTA |
| | | FREWFERLYSKSDNAQ | | | CCGAAATTTATCTGTGGGATGGCAAAGTGTTTC |
| | | RSITSFEVNGNVAEIK | | | GCACCCGCGCGGAATTTCGCGAATGGTTTGAAC |
| | | VILKANVNGEEKEVSL | | | GCCTGTATTCGAAAAGCGATAACGCCCAGCGCA |
| | | NHKTEFEGDKLVKVEV | | | GCATTACCAGCTTTGAAGTGAACGGCAACGTGG |
| | | DISPL | | | CGGAAATTAAAGTGATTCTGAAAGCGAACGTTA |
| | | | | | ACGGTGAAGAAAAAGAAGTGAGCCTGAACCATA |
| | | | | | AAACCGAATTTGAAGGCGATAAACTGGTGAAAG |
| | | | | | TGGAAGTGGATATTAGCCCGCTGGG(TAA) |
| luxsiti_ | 36 | MSEKEQREFWKKFYEA | 6. | 236 | (ATG)AGCGAGAAAGAACAGCGCGAATTTTGGA |
| 0.4_ | | LDAGDAETAAALFPDG | 803040774 | | AAAAGTTTTATGAAGCGCTGGATGCGGGCGATG |
| 613 | | TVIHLWDGKTFHTQEE | | | CGGAAACCGCAGCAGCCTTGTTTCCAGATGGTA |
| | | FKEWFIKIRSTSENAK | | | CCGTGATTCATCTGTGGGATGGCAAAACCTTTC |
| | | RKIISEKVDGNISYVE | | | ATACCCAGGAAGAATTTAAAGAATGGTTTATTA |
| | | VELKAKIKGESKVVKL | | | AACTGCGCAGCACCTCTGAAAACGCGAAACGCA |
| | | RHIFKFEGDKLVEVDV | | | AAATTATTAGTTTTAAAGTGGATGGTAACATTA |
| | | TIKPI | | | GCTATGTTGAAGTGGAACTGAAAGCGAAAATTA |
| | | | | | AAGGTGAAAGCAAAGTGGTGAAACTGAGACATA |
| | | | | | TTTTTAAATTTGAAGGTGATAAACTGGTGGAAG |
| | | | | | TCGATGTGACCATTAAACCGATTGG(TAA) |
| luxsiti_ | 37 | MSAEEIKEFCRRFYEA | 68. | 237 | (ATG)AGCGCGGAAGAAATTAAAGAATTTTGCC |
| 0.4_ | | LDAGDAETASALFPDG | 170698 | | GCCGCTTTTATGAAGCGCTGGATGCGGGCGATG |
| 614 | | TIIHLWDGRTFHTQAE | | | CGGAAACCGCAAGCGCGTTGTTTCCTGATGGCA |
| | | FRAWFRELRSTSEDAK | | | CCATTATTCATCTGTGGGATGGCCGTACCTTTC |
| | | REIERLEVDGNQAKVE | | | ATACCCAGGCGGAATTTCGCGCGTGGTTTCGCG |
| | | VILHANRDGEKKVVRL | | | AACTGCGCAGCACCAGCGAAGATGCGAAACGCG |
| | | THEFLFEGDRIVEVTV | | | AAATTGAACGCCTGGAAGTGGATGGCAACCAGG |
| | | AIRPL | | | CCAAAGTGGAAGTTATTCTGCATGCGAACCGCG |
| | | | | | ATGGCGAAAAGAAAGTGGTGCGCCTGACCCATG |
| | | | | | AATTTCTGTTTGAAGGCGATCGCCTGGTTGAAG |
| | | | | | TGACCGTGGCGATCCGCCCGCTGGG(TAA) |
| luxsiti_ | 38 | MSKQEIKDECEKFYNA | 1. | 238 | (ATG)AGCAAACAGGAAATTAAAGATTTTTGCG |
| 0.4_ | | LDAGDADTASALEKDG | 216309606 | | AAAAATTTTATAACGCGCTGGATGCGGGCGATG |
| 639 | | TKIYLWDGKTFETQAE | | | CGGATACCGCGAGCGCACTGTTCAAAGATGGCA |
| | | FKAWFTKLHSTSDNAK | | | CCAAAATTTATCTGTGGGATGGCAAAACCTTTG |
| | | RYITSLEVDGDKAFVK | | | AAACCCAGGCGGAATTTAAAGCGTGGTTTACCA |
| | | VVLKANVNGEEKTVEL | | | AACTGCATAGCACCAGCGATAATGCGAAACGCT |
| | | THIFEFEGDELVKVQV | | | ATATTACCAGCCTGGAAGTGGATGGCGATAAAG |
| | | KIKPL | | | CCTTTGTGAAAGTGGTGCTGAAAGCCAACGTGA |
| | | | | | ATGGTGAAGAAAAGACCGTGGAACTGACTCATA |
| | | | | | TTTTTGAATTTGAAGGCGATGAACTGGTTAAAG |
| | | | | | TGCAGGTTAAAATTAAGCCGCTGGG(TAA) |
| luxsiti_ | 39 | MSEEEIRQFIKRFYDA | 673. | 239 | (ATG)AGCGAAGAAGAAATTCGCCAGTTTATTA |
| 0.4_ | | LDAGDAETASALFPDG | 3462336 | | AACGCTTTTATGATGCGCTGGATGCGGGCGATG |
| 670 | | TEIDLWDGTTFHTQEE | | | CCGAAACTGCGAGCGCATTGTTTCCGGATGGCA |
| | | FRSWFVRLKSTSDNAK | | | CCGAAATTGATCTGTGGGATGGTACCACCTTTC |
| | | RHVVALEVQGNRAKVE | | | ATACCCAGGAAGAATTTCGCTCGTGGTTTGTGC |
| | | VVLEASIDGKKITVQL | | | GTCTGAAAAGCACCAGCGATAACGCGAAACGCC |
| | | THEFYFEGDKLVKVNV | | | ATGTGGTGGCGTTAGAAGTGCAGGGCAACCGTG |
| | | TIKPL | | | CGAAAGTGGAAGTGGTGCTGGAAGCCAGCATTG |
| | | | | | ATGGCAAGAAAATTACCGTGCAGCTGACCCATG |
| | | | | | AATTTTATTTTGAAGGCGATAAACTGGTGAAAG |
| | | | | | TGAACGTGACCATTAAACCGCTGGG(TAA) |
| luxsiti_ | 40 | MSEEEIREFVRRFYAA | 625. | 240 | (ATG)AGCGAAGAAGAAATCCGCGAATTTGTGC |
| 0.4_ | | LDAGDAETASALFPDG | 715273 | | GCCGCTTTTATGCGGCGCTGGATGCGGGTGATG |
| 679 | | TVIHLWDGRTEHTQAE | | | CGGAAACTGCAAGCGCGCTGTTTCCAGATGGCA |
| | | FRAWFEELYSRSENAK | | | CCGTGATTCATCTGTGGGATGGCCGCACCTTTC |
| | | REVTQLEVDGDHAFVK | | | ATACCCAGGCGGAATTTCGTGCGTGGTTTGAAG |
| | | VVLRANIHGEEKVVGL | | | AACTGTATAGCCGCAGCGAAAACGCGAAACGTG |
| | | EHYFHFEGDQLKEVEV | | | AAGTGACCCAGCTGGAAGTGGATGGCGATCATG |
| | | VIRPE | | | CGTTTGTGAAAGTGGTGCTGCGCGCGAACATTC |
| | | | | | ATGGTGAAGAAAAAGTTGTGGGCCTGGAACATT |
| | | | | | ATTTTCATTTTGAAGGTGATCAGCTGAAAGAAG |
| | | | | | TTGAAGTGGTTATTCGTCCGGAAGG(TAA) |
| luxsiti_ | 41 | MSKEEIEEFWKKFYQA | 2. | 241 | (ATG)AGCAAAGAAGAAATTGAAGAATTTTGGA |
| 0.4_ | | LDAGDAETASALFPDG | 568762958 | | AAAAGTTTTATCAGGCCCTGGATGCGGGCGATG |
| 687 | | TVIHLWDGKVEHTKAE | | | CGGAAACCGCAAGCGCCTTGTTTCCTGATGGCA |
| | | FREWFVKLKSESEDAK | | | CCGTGATTCATCTGTGGGATGGCAAAGTGTTTC |
| | | REIVSLRVDGNKAEVE | | | ATACCAAAGCGGAATTTCGCGAATGGTTTGTGA |
| | | VVLKAKYKGEEKEVRL | | | AACTGAAAAGCGAGAGCGAAGATGCGAAACGCG |
| | | KHESLFEGDRIVEVDV | | | AAATTGTGAGCCTGCGCGTGGATGGTAACAAAG |
| | | DIFPL | | | CCGAGGTGGAAGTGGTGCTGAAAGCGAAATATA |
| | | | | | AAGGCGAAGAAAAAGAAGTGCGCCTGAAACATG |
| | | | | | AATCCCTGTTTGAAGGCGATCGCCTGGTCGAAG |
| | | | | | TGGATGTGGATATTTTTCCGCTGGG(TAA) |
| luxsiti_ | 42 | MSEEEIREFWKRFYEA | 1. | 242 | (ATG)AGCGAAGAAGAAATCCGCGAATTTTGGA |
| 0.4_ | | LDAGDAETASALEKDG | 768486524 | | AACGCTTTTATGAAGCGCTGGATGCGGGCGATG |
| 695 | | TKIYLWDGITFFTRDE | | | CCGAAACCGCGAGCGCACTGTTTAAAGATGGCA |
| | | FRAWFEKLYSTSEDAK | | | CCAAAATTTATCTGTGGGATGGCATTACCTTCT |
| | | REIVKENVDGNKAKVE | | | TTACCCGCGATGAATTTCGCGCGTGGTTTGAAA |
| | | VVLHAIVDGQKKTVKL | | | AACTGTATAGCACCTCAGAAGATGCGAAACGCG |
| | | THTSYFEGDELKKVEV | | | AAATTGTGAAATTTAACGTGGATGGTAACAAAG |
| | | SIEPM | | | CGAAAGTGGAAGTGGTGCTGCATGCGATTGTTG |
| | | | | | ATGGCCAAAAGAAAACCGTGAAACTGACCCATA |
| | | | | | CCAGCTATTTTGAAGGTGATGAACTGAAAAAGG |
| | | | | | TCGAAGTGAGCATTGAACCGATGGG(TAA) |
| luxsiti_ | 43 | MSEEEIREFIKRFYEA | 89. | 243 | (ATG)AGCGAAGAAGAAATTCGCGAATTTATTA |
| 0.4_ | | LDAGDAETASALEPDG | 90393918 | | AACGCTTTTATGAAGCGCTGGATGCGGGCGATG |
| 707 | | TKIYLWDGTVESTQAE | | | CGGAAACCGCAAGCGCATTGTTTCCGGATGGCA |
| | | FRDWFVRLRSTSQDAR | | | CCAAAATTTATCTGTGGGATGGTACCGTGTTTT |
| | | RKVVDLKVDGDVADVE | | | CGACCCAGGCGGAATTTCGCGATTGGTTTGTGC |
| | | VVLRANVEGEERVVRL | | | GTCTGCGTAGCACCAGCCAGGATGCCCGCCGTA |
| | | RHVFHFEGDKVVEVEV | | | AAGTGGTTGATCTGAAAGTGGATGGTGATGTGG |
| | | EIEPE | | | CGGATGTGGAAGTGGTGCTGCGCGCGAACGTGG |
| | | | | | AAGGTGAAGAACGCGTGGTGCGACTGAGACATG |
| | | | | | TGTTTCATTTTGAAGGCGATAAAGTTGTTGAAG |
| | | | | | TCGAAGTGGAAATTGAACCGGAAGG(TAA) |
| luxsiti_ | 44 | MSEEEQREFWRRFYAA | 5. | 244 | (ATG)AGCGAAGAAGAACAGCGCGAATTTTGGC |
| 0.4_ | | LDAGDAETASALFPDG | 817553559 | | GCCGCTTTTATGCGGCGCTGGATGCGGGTGATG |
| 712 | | TKIYLWDGKTETTQAE | | | CGGAAACCGCATCAGCGCTGTTTCCTGATGGTA |
| | | FRAWERKLRSQSEGAS | | | CCAAAATTTATCTGTGGGATGGCAAAACCTTTA |
| | | RSVEYFEVDGNKSHVE | | | CCACCCAGGCGGAATTTCGCGCGTGGTTTCGCA |
| | | VVLRASVNGEQHVVKL | | | AACTGCGCAGCCAAAGCGAAGGCGCGTCTAGAA |
| | | RHEFLFEGDKLVEVEV | | | GCGTGGAATATTTTGAAGTGGATGGTAACAAAA |
| | | KIEPL | | | GCCATGTGGAAGTGGTGCTGCGCGCCAGCGTGA |
| | | | | | ACGGCGAACAGCATGTGGTGAAACTGAGACATG |
| | | | | | AATTTCTGTTTGAAGGCGATAAACTGGTTGAAG |
| | | | | | TCGAAGTGAAAATTGAACCGCTGGG(TAA) |
| luxsiti_ | 45 | MSEKAQREFWKKFYEA | 7. | 245 | (ATG)AGCGAAAAAGCGCAGCGCGAATTTTGGA |
| 0.4_ | | LDAGDADTASALFKDG | 188666206 | | AAAAGTTTTATGAAGCGCTGGATGCGGGCGATG |
| 718 | | TEIYLWDGKVEKKQEE | | | CCGATACCGCGAGCGCGCTGTTTAAAGATGGCA |
| | | FKEWFEKLHSTSENAK | | | CCGAAATTTATCTGTGGGATGGCAAAGTGTTCA |
| | | RKIVKFEVKGNVSYVE | | | AAAAGCAGGAAGAATTCAAAGAATGGTTTGAAA |
| | | VVLEASLAGEKKTVKL | | | AACTGCATAGCACCAGCGAGAACGCGAAACGTA |
| | | KHMFQFEGDKLVKVTV | | | AAATTGTGAAATTTGAAGTGAAAGGCAACGTGA |
| | | DIYPL | | | GCTATGTGGAAGTGGTGCTGGAAGCGAGCCTGG |
| | | | | | CGGGTGAAAAGAAAACCGTGAAACTGAAACACA |
| | | | | | TGTTTCAGTTTGAAGGCGATAAACTGGTGAAAG |
| | | | | | TGACCGTGGATATTTATCCGCTGGG(TAA) |
| luxsiti_ | 46 | MSEKAQREFVKRFYDA | 3. | 246 | (ATG)AGCGAGAAAGCGCAGCGCGAATTTGTGA |
| 0.4_ | | LDAGDADTASALEPDG | 718728404 | | AACGCTTTTATGATGCGTTGGATGCAGGCGATG |
| 721 | | TIIYLWDGKTERTQTE | | | CGGATACCGCGAGCGCACTGTTTCCGGATGGCA |
| | | FRRWEVDLKSTSENAK | | | CCATTATTTATCTGTGGGATGGTAAAACCTTTC |
| | | RKVTDFRVDGNVANVT | | | GCACCCAGACCGAATTTCGCCGCTGGTTTGTGG |
| | | VVLEANINGKKETVKL | | | ATCTGAAAAGCACCAGCGAAAACGCGAAACGCA |
| | | NHIFHWEGDRIVEVEV | | | AAGTTACCGATTTTCGCGTGGATGGAAACGTGG |
| | | TIEPL | | | CGAACGTGACCGTGGTTCTGGAAGCGAACATTA |
| | | | | | ACGGCAAGAAAGAAACCGTGAAACTGAACCATA |
| | | | | | TTTTTCATTGGGAAGGCGATCGCCTGGTGGAAG |
| | | | | | TTGAAGTGACCATTGAACCGCTGGG(TAA) |
| luxsiti_ | 47 | MSAEAIREFVRDFYEA | 201. | 247 | (ATG)TCTGCGGAAGCGATTCGCGAATTTGTGC |
| 0.4_ | | LDAGDADTASALFPDG | 8825155 | | GCGATTTTTATGAAGCGCTGGATGCGGGTGATG |
| 724 | | TEIHLWDGTVERTRAE | | | CGGATACCGCCAGCGCGTTATTTCCGGATGGCA |
| | | FKAWETKLYSTSEDAR | | | CGGAAATTCACCTGTGGGATGGTACCGTGTTTC |
| | | RKVVRLEVEGDVARVE | | | GTACCCGCGCGGAATTTAAAGCGTGGTTTACCA |
| | | VVLHASHAGKESTVKL | | | AACTGTATAGCACCAGCGAAGATGCGCGTCGCA |
| | | QHEFSFEGDRLVEVRV | | | AAGTGGTGCGTCTGGAAGTGGAAGGCGATGTGG |
| | | VIEPL | | | CGCGTGTTGAAGTGGTTCTGCATGCGAGCCATG |
| | | | | | CGGGCAAAGAAAGCACCGTGAAACTGCAGCATG |
| | | | | | AATTTAGCTTTGAAGGTGATCGCCTGGTGGAAG |
| | | | | | TTCGTGTGGTGATTGAACCGCTGGG(TAA) |
| luxsiti_ | 48 | MSEEEIKKECEKFYEA | 21. | 248 | (ATG)TCTGAAGAAGAAATCAAGAAATTTTGTG |
| 0.4_ | | LDAGDAETASALFPDG | 93365584 | | AAAAATTTTATGAAGCGCTGGATGCGGGCGATG |
| 747 | | TEIYLWDGRVEKTREE | | | CCGAAACTGCGAGCGCGTTGTTTCCTGATGGCA |
| | | FRAWFVELYSKSDNAQ | | | CCGAAATTTATCTGTGGGATGGCCGCGTGTTTA |
| | | RKIVDLKVDGNKAKVK | | | AAACCCGCGAAGAATTTCGCGCGTGGTTTGTGG |
| | | VVLHANHNGEQHKVAL | | | AACTGTATAGCAAAAGCGATAATGCGCAGCGCA |
| | | THEFLFEGDKLVEVNV | | | AAATTGTGGATCTGAAAGTGGATGGTAACAAAG |
| | | EIKPL | | | CGAAAGTGAAAGTTGTGCTGCATGCGAACCATA |
| | | | | | ACGGCGAACAGCATAAAGTGGCGCTGACCCATG |
| | | | | | AATTTCTGTTTGAAGGCGATAAACTGGTGGAAG |
| | | | | | TGAACGTGGAAATTAAACCGCTGGG(TAA) |
| luxsiti_ | 49 | MSEEATREFWKKFYEA | 1. | 249 | (ATG)AGCGAAGAAGCGATTCGCGAATTTTGGA |
| 0.4_ | | LDAGDAETASALEPDG | 249481686 | | AAAAGTTCTATGAAGCACTGGATGCGGGCGATG |
| 748 | | TIIHLWDGTTFTTQDE | | | CGGAAACCGCAAGCGCACTGTTTCCGGATGGCA |
| | | FKKWFVELFSKSENAS | | | CCATTATTCATCTGTGGGATGGTACCACCTTTA |
| | | RKIVKLEVKGNVAYVE | | | CCACCCAGGATGAATTTAAAAAGTGGTTTGTGG |
| | | VVLKAKIDGKEKTVRL | | | AACTGTTTAGTAAAAGCGAAAACGCGAGCCGCA |
| | | KHVTKFEGDKLVEVEV | | | AAATTGTGAAACTGGAAGTGAAAGGCAACGTGG |
| | | DIDPL | | | CGTATGTGGAAGTTGTGCTGAAAGCCAAAATCG |
| | | | | | ATGGCAAAGAAAAGACCGTGCGCCTGAAACATG |
| | | | | | TGACCAAATTTGAAGGCGATAAACTGGTTGAAG |
| | | | | | TCGAAGTGGATATTGATCCGCTGGG(TAA) |
| luxsiti_ | 50 | MSADDQKEFWKRFYEA | 1. | 250 | (ATG)AGCGCGGATGATCAGAAAGAATTTTGGA |
| 0.4_ | | LDAGNADEASALEPDG | 436074637 | | AACGTTTTTACGAAGCGCTGGATGCGGGCAATG |
| 750 | | TEIYLWDGTTFKTKAQ | | | CAGATGAAGCGAGCGCGCTGTTTCCGGATGGCA |
| | | FKAWFCQLRSTSENAK | | | CCGAAATCTATCTGTGGGATGGTACCACCTTTA |
| | | REIVKFEVDGNFSYVE | | | AAACCAAAGCGCAGTTTAAAGCGTGGTTCTGCC |
| | | VVLEATVNGKKFIVSL | | | AGCTGCGCAGCACCAGCGAAAACGCGAAACGTG |
| | | DHYFEFEGEQLVEVYV | | | AAATTGTTAAATTTGAAGTGGATGGGAACTTTA |
| | | KIRPL | | | GCTATGTGGAAGTGGTGCTGGAAGCGACCGTGA |
| | | | | | ACGGCAAGAAATTTATTGTGAGCCTGGATCATT |
| | | | | | ATTTTGAATTTGAGGGCGAACAGCTGGTTGAAG |
| | | | | | TCTATGTGAAAATTCGCCCGCTGGG(TAA) |
| luxsiti_ | 51 | MSAKAQREFWKKFYEA | 2. | 251 | (ATG)TCTGCGAAAGCGCAGCGTGAATTTTGGA |
| 0.4_ | | LDAGDADTAAALFPDG | 566689703 | | AAAAGTTTTATGAAGCGCTGGATGCGGGTGATG |
| 766 | | TRIHLWDGRTETTQAE | | | CGGATACCGCAGCAGCACTGTTTCCGGATGGCA |
| | | FRAWFQTLHSRSDNAK | | | CCCGCATTCATCTGTGGGATGGCCGTACCTTTA |
| | | RSIVAFNVSGDVSDVV | | | CCACCCAGGCGGAATTTCGCGCGTGGTTTCAGA |
| | | VVLRASYEGEERTVRL | | | CCCTGCATAGCCGCAGCGATAACGCGAAACGCT |
| | | RHTFRFEGDHLVEVDV | | | CTATCGTGGCGTTTAACGTGAGCGGTGATGTGA |
| | | AIDPL | | | GCGATGTGGTGGTTGTGCTGCGCGCGAGCTATG |
| | | | | | AAGGCGAAGAACGCACCGTGCGTCTGCGTCATA |
| | | | | | CCTTTCGCTTTGAAGGTGATCATCTGGTGGAAG |
| | | | | | TTGATGTGGCGATTGATCCGCTGGG(TAA) |
| luxsiti_ | 52 | MSEAEQREFWRRFYEA | 1. | 252 | (ATG)AGCGAAGCGGAACAGCGCGAATTTTGGC |
| 0.4_ | | LDAGDAETASALFPDG | 00601935 | | GCCGCTTTTATGAAGCGCTGGATGCGGGCGATG |
| 770 | | TEIHEWDGKTEHTQAE | | | CGGAAACTGCAAGCGCGTTGTTTCCAGATGGCA |
| | | FRAWFVELRSTSEAAR | | | CCGAAATTCATCTGTGGGATGGCAAAACCTTTC |
| | | RHVVAFNVDGDRARVE | | | ATACCCAGGCGGAATTTCGCGCGTGGTTTGTGG |
| | | VVLHAVRDGEARTVQL | | | AATTACGCAGCACCTCTGAAGCGGCGCGCCGTC |
| | | THETVFAGDQVVRVRV | | | ATGTGGTTGCGTTTAACGTGGATGGCGATCGCG |
| | | QIKPL | | | CTAGAGTGGAAGTCGTGCTGCATGCGGTGCGTG |
| | | | | | ATGGTGAAGCGAGAACCGTGCAGCTGACCCATG |
| | | | | | AAACCGTGTTTGCGGGTGATCAGGTGGTGCGCG |
| | | | | | TGCGTGTGCAGATTAAACCGCTGGG(TAA) |
| luxsiti_ | 53 | MSEEAVREFVARFYEA | 1. | 253 | (ATG)AGCGAAGAAGCGGTGCGCGAATTTGTTG |
| 0.4_ | | LDAGDADTASALEPDG | 843814789 | | CGCGCTTTTATGAAGCGCTGGATGCGGGTGATG |
| 812 | | TVIHLWSGQTFHTRAE | | | CGGATACCGCGAGCGCATTGTTTCCGGATGGCA |
| | | FRAWFERLYAESAAAR | | | CCGTGATTCATCTGTGGAGCGGCCAGACCTTTC |
| | | RRVVEMRVDGDVAFVE | | | ATACCCGCGCGGAATTTCGCGCGTGGTTTGAAC |
| | | VVLHASHQGQPQVVRL | | | GCCTGTATGCGGAAAGCGCGGGGCGAGAAGAA |
| | | RHIFRFEGDRLREVEV | | | GAGTGGTGGAAATGCGCGTTGATGGCGATGTGG |
| | | SIDPL | | | CGTTTGTGGAAGTGGTTCTGCATGCGAGCCATC |
| | | | | | AGGGCCAGCCGCAGGTGGTTCGTCTGAGACATA |
| | | | | | TTTTTCGCTTTGAAGGCGATCGTCTGCGTGAAG |
| | | | | | TCGAAGTGAGCATCGATCCGCTGGG(TAA) |
| luxsiti_ | 54 | MSAEQQREFWDRFYAA | 1. | 254 | (ATG)AGCGCGGAACAGCAGCGCGAATTTTGGG |
| 0.4_ | | LDAGDADTASALENDG | 08016586 | | ATCGCTTTTATGCGGCGCTGGATGCGGGCGATG |
| 837 | | TEIYLWDGKVFHTQAE | | | CGGATACCGCAAGCGCATTGTTTAACGATGGCA |
| | | FKAWFVDLRSRSTGAS | | | CCGAAATTTATCTGTGGGATGGCAAAGTGTTTC |
| | | REIVAFKVTGNRAEIE | | | ATACCCAGGCGGAATTTAAAGCGTGGTTTGTGG |
| | | VVLHADVDGQPKTVRE | | | ATCTGCGCAGCCGCAGCACCGGCGCGAGCAGAG |
| | | THYTEFEGDRLVRVEV | | | AAATTGTGGCGTTTAAAGTGACCGGCAACCGCG |
| | | KINPL | | | CCGAAATCGAAGTGGTGCTGCATGCAGATGTTG |
| | | | | | ATGGCCAGCCGAAAACCGTGCGCCTGACCCATT |
| | | | | | ATACCGAATTTGAAGGCGATCGCCTGGTGCGCG |
| | | | | | TAGAAGTGAAAATTAATCCGCTGGG(TAA) |
| luxsiti_ | 55 | MSEQAIREFVRRFYDA | 1. | 255 | (ATG)AGCGAACAGGCGATTCGTGAATTTGTGC |
| 0.4_ | | LDAGDAETAAALFPDG | 07601935 | | GCCGCTTTTATGATGCGCTGGATGCGGGTGATG |
| 838 | | TVIHLWDGRTFHTRAE | | | CCGAAACCGCAGCTGCACTGTTTCCGGATGGCA |
| | | FRAWFVRLRSTSDDAR | | | CCGTGATTCATCTGTGGGATGGCCGCACCTTTC |
| | | RRVVELRVVGDRARVR | | | ATACCCGCGCGGAATTTCGCGCGTGGTTTGTGA |
| | | VVLQANVDGEPRVVEL | | | GGCTGCGTAGCACTAGCGATGATGCCCGCCGTA |
| | | EHEFEFEGDRLREVRV | | | GGGTGGTGGAACTGCGTGTGGTTGGTGATCGTG |
| | | DIRPL | | | CTAGAGTGCGCGTTGTGCTGCAGGCCAACGTGG |
| | | | | | ATGGCGAACCGAGAGTGGTTGAACTGGAACACG |
| | | | | | AATTTGAATTCGAAGGCGATCGTCTGCGCGAAG |
| | | | | | TGCGTGTTGATATTCGCCCGCTGGG(TAA) |
| luxsiti_ | 56 | MSAEEQRKFVEKFYTA | 235. | 256 | (ATG)AGCGCGGAAGAACAGCGTAAATTTGTGG |
| 0.4_ | | LDSGDAETASSLEPDG | 7553559 | | AAAAATTTTATACCGCGCTGGATAGCGGCGATG |
| 841 | | TLIYLWDGRVFHTQAE | | | CCGAAACCGCGAGCAGCCTGTTTCCTGATGGCA |
| | | FRAWFEKLYSQSANAK | | | CCCTGATTTATCTGTGGGATGGCCGCGTGTTTC |
| | | RSVVRFEVEGNEAYVE | | | ATACCCAGGCGGAATTTCGCGCGTGGTTCGAAA |
| | | VVLHAVVDGKETVVRL | | | AACTGTATAGCCAGAGCGCGAACGCGAAACGCA |
| | | KHNYLFEGDRLVRVEV | | | GCGTGGTGCGCTTTGAAGTGGAAGGCAATGAAG |
| | | EIKPM | | | CGTACGTGGAAGTGGTGCTGCATGCGGTGGTGG |
| | | | | | ATGGCAAAGAAACCGTGGTGAGACTGAAACATA |
| | | | | | ACTATCTATTTGAAGGCGATCGCCTGGTTCGCG |
| | | | | | TCGAAGTTGAAATTAAACCGATGGG(TAA) |
| luxsiti_ | 57 | MSEEAQREFAKRFYAA | 13. | 257 | (ATG)AGCGAAGAAGCGCAGCGCGAATTTGCGA |
| 0.4_ | | LDAGDADTAAALFPDG | 13061507 | | AACGCTTTTATGCGGCGCTGGATGCGGGTGATG |
| 842 | | TEIYLWDGKTETTRAE | | | CGGATACCGCAGCAGCACTGTTTCCAGATGGCA |
| | | FRAWFEELRSTSDRAK | | | CCGAAATTTATCTGTGGGATGGCAAAACCTTTA |
| | | RRVDRFEVNGDTAHVE | | | CCACCCGCGCGGAATTTCGCGCGTGGTTTGAAG |
| | | VTLDAYVNGENRTVRL | | | AACTGCGCAGCACCAGCGATCGCGCGAAAAGGC |
| | | RHVFHEEGDRVKRVEV | | | GCGTTGATCGTTTTGAAGTGAACGGCGATACGG |
| | | EIKPL | | | CGCATGTGGAAGTGACCCTGGACGCGTATGTTA |
| | | | | | ACGGTGAAAACCGTACCGTGCGCCTGCGCCATG |
| | | | | | TGTTTCATTTCGAAGGCGATCGTGTGAAACGCG |
| | | | | | TCGAAGTGGAAATTAAACCGCTGGG(TAA) |
| luxsiti_ | 58 | MSEEEIREFVRRFYEA | 1953. | 258 | (ATG)TCCGAAGAAGAAATTCGCGAATTTGTGC |
| 0.4_ | | LDAGDAATASALFPDG | 540428 | | GCCGCTTTTATGAAGCGCTGGATGCGGGTGATG |
| 844 | | TEIHLWDGTTERTQAQ | | | CGGCAACCGCAAGCGCATTGTTTCCGGATGGCA |
| | | FRAWFERLRAQSANAR | | | CCGAAATTCATCTGTGGGATGGTACCACCTTTC |
| | | REIVDLKVEGDRAKVE | | | GCACCCAGGCGCAGTTTCGCGCGTGGTTTGAAC |
| | | VILRASFDGEEKVVNL | | | GCCTGCGTGCACAAAGCGCAAACGCGCGCAGAG |
| | | THEFLFEGDRLVRVSV | | | AAATTGTCGATCTGAAAGTGGAAGGCGATCGCG |
| | | TITPL | | | CGAAAGTTGAAGTGATTCTGCGCGCGAGCTTTG |
| | | | | | ATGGTGAAGAAAAAGTGGTGAACCTGACCCATG |
| | | | | | AATTTCTGTTTGAAGGTGATCGCCTGGTGCGCG |
| | | | | | TGAGCGTGACCATTACCCCGCTGGG(TAA) |
| luxsiti_ | 59 | MSEEEIKEFWKRFYEA | 7. | 259 | (ATG)AGCGAAGAAGAAATTAAAGAATTTTGGA |
| 0.4_ | | LDAGDAETASALFPDG | 789219074 | | AACGCTTTTATGAAGCGCTGGATGCGGGCGATG |
| 862 | | TEIHLWSGKTERTREE | | | CGGAAACCGCAAGCGCATTGTTTCCGGATGGCA |
| | | FRAWFEELYSQSENAK | | | CCGAAATTCATCTGTGGAGCGGCAAAACCTTTC |
| | | RHIVSLEVDGDEAYVR | | | GCACCCGTGAAGAATTCCGCGCGTGGTTTGAAG |
| | | VVLHAFVKGESRTVEL | | | AACTGTATAGCCAGAGCGAAAACGCGAAACGCC |
| | | EHFFRFEGDHLVEVKV | | | ATATTGTGAGCCTGGAAGTGGATGGCGATGAAG |
| | | DIIPL | | | CCTATGTGCGCGTTGTGCTGCATGCGTTTGTGA |
| | | | | | AAGGCGAAAGCCGCACCGTGGAACTGGAACATT |
| | | | | | TCTTTCGCTTTGAAGGTGATCATCTGGTGGAAG |
| | | | | | TTAAAGTGGACATTATTCCGCTGGG(TAA) |
| luxsiti_ | 60 | MSEAAIREFVDKEYAA | 2361. | 260 | (ATG)AGCGAAGCGGCGATTCGCGAATTTGTTG |
| 0.4_ | | LDAGDAETAASLEPDG | 967519 | | ATAAATTTTATGCGGCGCTGGATGCGGGCGATG |
| 868 | | TKIYLWDGRTFTTQAE | | | CGGAAACAGCAGCAAGCCTGTTTCCAGATGGCA |
| | | FKAWFEELHATSDNAR | | | CCAAAATTTATCTGTGGGATGGCCGCACCTTTA |
| | | REVVSMEVDGDVADVE | | | CCACCCAGGCGGAATTTAAAGCGTGGTTTGAAG |
| | | VVLHASERGEQREVRL | | | AACTGCATGCGACCAGCGATAACGCGCGCCGCG |
| | | RHVFHFEGDELREVEV | | | AAGTGGTGAGCATGGAAGTTGATGGCGATGTGG |
| | | EILPL | | | CAGATGTCGAAGTCGTGCTGCACGCGAGCTTTC |
| | | | | | GCGGCGAACAGCGTGAAGTTCGTCTGCGTCATG |
| | | | | | TGTTTCATTTTGAAGGTGATGAACTGCGGGAAG |
| | | | | | TGGAAGTAGAAATTCTGCCGCTGGG(TAA) |
| luxsiti_ | 61 | MSEEQQREFWARFYAA | 2. | 261 | (ATG)AGCGAAGAACAGCAGCGCGAATTTTGGG |
| 0.4_ | | LDAGDADTASALEPDG | 849343469 | | CGCGCTTTTATGCGGCGTTAGATGCGGGTGATG |
| 871 | | TKIHLWDGKTFTSRAE | | | CGGATACCGCGAGCGCGCTGTTTCCAGATGGCA |
| | | FRDWFVRLHSRSENAK | | | CCAAAATCCATCTGTGGGATGGCAAAACCTTTA |
| | | RRITSFKVEGDISYVE | | | CCAGCCGCGCGGAATTTCGCGATTGGTTTGTGC |
| | | VVLHASRDGQEHVVKE | | | GCCTGCATAGCCGCTCTGAAAACGCCAAACGCC |
| | | KHVAKFEGDELVEVKV | | | GCATTACGAGCTTTAAAGTGGAAGGCGATATTA |
| | | EITPL | | | GCTATGTGGAAGTGGTGCTGCATGCGAGCCGCG |
| | | | | | ATGGCCAGGAACATGTAGTGAAACTGAAACATG |
| | | | | | TGGCGAAATTTGAAGGTGATGAACTGGTTGAAG |
| | | | | | TGAAAGTTGAAATTACCCCGCTGGG(TAA) |
| luxsiti_ | 62 | MSKEEQKNFCKKFYEA | 1. | 262 | (ATG)AGCAAAGAAGAACAAAAGAACTTTTGCA |
| 0.4_ | | LDAGDAETASSLFPDG | 567380788 | | AAAAGTTTTATGAAGCGCTGGATGCGGGTGATG |
| 872 | | TKIYLWDGKTESTQEE | | | CGGAAACCGCTAGCAGCCTGTTTCCGGATGGCA |
| | | FRAWFEKLRSESANAS | | | CCAAAATTTATCTGTGGGATGGTAAAACCTTTA |
| | | RRIVSFNVDGNVAKVK | | | GCACCCAGGAAGAATTTCGCGCGTGGTTTGAAA |
| | | VVLKANYRGKKSTVKL | | | AACTGCGCAGCGAATCGGCGAATGCGAGCCGCC |
| | | EHKFEFEGDKLVKVEV | | | GTATTGTGAGCTTTAACGTGGATGGGAACGTGG |
| | | TIEPL | | | CGAAAGTGAAAGTGGTGCTGAAAGCGAACTATC |
| | | | | | GCGGCAAGAAAAGCACCGTGAAACTGGAACATA |
| | | | | | AATTTGAATTCGAAGGCGATAAACTGGTTAAAG |
| | | | | | TAGAAGTGACCATTGAACCGCTGGG(TAA) |
| luxsiti_ | 63 | MSEEAIREFVKRFYEA | 1070. | 263 | (ATG)AGCGAAGAAGCGATTCGCGAATTTGTGA |
| 0.4_ | | LDAGDADTASALFPDG | 718728 | | AACGCTTTTATGAAGCGCTGGATGCGGGTGATG |
| 880 | | TRIYLWDGTTERTQAE | | | CGGATACCGCAAGCGCGTTGTTTCCGGATGGCA |
| | | FRAWFRELYSKSEGAR | | | CCCGCATTTATCTGTGGGATGGTACCACCTTTC |
| | | REVINLKVDGDVAYVE | | | GCACCCAGGCGGAATTTCGCGCGTGGTTTCGTG |
| | | VVLHAVYKGEEKTVKL | | | AACTGTATAGCAAAAGCGAAGGCGCGCGCCGCG |
| | | VHVFKFEGDKVVEVHV | | | AAGTTATTAACCTGAAAGTGGATGGTGATGTGG |
| | | EIVPL | | | CGTATGTGGAAGTGGTGCTGCATGCGGTGTATA |
| | | | | | AAGGTGAAGAAAAGACCGTGAAACTGGTGCATG |
| | | | | | TGTTTAAATTTGAAGGCGATAAAGTGGTTGAAG |
| | | | | | TGCACGTGGAAATTGTGCCGCTGGG(TAA) |
| luxsiti_ | 64 | MSEEEIREFVKRFYTA | 42. | 264 | (ATG)AGCGAAGAAGAAATTCGCGAATTTGTGA |
| 0.4_ | | LDAGDAETASALEPDG | 49136144 | | AACGCTTTTATACCGCGCTGGATGCGGGCGATG |
| 887 | | TKIYLWDGTTFETREG | | | CGGAAACTGCGAGCGCACTGTTTCCAGATGGCA |
| | | FAAWERKLKSQSEEAK | | | CGAAAATTTATCTGTGGGATGGTACCACCTTTG |
| | | RHVVKLKVEGNVAYIE | | | AAACCCGTGAAGGCTTTGCGGCGTGGTTTCGCA |
| | | VVLHAKHKGEEKTVRL | | | AACTGAAAAGCCAGTCGGAAGAAGCGAAACGCC |
| | | THIYQFEGDKLVEVRV | | | ATGTGGTGAAATTGAAAGTGGAAGGCAACGTGG |
| | | EIKPL | | | CCTATATTGAAGTGGTGCTGCATGCGAAACATA |
| | | | | | AAGGTGAAGAAAAGACCGTGCGCCTGACCCATA |
| | | | | | TCTATCAGTTTGAAGGCGATAAACTGGTTGAAG |
| | | | | | TTCGCGTGGAAATTAAACCGCTGGG{TAA) |
| luxsiti_ | 65 | MSEAEIKNFVKRFYEA | 779. | 265 | (ATG)AGCGAAGCGGAAATTAAAAACTTTGTGA |
| 0.4_ | | LDAGDAETASALEPDG | 227367 | | AACGCTTTTATGAAGCGCTGGATGCAGGCGATG |
| 889 | | TEIHLWDGTTERTRAE | | | CGGAAACCGCTAGCGCACTGTTTCCGGATGGCA |
| | | FRAWFEELYSRSEDAK | | | CCGAAATTCATCTGTGGGATGGTACCACCTTTC |
| | | REVTKLEVKGNVAFVE | | | GCACCCGCGCCGAATTTCGCGCGTGGTTTGAAG |
| | | VVLKANLNGKERTVKL | | | AACTGTATAGCCGCAGCGAAGATGCGAAACGCG |
| | | KHIFHFEGDSLVRVEV | | | AAGTGACCAAACTGGAAGTGAAAGGCAACGTGG |
| | | SIVPL | | | CGTTTGTGGAAGTTGTGCTGAAAGCGAACCTGA |
| | | | | | ACGGCAAAGAACGCACCGTGAAACTGAAACATA |
| | | | | | TTTTTCATTTTGAAGGCGATAGCCTGGTGCGCG |
| | | | | | TTGAAGTGTCGATTGTGCCGCTGGG(TAA) |
| luxsiti_ | 66 | MSEQEQKEFWEKFYQA | 60. | 266 | (ATG)AGCGAACAGGAACAGAAAGAATTTTGGG |
| 0.4_ | | LDAGDAETASALFKDG | 65169316 | | AAAAATTTTATCAGGCGCTGGATGCGGGCGATG |
| 893 | | TEIYLWDGKVEKTQEE | | | CGGAAACCGCGAGCGCGTTGTTTAAAGATGGCA |
| | | FKAWEVELYSKSLNAK | | | CCGAAATTTATCTGTGGGATGGCAAAGTGTTCA |
| | | RKIVEHEVKGNESYVK | | | AAACCCAGGAAGAATTCAAAGCGTGGTTCGTGG |
| | | VVLRASVDGEERTVLL | | | AACTGTATAGCAAAAGCCTGAACGCGAAACGCA |
| | | EHKFLFEGDKLVKVSV | | | AAATTGTGGAACATGAAGTGAAAGGCAACGAAT |
| | | EITPL | | | CTTATGTGAAAGTGGTGCTGCGCGCCAGCGTGG |
| | | | | | ATGGCGAAGAACGCACCGTGTTGTTGGAACACA |
| | | | | | AATTTCTGTTTGAAGGCGATAAACTGGTTAAAG |
| | | | | | TGAGCGTTGAAATTACCCCGCTGGG(TAA) |
| luxsiti_ | 67 | MTAEEQREFVKRFYEA | 3. | 267 | (ATG)ACCGCGGAAGAACAGCGCGAATTTGTGA |
| 0.4_ | | LDAGDAETASALFPDG | 093296475 | | AACGCTTTTATGAAGCGCTGGATGCGGGCGATG |
| 894 | | TLIHLWDGKTFHTREE | | | CGGAAACTGCGAGCGCTCTGTTTCCAGATGGCA |
| | | FRAWEVELRSTSDDAK | | | CCCTGATTCATCTGTGGGATGGCAAAACCTTTC |
| | | REVTAFEVDGDVARVE | | | ATACCCGCGAAGAATTTCGCGCGTGGTTTGTGG |
| | | VVLHANIQGNKKTVAL | | | AACTGCGCAGCACCAGCGATGATGCGAAACGCG |
| | | THEFLFEGDKLVEVRV | | | AAGTGACCGCCTTTGAAGTGGATGGCGATGTGG |
| | | DIKPL | | | CGCGCGTTGAAGTTGTGCTGCATGCGAACATTC |
| | | | | | AGGGCAACAAAAAGACCGTCGCGCTGACCCACG |
| | | | | | AATTTCTGTTTGAAGGCGATAAACTGGTGGAAG |
| | | | | | TGCGTGTGGATATTAAACCGCTGGG(TAA) |
| luxsiti_ | 68 | MSEEEQKNFVRKFYDA | 652. | 268 | (ATG)AGCGAAGAAGAACAGAAAAACTTTGTGC |
| 0.4_ | | LDAGDSETASALEKDG | 4934347 | | GCAAATTTTATGATGCGCTGGATGCGGGCGATA |
| 914 | | TKIYLWDGQVFETQEE | | | GCGAAACCGCGAGCGCGCTGTTTAAAGATGGCA |
| | | FRAWFEKLYSTSTDAK | | | CCAAAATTTATCTGTGGGATGGCCAGGTTTTTG |
| | | REIVSFKVDGNKAVVE | | | AAACCCAGGAAGAATTTCGCGCGTGGTTTGAAA |
| | | VVLKAIKDGEEKVVNL | | | AATTGTATAGCACCAGCACCGATGCCAAACGCG |
| | | KHVFLEEGDEVVEVEV | | | AAATTGTGAGCTTTAAAGTGGATGGCAACAAAG |
| | | DIVPS | | | CGGTGGTTGAGGTGGTGCTGAAAGCGATTAAAG |
| | | | | | ACGGTGAAGAAAAAGTGGTGAACCTGAAACATG |
| | | | | | TGTTTCTGTTTGAAGGTGATGAAGTGGTAGAAG |
| | | | | | TGGAAGTTGATATTGTGCCGAGCGG(TAA) |
| luxsiti_ | 69 | MTAEQQRNFWARFYAA | 4. | 269 | (ATG)ACCGCGGAACAGCAGCGCAACTTTTGGG |
| 0.4_ | | LDAGDAETAAALFPDG | 01520387 | | CGCGCTTTTATGCGGCGCTGGATGCGGGTGATG |
| 915 | | TVIHLWDGRTFHTQAE | | | CGGAAACTGCAGCAGCCTTATTTCCTGATGGCA |
| | | FRAWFEALRSTSENAR | | | CCGTGATTCATCTGTGGGATGGCCGCACCTTTC |
| | | REIVFFQVDGNTADVE | | | ATACCCAGGCGGAATTTCGCGCGTGGTTTGAAG |
| | | VVLHASVDGDARTVRL | | | CGCTGCGTAGCACCAGCGAAAACGCGCGTCGTG |
| | | RHIFQFEGDHLVEVHV | | | AAATTGTGTTTTTCCAGGTGGATGGCAACACCG |
| | | DIRPL | | | CCGATGTGGAAGTGGTGCTGCATGCGAGCGTGG |
| | | | | | ACGGTGACGCAAGAACCGTGCGTCTGAGACATA |
| | | | | | TTTTTCAGTTCGAAGGCGATCATCTGGTTGAAG |
| | | | | | TGCATGTGGATATTCGCCCGCTGGG(TAA) |
| luxsiti_ | 70 | MSEEEQKNFVAAFYKA | 907. | 270 | (ATG)AGCGAAGAAGAACAAAAGAACTTTGTGG |
| 0.4_ | | LDAGDAETASALFPDG | 3780235 | | CGGCGTTTTATAAAGCGCTGGATGCGGGCGATG |
| 921 | | TEIHLWDGKTFKTQAE | | | CCGAAACTGCGAGCGCGTTGTTTCCAGATGGCA |
| | | FKAWFEKLFSTSDNAK | | | CCGAAATTCATCTGTGGGATGGCAAAACCTTTA |
| | | RKVVSFKVNGNIADVQ | | | AAACCCAGGCCGAATTTAAAGCGTGGTTTGAAA |
| | | VVLEANINGEPVKVKL | | | AACTGTTTAGCACCAGCGATAACGCGAAACGCA |
| | | NHTFKFKGDKLVEVNV | | | AAGTGGTGAGCTTTAAAGTGAACGGCAACATTG |
| | | QIEPL | | | CGGATGTGCAGGTGGTGCTGGAAGCGAATATTA |
| | | | | | ACGGCGAACCAGTGAAAGTTAAACTGAACCATA |
| | | | | | CCTTCAAATTCAAAGGCGATAAACTGGTGGAAG |
| | | | | | TTAACGTGCAGATTGAACCGCTGGG(TAA) |
| luxsiti_ | 71 | MSEEEIREFVRKFYAA | 366. | 271 | (ATG)AGCGAAGAAGAAATTCGTGAATTTGTGC |
| 0.4_ | | LDAGDADTASALEPDG | 0525225 | | GCAAATTTTATGCGGCGCTGGATGCGGGTGATG |
| 923 | | TEIHLWDGVTFKTQAE | | | CGGATACCGCAAGCGCATTGTTTCCGGATGGCA |
| | | FKAWFTELKSKSDNAK | | | CCGAAATTCATCTGTGGGATGGCGTGACCTTTA |
| | | REVVKLKVDGNKADVE | | | AAACCCAGGCGGAATTTAAAGCGTGGTTTACCG |
| | | VVLHATIDGKEVTVHL | | | AACTGAAAAGCAAAAGCGATAACGCGAAACGCG |
| | | RHIFEFEGDKLVKVEV | | | AGGTGGTGAAATTGAAAGTGGATGGTAACAAAG |
| | | VIEPL | | | CGGATGTGGAAGTGGTGCTGCATGCGACCATTG |
| | | | | | ATGGCAAAGAAGTGACCGTGCATCTGCGCCATA |
| | | | | | TTTTTGAATTCGAAGGCGATAAACTGGTTAAAG |
| | | | | | TTGAAGTTGTGATTGAACCGCTGGG(TAA) |
| luxsiti_ | 72 | MSAEAQREFVKRFYDA | 4. | 272 | (ATG)AGCGCGGAAGCGCAGCGTGAATTTGTGA |
| 0.4_ | | LDAGDADTASALFADG | 075328265 | | AACGCTTTTATGATGCCCTGGATGCGGGTGATG |
| 945 | | TRIYLWDGREFRTRAE | | | CGGATACCGCGAGTGCGTTGTTTGCCGATGGCA |
| | | FRAWFERLRSRSDAAK | | | CCCGCATTTATCTGTGGGATGGCCGCGAATTTC |
| | | RHVTSFKVEGNVAQVV | | | GCACCCGTGCGGAATTTAGAGCGTGGTTTGAAC |
| | | VVLKANFRGKEHVVEL | | | GTCTGCGCAGCCGCAGCGATGCGGCGAAACGTC |
| | | LHEFVFEGDRIVEVHV | | | ATGTGACCAGCTTTAAAGTGGAAGGCAACGTGG |
| | | EIRPR | | | CGCAGGTGGTGGTTGTGCTGAAAGCGAACTTTC |
| | | | | | GCGGCAAAGAACATGTGGTGGAACTGCTGCATG |
| | | | | | AATTCGTGTTTGAAGGCGATCGCCTGGTTGAAG |
| | | | | | TGCATGTGGAAATTCGCCCGCGCGG(TAA) |
| luxsiti_ | 73 | MSEEEIREFVKRFYEA | 32. | 273 | (ATG)AGCGAAGAAGAAATCCGCGAATTTGTGA |
| 0.4_ | | LDAGDAETASALFPDG | 48928818 | | AACGCTTTTATGAAGCGCTGGATGCGGGTGATG |
| 955 | | TKIHLWDGITENTQEE | | | CGGAAACCGCAAGCGCACTGTTTCCGGATGGGA |
| | | FQKWEVELRSQSDNAK | | | CCAAAATTCATCTGTGGGATGGCATTACCTTTA |
| | | REVVSLKVDGNHAKVE | | | ACACCCAGGAAGAATTTCAGAAATGGTTTGTGG |
| | | VVLKANIGGEDRVVKL | | | AACTGCGCAGCCAGAGCGATAACGCAAAACGTG |
| | | THHELFEGDKLIEVRV | | | AAGTGGTGAGCCTGAAAGTGGATGGTAACCATG |
| | | EIEPL | | | CGAAAGTTGAAGTTGTGCTGAAAGCGAACATTG |
| | | | | | GCGGCGAAGATCGCGTGGTGAAACTGACCCATC |
| | | | | | ATTTTCTGTTTGAAGGCGATAAACTGATTGAAG |
| | | | | | TGCGCGTGGAAATTGAACCGCTGGG(TAA) |
| luxsiti_ | 74 | MSAEAIREFVERFYAA | 11. | 274 | (ATG)AGCGCGGAAGCGATTCGTGAATTTGTTG |
| 0.4_ | | LDAGDAETASALFPDG | 68071873 | | AACGTTTTTATGCGGCGCTGGATGCGGGCGATG |
| 957 | | TIIHLWDGRVFHTREE | | | CGGAAACTGCAAGCGCACTGTTTCCTGATGGCA |
| | | FRRWFVDLFSTSDNAS | | | CCATTATTCATCTGTGGGATGGCCGCGTGTTTC |
| | | RSVVDLHVDGNVAHVV | | | ATACCCGCGAAGAATTTCGCCGCTGGTTTGTGG |
| | | VRLKASWRGKKRTVLL | | | ATTTGTTTAGCACCAGCGATAACGCGAGCCGCA |
| | | THVFEFEGDHLVKVTV | | | GCGTGGTGGATCTGCATGTGGATGGCAACGTGG |
| | | SIQPV | | | CTCATGTGGTGGTGCGCCTGAAAGCGAGCTGGC |
| | | | | | GTGGCAAGAAACGCACCGTGTTGCTGACCCATG |
| | | | | | TGTTTGAATTCGAAGGTGATCATCTGGTGAAAG |
| | | | | | TGACCGTGAGCATTCAGCCGGTGGG(TAA) |
| luxsiti_ | 75 | MSABEIREFVARFYAA | 11. | 275 | (ATG)AGCGCGGAAGAAATTCGCGAATTTGTGG |
| 0.4_ | | LDAGDADTASALEPNG | 32826538 | | CGCGCTTTTATGCGGCGTTGGATGCGGGTGATG |
| 967 | | TKIYLWDGTVETTQAE | | | CGGATACCGCAAGCGCGCTGTTTCCGAACGGTA |
| | | FRAWFVKLRSTSENAK | | | CCAAAATTTATCTGTGGGATGGCACCGTGTTTA |
| | | REVVELEVEGNEAFVE | | | CCACCCAGGCGGAATTTCGCGCGTGGTTTGTGA |
| | | VVLHAEIKGQKKTVRL | | | AACTGCGCAGCACCAGCGAAAACGCGAAACGTG |
| | | RHVFKFEGDHLVEVSV | | | AAGTGGTGTTTCTGGAAGTGGAAGGCAACGAAG |
| | | DIEPL | | | CGTTTGTGGAAGTTGTGCTGCATGCGGAAATTA |
| | | | | | AAGGCCAGAAGAAAACCGTGCGCCTGCGCCATG |
| | | | | | TGTTTAAATTTGAAGGCGATCATCTGGTTGAAG |
| | | | | | TGTCTGTGGATATTGAACCGCTGGG(TAA) |
| luxsiti_ | 76 | MSKEDIEEFCKKFYDA | 97. | 276 | (ATG)AGCAAAGAAGATATTGAAGAATTTTGCA |
| 0.4_ | | LDAGDAETASALFPDG | 61368348 | | AAAAGTTTTATGATGCGCTGGATGCGGGCGATG |
| 969 | | TEINLWDGKVFTTKSE | | | CCGAAACAGCAAGCGCCCTGTTTCCAGATGGCA |
| | | FRAWFRELYARSDHAK | | | CCGAAATTAACCTGTGGGATGGCAAAGTGTTTA |
| | | REITHLEVDGNFATIE | | | CCACCAAAAGCGAATTTCGCGCGTGGTTTCGCG |
| | | VVLNASVDGEQKTVSE | | | AACTGTATGCCCGCAGCGATCATGCCAAACGTG |
| | | KHFFQFEGDRLVRVDV | | | AAATTACCCATCTGGAAGTGGATGGTAACTTTG |
| | | KINPL | | | CGACCATTGAAGTGGTGCTGAACGCGAGCGTGG |
| | | | | | ACGGCGAACAGAAAACCGTGAGCCTGAAACATT |
| | | | | | TCTTTCAGTTTGAAGGCGATCGCCTGGTGCGCG |
| | | | | | TTGATGTGAAAATCAACCCGCTGGG(TAA) |
| luxsiti_ | 77 | MSEEEIREFWQRFYEA | 2. | 277 | (ATG)AGCGAAGAAGAAATTCGCGAATTTTGGC |
| 0.4_ | | LDAGDAETASALFPDG | 067035245 | | AGCGCTTTTATGAAGCGCTGGATGCGGGCGATG |
| 977 | | TEIYLWDGKTERTRAE | | | CGGAAACCGCGAGCGCATTGTTTCCTGATGGCA |
| | | FRAWFEELHSTSENAK | | | CCGAAATTTATCTGTGGGATGGCAAAACCTTTC |
| | | RHVTALEVDGNVARVQ | | | GCACCCGCGCGGAATTTCGCGCGTGGTTTGAAG |
| | | VVLHASINGEEKTVKL | | | AACTGCATAGCACCAGCGAAAACGCCAAACGTC |
| | | DHETHFEGDRLVRVTV | | | ATGTGACCGCTCTGGAAGTGGATGGTAACGTGG |
| | | DIKPL | | | CGCGCGTTCAGGTGGTGCTGCATGCGAGCATTA |
| | | | | | ATGGTGAAGAAAAGACCGTGAAACTGGATCATG |
| | | | | | AAACCCATTTTGAAGGTGATCGCCTGGTGCGCG |
| | | | | | TGACCGTGGATATTAAACCGCTGGG(TAA) |
| luxsiti_ | 78 | MSEEEQRNFVKRFYEA | 4. | 278 | (ATG)AGTGAAGAAGAACAGCGCAACTTTGTGA |
| 0.4_ | | LDAGDAETASALFPDG | 937111265 | | AACGCTTTTATGAAGCGCTGGATGCGGGCGATG |
| 998 | | TVIHLWDGTTFHTQAE | | | CGGAAACCGCGAGCGCGTTATTTCCTGATGGCA |
| | | FRAWFTELRSTSENAK | | | CCGTGATTCATCTGTGGGATGGTACCACCTTTC |
| | | REVTKFAVDGNVAHVE | | | ATACCCAGGCGGAATTTCGCGCGTGGTTTACCG |
| | | VVLHANHNGEPRVVRL | | | AACTGCGCAGCACCAGCGAAAACGCGAAACGTG |
| | | THEFHFEGDHLVEVTV | | | AAGTGACCAAATTTGCGGTGGATGGCAACGTGG |
| | | RIDPL | | | CGCATGTGGAAGTTGTGCTGCATGCGAACCATA |
| | | | | | ACGGTGAACCGCGCGTTGTGCGCCTGACCCATG |
| | | | | | AATTTCATTTTGAAGGCGATCATCTGGTTGAAG |
| | | | | | TTACCGTGCGCATCGATCCGCTGGG(TAA) |
| luxsiti_ | 79 | MSEEEQREFVRRFYEA | 78. | 279 | (ATG)AGCGAAGAAGAACAGCGCGAATTTGTGC |
| 0.2_ | | LDAGDAETASALFPDG | 33724948 | | GCCGCTTTTATGAAGCGCTGGATGCGGGCGATG |
| 7 | | TVIDLWDGKTFHTRAE | | | CGGAAACTGCAAGCGCGCTGTTTCCAGATGGCA |
| | | FREWFVKLKSTSDNAK | | | CCGTGATCGATCTGTGGGATGGCAAAACCTTTC |
| | | REVTNFKVDGDVAHVE | | | ATACCCGCGCCGAATTTCGCGAATGGTTTGTGA |
| | | VVLKASINGEEKVVKL | | | AACTGAAAAGCACCAGCGATAACGCGAAACGTG |
| | | THTFQFEGDRLVRVSV | | | AAGTGACCAACTTTAAAGTGGATGGCGATGTGG |
| | | KIEPL | | | CGCATGTGGAAGTGGTGCTGAAAGCGAGCATTA |
| | | | | | ACGGTGAAGAAAAAGTGGTTAAACTGACCCATA |
| | | | | | CCTTCCAGTTTGAAGGCGATCGCCTGGTGCGCG |
| | | | | | TGAGCGTGAAAATTGAACCGCTGGG(TAA) |
| luxsiti_ | 80 | MSEEEQKEFCKKFYEA | 170. | 280 | (ATG)TCGGAAGAAGAACAGAAAGAATTTTGCA |
| 0.2_ | | LDAGDADTASALFPDG | 6966137 | | AAAAGTTTTATGAAGCATTGGATGCGGGTGATG |
| 35 | | TKIHLWDGKTETTQAQ | | | CCGATACGGCGAGCGCCCTGTTTCCAGATGGCA |
| | | FKAWFVELYSKSENAK | | | CCAAAATCCATCTGTGGGATGGCAAAACCTTTA |
| | | REITKFEVDGNVADVE | | | CCACCCAGGCGCAGTTTAAAGCCTGGTTTGTGG |
| | | VVLHAIVNGEKKTVKL | | | AACTGTATAGCAAAAGCGAAAACGCCAAACGTG |
| | | KHVFKFEGDKLVEVNV | | | AAATTACGAAATTTGAAGTGGATGGTAACGTGG |
| | | EIKPL | | | CGGATGTGGAAGTGGTGCTGCATGCGATCGTGA |
| | | | | | ACGGCGAAAAGAAAACCGTGAAACTGAAACATG |
| | | | | | TTTTTAAATTCGAAGGTGATAAACTGGTTGAAG |
| | | | | | TCAACGTCGAAATCAAACCGCTGGG(TAA) |
| luxsiti_ | 81 | MSAEEIKNFCDKEYKA | 1348. | 281 | (ATG)AGCGCGGAAGAAATTAAAAACTTCTGCG |
| 0.2_ | | LDAGDADTASALEPDG | 814098 | | ACAAATTTTATAAAGCGCTGGATGCGGGCGATG |
| 41 | | TEIYLWDGKTFKTQAE | | | CGGATACCGCAAGCGCCTTGTTTCCAGATGGCA |
| | | FKAWFEKLYSTSKDAK | | | CCGAAATTTATCTGTGGGATGGCAAAACCTTTA |
| | | RKITSLEVNGNVAKVK | | | AAACCCAGGCGGAATTTAAAGCGTGGTTTGAAA |
| | | VVLEAIIDGEKKTVNL | | | AACTGTATAGCACCAGCAAAGATGCGAAACGCA |
| | | EHIFYFEGDKLVKVEV | | | AAATTACCAGCCTGGAAGTGAATGGCAATGTTG |
| | | SIEPL | | | CGAAAGTGAAAGTGGTGCTGGAAGCGATTATTG |
| | | | | | ATGGCGAAAAGAAAACCGTGAACCTGGAACATA |
| | | | | | TTTTCTATTTTGAAGGCGATAAACTGGTCAAAG |
| | | | | | TTGAAGTGAGCATTGAACCGCTGGG(TAA) |
| luxsiti_ | 82 | MSEEEQREFVARFYAA | 5. | 282 | (ATG)AGCGAAGAAGAACAGCGCGAATTTGTGG |
| 0.2_ | | LDAGDAETASALFPDG | 905321355 | | CGCGCTTTTATGCGGCGCTGGATGCGGGTGATG |
| 44 | | TEIHLWDGKTFTTRAE | | | CGGAAACTGCAAGCGCGTTGTTTCCGGATGGCA |
| | | FRAWFEKLHSLSDNAS | | | CCGAAATTCATCTGTGGGACGGCAAAACCTTTA |
| | | RHVTSFKVDGNVAEVE | | | CCACCCGCGCGGAATTTCGCGCGTGGTTTGAAA |
| | | VVLHADFKGKKLTVKL | | | AATTGCATAGCCTGAGCGATAATGCGAGCCGCC |
| | | RHRYQFEGDRLVRVDV | | | ATGTGACCAGCTTTAAAGTGGATGGTAACGTGG |
| | | EIFPL | | | CGGAAGTGGAAGTTGTGCTGCATGCGGATTTTA |
| | | | | | AAGGCAAGAAACTGACCGTGAAATTGCGCCATC |
| | | | | | GCTATCAGTTTGAAGGCGATCGCCTGGTGCGCG |
| | | | | | TCGATGTGGAAATTTTTCCGCTGGG(TAA) |
| luxsiti_ | 83 | MSADAQREFVRRFYEA | 1. | 283 | (ATG)AGCGCGGATGCGCAGCGCGAATTTGTGC |
| 0.3_ | | LDAGDAETAAALEPDG | 489979267 | | GCCGCTTTTATGAAGCGCTGGATGCGGGTGATG |
| 478 | | TEIHLWDGKTFRTQAE | | | CGGAAACCGCAGCCGCGTTGTTTCCTGATGGCA |
| | | FRAWFEKLRAQSENAK | | | CCGAAATTCATCTGTGGGATGGCAAAACCTTTC |
| | | RHVVNFQVDGNDADVE | | | GCACCCAGGCGGAATTTCGTGCGTGGTTTGAAA |
| | | VVLHATYNGEQKTVRE | | | AACTGCGCGCGCAGAGCGAAAACGCGAAACGCC |
| | | KHKFHFEGDQLVRVDV | | | ATGTGGTGAACTTTCAGGTGGATGGTAACGATG |
| | | TIEPL | | | CCGATGTGGAAGTGGTGCTGCATGCGACCTATA |
| | | | | | ACGGCGAACAGAAAACCGTGCGCCTGAAACATA |
| | | | | | AATTTCATTTTGAAGGCGATCAGCTGGTGCGCG |
| | | | | | TTGATGTGACCATTGAACCGCTGGG(TAA) |
| luxsit_ | 84 | MSEEQIRQFLRRFYEA | 1 | 284 | (ATG)AGCGAAGAACAAATTCGCCAGTTTCTGC |
| R65A | | LDSGDADTAASLFHPG | | | GCCGCTTTTATGAAGCGTTAGATTCTGGCGATG |
| | | VTIHLWDGVTFTSREE | | | CGGATACCGCGGCCAGCCTCTTTCATCCGGGCG |
| | | FREWFERLFSTRKDAQ | | | TGACCATTCATCTGTGGGATGGCGTTACCTTTA |
| | | AEIKSLEVRGDTVEVH | | | CCAGCCGTGAAGAATTTCGCGAATGGTTTGAAC |
| | | VQLHATHNGQKHTVDA | | | GCCTGTTTAGCACCCGCAAAGATGCGCAGGCGG |
| | | THHWHFRGNRVTEMRV | | | AAATTAAAAGCCTGGAAGTGCGCGGCGATACCG |
| | | HINPT | | | TGGAAGTTCACGTGCAGCTGCATGCGACCCATA |
| | | | | | ACGGCCAGAAACATACCGTTGATGCGACGCATC |
| | | | | | ATTGGCATTTTCGCGGCAACCGCGTGACGGAAA |
| | | | | | TGCGCGTGCATATTAACCCGACCGG(TAA) |
| luxsiti | 85 | MSEEQIRQFLRRFYEA | 633. | 285 | (ATG)AGCGAAGAACAAATCCGCCAGTTTCTGC |
| | | LDSGDADTAASLFHPG | 2411887 | | GCCGCTTTTATGAAGCGCTGGATAGCGGCGACG |
| | | VTIHLWDGVTETSREE | | | CGGATACCGCAGCGAGCTTATTTCATCCGGGCG |
| | | FREWFERLFSTSKDAQ | | | TGACCATTCATCTGTGGGATGGCGTTACCTTTA |
| | | REIKSLEVRGDTVEVH | | | CCAGCCGTGAAGAATTTCGCGAATGGTTTGAAC |
| | | VQLHATHNGQKHTVDL | | | GCCTGTTTAGCACCAGCAAAGATGCGCAGCGCG |
| | | THHWHERGNRVTEVRV | | | AAATTAAAAGCCTGGAAGTGCGCGGCGATACCG |
| | | HINPT | | | TGGAAGTTCATGTGCAGCTGCATGCGACCCATA |
| | | | | | ACGGCCAGAAACATACCGTTGATCTGACCCATC |
| | | | | | ATTGGCATTTTCGCGGCAACCGCGTGACGGAAG |
| | | | | | TGAGAGTGCATATTAACCCGACCGG(TAA) |
| luxsiti_ | 86 | MSEEEQKEFCKKFYEA | 318. | 286 | (ATG)AGCGAAGAAGAACAGAAAGAATTTTGCA |
| 0.2_ | | LDAGDADTASALEPDG | 4996545 | | AGAAATTTTATGAAGCGCTGGATGCGGGTGATG |
| 35 | | TKIHLWDGKTFTTQAQ | | | CGGATACCGCAAGCGCACTGTTTCCCGATGGTA |
| | | FKAWFVELYSKSENAK | | | CCAAAATCCATCTGTGGGATGGCAAAACCTTTA |
| | | REITKFEVDGNVADVE | | | CCACCCAGGCGCAGTTTAAAGCGTGGTTTGTGG |
| | | VVLHAIVNGEKKTVKL | | | AACTGTACAGCAAAAGCGAAAATGCGAAACGTG |
| | | KHVFKFEGDKLVEVNV | | | AAATTACCAAATTTGAAGTGGATGGTAACGTGG |
| | | EIKPL | | | CGGATGTGGAAGTGGTGCTGCATGCGATTGTGA |
| | | | | | ACGGCGAAAAGAAAACCGTGAAACTGAAACATG |
| | | | | | TGTTTAAATTCGAAGGCGATAAACTGGTTGAAG |
| | | | | | TTAATGTTGAAATCAAACCGCTGGG(TAA) |
| luxsiti_ | 87 | MSAEEIKNFCDKFYKA | 1719. | 287 | (ATG)AGCGCGGAAGAAATTAAAAACTTTTGCG |
| 0.2_ | | IDAGDADTASALEPDG | 627505 | | ATAAATTTTATAAAGCGCTGGATGCGGGTGATG |
| 41 | | TEIYLWDGKTFKTQAE | | | CGGATACCGCGAGTGCACTGTTTCCTGATGGCA |
| | | FKAWFEKLYSTSKDAK | | | CCGAAATTTATCTGTGGGATGGCAAAACCTTTA |
| | | RKITSLEVNGNVAKVK | | | AAACCCAGGCGGAATTTAAAGCGTGGTTTGAAA |
| | | VVLEAIIDGEKKTVNL | | | AACTGTATAGCACCAGCAAAGATGCGAAACGTA |
| | | EHIFYFEGDKLVKVEV | | | AAATTACCAGCCTGGAAGTGAACGGCAACGTTG |
| | | SIEPL | | | CGAAAGTGAAAGTGGTGCTGGAAGCGATTATTG |
| | | | | | ATGGCGAAAAGAAAACCGTTAACCTGGAACATA |
| | | | | | TTTTCTATTTTGAAGGTGATAAACTGGTTAAAG |
| | | | | | TGGAAGTATCTATTGAACCGCTGGG(TAA) |
| luxsiti_ | 88 | MSEEEQREFVARFYAA | 1317. | 288 | (ATG)AGCGAAGAAGAACAGCGTGAATTTGTGG |
| 0.2_ | | LDAGDAETASALFPDG | 533518 | | CGCGCTTTTATGCGGCGCTGGATGCGGGTGATG |
| 44 | | TEIHLWDGKTETTRAE | | | CGGAAACTGCAAGCGCGCTGTTTCCTGATGGCA |
| | | FRAWFEKLHSLSDNAS | | | CCGAAATTCATCTGTGGGATGGCAAAACCTTTA |
| | | RHVTSFKVDGNVAEVE | | | CCACCCGCGCGGAATTTCGCGCGTGGTTTGAAA |
| | | VVLHADFKGKKLTVKL | | | AATTGCATAGCCTGAGCGATAACGCGAGCCGCC |
| | | RHRYQFEGDRLVRVDV | | | ATGTGACCAGCTTTAAAGTGGATGGTAACGTGG |
| | | EIFPL | | | CGGAAGTGGAAGTTGTGCTGCATGCGGATTTTA |
| | | | | | AAGGCAAAAAGCTGACCGTGAAACTGAGACATC |
| | | | | | GCTATCAGTTTGAAGGCGATCGCCTGGTGCGCG |
| | | | | | TTGATGTGGAAATTTTTCCGCTGGG(TAA) |
| luxsiti_ | 89 | MSEEEIRQFVKKEYEA | 228. | 289 | (ATG)AGCGAAGAAGAGATTCGCCAGTTTGTGA |
| 0.2_ | | LDAGDAETASALFPDG | 1472 | | AGAAATTTTATGAAGCGCTGGATGCAGGTGATG |
| 170 | | TKIYLWDGTVENTQAE | 011 | | CGGAAACCGCAAGCGCCCTGTTTCCGGATGGCA |
| | | FKAWFVELYSTSENAK | | | CCAAAATTTATCTGTGGGATGGTACCGTGTTTA |
| | | REVVKFEVDGNKAKVE | | | ACACCCAGGCGGAATTTAAAGCGTGGTTTGTGG |
| | | VVLHASIDGEEKTVKL | | | AACTGTATTCGACCAGCGAAAACGCGAAACGTG |
| | | KHEFYFEGDKVKEVKV | | | AAGTGGTGAAATTTGAAGTCGATGGTAACAAAG |
| | | EIEPL | | | CGAAAGTGGAAGTCGTGCTGCATGCGAGCATTG |
| | | | | | ATGGCGAGGAAAAGACCGTGAAACTGAAACATG |
| | | | | | AATTTTACTTTGAAGGCGATAAAGTGAAAGAAG |
| | | | | | TTAAAGTTGAAATTGAACCGCTGGG(TAA) |
| luxsiti_ | 90 | MSEEEQREFWRRFYEA | 684. | 290 | (ATG)TCCGAAGAAGAACAGCGCGAATTTTGGC |
| 0.2_ | | LDAGDAETASALFPDG | 0103663 | | GCCGCTTTTATGAAGCCCTGGATGCAGGCGATG |
| 196 | | TEIYLWDGRTERTQAE | | | CTGAAACCGCGAGCGCGTTATTTCCGGATGGCA |
| | | FRAWFERLRATSEDAR | | | CCGAAATTTATCTGTGGGATGGCCGTACCTTTC |
| | | REIVSERVEGNRSEVE | | | GCACCCAGGCGGAATTTCGCGCATGGTTTGAAC |
| | | VVLHANVNGEKKTVRL | | | GCCTGCGCGCCACTAGCGAAGATGCGCGCAGAG |
| | | RHYFEFEGDRLVRVEV | | | AAATTGTGAGCTTTCGCGTGGAAGGCAATCGCA |
| | | EIVPL | | | GCGAAGTGGAAGTTGTGCTGCATGCGAACGTGA |
| | | | | | ACGGCGAAAAGAAAACCGTGCGCCTGAGACATT |
| | | | | | ATTTTGAATTTGAAGGCGATCGCCTGGTGCGCG |
| | | | | | TTGAAGTTGAAATCGTGCCGCTGGG(TAA) |
| luxsiti_ | 91 | MSEEEIKEFCKKFYEA | 71. | 291 | (ATG)AGCGAAGAAGAAATTAAAGAATTTTGCA |
| 0.3_ | | LDAGDADTASALEKDG | 81271596 | | AAAAGTTTTATGAAGCGCTGGATGCGGGCGATG |
| 453 | | TEIYLWDGRTFKTRAE | | | CGGATACCGCGAGCGCGCTGTTTAAAGATGGCA |
| | | FKAWFTRLRSTSDNAK | | | CCGAAATTTATCTGTGGGATGGTCGTACCTTTA |
| | | REIVSLSVNGNVAKVK | | | AAACCCGCGCGGAATTTAAAGCGTGGTTTACCC |
| | | VVLRASINGEERTVLL | | | GTCTGCGCAGCACCTCTGATAACGCGAAACGTG |
| | | THEFLFEGDELVEVRV | | | AAATTGTGAGCCTGTCGGTGAACGGCAACGTGG |
| | | SIKPL | | | CGAAAGTGAAAGTGGTGCTGCGCGCGAGCATTA |
| | | | | | ACGGTGAAGAACGCACCGTGCTGCTGACCCATG |
| | | | | | AATTTCTGTTTGAAGGCGATGAACTGGTGGAAG |
| | | | | | TGCGCGTGAGCATCAAACCGCTGGG(TAA) |
| luxsiti_ | 92 | MSKEEQEEFVKQFYEA | 281. | 292 | (ATG)AGCAAAGAAGAACAAGAGGAATTTGTGA |
| 0.2_ | | LDAGDAETASALFPDG | 5210 | | AACAGTTTTATGAAGCGCTGGATGCGGGCGATG |
| 63 | | TVIHLWDGKTFHTQAE | 781 | | CGGAAACCGCAAGCGCACTGTTTCCAGATGGCA |
| | | FRAWFEELKSTSENAK | | | CCGTGATCCATCTGTGGGATGGCAAAACCTTTC |
| | | REVTKFEVDGDVADVE | | | ATACCCAGGCGGAATTTCGCGCGTGGTTTGAAG |
| | | VVLKANINGEEKVVNL | | | AACTGAAAAGCACCAGCGAAAACGCGAAACGTG |
| | | KHKFKFEGDKVVEVWV | | | AAGTGACCAAATTTGAAGTGGATGGCGATGTGG |
| | | EIEPL | | | CGGATGTGGAAGTGGTGCTGAAAGCGAACATTA |
| | | | | | ACGGCGAAGAAAAAGTGGTTAACCTGAAACATA |
| | | | | | AATTTAAATTCGAAGGCGATAAAGTTGTCGAAG |
| | | | | | TTTGGGTCGAAATTGAACCGCTGGG(TAA) |
| luxsiti_ | 93 | MSEDATREFVKRFYEA | 73. | 293 | (ATG)AGCGAAGATGCCATTCGCGAATTTGTGA |
| 0.2_ | | LDAGDADTASALEPDG | 01105736 | | AACGTTTTTATGAAGCGCTGGATGCGGGCGATG |
| 138 | | TEIHLWDGRVFRTRAE | | | CGGATACCGCGAGCGCATTATTTCCGGATGGCA |
| | | FRAWFVELRSQSENAK | | | CCGAAATTCATCTGTGGGATGGCCGCGTGTTTC |
| | | REVTDLEVEGNVARVE | | | GCACCCGCGCGGAATTTCGTGCCTGGTTTGTGG |
| | | VVLHANYKGEERTVRL | | | AACTGCGCAGCCAGAGCGAAAACGCGAAACGTG |
| | | RHEFQFEGDKVVEVRV | | | AAGTGACCGATCTGGAAGTGGAAGGCAACGTGG |
| | | EIEPL | | | CGCGCGTGGAAGTCGTGCTGCATGCGAACTATA |
| | | | | | AAGGCGAAGAACGCACCGTGCGCCTGCGCCATG |
| | | | | | AATTTCAGTTTGAAGGCGATAAAGTGGTTGAAG |
| | | | | | TGCGCGTTGAAATTGAACCGCTGGG(TAA) |
| luxsiti_ | 94 | MSEEEQKEFCRRFYLA | 10. | 294 | (ATG)TCAGAAGAAGAACAGAAAGAATTCTGCC |
| 0.4_ | | LDAGDADTASALEKDG | 52246026 | | GCCGCTTTTTATCTGGCGCTGGATGCGGGTGATG |
| 1046 | | TKIYLWDGKVENTQEE | | | CGGATACCGCAAGCGCACTGTTTAAAGATGGCA |
| | | FRKWFVELRSTSDQAK | | | CCAAAATTTATCTTTGGGATGGCAAAGTGTTTA |
| | | RSIEDFKVEGNTAKVK | | | ACACCCAGGAAGAATTTCGCAAATGGTTTGTGG |
| | | VVLEASKNGEKRTVGL | | | AACTGCGCAGCACCAGCGATCAGGCGAAACGCA |
| | | EHIFEFEGDEVKEVYV | | | GCATTGAAGATTTTAAAGTGGAAGGCAACACCG |
| | | KIKPL | | | CGAAAGTGAAAGTGGTGCTGGAAGCGAGCAAAA |
| | | | | | ACGGCGAAAAACGCACGGTGGGCCTGGAACATA |
| | | | | | TTTTTGAATTTGAAGGCGATGAGGTGAAAGAAG |
| | | | | | TGTATGTGAAAATTAAACCGCTGGG(TAA) |
| luxsiti_ | 95 | MSATEQREENKRFYEA | 17. | 295 | (ATG)AGCGCGACCGAACAGCGCGAATTTAACA |
| 0.4_ | | LDAGDADTASALFPDG | 71596406 | | AACGCTTTTATGAAGCGCTGGATGCGGGTGATG |
| 1061 | | TEIYLWDGTTEHTQEE | | | CGGATACCGCAAGCGCACTGTTTCCGGATGGCA |
| | | FRAWEVELRSTSENAR | | | CCGAAATTTATCTGTGGGATGGTACCACCTTTC |
| | | RHIVSFTVDGNKADVE | | | ATACCCAGGAAGAATTTCGCGCGTGGTTTGTGG |
| | | VVLEATVKGEPKRVRL | | | AACTGCGCAGCACCTCTGAAAACGCGCGCAGAC |
| | | THNYQWEGDKLVKVTV | | | ATATTGTGAGCTTTACCGTGGACGGCAACAAAG |
| | | KIVPL | | | CGGATGTGGAAGTGGTGCTGGAAGCGACCGTGA |
| | | | | | AAGGCGAACCGAAACGCGTGCGCCTGACTCATA |
| | | | | | ACTATCAGTGGGAAGGCGATAAACTGGTGAAAG |
| | | | | | TGACCGTTAAAATTGTGCCGCTGGG(TAA) |
| luxsiti_ | 96 | MSEEEIREFVKREYQA | 4. | 296 | (ATG)AGCGAAGAAGAAATTCGCGAATTTGTGA |
| 0.4_ | | LDAGDADTASALFPDG | 928818245 | | AACGCTTTTATCAGGCGCTGGATGCGGGTGATG |
| 1066 | | TKIYLWDGVIFTKREE | | | CGGATACCGCAAGTGCGCTGTTTCCGGATGGTA |
| | | FRKWFVELKSKSDNAK | | | CCAAAATTTATCTGTGGGATGGCGTTATTTTTA |
| | | REVVDLRVEGDTADVS | | | CCAAACGCGAGGAATTTCGCAAATGGTTCGTGG |
| | | VVLKAKYKGEPKVVKL | | | AACTGAAAAGCAAAAGCGATAACGCGAAACGAG |
| | | NHKFYFEGDKLVEVEV | | | AAGTGGTGGATCTGCGCGTGGAAGGCGATACGG |
| | | EIVPM | | | CGGATGTGAGCGTGGTGCTGAAAGCGAAATATA |
| | | | | | AAGGCGAACCGAAAGTGGTTAAACTGAACCATA |
| | | | | | AATTTTATTTTGAAGGTGATAAACTGGTTGAAG |
| | | | | | TGGAAGTTGAAATTGTGCCGATGGG(TAA) |
| luxsiti_ | 97 | MTEEEQREFWERFYKA | 1. | 297 | (ATG)ACCGAAGAAGAACAGCGCGAATTTTGGG |
| 0.4_ | | LDAGDAETASALEPDG | 319281272 | | AACGTTTTTATAAAGCGCTGGATGCGGGTGATG |
| 1081 | | TEIYLWDGTVEKTQEE | | | CGGAAACCGCGAGCGCATTGTTTCCGGATGGCA |
| | | FRSWFVDLYSKSNNAK | | | CCGAAATCTATCTGTGGGATGGTACCGTGTTTA |
| | | RHIVEFRVEGDVSYVE | | | AAACCCAGGAAGAATTTCGCAGCTGGTTTGTGG |
| | | VVLHASENGTEKTVRL | | | ATCTGTATAGCAAAAGCAACAACGCCAAACGCC |
| | | THITEFEGDKVVKVEV | | | ATATTGTGGAATTTAGGGTGGAAGGCGATGTGA |
| | | SITPL | | | GCTATGTGGAAGTTGTGCTGCATGCGAGCGAAA |
| | | | | | ACGGCACGGAAAAGACCGTGCGCCTGACCCATA |
| | | | | | TTACCGAATTTGAAGGTGATAAAGTGGTTAAAG |
| | | | | | TTGAAGTGAGCATTACCCCGCTGGG(TAA) |
| luxsiti_ | 98 | MSEDEIREFWKKFYEA | 1. | 298 | (ATG)AGCGAAGATGAAATTCGCGAATTTTGGA |
| 0.4_ | | IDAGDADTASALEPDG | 214927436 | | AAAAGTTTTATGAAGCGCTGGATGCGGGTGATG |
| 1100 | | TKIYLWDGKVFHTQAE | | | CGGATACCGCGAGCGCGTTGTTTCCTGATGGCA |
| | | FREWFVKLYSTSENAK | | | CCAAAATTTATCTGTGGGATGGCAAAGTGTTTC |
| | | REITRFEVDGNVANIE | | | ATACCCAGGCGGAATTCCGCGAATGGTTTGTGA |
| | | VVLEANENGEQKKVKL | | | AACTGTATAGTACCAGCGAAAATGCGAAACGTG |
| | | RHIAEFEGDKLVEVNV | | | AAATCACCCGCTTTGAAGTGGATGGTAACGTGG |
| | | EIEPL | | | CGAACATTGAAGTTGTGCTGGAAGCGAATGAAA |
| | | | | | ACGGTGAACAGAAGAAAGTTAAACTGCGTCATA |
| | | | | | TTGCCGAATTTGAAGGCGATAAACTGGTGGAAG |
| | | | | | TGAACGTGGAAATTGAACCGCTGGG(TAA) |
| luxsiti_ | 99 | MSEKEIREFVERFYKA | 7. | 299 | (ATG)AGCGAAAAAGAAATTCGCGAATTTGTTG |
| 0.4_ | | LDDGDAETASSLEPDG | 232895646 | | AACGTTTTTATAAAGCGCTGGATGATGGCGATG |
| 1111 | | TRIYLWDGTEFSTQAE | | | CGGAAACCGCGAGCAGCCTGTTTCCGGATGGCA |
| | | FRAWFEELHSRSEAAR | | | CCCGCATTTATCTGTGGGATGGTACCGAATTTA |
| | | RRVVRLEVDGDEADVE | | | GCACCCAGGCGGAATTTCGCGCGTGGTTTGAAG |
| | | VVLDADFEGEHHRVRL | | | AACTGCATAGCCGCAGCGAAGCCGCGCGCAGAA |
| | | RHRFFFEGDRLVEVEV | | | GAGTGGTGCGTTTGGAAGTTGATGGTGATGAAG |
| | | TIEPL | | | CGGATGTGGAAGTGGTTCTGGATGCTGATTTTG |
| | | | | | AAGGCGAACATCATCGCGTGCGCCTGCGCCATC |
| | | | | | GCTTTTTCTTCGAAGGTGATCGCCTGGTTGAAG |
| | | | | | TCGAAGTGACCATTGAACCGCTGGG(TAA) |
| luxsiti_ | 100 | MSEKEIREFVDKFYKA | 296. | 300 | (ATG)AGCGAAAAAGAAATTCGCGAATTTGTGG |
| 0.4_ | | LDSGDAETASALFPDG | 7740152 | | ATAAATTTTATAAAGCGCTGGATAGCGGCGATG |
| 1122 | | TNIYLWDGTVEKTKEE | | | CCGAAACCGCTAGCGCGTTGTTTCCGGATGGCA |
| | | FRKWFVELKSTSDNAK | | | CCAACATTTATCTGTGGGATGGTACCGTGTTTA |
| | | RKVTKLEVHGNTAHVE | | | AAACCAAAGAAGAATTTCGCAAATGGTTTGTTG |
| | | VVLKASVNGEEKVVKL | | | AACTGAAAAGCACCAGCGATAACGCGAAACGCA |
| | | KHIFLFEGDKLREVYV | | | AAGTGACCAAACTGGAAGTGCATGGTAATACCG |
| | | EIEPL | | | CGCATGTGGAAGTTGTGCTGAAAGCGAGCGTGA |
| | | | | | ACGGTGAAGAAAAAGTGGTGAAATTGAAACATA |
| | | | | | TTTTTCTGTTTGAAGGCGATAAACTGCGTGAAG |
| | | | | | TTTATGTTGAAATCGAACCGCTGGG(TAA) |
| luxsiti_ | 101 | MSEEEQREFWRKFYEA | 385. | 301 | (ATG)AGCGAAGAAGAACAGCGCGAATTTTGGC |
| 0.4_ | | LDAGDAETASALFPDG | 2584658 | | GCAAATTTTATGAAGCGCTGGATGCGGGCGATG |
| 1125 | | TEIYLWDGIVERTRAE | | | CCGAAACTGCTAGCGCACTGTTTCCGGATGGCA |
| | | FRAWFRQLYSQSDNAK | | | CCGAAATTTATCTGTGGGATGGTACCGTGTTTC |
| | | RRITSFVVEGNRAYVE | | | GCACCCGCGCGGAATTTCGCGCGTGGTTTCGCC |
| | | VVLVANIKGKKVEVSL | | | AGCTGTATAGCCAGAGCGATAACGCGAAACGCC |
| | | KHLFEFEGSRLVRVYV | | | GCATTACCAGCTTTGTGGTGGAAGGCAACCGCG |
| | | EIKPL | | | CGTATGTGGAAGTGGTGCTGGTTGCGAACATTA |
| | | | | | AAGGCAAGAAAGTTGAAGTGAGCCTGAAACATT |
| | | | | | TGTTTGAATTTGAAGGCAGCCGCCTGGTGCGCG |
| | | | | | TGTATGTTGAAATTAAACCGCTGGG(TAA) |
| luxsiti_ | 102 | MSEQEQRDEVARFYAA | 2. | 302 | (ATG)AGCGAACAGGAACAGCGCGATTTTGTGG |
| 0.4_ | | LDAGDAETASALFRDG | 891499654 | | CGCGCTTTTATGCGGCGCTGGATGCGGGTGATG |
| 1126 | | TKIYLWDGKTERTQAE | | | CGGAAACTGCAAGCGCGCTGTTTCGTGATGGCA |
| | | FRSWFVELHSQSKDAK | | | CCAAAATTTACCTGTGGGATGGCAAAACCTTTC |
| | | RRVVSFRVDGNVADVN | | | GCACCCAGGCGGAATTTCGCAGCTGGTTTGTGG |
| | | VILHASVEGEERTVRL | | | AACTGCATAGCCAGAGCAAAGATGCGAAACGCC |
| | | NHVFKFEGDHLVEVRV | | | GCGTGGTGAGCTTTCGCGTGGATGGTAACGTGG |
| | | DIEPL | | | CGGATGTGAACGTGATTCTGCATGCGAGCGTGG |
| | | | | | AAGGCGAAGAACGCACCGTTCGCCTGAATCATG |
| | | | | | TGTTTAAATTTGAAGGTGATCATCTGGTGGAAG |
| | | | | | TGCGCGTTGATATCGAACCGCTGGG(TAA) |
| luxsiti_ | 103 | MSEEEIRAFWERFYSA | 2. | 303 | (ATG)AGCGAAGAAGAAATTCGCGCGTTTTGGG |
| 0.4_ | | LDAGDAETASSLFPDG | 270905321 | | AACGCTTTTATAGCGCGCTGGATGCAGGCGATG |
| 1134 | | TEIYLWDGKTERTQEE | | | CCGAAACGGCGAGCAGCCTGTTTCCAGATGGCA |
| | | FRAWFEELRSTSADAS | | | CCGAAATTTATCTGTGGGATGGCAAAACCTTTC |
| | | RHITSLKVEGNRADIE | | | GCACCCAGGAAGAATTTCGCGCCTGGTTTGAAG |
| | | VVLRANFKGEEKTVRL | | | AACTGCGCAGCACCAGCGCGGATGCAAGCAGAC |
| | | RHEAEFEGDRLVRVEV | | | ATATTACCAGCCTGAAAGTGGAAGGCAACCGCG |
| | | RIDPL | | | CGGACATTGAAGTGGTGCTGCGCGCGAACTTTA |
| | | | | | AAGGTGAAGAAAAGACCGTGCGCCTGCGCCATG |
| | | | | | AAGCGGAATTTGAAGGCGATCGCTTGGTGCGCG |
| | | | | | TGGAAGTGCGCATTGATCCGCTGGG(TAA) |
| luxsiti_ | 104 | MSAAEQREFVDRFYKA | 24. | 304 | (ATG)AGCGCGGCGGAACAGCGCGAATTTGTTG |
| 0.4_ | | LDAGDAETASALEPDG | 61161023 | | ATCGCTTTTATAAAGCGCTGGATGCGGGCGATG |
| 1135 | | TVIDLWDGRTERTRAE | | | CTGAAACCGCAAGCGCGTTGTTCCCTGATGGCA |
| | | FRAWFERLHATSTDAK | | | CCGTGATTGATCTGTGGGATGGCCGCACCTTTC |
| | | REVTQMKVDGNTVDVE | | | GCACCCGCGCAGAATTTCGCGCGTGGTTTGAAC |
| | | VVLRATVNGEEKVVKL | | | GCCTGCATGCGACCAGCACCGATGCGAAACGCG |
| | | RHQFHEEGDQLVRVRV | | | AAGTGACCCAGATGAAAGTGGATGGCAACACCG |
| | | DITPL | | | TGGATGTTGAAGTGGTGCTGCGCGCGACCGTGA |
| | | | | | ACGGTGAAGAAAAAGTGGTTAAACTGCGCCATC |
| | | | | | AGTTTCATTTTGAAGGCGATCAGCTGGTGCGCG |
| | | | | | TGCGTGTGGATATTACCCCGCTGGG(TAA) |
| luxsiti_ | 105 | MSESEIKEFVRKFYEA | 530. | 305 | (ATG)AGCGAAAGCGAAATTAAAGAATTTGTGC |
| 0.4_ | | LDAGDADTAAALFPDG | 8161714 | | GCAAATTTTATGAAGCGCTGGATGCAGGTGATG |
| 1139 | | TVIKLWDGTTFYTQAE | | | CCGATACCGCAGCGGCACTGTTTCCGGATGGCA |
| | | FRAWFVKLYSTSDEAS | | | CCGTGATTAAACTGTGGGATGGTACCACCTTTT |
| | | REVTSLKVEGDKAEVE | | | ATACCCAGGCGGAATTTCGCGCGTGGTTTGTGA |
| | | VVLKAKINGEEKTVKL | | | AACTGTATAGCACCAGCGATGAAGCCAGCCGCG |
| | | KHIFEFKGDKLVRVEV | | | AAGTGACCAGCCTGAAAGTGGAAGGCGATAAAG |
| | | SISPL | | | CGGAAGTTGAAGTGGTGCTGAAAGCCAAAATTA |
| | | | | | ACGGTGAAGAAAAGACCGTGAAATTGAAACATA |
| | | | | | TTTTTGAATTTAAAGGCGACAAACTGGTGCGCG |
| | | | | | TCGAAGTTAGCATTAGCCCGCTGGG(TAA) |
| luxsiti_ | 106 | MSEEEQREFWKRFYEA | 2. | 306 | (ATG)AGCGAAGAAGAACAGCGCGAATTTTGGA |
| 0.4_ | | LDAGDAETAAALFPDG | 404284727 | | AACGCTTTTATGAAGCGCTGGATGCGGGCGATG |
| 1141 | | TEIHLWDGKTERTQAE | | | CGGAAACCGCAGCAGCGTTGTTTCCAGATGGCA |
| | | FRAWFVQLRSTSEAAK | | | CCGAAATTCATCTGTGGGATGGCAAAACCTTTC |
| | | RKIIKFEVIGNKSFVE | | | GCACCCAGGCTGAATTTCGCGCGTGGTTTGTGC |
| | | VVLKASINGEEKEVEL | | | AGCTGCGCAGCACCAGTGAAGCGGCGAAACGCA |
| | | RHYFEFEGDKLVRVYV | | | AAATTATTAAATTTGAAGTGATTGGCAACAAAA |
| | | TIKPL | | | GCTTTGTGGAAGTGGTGCTGAAAGCGAGCATTA |
| | | | | | ACGGTGAAGAAAAAGAAGTGTTTCTGCGCCATT |
| | | | | | ATTTTGAATTCGAAGGCGATAAACTGGTGCGCG |
| | | | | | TGTATGTGACCATCAAACCGCTGGG(TAA) |
| luxsiti_ | 107 | MSEREIRQFVDREYAA | 1. | 307 | (ATG)AGCGAACGCGAAATTCGCCAGTTTGTGG |
| 0.4_ | | LDAGDAETAAALFPDG | 348306842 | | ATCGCTTTTATGCGGCGCTGGATGCGGGCGATG |
| 1160 | | TEIHLWDGRTETTQAE | | | CGGAAACTGCAGCAGCGTTGTTTCCAGATGGCA |
| | | FRAWFEKLRARSDNAR | | | CCGAAATCCATCTGTGGGATGGCCGCACCTTTA |
| | | REVVDLQVDGDEADVR | | | CCACCCAGGCGGAATTTCGCGCGTGGTTTGAAA |
| | | VVLDATFRGEARRVRL | | | AACTGCGTGCGCGCAGCGATAACGCGCGTCGCG |
| | | THRFLFEGDQVTRVEV | | | AAGTGGTTGATCTGCAGGTTGATGGCGATGAAG |
| | | EIRPD | | | CGGATGTGCGCGTTGTGCTGGACGCGACCTTTC |
| | | | | | GCGGTGAAGCGAGAAGAGTGCGTCTGACCCATC |
| | | | | | GCTTCCTGTTTGAAGGCGATCAGGTGACCCGCG |
| | | | | | TGGAAGTGGAAATTAGGCCGGATGG(TAA) |
| luxsiti_ | 108 | MSEAAQREFWDRFYRA | 3. | 308 | (ATG)AGCGAAGCGGCGCAGCGCGAATTTTGGG |
| 0.4_ | | LDAGDAETASALFPDG | 529371113 | | ATCGCTTTTATCGTGCGCTGGATGCGGGTGATG |
| 1162 | | TEIHLWDGTTERTRAE | | | CGGAAACCGCAAGCGCACTGTTTCCGGATGGCA |
| | | FRAWERDLHARSDAAR | | | CCGAAATCCATCTGTGGGATGGTACCACCTTTC |
| | | RSIVSFEVDGDDARVE | | | GCACCCGTGCGGAATTTCGCGCGTGGTTTCGCG |
| | | VVLHANVDGQERTVRL | | | ATCTGCATGCGAGAAGCGATGCGGCCCGTAGAA |
| | | RHEAHFEGDRLVRVHV | | | GCATTGTGAGCTTTGAAGTGGATGGCGATGATG |
| | | VIQPL | | | CCCGTGTGGAAGTGGTGCTGCACGCCAACGTGG |
| | | | | | ACGGTCAGGAACGTACCGTGCGTCTGAGACATG |
| | | | | | AAGCGCATTTTGAAGGCGACCGCCTGGTGCGCG |
| | | | | | TGCATGTGGTGATTCAGCCGCTGGG(TAA) |
| luxsiti_ | 109 | MSETAIRQFVERFYEA | 786. | 309 | (ATG)AGCGAAACCGCGATCCGCCAGTTTGTGG |
| 0.4_ | | LDAGDAETAAALFPDG | 2695232 | | AACGCTTTTATGAAGCGCTGGATGCGGGCGATG |
| 1169 | | TRIYLWDGTTFHTQAE | | | CGGAAACTGCAGCAGCCCTGTTTCCAGATGGCA |
| | | FRAWFEALHATSSGAK | | | CCCGCATTTATCTGTGGGATGGTACCACCTTTC |
| | | RHVVALQVDGDVADVE | | | ATACCCAGGCGGAATTTCGCGCGTGGTTTGAAG |
| | | VVLHADVDGEKRTVHL | | | CCCTGCATGCGACCAGCAGCGGTGCGAAACGTC |
| | | KHTFKERGDRIVEVEV | | | ATGTGGTGGCGTTGCAGGTTGATGGCGATGTGG |
| | | HIQPL | | | CGGATGTGGAAGTGGTGCTGCACGCCGATGTAG |
| | | | | | ATGGTGAAAAACGCACCGTTCATCTGAAACATA |
| | | | | | CCTTTAAATTTCGTGGCGATCGCCTGGTTGAAG |
| | | | | | TCGAAGTGCATATTCAGCCGCTGGG(TAA) |
| luxsiti_ | 110 | MSSDAQRAFVDRFYRA | 571. | 310 | (ATG)AGCAGCGATGCGCAGCGCGCCTTTGTGG |
| 0.4_ | | LDAGDAETASALFPDG | 707671 | | ATCGCTTTTATCGTGCGCTGGATGCGGGCGATG |
| 1172 | | TRIHLWDGTTETTREE | | | CCGAAACTGCGAGCGCGTTGTTTCCAGATGGCA |
| | | FRAWFVDLRSRSENAA | | | CCCGCATTCATCTGTGGGATGGTACCACCTTTA |
| | | REVVSFDVDGDVAHVE | | | CCACCCGCGAAGAATTTCGCGCGTGGTTTGTTG |
| | | VVLKAVIEGEEVVVRL | | | ATCTGCGCTCTCGCAGCGAAAACGCGGCGCGTG |
| | | RHVFEWEGDRIVEVYV | | | AAGTGGTGAGTTTTGATGTGGATGGCGATGTGG |
| | | EIDPL | | | CGCATGTGGAAGTTGTGCTGAAAGCGGTGATTG |
| | | | | | AAGGTGAAGAAGTCGTGGTTCGCCTGCGCCATG |
| | | | | | TGTTTGAATGGGAAGGCGATCGCCTGGTTGAAG |
| | | | | | TGTATGTAGAAATTGATCCGCTGGG(TAA) |
| luxsiti_ | 111 | MSEKDQKEFVKKFYEA | 423. | 311 | (ATG)AGCGAAAAAGATCAGAAAGAATTTGTGA |
| 0.4_ | | LDAGDAETASSLEPDG | 7816171 | | AGAAATTTTATGAAGCGCTGGATGCGGGCGATG |
| 1177 | | TEIHLWDGKVFHTQAE | | | CGGAAACCGCAAGCAGCTTGTTTCCGGATGGCA |
| | | FKAWFEELYSRSDDAK | | | CCGAAATCCATCTGTGGGATGGTAAAGTGTTTC |
| | | RSIISEKVDGNIAKVI | | | ATACCCAGGCGGAATTTAAAGCGTGGTTTGAAG |
| | | VVLKAFVDGEKLEVLL | | | AACTGTATAGCCGCAGCGATGATGCGAAACGCA |
| | | EHEFKFEGDKLVEVKV | | | GCATTATTAGCTTCAAAGTGGACGGCAACATTG |
| | | KIYPL | | | CGAAAGTGATTGTGGTGCTGAAAGCGTTTGTTG |
| | | | | | ATGGTGAAAAACTGGAAGTGCTGCTGGAACATG |
| | | | | | AATTCAAATTTGAAGGCGATAAACTGGTGGAAG |
| | | | | | TAAAAGTGAAAATTTATCCGCTGGG{TAA) |
| luxsiti_ | 112 | MSEEEQREFVRRFYDA | 417. | 312 | (ATG)AGCGAAGAAGAACAGCGTGAATTTGTGC |
| 0.4_ | | LDAGDAETASALFPDG | 7097443 | | GCCGCTTTTATGATGCGCTGGATGCGGGCGATG |
| 1178 | | TQIELWDGKTFHTREE | | | CGGAAACTGCAAGCGCGCTGTTTCCAGATGGCA |
| | | FRAWFVKLHSTSDDAR | | | CCCAGATTGAACTGTGGGATGGCAAAACCTTTC |
| | | REVVSESVAGDVANVE | | | ATACCCGTGAAGAATTTCGCGCGTGGTTTGTGA |
| | | VVLRANIRGEKKVVRL | | | AATTGCATAGCACCTCGGATGATGCCCGCCGCG |
| | | RHIFEFEGDRLVRVRV | | | AAGTGGTGAGCTTTAGCGTGGCAGGTGATGTGG |
| | | DIEPL | | | CGAACGTGGAAGTTGTGCTGCGTGCGAACATTC |
| | | | | | GCGGCGAAAAGAAAGTGGTTCGCCTGCGCCATA |
| | | | | | TTTTTGAATTCGAAGGCGATCGCCTGGTGCGCG |
| | | | | | TGCGTGTGGATATTGAACCGCTGGG(TAA) |
| luxsiti_ | 113 | MSEDEIREFVARFYAA | 732. | 313 | (ATG)AGCGAAGATGAAATTCGCGAATTTGTGG |
| 0.4_ | | LDAGDANTASALFPDG | 0027643 | | CGCGCTTTTATGCGGCGCTGGATGCGGGTGATG |
| 1186 | | TVIHLWDGITFHTQAE | | | CCAACACTGCAAGCGCGCTGTTTCCTGATGGCA |
| | | FRAWFEKLHSTSENAS | | | CCGTGATTCATCTGTGGGATGGTATTACCTTTC |
| | | REVVSLKVDGNVAHVE | | | ATACCCAGGCGGAATTTCGCGCGTGGTTTGAAA |
| | | VVLRASVDGEERTVRL | | | AACTGCATAGCACCAGCGAAAACGCGAGCCGCG |
| | | RHVFRFEGDKLVEVSV | | | AAGTGGTGAGCCTGAAAGTGGATGGCAACGTGG |
| | | SITPL | | | CGCATGTGGAAGTTGTTCTGCGCGCGAGCGTGG |
| | | | | | ACGGTGAAGAACGCACGGTTCGTCTGCGTCATG |
| | | | | | TGTTTCGCTTTGAAGGCGATAAACTGGTTGAAG |
| | | | | | TGAGCGTGAGCATTACCCCGCTGGG(TAA) |
| luxsiti_ | 114 | MSEEEIREFWKRFYEA | 6. | 314 | (ATG)TCAGAAGAAGAAATTCGCGAATTTTGGA |
| 0.2_ | | LDAGDAETASALFPDG | 0691085 | | AACGCTTTTATGAAGCGTTAGATGCGGGTGATG |
| 4 | | TRIYLWDGKEFTTQAE | | | CGGAAACCGCTAGTGCCCTGTTTCCGGATGGCA |
| | | FRAWFEELYSTSEDAS | | | CCCGCATTTATCTGTGGGATGGTAAAGAATTTA |
| | | REIVKLEVEGNVAYVE | | | CCACCCAGGCGGAATTTCGCGCGTGGTTTGAAG |
| | | VVLKANINGEEKVVKL | | | AACTGTATAGCACCAGCGAAGATGCTAGCCGCG |
| | | KHVFHFEGDRIVEVEV | | | AAATTGTGAAACTGGAAGTGGAAGGCAACGTGG |
| | | EIEPL | | | CGTATGTGGAAGTTGTGCTGAAAGCGAACATTA |
| | | | | | ACGGCGAAGAAAAAGTGGTTAAACTGAAACATG |
| | | | | | TGTTTCATTTTGAAGGCGATCGCCTGGTTGAAG |
| | | | | | TCGAAGTTGAAATTGAACCGCTGGG(TAA) |
| luxsiti_ | 115 | MSAEAQRREVDREYAA | 2569. | 315 | (ATG)AGCGCGGAAGCGCAGCGCCGCTTTGTGG |
| 0.2_ | | LDAGDADTASALEPDG | 767795 | | ATCGCTTTTATGCGGCGTTGGATGCGGGCGATG |
| 16 | | TEIHLWDGRTERTRAE | | | CGGATACCGCAAGTGCGCTGTTTCCAGATGGCA |
| | | FRAWFRELRARSDNAR | | | CCGAAATTCATCTGTGGGATGGCCGCACCTTTC |
| | | REVVAFEVDGDTAHVE | | | GCACCCGCGCCGAATTTCGCGCATGGTTTCGTG |
| | | VVLRASIDGEERVVRL | | | AACTGCGTGCGCGTAGCGATAACGCGCGCCGTG |
| | | RHTFYFEGDRLVRVEV | | | AAGTGGTGGCGTTTGAAGTTGATGGCGATACGG |
| | | EIEPL | | | CGCATGTGGAAGTTGTGCTGCGCGCGAGCATTG |
| | | | | | ATGGTGAAGAACGCGTGGTGCGCCTGCGTCATA |
| | | | | | CCTTTTATTTTGAAGGTGATCGCCTGGTGCGTG |
| | | | | | TTGAAGTCGAAATTGAACCGCTGGG(TAA) |
| luxsiti_ | 116 | MSEEEQREFVDRFYAA | 1702. | 316 | (ATG)AGCGAAGAAGAACAGCGCGAATTTGTTG |
| 0.2_ | | LDAGDAETASALFPDG | 83414 | | ATCGCTTTTATGCGGCGCTGGATGCGGGCGATG |
| 37 | | TKIYLWDGKVFTTREE | | | CAGAAACCGCGAGCGCATTGTTTCCTGATGGCA |
| | | FRAWFEKLYSTSENAK | | | CCAAAATTTATCTGTGGGATGGCAAAGTGTTTA |
| | | RHVVSFKVDGNKADVE | | | CCACCCGTGAAGAATTTCGCGCGTGGTTTGAAA |
| | | VVLHANINGEKKTVRL | | | AACTGTATAGCACCAGCGAAAACGCGAAACGCC |
| | | RHVFYFEGDKLVEVKV | | | ATGTTGTGAGCTTTAAAGTGGATGGTAACAAAG |
| | | EIKPL | | | CGGATGTGGAAGTGGTGCTGCATGCGAACATTA |
| | | | | | ACGGCGAAAAGAAAACCGTGCGCCTGCGCCACG |
| | | | | | TGTTTTATTTTGAAGGCGATAAACTGGTTGAAG |
| | | | | | TGAAAGTTGAAATTAAACCGCTGGG(TAA) |
| luxsiti_ | 117 | MSEEEQREFVKRFYEA | 448. | 317 | (ATG)AGCGAAGAAGAACAGCGTGAATTTGTGA |
| 0.2_ | | LDAGDAETASALFPDG | 5729095 | | AACGCTTTTATGAAGCGTTAGATGCGGGCGATG |
| 38 | | TEIHLWDGKTFYTREE | | | CCGAAACCGCGAGCGCATTGTTTCCGGATGGCA |
| | | FRAWFEKLYSTSDNAS | | | CCGAAATTCATCTGTGGGATGGTAAAACCTTTT |
| | | RSVTEFKVDGNKAKVK | | | ATACCCGCGAAGAGTTTCGCGCGTGGTTTGAAA |
| | | VVLKANINGEKKTVKL | | | AACTGTATAGCACCAGCGATAACGCGAGCCGTA |
| | | EHYFEFEGDKLVRVNV | | | GCGTGACCGAATTTAAAGTGGATGGGAACAAAG |
| | | TIKPL | | | CGAAAGTGAAAGTGGTGCTGAAAGCCAACATTA |
| | | | | | ACGGCGAAAAGAAAACCGTGAAACTGGAACATT |
| | | | | | ATTTTGAATTCGAAGGCGATAAACTGGTGCGCG |
| | | | | | TGAACGTGACCATTAAACCGCTGGG(TAA) |
| luxsiti_ | 118 | MSEEEQRRFVERFYAA | 2. | 318 | (ATG)AGCGAAGAAGAACAGCGCCGCTTTGTGG |
| 0.2_ | | LDAGDADTASALFPDG | 422944022 | | AACGCTTTTATGCGGCCCTGGATGCGGGTGATG |
| 39 | | TEIHLWDGTVERTRAE | | | CGGATACCGCAAGCGCATTGTTTCCGGATGGCA |
| | | FRAWFERLYSTSENAK | | | CCGAAATTCATCTGTGGGATGGTACCGTGTTTC |
| | | RHVVRFEVEGNVARVE | | | GCACCCGCGCGGAATTTCGCGCGTGGTTTGAAC |
| | | VVLHANIDGEERTVRL | | | GCCTGTATAGTACCAGCGAAAACGCGAAACGCC |
| | | THVFEFEGDRLVRVNV | | | ATGTGGTGCGCTTTGAAGTGGAAGGCAACGTGG |
| | | TINPL | | | CGCGCGTTGAAGTTGTGCTGCATGCGAACATTG |
| | | | | | ATGGTGAAGAACGCACCGTGCGCCTGACCCATG |
| | | | | | TGTTTGAATTTGAAGGCGACCGCCTGGTGCGTG |
| | | | | | TGAACGTGACCATTAACCCGCTGGG(TAA) |
| luxsiti_ | 119 | MSEEEIKEFVKRFYEA | 1532. | 319 | (ATG)AGTGAAGAAGAGATTAAAGAATTTGTGA |
| 0.2_ | | LDAGDAETASALFPDG | 327574 | | AACGCTTTTATGAAGCCCTGGATGCGGGCGATG |
| 65 | | TRIYLWDGRVFRTRAE | | | CGGAAACCGCGAGCGCTTTGTTTCCAGATGGCA |
| | | FRAWEVELHSTSEDAK | | | CCCGCATTTATCTGTGGGATGGTCGCGTGTTTC |
| | | REVIELKVEGNVAKVK | | | GCACCCGTGCGGAATTTCGCGCATGGTTTGTGG |
| | | VVLHANINGEKKTVLL | | | AACTGCATAGCACCAGCGAAGATGCGAAACGCG |
| | | EHYFEFEGDRIVEVRV | | | AAGTGATTGAACTGAAAGTGGAAGGCAACGTGG |
| | | EIKPL | | | CGAAAGTGAAAGTTGTGCTGCATGCCAACATCA |
| | | | | | ACGGCGAAAAGAAAACCGTTCTGCTGGAACATT |
| | | | | | ATTTTGAATTTGAAGGCGATCGCCTGGTGGAAG |
| | | | | | TGCGCGTGGAAATTAAACCGCTGGG(TAA) |
| luxsiti_ | 120 | MSEEAQKEFVKKFYEA | 11. | 320 | (ATG)AGCGAAGAAGCGCAGAAAGAATTTGTGA |
| 0.2_ | | LDAGDADTASALEPDG | 04077402 | | AGAAATTTTATGAAGCGCTGGATGCGGGCGATG |
| 66 | | TEIYLWDGKTFHTKAE | | | CGGATACCGCATCGGCTTTGTTTCCGGATGGCA |
| | | FKAWFVKLKSTSDNAK | | | CCGAAATTTATCTGTGGGATGGTAAAACCTTTC |
| | | RSVVKFEVDGNVAYVE | | | ATACCAAAGCGGAATTTAAAGCGTGGTTTGTTA |
| | | VVLHANINGEEKVVKL | | | AACTGAAAAGCACCAGCGATAACGCCAAACGCA |
| | | THIFEFEGDKLVKVNV | | | GCGTTGTGAAATTTGAAGTGGATGGGAACGTGG |
| | | TIKPL | | | CGTATGTGGAAGTGGTGCTGCATGCGAACATTA |
| | | | | | ACGGTGAAGAGAAAGTGGTCAAACTGACCCATA |
| | | | | | TTTTTGAATTCGAAGGCGATAAATTAGTGAAAG |
| | | | | | TGAACGTGACCATTAAACCGCTGGG(TAA) |
| luxsiti_ | 121 | MSEEEIREFVRRFYEA | 246. | 321 | (ATG)AGCGAAGAAGAAATTCGCGAATTTGTGC |
| 0.2_ | | LDAGDAETASALFPDG | 8369039 | | GCCGCTTTTATGAAGCGCTGGATGCGGGCGATG |
| 73 | | TVIYLWDGKTFHTREE | | | CGGAAACTGCGAGCGCATTGTTTCCTGATGGCA |
| | | FRAWFVELRSKSENAK | | | CCGTGATTTATCTGTGGGATGGCAAAACCTTTC |
| | | RHVVSLRVDGNVADVE | | | ATACCCGTGAAGAATTTCGTGCGTGGTTTGTGG |
| | | VVLDADINGEKKTVKL | | | AACTGCGCAGCAAAAGCGAAAACGCGAAACGCC |
| | | RHEFRFEGDRLVEVRV | | | ATGTGGTGAGCCTGCGCGTGGATGGTAATGTGG |
| | | EIEPL | | | CGGATGTGGAAGTGGTGCTGGACGCGGATATTA |
| | | | | | ACGGCGAAAAGAAAACCGTGAAACTGAGACATG |
| | | | | | AATTTAGATTTGAAGGCGATCGCCTGGTTGAAG |
| | | | | | TGCGCGTCGAAATTGAACCGCTGGG(TAA) |
| luxsiti_ | 122 | MSEEEIREFVKRFYEA | 211. | 322 | (ATG)AGCGAAGAAGAAATTCGCGAATTTGTGA |
| 0.2_ | | LDAGDADTASALFPDG | 3918452 | | AACGTTTTTATGAAGCGCTGGATGCGGGTGATG |
| 76 | | TKIYLWDGKTESTQAE | | | CGGATACCGCATCTGCGCTGTTTCCGGATGGCA |
| | | FKAWFVKLKSTSENAK | | | CCAAAATTTACCTGTGGGATGGTAAAACCTTTA |
| | | RKIVKLKVDGDVAEVE | | | GCACCCAGGCGGAATTTAAAGCCTGGTTTGTTA |
| | | VVLHANVNGEEKVVKL | | | AACTGAAAAGCACCAGCGAAAACGCCAAACGCA |
| | | KHKFKFEGDKLVEVEV | | | AAATTGTGAAGCTGAAAGTGGATGGCGATGTGG |
| | | EIKPL | | | CGGAAGTGGAAGTTGTGCTGCATGCGAACGTGA |
| | | | | | ACGGTGAAGAAAAAGTGGTGAAATTGAAACATA |
| | | | | | AATTCAAATTTGAAGGCGATAAACTGGTTGAGG |
| | | | | | TTGAAGTTGAAATTAAACCGCTGGG(TAA) |
| luxsiti_ | 123 | MSEEEQREFWKRFYEA | 1. | 323 | (ATG)AGCGAAGAAGAACAGCGCGAATTTTGGA |
| 0.2_ | | LDAGDADTASALFPDG | 211472011 | | AACGCTTTTATGAAGCGCTGGATGCGGGTGATG |
| 83 | | TKIHLWDGKTETTQAE | | | CGGATACCGCGAGCGCGTTGTTTCCAGATGGCA |
| | | FRAWFVELHSTSDDAK | | | CCAAAATTCATCTGTGGGATGGCAAAACCTTTA |
| | | REIVKFEVDGNKSYVE | | | CCACCCAGGCGGAATTTCGCGCGTGGTTTGTTG |
| | | VVLRANINGEEKVVRL | | | AACTGCATAGCACCAGCGATGATGCGAAACGCG |
| | | THETQFEGDKLVEVKV | | | AAATTGTGAAATTTGAAGTGGATGGTAACAAAA |
| | | TIEPL | | | GCTATGTGGAAGTGGTGCTGCGCGCGAACATTA |
| | | | | | ACGGTGAAGAAAAAGTGGTTCGCCTGACCCATG |
| | | | | | AAACCCAGTTTGAAGGCGATAAACTGGTTGAAG |
| | | | | | TTAAAGTGACCATTGAACCGCTGGG(TAA) |
| luxsiti_ | 124 | MSEEAQREFVRRFYAA | 184. | 324 | (ATG)AGTGAAGAAGCGCAGCGCGAATTTGTGC |
| 0.2_ | | LDAGDAETAAALFPDG | 6724 | | GCCGCTTTTATGCGGCGCTGGATGCGGGTGATG |
| 98 | | TVIHLWDGRTERTQAE | 257 | | CGGAAACAGCAGCAGCGTTGTTTCCGGATGGCA |
| | | FRAWEVELYSKSEDAK | | | CCGTGATTCATCTGTGGGATGGCCGCACCTTTC |
| | | REVVRFEVDGDRARVE | | | GCACCCAGGCGGAATTTCGCGCCTGGTTTGTGG |
| | | VVLKANFKGKKEVVKL | | | AACTGTATAGCAAAAGCGAAGATGCGAAACGTG |
| | | VHYFLFEGDRLVEVRV | | | AAGTGGTGCGTTTTGAAGTTGATGGCGATCGCG |
| | | EIKPL | | | CGCGTGTGGAAGTTGTGCTGAAAGCCAATTTTA |
| | | | | | AAGGCAAGAAAGAAGTCGTGAAACTGGTGCATT |
| | | | | | ATTTTCTGTTTGAAGGCGATAGACTGGTTGAAG |
| | | | | | TGCGCGTGGAAATTAAACCGCTGGG(TAA) |
| luxsiti_ | 125 | MSAEAQREFVRRFYAA | 746. | 325 | (ATG)AGCGCGGAAGCGCAGCGCGAATTTGTGC |
| 0.2_ | | LDAGDAETASALFPDG | 0829302 | | GCCGCTTTTATGCGGCGTTGGATGCGGGTGATG |
| 100 | | TRIHLWDGRVERTREE | | | CGGAAACCGCAAGCGCGCTGTTTCCAGATGGCA |
| | | FRAWFVKLYSTSEDAR | | | CCCGCATTCATCTGTGGGATGGCCGCGTGTTTC |
| | | REVTAFRVDGDRADVE | | | GCACCCGTGAAGAATTTCGCGCGTGGTTTGTGA |
| | | VVLRASINGEEKTVRL | | | AACTGTATAGCACCAGTGAAGATGCGCGCCGCG |
| | | RHVFQFEGDRLREVRV | | | AAGTGACGGCGTTTCGTGTTGATGGCGATCGTG |
| | | AIEPL | | | CCGATGTGGAAGTGGTGCTGCGCGCGAGCATTA |
| | | | | | ACGGCGAAGAAAAGACCGTGCGCCTGCGTCATG |
| | | | | | TGTTTCAGTTTGAAGGGGATCGTCTGCGTGAAG |
| | | | | | TGCGCGTCGCCATTGAACCGCTGGG(TAA) |
| luxsiti_ | 126 | MSEEEIREFCKRFYEA | 64. | 326 | (ATG)AGCGAAGAAGAAATTCGCGAATTTTGCA |
| 0.2_ | | LDAGDADTASALEPDG | 78921907 | | AACGCTTTTATGAAGCGCTGGATGCGGGCGATG |
| 108 | | TEIYLWDGRTFRTREE | | | CGGATACCGCAAGTGCGCTGTTTCCAGATGGCA |
| | | FRAWFVELHSTSDDAR | | | CCGAAATTTATCTGTGGGATGGCCGCACCTTTC |
| | | RSIVKLEVEGNVAYVE | | | GCACCCGTGAAGAATTTCGCGCGTGGTTTGTGG |
| | | VVLRANVDGEEKVVRL | | | AACTGCATAGCACCAGCGATGATGCCCGCCGCA |
| | | VHIFYFEGDKLVKVYV | | | GCATTGTGAAACTGGAAGTGGAAGGCAACGTGG |
| | | SIRPL | | | CGTATGTGGAAGTTGTGCTGCGCGCGAACGTGG |
| | | | | | ATGGGGAAGAAAAAGTGGTGCGCCTGGTGCATA |
| | | | | | TTTTCTATTTTGAAGGCGATAAACTGGTGAAAG |
| | | | | | TGTATGTGAGCATTCGCCCGCTGGG(TAA) |
| luxsiti_ | 127 | MSEEEIREFWKKFYEA | 106. | 327 | (ATG)AGCGAAGAAGAAATTCGCGAATTTTGGA |
| 0.2_ | | LDAGDAETASALFPDG | 1672426 | | AAAAGTTTTTATGAAGCGCTGGATGCGGGTGATG |
| 118 | | TKIHLWDGKTENTQAE | | | CGGAAACCGCGAGCGCACTGTTTCCTGATGGTA |
| | | FKAWFVKLYSESDDAK | | | CCAAAATTCATCTGTGGGATGGCAAAACCTTTA |
| | | REIVELEVDGNVAYVK | | | ACACCCAGGCGGAATTTAAAGCGTGGTTTGTGA |
| | | VVLHANYKGEQKTVEL | | | AACTGTATAGCGAAAGCGATGATGCGAAACGCG |
| | | KHIFKFEGDKLVEVNV | | | AAATTGTGGAACTGGAAGTGGATGGTAACGTGG |
| | | EIKPL | | | CGTATGTGAAAGTGGTGCTGCATGCGAACTATA |
| | | | | | AAGGCGAACAGAAAACCGTTGAACTGAAACATA |
| | | | | | TTTTTAAATTTGAAGGCGATAAACTGGTGGAAG |
| | | | | | TTAACGTTGAAATTAAACCGCTGGG(TAA) |
| luxsiti_ | 128 | MSEEEQREFWKKFYEA | 1059. | 328 | (ATG)AGCGAAGAAGAACAGCGCGAATTTTGGA |
| 0.2_ | | LDAGDAETASALFPDG | 310297 | | AAAAGTTTTATGAAGCGCTGGATGCGGGCGATG |
| 119 | | TKIYLWDGKVFYTQEE | | | CGGAAACCGCAAGCGCCTTGTTTCCAGATGGCA |
| | | FRAWFVELRSQSDDAK | | | CCAAAATTTATCTGTGGGATGGCAAAGTGTTCT |
| | | RKIVDFKVEGDVAYVE | | | ATACCCAGGAAGAATTTCGCGCGTGGTTTGTGG |
| | | VVLEANINGEKKTVKL | | | AACTGCGCAGCCAGAGCGATGATGCGAAACGCA |
| | | KHIYKFEGDKLVEVKV | | | AAATTGTGGATTTTAAAGTGGAAGGCGATGTGG |
| | | KIEPL | | | CGTATGTGGAAGTGGTGCTGGAAGCGAACATTA |
| | | | | | ATGGCGAAAAGAAAACCGTGAAACTGAAACATA |
| | | | | | TTTATAAATTCGAAGGTGATAAACTGGTTGAAG |
| | | | | | TGAAAGTTAAAATCGAACCGCTGGG(TAA) |
| luxsiti_ | 129 | MSEEEQREFWRRFYEA | 2. | 329 | (ATG)AGCGAAGAAGAACAGCGCGAATTTTGGC |
| 0.2_ | | LDAGDAETASALFPDG | 00829302 | | GCCGCTTTTATGAAGCGCTGGATGCGGGCGATG |
| 123 | | TKIHLWDGRTETTQEE | | | CGGAAACCGCCTCTGCACTGTTTCCAGATGGCA |
| | | FRAWFVDLHSRSDDAK | | | CCAAAATTCATCTGTGGGATGGCCGCACCTTTA |
| | | REIVSEKVEGNKARVE | | | CCACCCAGGAAGAATTTCGCGCGTGGTTTGTGG |
| | | VVLRAREDGEERVVAL | | | ATCTGCATAGCCGCAGCGATGATGCGAAACGCG |
| | | RHETEFEGDRIVEVNV | | | AAATTGTGAGCTTTAAAGTGGAAGGCAACAAAG |
| | | DIRPL | | | CGCGCGTTGAAGTGGTGCTGCGCGCACGTGAAG |
| | | | | | ATGGTGAAGAACGCGTTGTGGCGCTGCGTCATG |
| | | | | | AAACCGAATTTGAAGGCGATCGCCTGGTGGAAG |
| | | | | | TGAACGTGGATATTCGCCCGCTGGG(TAA) |
| luxsiti_ | 130 | MSEEAQREFVRRFYAA | 12. | 330 | (ATG)AGCGAAGAAGCGCAGCGTGAATTTGTGC |
| 0.2_ | | LDAGDADTASALEPDG | 30131306 | | GCCGCTTTTATGCGGCGTTGGATGCGGGTGATG |
| 125 | | TRIYLWDGRTFTTRAE | | | CGGATACCGCTAGCGCCTTATTTCCGGATGGCA |
| | | FRAWFERLHATSADAK | | | CCCGCATTTATCTGTGGGATGGCCGCACCTTTA |
| | | REVVDFEVEGNKAKVK | | | CCACGCGCGCGGAATTTCGCGCATGGTTTGAAC |
| | | VVLHANVNGEEKTVLL | | | GCCTGCATGCGACCAGCGCAGATGCGAAACGGG |
| | | EHWFEFEGDRLVRVEV | | | AAGTGGTGGATTTTGAAGTGGAAGGCAACAAAG |
| | | TIKPL | | | CGAAAGTGAAAGTGGTTCTGCACGCGAACGTGA |
| | | | | | ACGGTGAAGAAAAGACCGTGCTGCTGGAACATT |
| | | | | | GGTTCGAATTTGAAGGCGATCGCCTGGTGCGCG |
| | | | | | TGGAAGTGACCATTAAACCGCTGGG(TAA) |
| luxsiti_ | 131 | MSEKEQREFWKKFYEA | 159. | 331 | (ATG)AGCGAAAAAGAACAGCGCGAATTTTGGA |
| 0.2_ | | LDAGDAETASALFPDG | 0704907 | | AAAAGTTTTATGAAGCGTTGGATGCGGGTGATG |
| 126 | | TKIYLWDGKTFTTQAE | | | CCGAAACCGCGAGCGCGCTGTTTCCAGATGGCA |
| | | FRAWFVELRSTSENAK | | | CCAAAATTTATCTGTGGGACGGCAAAACCTTTA |
| | | RKIVSFKVDGNKSEVE | | | CCACCCAGGCCGAATTTCGCGCGTGGTTTGTGG |
| | | VVLEANINGEKKVVKL | | | AACTGCGCAGCACCAGCGAGAACGCCAAACGCA |
| | | KHIFKFEGDKLVEVKV | | | AAATTGTGAGCTTTAAAGTGGATGGCAACAAAA |
| | | EIIPL | | | GCGAAGTGGAAGTGGTCCTGGAAGCGAACATCA |
| | | | | | ACGGCGAAAAGAAAGTGGTTAAACTGAAACACA |
| | | | | | TTTTTAAATTTGAAGGCGATAAACTGGTTGAAG |
| | | | | | TGAAAGTTGAAATTATTCCGCTGGG(TAA) |
| luxsiti_ | 132 | MSEEEQREFWRKFYEA | 1. | 332 | (ATG)TCCGAAGAAGAACAGCGCGAATTTTGGC |
| 0.2_ | | LDAGDADTASALFPDG | 08707671 | | GCAAATTTTATGAAGCGCTGGATGCGGGTGATG |
| 127 | | TEIHLWDGKTERTREE | | | CGGATACCGCGTCTGCGCTGTTTCCAGATGGCA |
| | | FRAWFEELYSTSEDAK | | | CCGAAATTCATCTGTGGGATGGCAAAACCTTTC |
| | | REIVKFEVEGNKSFVE | | | GCACCCGCGAAGAATTTCGCGCGTGGTTTGAAG |
| | | VVLKANVNGKKVVVKL | | | AACTGTATAGCACCAGCGAAGATGCGAAACGCG |
| | | RHVFEFEGDKVVKVEV | | | AAATTGTGAAATTTGAAGTGGAAGGCAACAAAA |
| | | EIEPL | | | GCTTTGTGGAAGTGGTGCTGAAAGCGAACGTCA |
| | | | | | ACGGCAAGAAAGTGGTTGTCAAACTGCGCCATG |
| | | | | | TGTTTGAATTCGAAGGCGATAAAGTTGTGAAGG |
| | | | | | TTGAAGTTGAAATTGAACCGCTGGG(TAA) |
| luxsiti_ | 133 | MSEEEIKKEWEKFYNA | 1. | 333 | (ATG)AGCGAAGAAGAAATTAAAAAGTTTTGGG |
| 0.2_ | | LDAGDADTASALFPDG | 702833449 | | AAAAATTTTATAACGCCCTGGATGCGGGTGATG |
| 132 | | TEIHLWDGKTFKTQAE | | | CGGATACTGCAAGCGCTCTGTTTCCGGACGGTA |
| | | FREWFVKLYSTSDDAK | | | CCGAAATTCATCTGTGGGATGGCAAAACCTTTA |
| | | REIVKLEVDGNVAYVE | | | AAACCCAGGCGGAATTTCGCGAATGGTTTGTGA |
| | | VVLRANVDGEEKTVKL | | | AACTGTATAGCACCAGCGATGATGCGAAACGCG |
| | | RHVAVFEGDKLKEVKV | | | AAATTGTTAAACTGGAAGTGGATGGTAACGTGG |
| | | TIKPL | | | CGTATGTGGAAGTTGTGCTGCGCGCCAACGTGG |
| | | | | | ACGGTGAAGAAAAGACCGTAAAACTGCGCCATG |
| | | | | | TGGCGGTGTTTGAAGGCGATAAACTGAAAGAAG |
| | | | | | TGAAAGTGACCATTAAACCGCTGGG(TAA) |
| luxsiti_ | 134 | MSEEEQREFVRRFYEA | 100. | 334 | (ATG)AGCGAAGAAGAACAGCGCGAATTTGTGC |
| 0.2_ | | LDAGDAETASALFPDG | 1679 | | GCCGCTTTTATGAAGCGCTGGATGCGGGCGATG |
| 142 | | TKIHLWDGKTESTRAE | 337 | | CGGAAACTGCGAGCGCGTTGTTTCCAGATGGCA |
| | | FRAWEVELRSTSENAK | | | CCAAAATTCATCTGTGGGATGGCAAAACCTTTA |
| | | RRVTSFRVEGNEADVE | | | GCACCCGCGCGGAATTTCGTGCGTGGTTTGTGG |
| | | VVLEATVNGEKKRVRL | | | AACTGCGTAGCACCAGCGAAAACGCCAAACGCC |
| | | RHRFLEEGDRIVEVYV | | | GCGTGACCAGCTTTCGTGTGGAAGGCAACGAAG |
| | | EIKPL | | | CGGATGTTGAAGTGGTGCTGGAAGCGACCGTGA |
| | | | | | ACGGCGAAAAGAAACGCGTGCGCCTGCGTCACC |
| | | | | | GCTTTCTGTTTGAAGGCGATCGCCTGGTGGAAG |
| | | | | | TGTATGTGGAAATTAAACCGCTGGG(TAA) |
| luxsiti_ | 135 | MSEEEQREFVKRFYEA | 257. | 335 | (ATG)AGCGAAGAAGAACAGCGCGAATTTGTGA |
| 0.2_ | | LDAGDAETASALFPDG | 4740843 | | AACGTTTTTATGAAGCGCTGGATGCGGGCGATG |
| 144 | | TLIYLWDGKTETTQAE | | | CGGAAACTGCAAGCGCACTGTTTCCGGATGGCA |
| | | FRAWFEKLRSTSEDAK | | | CCCTGATTTATCTGTGGGATGGGAAAACCTTTA |
| | | REVVEFKVDGNVADVV | | | CCACCCAGGCGGAATTTCGCGCATGGTTTGAAA |
| | | VVLEANINGEKKVVKL | | | AACTGCGCAGCACCAGCGAGGATGCGAAACGCG |
| | | RHRFHFEGDKLVKVEV | | | AAGTGGTGGAATTTAAAGTTGATGGTAACGTGG |
| | | EIEPL | | | CGGATGTGGTGGTTGTGCTGGAAGCGAACATTA |
| | | | | | ACGGCGAAAAGAAAGTGGTTAAGCTGCGTCATC |
| | | | | | GCTTTCATTTTGAAGGCGATAAACTGGTCAAAG |
| | | | | | TGGAAGTTGAAATTGAACCGCTGGG(TAA) |
| luxsiti_ | 136 | MSEEEQREFVKRFYEA | 376. | 336 | (ATG)AGCGAAGAAGAACAGCGCGAATTTGTAA |
| 0.2_ | | LDAGDADTASALFPDG | 0767104 | | AACGCTTTTATGAAGCGTTGGATGCCGGTGATG |
| 147 | | TKIHLWDGTTETTQAE | | | CGGATACCGCGAGCGCGTTGTTTCCGGATGGCA |
| | | FKAWEEKLYSKSDNAK | | | CCAAAATTCATCTGTGGGATGGTACCACCTTTA |
| | | RHVVSFKVEGNVAYVE | | | CCACCCAGGCGGAATTTAAAGCGTGGTTTGAAA |
| | | VVLHANFKGEEKTVSL | | | AACTGTATAGCAAAAGCGATAACGCCAAACGCC |
| | | KHIFKFEGDKLVEVEV | | | ATGTGGTGAGCTTTAAAGTGGAAGGCAACGTGG |
| | | KIKPL | | | CGTATGTGGAAGTGGTGCTGCATGCCAATTTTA |
| | | | | | AAGGTGAAGAAAAGACCGTGAGCCTGAAACATA |
| | | | | | TCTTTAAATTTGAAGGCGATAAACTGGTTGAAG |
| | | | | | TCGAAGTGAAAATTAAACCGCTGGG(TAA) |
| luxsiti_ | 137 | MSEEEQREFVRRFYEA | 3. | 337 | (ATG)AGCGAAGAAGAACAGCGCGAATTTGTGC |
| 0.2_ | | LDAGDAETASALEPDG | 706288874 | | GCCGTTTTTATGAAGCGCTGGATGCGGGTGATG |
| 149 | | TVIHLWDGKTERTQAE | | | CGGAAACTGCGAGCGCACTGTTTCCTGATGGCA |
| | | FRAWFVELKSTSEDAK | | | CCGTGATTCATCTGTGGGATGGCAAAACCTTTC |
| | | RRVVSFRVDGDEAEVV | | | GCACCCAGGCGGAATTTCGCGCGTGGTTTGTGG |
| | | VVLHANIDGEERVVRL | | | AACTGAAAAGCACCAGCGAGGATGCGAAACGTC |
| | | THRFLEEGDRVVEVWV | | | GCGTTGTGAGCTTTCGTGTGGATGGCGATGAAG |
| | | EIEPL | | | CCGAAGTGGTGGTTGTGCTGCATGCGAACATTG |
| | | | | | ATGGTGAAGAACGCGTCGTGCGCCTGACCCATC |
| | | | | | GCTTTCTGTTTGAAGGTGATCGCGTAGTGGAAG |
| | | | | | TGTGGGTGGAAATTGAACCGCTGGG(TAA) |
| luxsiti_ | 138 | MSEQEQREFVDRFYRA | 1. | 338 | (ATG)AGCGAACAGGAACAGCGCGAATTTGTGG |
| 0.2_ | | LDAGDADTASALFPDG | 767104354 | | ATCGCTTTTATCGTGCGCTGGATGCGGGTGATG |
| 158 | | TEIYLWDGKTERTQAE | | | CGGATACCGCGAGCGCACTGTTTCCTGATGGTA |
| | | FREWFVKLHSTSEDAK | | | CCGAAATTTATCTGTGGGATGGCAAAACCTTTC |
| | | RRVVSFSVDGNVADVE | | | GCACCCAGGCGGAATTTCGCGAATGGTTTGTGA |
| | | VVLEANVNGEKKTVKL | | | AACTGCATAGCACCAGCGAAGATGCCAAACGCC |
| | | KHRFHFEGDKVVRVEV | | | GCGTGGTGAGCTTTAGCGTGGATGGTAACGTGG |
| | | SIEPL | | | CGGATGTGGAAGTGGTGCTGGAAGCGAATGTGA |
| | | | | | ACGGCGAAAAGAAAACCGTCAAACTGAAACATC |
| | | | | | GCTTCCATTTTGAAGGCGATAAAGTGGTTCGCG |
| | | | | | TTGAAGTGAGCATTGAACCGCTGGG(TAA) |
| luxsiti_ | 139 | MSEEEIREFVRRFYAA | 397. | 339 | (ATG)AGCGAAGAAGAAATTCGCGAATTTGTGC |
| 0.2_ | | LDAGDAETASALFPDG | 2411887 | | GCCGCTTTTATGCGGCGCTGGATGCGGGTGATG |
| 161 | | TRIHLWDGRTETTQAE | | | CGGAAACTGCAAGCGCGTTGTTTCCGGATGGCA |
| | | FREWEVKLYSESDDAR | | | CCCGCATTCATCTGTGGGATGGCCGCACCTTTA |
| | | REVVSLEVDGDRALVR | | | CCACCCAGGCGGAATTTCGTGAATGGTTTGTGA |
| | | VVLRASYKGEERVVEL | | | AACTGTATAGCGAAAGCGATGATGCGCGCCGCG |
| | | RHEFLFEGDELVEVRV | | | AAGTGGTGAGCCTGGAAGTGGATGGCGATCGTG |
| | | EIRPL | | | CATTGGTGCGCGTGGTCCTGCGTGCAAGCTATA |
| | | | | | AAGGTGAAGAACGCGTCGTGGAACTGCGCCATG |
| | | | | | AATTTCTGTTTGAAGGCGATGAACTGGTGGAAG |
| | | | | | TTCGCGTGGAAATTAGACCGCTGGG{TAA) |
| luxsiti_ | 140 | MSEEEQREFWKRFYEA | 174. | 340 | (ATG)AGCGAAGAAGAACAGCGCGAATTTTGGA |
| 0.2_ | | LDAGDAETASALFPDG | 252246 | | AACGCTTTTATGAAGCGCTGGATGCGGGCGATG |
| 164 | | TEIHLWDGTVERTRAE | | | CGGAAACTGCAAGCGCGCTGTTTCCAGATGGCA |
| | | FRAWFEQLYAESDDAK | | | CCGAAATTCATCTGTGGGATGGTACCGTGTTTC |
| | | REIVSFKVEGNVSDVE | | | GCACCCGCGCGGAATTTCGCGCGTGGTTTGAAC |
| | | VVLHASYKGEKKTVKL | | | AGCTGTATGCCGAAAGCGATGATGCGAAACGCG |
| | | RHRFFFEGDRIVEVEV | | | AAATTGTGAGCTTTAAAGTGGAAGGCAACGTTA |
| | | EIEPL | | | GCGATGTGGAAGTGGTGCTGCATGCGAGCTATA |
| | | | | | AAGGCGAAAAGAAAACCGTGAAACTGAGACATC |
| | | | | | GCTTTTTCTTTGAAGGCGATCGTCTGGTTGAAG |
| | | | | | TCGAAGTTGAAATTGAACCGCTGGG(TAA) |
| luxsiti_ | 141 | MSEEEQREFVKRFYEA | 1. | 341 | (ATG)AGCGAAGAAGAACAGCGTGAATTTGTGA |
| 0.2_ | | LDAGDAETASALEPDG | 134070491 | | AACGCTTTTATGAAGCGCTGGATGCGGGTGATG |
| 165 | | TEIHLWDGITFHTQAE | | | CGGAAACCGCAAGCGCGCTGTTTCCAGATGGCA |
| | | FREWFVKLRSTSENAK | | | CCGAAATTCATCTGTGGGATGGCATTACCTTTC |
| | | REVVKFEVDGNKAKVE | | | ATACCCAGGCGGAATTTCGCGAATGGTTTGTTA |
| | | VVLKANINGEEKEVKL | | | AACTGCGCAGCACCAGCGAAAACGCGAAACGTG |
| | | THTFEFEGDKLVKVNV | | | AAGTGGTGAAATTTGAAGTTGATGGTAACAAAG |
| | | DIKPL | | | CGAAAGTGGAAGTTGTGCTGAAAGCAAACATTA |
| | | | | | ACGGTGAAGAAAAAGAAGTGAAACTGACCCATA |
| | | | | | CCTTTGAATTCGAAGGCGATAAATTAGTGAAAG |
| | | | | | TGAACGTGGATATTAAACCGCTGGG(TAA) |
| luxsiti_ | 142 | MSAEEIREFVRRFYEA | 25. | 342 | (ATG)AGCGCGGAAGAAATTCGCGAATTTGTGC |
| 0.2_ | | IDAGDADTASALEPDG | 31720802 | | GTCGCTTTTATGAAGCGCTGGATGCGGGTGATG |
| 173 | | TEIHLWDGRTERTQAE | | | CCGATACCGCGAGTGCGCTGTTTCCTGATGGCA |
| | | FRAWFVRLRATSDDAS | | | CCGAAATTCATCTGTGGGATGGCCGCACCTTTC |
| | | REVVSLEVDGNVADVE | | | GCACCCAGGCGGAATTTCGCGCATGGTTTGTGA |
| | | VVLHANVNGEKKVVRE | | | GGCTGCGCGCGACAAGCGATGATGCGAGCCGTG |
| | | RHRFYFEGDRLVRVEV | | | AAGTGGTGAGCTTGGAAGTGGATGGCAACGTTG |
| | | TIVPL | | | CGGATGTGGAAGTTGTGCTGCATGCGAACGTGA |
| | | | | | ACGGCGAAAAGAAAGTGGTTCGCCTGCGCCATC |
| | | | | | GCTTCTATTTTGAAGGCGATCGCCTGGTGCGCG |
| | | | | | TTGAAGTGACCATTGTGCCGCTGGG(TAA) |
| luxsiti_ | 143 | MSKEEIKEFVKRFYQA | 127. | 343 | (ATG)AGCAAAGAAGAAATTAAAGAATTTGTTA |
| 0.2_ | | LDAGDAETASSLEPDG | 8949551 | | AACGCTTTTATCAGGCGCTGGATGCGGGCGATG |
| 176 | | TKIYLWDGKVEKTQEE | | | CGGAAACAGCGAGCAGCCTGTTTCCAGATGGCA |
| | | FRAWFEKLHSTSENAK | | | CCAAAATTTATCTGTGGGATGGCAAAGTGTTTA |
| | | REVTKLEVEGNVAYIE | | | AAACCCAGGAAGAGTTTCGCGCGTGGTTTGAAA |
| | | VVLKANVNGEEKIVNL | | | AACTGCATAGCACCAGCGAAAACGCGAAACGTG |
| | | THKFYFEGDKLVEVEV | | | AAGTGACCAAACTGGAAGTTGAAGGCAACGTGG |
| | | KIVPL | | | CGTATATTGAAGTGGTGCTGAAAGCGAACGTTA |
| | | | | | ACGGCGAAGAAAAGATTGTGAACCTGACCCATA |
| | | | | | AATTTTATTTCGAAGGCGATAAACTGGTCGAAG |
| | | | | | TGGAAGTGAAAATTGTTCCGCTGGG(TAA) |
| luxsiti_ | 144 | MSEEEIREFVKRFYEA | 246. | 344 | (ATG)AGCGAAGAAGAAATCCGCGAATTTGTGA |
| 0.2_ | | LDAGDAETASALFPDG | 1610228 | | AACGCTTTTATGAAGCGCTGGATGCGGGCGATG |
| 184 | | TKIHLWDGTVETTQAE | | | CGGAAACGGCGAGCGCATTGTTTCCAGATGGCA |
| | | FRAWEEKLYSTSDNAS | | | CCAAAATTCATCTGTGGGATGGTACCGTGTTTA |
| | | RSVVSFKVDGNVAEVE | | | CCACCCAGGCGGAATTTCGCGCGTGGTTTGAAA |
| | | VVLKANIKGREKVVKL | | | AACTGTATAGCACCAGCGATAACGCGAGCCGCA |
| | | KHIFHFEGDKLVEVEV | | | GCGTGGTGAGCTTTAAAGTGGATGGCAACGTGG |
| | | SIEPL | | | CGGAAGTGGAAGTTGTGCTGAAAGCGAACATTA |
| | | | | | AAGGCCGCGAAAAAGTGGTGAAACTGAAACATA |
| | | | | | TTTTTCATTTTGAAGGCGATAAACTGGTTGAAG |
| | | | | | TCGAAGTGAGCATTGAACCGCTGGG(TAA) |
| luxsiti_ | 145 | MSEEEIREFVKRFYEA | 800. | 345 | (ATG)AGCGAAGAAGAAATTCGCGAATTTGTGA |
| 0.2_ | | LDAGDAETASALFPDG | 6247408 | | AACGCTTTTATGAAGCGCTGGATGCGGGTGATG |
| 186 | | TVIHLWDGITEHTQAE | | | CCGAAACAGCGTCCGCGCTGTTTCCAGATGGCA |
| | | FRAWFEALYSTSENAR | | | CCGTGATTCATCTGTGGGATGGCATTACCTTTC |
| | | REVVALRVEGNRARVE | | | ATACCCAGGCGGAATTTCGCGCGTGGTTTGAAG |
| | | VVLHANVDGEERTVRL | | | CCCTGTATAGCACCAGCGAAAATGCGCGCCGCG |
| | | RHEFLFEGDRIVEVRV | | | AAGTGGTGGCGTTGAGAGTGGAAGGTAACCGTG |
| | | EIEPL | | | CTCGTGTGGAAGTTGTGCTGCATGCGAACGTGG |
| | | | | | ATGGTGAAGAACGCACCGTTCGCCTGCGCCATG |
| | | | | | AATTTCTGTTCGAAGGCGATCGCCTGGTCGAAG |
| | | | | | TGCGCGTTGAAATTGAACCGCTGGG(TAA) |
| luxsiti_ | 146 | MSEEAQREFVKRFYEA | 221. | 346 | (ATG)TCCGAAGAAGCGCAGCGCGAATTTGTGA |
| 0.2_ | | LDAGDAETASALFPDG | 3282654 | | AACGCTTTTATGAAGCGCTGGATGCGGGTGATG |
| 191 | | TRIHLWDGRTFTTREE | | | CGGAAACCGCCAGCGCGTTGTTTCCAGATGGCA |
| | | FRAWFEELRSKSEDAK | | | CCCGCATTCATCTGTGGGATGGCCGCACCTTTA |
| | | REVTSFRVDGNVAEVE | | | CCACCCGTGAAGAATTTCGCGCGTGGTTTGAAG |
| | | VVLHANINGEEKTVLL | | | AACTGCGCAGCAAAAGCGAAGATGCGAAACGCG |
| | | KHRFVFEGDRLVEVHV | | | AAGTGACCAGCTTTCGCGTGGATGGCAACGTGG |
| | | EIKPL | | | CGGAAGTGGAAGTTGTGCTGCATGCCAACATCA |
| | | | | | ACGGCGAAGAAAAGACCGTGCTGCTGAAACATC |
| | | | | | GCTTTGTGTTCGAAGGCGATCGCCTGGTTGAAG |
| | | | | | TGCATGTGGAAATTAAACCGCTGGG(TAA) |
| luxsiti_ | 147 | MSEEAQRQFVRREYEA | 33. | 347 | (ATG)AGCGAAGAAGCCCAGCGTCAGTTTGTGC |
| 0.2_ | | LDAGDADTASALFPDG | 65376641 | | GCCGCTTTTATGAAGCCCTGGATGCGGGCGATG |
| 193 | | TEIHLWDGRTERTRAE | | | CGGATACCGCTAGCGCACTGTTTCCAGATGGCA |
| | | FRAWFERLRATSADAR | | | CCGAAATTCATCTGTGGGATGGCCGCACCTTTC |
| | | RSVTSFEVDGDFARVE | | | GCACCCGCGCGGAATTTCGTGCATGGTTTGAAC |
| | | VVLRASFAGEERVVRL | | | GCCTGCGCGCGACAAGCGCAGATGCCCGCAGAA |
| | | RHYFQFEGDRLVRVEV | | | GCGTGACTTCTTTTGAAGTGGATGGCGATTTTG |
| | | EIRPL | | | CGCGTGTGGAAGTGGTGCTGCGTGCGAGCTTTG |
| | | | | | CCGGAGAAGAACGTGTGGTGCGTCTGCGTCATT |
| | | | | | ATTTTCAGTTCGAAGGCGATCGCCTGGTGCGCG |
| | | | | | TTGAAGTTGAAATTCGCCCGCTGGG(TAA) |
| luxsiti_ | 148 | MSKKAQEEFWKRFYEA | 1. | 348 | (ATG)AGCAAAAAGGCGCAGGAAGAATTTTGGA |
| 0.2_ | | LDAGDADTASALEPDG | 765031099 | | AACGCTTTTACGAAGCGCTGGATGCGGGTGATG |
| 194 | | TKIYLWDGKTFTTQAE | | | CGGATACCGCAAGCGCGCTGTTTCCTGATGGCA |
| | | FRAWFVELHSKSDNAK | | | CCAAAATTTATCTGTGGGATGGCAAAACCTTTA |
| | | RRITKFEVDGNKSYVE | | | CCACCCAGGCCGAATTTCGCGCGTGGTTTGTGG |
| | | VVLEANVNGKKEVVKL | | | AACTGCATAGCAAAAGCGATAACGCGAAACGCC |
| | | VHETLFEGDKLVEVRV | | | GCATTACCAAATTTGAAGTGGATGGTAACAAAA |
| | | DIKPL | | | GCTATGTGGAAGTGGTGCTGGAAGCGAACGTGA |
| | | | | | ACGGCAAGAAAGAAGTTGTGAAACTGGTGCATG |
| | | | | | AAACCCTGTTTGAAGGCGATAAATTGGTTGAAG |
| | | | | | TTCGCGTGGATATTAAACCGCTGGG(TAA) |
| luxsiti_ | 149 | MSEEEQREFWRRFYEA | 959. | 349 | (ATG)AGCGAAGAAGAACAGCGCGAATTTTGGC |
| 0.2_ | | LDAGDAETASALEPDG | 2121631 | | GTCGCTTTTATGAAGCGCTGGATGCGGGCGATG |
| 198 | | TEIHLWDGKVERTQAE | | | CCGAAACTGCTAGCGCACTGTTTCCAGATGGCA |
| | | FRAWFERLRATSADAK | | | CCGAAATTCATCTGTGGGATGGCAAAGTGTTTC |
| | | REIVSFRVDGDRADVE | | | GCACCCAGGCGGAATTTCGCGCGTGGTTTGAAC |
| | | VVLRAVVDGEEKEVKL | | | GTCTGCGCGCGACAAGCGCAGATGCGAAACGCG |
| | | NHRFFFEGDRLVRVEV | | | AAATTGTGAGCTTTCGCGTGGATGGCGATCGCG |
| | | EIKPL | | | CGGATGTGGAAGTGGTGCTGCGTGCAGTTGTTG |
| | | | | | ATGGTGAAGAAAAAGAAGTGAAACTGAACCATC |
| | | | | | GCTTCTTTTTCGAAGGCGATAGACTGGTGCGCG |
| | | | | | TTGAAGTGGAAATTAAACCGCTGGG(TAA) |
| luxsiti_ | 150 | MSEEAIREFWKRFYEA | 37. | 350 | (ATG)TCCGAAGAAGCGATTCGCGAATTTTGGA |
| 0.2_ | | LDAGDAETASALFPDG | 89011748 | | AACGCTTTTATGAAGCCCTGGATGCGGGCGATG |
| 201 | | TEIHLWDGKTERTRAE | | | CCGAAACCGCGAGCGCATTGTTTCCAGATGGCA |
| | | FKAWFEELYSTSENAK | | | CCGAAATTCATCTGTGGGATGGCAAAACCTTTC |
| | | REITSLKVEGNRAEVE | | | GCACCCGCGCGGAATTTAAAGCGTGGTTTGAAG |
| | | VVLHANIEGKEKTVNL | | | AACTGTATAGCACCAGCGAAAATGCCAAACGTG |
| | | KHWFEFEGDHLVRVRV | | | AAATTACCAGCCTGAAAGTGGAAGGCAACCGCG |
| | | DIKPL | | | CCGAAGTTGAAGTGGTGCTGCATGCGAACATTG |
| | | | | | AAGGTAAAGAAAAGACCGTGAACCTGAAACATT |
| | | | | | GGTTCGAATTTGAAGGCGATCATCTGGTGCGCG |
| | | | | | TGCGTGTGGATATTAAACCGCTGGG(TAA) |
| luxsiti_ | 151 | MSEEEQREFVRRFYEA | 82. | 351 | (ATG)TCGGAAGAAGAACAGCGCGAATTTGTGC |
| 0.2_ | | LDAGDAETASALFPDG | 49067035 | | GCCGTTTTTATGAAGCGCTGGATGCGGGCGATG |
| 202 | | TVIHLWDGRTERTQAE | | | CCGAAACTGCGAGCGCTCTGTTTCCAGATGGCA |
| | | FRAWFEELYSTSEDAS | | | CCGTGATTCATCTGTGGGATGGCCGCACCTTTC |
| | | RHVVSFKVDGNKARVE | | | GCACCCAGGCGGAATTTCGCGCCTGGTTTGAAG |
| | | VVLHANINGEEHTVNL | | | AACTGTATAGCACCAGCGAAGATGCGTCTCGCC |
| | | THVFHFEGDKLVEVEV | | | ATGTGGTGAGCTTTAAAGTGGATGGCAACAAAG |
| | | DIRPL | | | CGCGCGTGGAAGTGGTGCTGCATGCCAACATTA |
| | | | | | ACGGCGAAGAGCATACCGTTAACCTGACCCATG |
| | | | | | TGTTTCATTTTGAAGGCGATAAACTGGTTGAAG |
| | | | | | TCGAAGTGGACATTCGTCCGCTGGG(TAA) |
| luxsiti_ | 152 | MSEEEQREFWKRFYEA | 133. | 352 | (ATG)AGCGAGGAAGAACAGCGCGAATTTTGGA |
| 0.2_ | | LDAGDAETASALFPDG | 8949551 | | AACGCTTTTATGAAGCGCTGGATGCGGGCGATG |
| 204 | | TVIHLWDGKTFHTREE | | | CGGAAACTGCGAGCGCGTTGTTTCCTGATGGCA |
| | | FRAWFVELKSKSEDAK | | | CCGTGATTCATCTGTGGGATGGCAAAACCTTCC |
| | | REIVDFEVDGNVSRVK | | | ATACCCGTGAAGAATTTCGCGCGTGGTTTGTGG |
| | | VVLHANYKGEEKVVEL | | | AACTGAAAAGCAAAAGCGAAGATGCGAAACGCG |
| | | EHVFHEEGDRIVEVEV | | | AAATTGTGGATTTTGAAGTTGACGGCAACGTGA |
| | | KIKPL | | | GCCGCGTGAAAGTGGTGCTGCATGCCAACTATA |
| | | | | | AAGGCGAAGAAAAAGTTGTTGAACTGGAACATG |
| | | | | | TGTTTCATTTCGAAGGCGATCGCCTGGTGGAAG |
| | | | | | TCGAAGTTAAAATTAAACCGCTGGG(TAA) |
| luxsiti_ | 153 | MSEEEIREFVKRFYAA | 126. | 353 | (ATG)AGCGAAGAAGAAATTCGTGAATTTGTGA |
| 0.2_ | | LDAGDAETASALFPDG | 0138217 | | AACGCTTTTATGCGGCGCTGGATGCGGGCGATG |
| 206 | | TEIYLWDGTTERTQAE | | | CGGAAACCGCAAGCGCACTTTTTCCTGATGGCA |
| | | FRAWFVELYSRSDDAS | | | CCGAAATTTATCTGTGGGATGGTACCACCTTTC |
| | | REVVDLKVDGNKAEVE | | | GCACCCAGGCGGAATTTCGCGCGTGGTTTGTGG |
| | | VVLKASVDGEERVVKL | | | AACTGTATAGCCGCAGCGATGATGCGAGCCGCG |
| | | KHFFEFEGDKLVKVEV | | | AAGTGGTGGATCTGAAAGTGGATGGCAATAAAG |
| | | TIEPL | | | CGGAAGTGGAAGTTGTGCTGAAAGCGAGCGTAG |
| | | | | | ATGGTGAAGAACGCGTAGTGAAACTGAAACATT |
| | | | | | TCTTTGAATTCGAAGGCGATAAACTGGTGAAAG |
| | | | | | TTGAAGTGACCATTGAACCGCTGGG(TAA) |
| luxsiti_ | 154 | MSEEEQREFVKKFYEA | 1279. | 354 | (ATG)AGCGAAGAAGAACAGCGCGAATTTGTGA |
| 0.2_ | | LDAGDAETASALFPDG | 81548 | | AGAAATTTTATGAAGCGCTGGATGCGGGCGATG |
| 229 | | TEIHLWDGKTERTQAE | | | CGGAAACCGCAAGCGCGTTGTTTCCAGATGGCA |
| | | FRAWFEKLKSTSENAK | | | CCGAAATTCATCTGTGGGATGGTAAAACCTTTC |
| | | RKIVSFKVDGNKADVE | | | GCACCCAGGCGGAATTTCGTGCGTGGTTTGAAA |
| | | VVLKANINGEEKTVKL | | | AACTGAAAAGCACCAGCGAAAACGCGAAACGCA |
| | | KHTFEFEGDKLKKVNV | | | AAATTGTGAGCTTTAAAGTGGATGGCAACAAAG |
| | | EIVPL | | | CGGATGTGGAAGTTGTGCTGAAAGCGAATATTA |
| | | | | | ACGGGGAAGAAAAGACCGTGAAATTGAAACATA |
| | | | | | CCTTTGAATTTGAAGGCGATAAGCTGAAAAAGG |
| | | | | | TGAACGTGGAAATTGTTCCGCTGGG(TAA) |
| luxsiti_ | 155 | MSEEAQREFVRRFYEA | 1. | 355 | (ATG)AGCGAAGAAGCGCAGCGCGAATTTGTGC |
| 0.2_ | | LDAGDAETASALFPDG | 878369039 | | GCCGCTTTTATGAAGCGCTGGATGCCGGCGATG |
| 237 | | TKIYLWDGRVFETQEE | | | CGGAAACCGCAAGCGCACTGTTTCCTGATGGCA |
| | | FRAWEVELRSTSENAR | | | CCAAAATTTATCTGTGGGATGGCCGCGTGTTTG |
| | | RRVVDFEVDGNVARVE | | | AAACCCAGGAAGAATTTCGCGCGTGGTTTGTGG |
| | | VVLHANIDGEEKTVRL | | | AACTGCGCAGCACCAGCGAAAACGCCCGCAGAA |
| | | RHIFKFEGDKLVEVEV | | | GGGTGGTGGATTTTGAAGTGGATGGCAATGTTG |
| | | TIEPL | | | CGCGCGTTGAAGTTGTGCTGCATGCGAACATTG |
| | | | | | ATGGTGAAGAAAAGACCGTGCGCCTGCGCCATA |
| | | | | | TTTTTAAATTTGAAGGCGATAAACTGGTGGAAG |
| | | | | | TCGAAGTGACCATTGAACCGCTGGG(TAA) |
| luxsiti_ | 156 | MSEEEIREFVRRFYEA | 1. | 356 | (ATG)AGCGAAGAAGAAATTCGCGAATTTGTGC |
| 0.2_ | | LDAGDAETASALFPDG | 300621977 | | GCCGCTTTTATGAAGCGCTGGATGCGGGTGATG |
| 243 | | TKIYLWDGITETTQAE | | | CGGAAACAGCAAGCGCGCTGTTTCCCGATGGCA |
| | | FRAWEVELRSTSDAAR | | | CCAAAATTTATCTGTGGGATGGCATTACCTTTA |
| | | REVVRLEVDGNVAFVE | | | CCACCCAGGCGGAATTTCGCGCGTGGTTTGTGG |
| | | VVLHASHRGEERTVLL | | | AACTGCGCAGCACCAGCGATGCGGCGAGAAGAG |
| | | RHVFQFEGDKLVKVEV | | | AAGTGGTGCGCTTGGAAGTTGGATGGTAACGTGG |
| | | SIDPL | | | CGTTTGTTGAAGTTGTGCTGCATGCGAGCCATC |
| | | | | | GCGGTGAAGAACGCACCGTGCTGCTGCGTCATG |
| | | | | | TGTTTCAGTTTGAAGGCGATAAACTGGTGAAAG |
| | | | | | TGGAAGTTAGCATCGATCCGCTGGG(TAA) |
| luxsiti_ | 157 | MSEKEQKEFWKKFYDA | 30. | 357 | (ATG)AGCGAAAAAGAACAGAAAGAATTTTGGA |
| 0.2_ | | LDAGDAETASALFPDG | 50172771 | | AAAAGTTTTATGATGCGCTGGATGCGGGCGATG |
| 248 | | TIIYLWDGIVERTREE | | | CGGAAACTGCGAGCGCACTGTTTCCAGATGGCA |
| | | FKEWFVKLKSTSENAK | | | CCATTATCTATCTGTGGGATGGCATTGTGTTTC |
| | | RKIVSFKVDGNKSDVE | | | GCACGCGCGAAGAATTCAAAGAATGGTTTGTGA |
| | | VVLEANVNGEKKKVKL | | | AACTGAAAAGCACCTCGGAAAACGCCAAACGCA |
| | | KHVFHFEGDKLVEVEV | | | AAATTGTGAGCTTTAAAGTGGATGGTAACAAAT |
| | | KIDPL | | | CTGATGTGGAAGTGGTGCTGGAAGCGAACGTGA |
| | | | | | ACGGCGAAAAGAAAAAGGTTAAATTGAAACATG |
| | | | | | TGTTCCATTTTGAAGGCGATAAACTGGTTGAAG |
| | | | | | TCGAAGTGAAAATTGATCCGCTGGG{TAA) |
| luxsiti_ | 158 | MSEEEIRNFVKRFYEA | 29. | 358 | (ATG)AGCGAAGAAGAAATTCGTAACTTTGTGA |
| 0.2_ | | LDAGDAETASALFPDG | 51209399 | | AACGCTTTTATGAAGCGCTGGATGCGGGCGATG |
| 258 | | TEIYLWDGKTERTQAE | | | CGGAAACCGCCAGTGCGTTGTTTCCAGATGGCA |
| | | FRAWFEELRSTSDDAK | | | CCGAAATTTATCTGTGGGATGGCAAAACCTTTC |
| | | REVVKFEVEGNVAYVE | | | GCACCCAGGCGGAATTTCGCGCGTGGTTTGAAG |
| | | VVLKANHKGKKEVVKL | | | AACTGCGCAGCACCAGTGATGATGCGAAACGTG |
| | | KHEFEFEGDKVVKVRV | | | AAGTGGTTAAATTTGAAGTTGAAGGCAACGTGG |
| | | EIKPL | | | CGTATGTGGAAGTTGTGCTGAAAGCGAACCATA |
| | | | | | AAGGCAAGAAAGAAGTCGTGAAACTGAAACATG |
| | | | | | AATTTGAGTTCGAAGGCGATAAAGTGGTGAAAG |
| | | | | | TGCGCGTTGAAATCAAACCGCTGGG(TAA) |
| luxsiti_ | 159 | MSEEEQREFWKRFYEA | 5. | 359 | (ATG)AGCGAAGAAGAACAGCGCGAATTTTGGA |
| 0.2_ | | LDAGDAETASALFPDG | 559778853 | | AACGCTTTTATGAAGCGCTGGATGCGGGCGATG |
| 262 | | TEIHLWDGRTFRTRAE | | | CGGAAACCGCAAGCGCGTTGTTTCCTGATGGCA |
| | | FRAWFEELYSQSENAR | | | CCGAAATTCATCTGTGGGATGGCCGCACCTTTC |
| | | REITRFEVDGNVSHVE | | | GCACCCGCGCCGAATTTCGTGCGTGGTTTGAAG |
| | | VVLEAEYEGEKRVVRL | | | AACTGTATAGCCAGAGCGAAAACGCGCGCCGCG |
| | | RHVFEFEGDRLVRVEV | | | AAATTACCCGCTTTGAAGTGGATGGCAACGTGT |
| | | EIEPL | | | CACATGTGGAAGTGGTGCTGGAAGCGGAATATG |
| | | | | | AAGGCGAAAAACGTGTGGTGCGCCTGCGCCATG |
| | | | | | TGTTTGAATTTGAAGGTGATCGCCTGGTGCGTG |
| | | | | | TTGAAGTCGAAATTGAACCGCTGGG(TAA) |
| luxsiti_ | 160 | MSEEEIREFVKRFYEA | 7. | 360 | (ATG)AGCGAAGAAGAAATTCGCGAATTTGTGA |
| 0.2_ | | LDAGDAETASALEPDG | 667588113 | | AACGCTTTTTATGAAGCGCTGGATGCGGGCGATG |
| 263 | | TEIHLWDGTVFRTREE | | | CCGAAACTGCGAGCGCACTATTTCCGGATGGCA |
| | | FRAWFVELRSKSDDAK | | | CCGAAATTCATCTGTGGGATGGTACCGTGTTTC |
| | | REVVKFEVDGNKAYVE | | | GCACCCGTGAAGAATTTCGCGCGTGGTTTGTGG |
| | | VVLHANVDGEEKTVRL | | | AACTGCGCTCAAAAAGCGATGATGCGAAACGTG |
| | | VHEFLFEGDKLVEVKV | | | AAGTGGTGAAATTTGAAGTTGATGGTAACAAAG |
| | | DIEPL | | | CGTATGTGGAAGTTGTGCTGCATGCGAACGTGG |
| | | | | | ATGGGGAAGAAAAGACCGTGCGCCTGGTGCATG |
| | | | | | AATTTCTGTTTGAAGGCGATAAACTGGTCGAAG |
| | | | | | TGAAAGTGGATATTGAACCGCTGGG(TAA) |
| luxsiti_ | 161 | MSEEDIREFVRRFYEA | 574. | 361 | (ATG)AGCGAAGAAGATATTCGCGAATTTGTGC |
| 0.2_ | | LDAGDADTASALFKDG | 8009 | | GCCGCTTTTATGAAGCGCTGGATGCGGGCGATG |
| 273 | | TKIHLWDGKTETTREE | 675 | | CGGATACCGCAAGCGCGCTGTTTAAAGATGGCA |
| | | FREWFVRLHSTSDDAR | | | CCAAAATTCATCTGTGGGATGGCAAAACCTTTA |
| | | REVVRLEVEGNRAHVE | | | CCACCCGTGAAGAATTTCGTGAATGGTTTGTGA |
| | | VVLRASYQGEDRVVRL | | | GACTGCATAGCACCAGCGATGATGCGCGCCGGG |
| | | THEFLFEGDEVVEVHV | | | AAGTGGTACGCCTGGAAGTTGAAGGCAACCGTG |
| | | RIEPL | | | CACATGTGGAAGTCGTGCTGCGCGCGAGCTATC |
| | | | | | AGGGCGAAGATCGCGTGGTTAGACTGACCCATG |
| | | | | | AATTTCTGTTTGAAGGTGATGAAGTTGTTGAAG |
| | | | | | TGCATGTGCGCATTGAACCGCTGGG(TAA) |
| luxsiti_ | 162 | MSEEAQREFWRRFYEA | 4. | 362 | (ATG)AGCGAAGAAGCGCAGCGCGAATTCTGGC |
| 0.2_ | | LDAGDAETASALFPDG | 376641327 | | GCCGCTTTTATGAAGCGCTGGATGCGGGTGATG |
| 276 | | TEIHLWDGRTERTQAE | | | CGGAAACCGCCTCTGCACTGTTTCCGGATGGTA |
| | | FRDWFVKLHSTSADAR | | | CCGAAATTCATCTGTGGGATGGCCGCACCTTTC |
| | | REITRERVEGDVAHVE | | | GCACCCAGGCAGAATTTCGCGATTGGTTTGTGA |
| | | VVLHASIDGEKKTVKL | | | AATTGCATAGCACCAGCGCGGATGCGCGCCGCG |
| | | RHVAHFEGDKLVEVHV | | | AAATTACCCGCTTTAGAGTGGAAGGTGATGTGG |
| | | DIEPL | | | CGCATGTTGAAGTGGTCCTGCATGCGAGCATTG |
| | | | | | ATGGCGAAAAGAAAACCGTGAAACTGCGCCATG |
| | | | | | TGGCCCATTTTGAAGGCGATAAACTGGTGGAAG |
| | | | | | TGCATGTGGATATTGAACCGCTGGG(TAA) |
| luxsiti_ | 163 | MSEEEIREFVRRFYEA | 1922. | 363 | (ATG)AGCGAAGAAGAAATTCGCGAATTTGTGC |
| 0.2_ | | LDAGDAETASALEKDG | 585349 | | GCCGCTTTTATGAAGCGCTGGATGCGGGCGATG |
| 277 | | TKIYLWDGTVFETREE | | | CGGAAACCGCAAGCGCGCTGTTTAAAGATGGCA |
| | | FRAWFVELYSKSENAR | | | CCAAAATTTATCTGTGGGATGGTACCGTGTTTG |
| | | RRVVSFKVDGNVAEVE | | | AAACCCGTGAAGAATTTCGCGCGTGGTTTGTGG |
| | | VVLHASFQGEDKVVRL | | | AACTGTATAGCAAAAGCGAAAACGCGCGCCGCC |
| | | KHRFKFEGDEVVEVEV | | | GAGTGGTGAGCTTTAAAGTGGATGGCAACGTGG |
| | | DIEPL | | | CTGAAGTGGAAGTGGTGCTGCATGCGAGCTTTC |
| | | | | | AGGGCGAAGATAAAGTTGTGCGTCTGAAACATC |
| | | | | | GTTTTAAATTTGAAGGCGATGAAGTTGTTGAAG |
| | | | | | TAGAAGTTGATATTGAACCGCTGGG(TAA) |
| luxsiti_ | 164 | MSEEAQREFWKRFYEA | 3. | 364 | (ATG)AGCGAAGAAGCGCAGCGCGAATTTTGGA |
| 0.2_ | | LDAGDAETASALFPDG | 356599862 | | AACGCTTTTATGAAGCGCTGGATGCGGGCGATG |
| 280 | | TRIYLWDGRVETTQAE | | | CGGAAACCGCGAGCGCATTGTTTCCAGATGGCA |
| | | FRAWFVELRSTSADAK | | | CCCGCATTTATCTGTGGGATGGCCGCGTTTTTA |
| | | REIVKFEVDGDVSYVE | | | CCACCCAGGCGGAATTTCGCGCGTGGTTTGTGG |
| | | VVLKANVDGEEKIVRL | | | AACTGCGTTCGACCAGCGCCGATGCGAAACGTG |
| | | RHVFHFEGDRIVEVEV | | | AAATTGTTAAATTTGAAGTGGATGGCGATGTGT |
| | | EIEPL | | | CCTATGTGGAAGTGGTGCTGAAAGCGAACGTAG |
| | | | | | ATGGTGAAGAAAAGATTGTGCGCCTGCGCCATG |
| | | | | | TGTTTCATTTTGAAGGCGATCGCCTGGTTGAAG |
| | | | | | TCGAAGTTGAAATCGAACCGCTGGG(TAA) |
| luxsiti_ | 165 | MSEEEQREFWKRFYEA | 143. | 365 | (ATG)TCTGAAGAAGAACAGCGCGAATTTTGGA |
| 0.2_ | | LDAGDAETASALFPDG | 1679337 | | AACGCTTTTATGAAGCGCTGGATGCGGGCGATG |
| 283 | | TRIYLWDGRTETTRAE | | | CGGAAACCGCGAGCGCACTTTTTCCAGATGGCA |
| | | FRAWFEELHSTSENAR | | | CCCGCATTTATCTGTGGGATGGCCGCACCTTTA |
| | | REIVSFRVDGDVADVE | | | CCACCCGCGCCGAATTTCGCGCGTGGTTTGAAG |
| | | VVLRANVNGEERTVRL | | | AACTGCATAGCACCAGTGAAAACGCGCGCCGCG |
| | | RHVFHFEGDRIVEVHV | | | AAATTGTGAGCTTTCGTGTGGATGGCGATGTGG |
| | | EIEPL | | | CGGATGTGGAAGTGGTGCTGCGTGCGAATGTGA |
| | | | | | ATGGCGAAGAACGTACCGTGCGCCTGCGTCATG |
| | | | | | TGTTTCATTTTGAAGGCGATCGCCTGGTTGAAG |
| | | | | | TGCATGTTGAAATTGAACCGCTGGG(TAA) |
| luxsiti_ | 166 | MSEEAQREFWRRFYDA | 2. | 366 | (ATG)AGCGAAGAAGCGCAGCGCGAATTTTGGC |
| 0.2_ | | LDAGDAETASALFPDG | 299930891 | | GCCGCTTTTATGATGCGCTGGATGCGGGCGATG |
| 286 | | TRIHLWDGRTFHTRAE | | | CGGAAACCGCAAGCGCGTTGTTTCCTGATGGCA |
| | | FRAWEVELRSTSDDAK | | | CCCGCATTCATCTGTGGGATGGCCGCACCTTTC |
| | | REIISFEVDGNRSRVE | | | ATACCCGCGCCGAATTTCGCGCGTGGTTTGTGG |
| | | VVLHANIDGEEKKVLL | | | AACTGCGTAGCACGAGCGATGATGCCAAACGCG |
| | | RHEFLFEGDRLVEVHV | | | AAATTATTAGCTTTGAAGTGGATGGCAACCGCA |
| | | EIKPL | | | GCCGCGTGGAAGTGGTGCTGCATGCGAACATCG |
| | | | | | ATGGTGAAGAAAAGAAAGTGCTGCTGCGCCATG |
| | | | | | AATTTCTGTTTGAAGGCGATCGCCTGGTTGAAG |
| | | | | | TTCATGTGGAAATTAAACCGCTGGG{TAA) |
| luxsiti_ | 167 | MSEEEIREFVDRFYKA | 26. | 367 | (ATG)AGCGAAGAAGAAATCCGCGAATTTGTTG |
| 0.2_ | | LDAGDAETASALFPDG | 87145819 | | ATCGCTTTTATAAAGCGCTGGATGCGGGTGATG |
| 290 | | TEIHLWDGTTFHTQAE | | | CGGAAACCGCAAGCGCGTTATTTCCCGATGGCA |
| | | FRAWFVKLKSTSDNAK | | | CCGAAATTCATCTGTGGGATGGTACCACCTTTC |
| | | REIVKLEVDGNKAKVE | | | ATACCCAGGCGGAATTTCGCGCGTGGTTTGTGA |
| | | VVLKANVNGEEKVVKL | | | AACTGAAATCGACCAGCGATAACGCGAAACGTG |
| | | THEFEFEGDRLVKVTV | | | AAATTGTTAAACTGGAAGTGGATGGCAACAAAG |
| | | KIEPE | | | CGAAAGTGGAAGTTGTGCTGAAAGCCAACGTTA |
| | | | | | ACGGTGAAGAAAAAGTGGTCAAACTGACCCATG |
| | | | | | AATTTGAATTCGAAGGCGATCGCCTGGTGAAAG |
| | | | | | TGACCGTGAAAATTGAACCGCTGGG(TAA) |
| luxsiti_ | 168 | MSAEAIRQFVERFYAA | 54. | 368 | (ATG)AGCGCGGAAGCAATTCGCCAGTTTGTGG |
| 0.3_ | | LDAGDADTASALFPDG | 50034554 | | AACGCTTTTATGCGGCGCTGGATGCGGGTGATG |
| 305 | | TEIHLWDGRTERTQAE | | | CGGATACTGCATCAGCGCTGTTTCCGGATGGTA |
| | | FRAWFEELYSTSDGAS | | | CCGAAATTCATCTGTGGGATGGCCGTACCTTTC |
| | | REVVALEVDGNRARVE | | | GCACCCAGGCGGAATTTCGCGCGTGGTTTGAAG |
| | | VVLHASVGGEEKTVRL | | | AACTGTATAGCACCAGCGATGGCGCGAGCCGCG |
| | | RHEFEFEGDQLVRVTV | | | AAGTGGTGGCACTTGAAGTTGATGGTAACCGCG |
| | | EIEPL | | | CGAGAGTGGAAGTTGTGCTGCATGCGAGCGTTG |
| | | | | | GCGGCGAAGAAAAGACCGTGCGCCTGCGTCATG |
| | | | | | AATTTGAATTCGAAGGCGATCAGCTGGTGCGCG |
| | | | | | TGACCGTGGAAATTGAACCGCTGGG(TAA) |
| luxsiti_ | 169 | MSEKEQKEFWKKFYDA | 439. | 369 | (ATG)AGCGAAAAAGAACAGAAAGAATTTTGGA |
| 0.3_ | | LDAGDADTASALFPDG | 91085 | | AAAAGTTTTATGATGCGCTGGATGCGGGTGATG |
| 306 | | TKIYLWDGKVERTQEE | | | CGGATACCGCGAGCGCACTGTTTCCAGATGGCA |
| | | FREWEEKLYSKSDNAK | | | CCAAAATTTATCTGTGGGATGGCAAAGTGTTTC |
| | | REIVKFKVDGNIAYVE | | | GCACCCAGGAAGAATTCCGTGAATGGTTTGAAA |
| | | VILKANVDGEEKVVKL | | | AACTGTATAGCAAAAGCGATAACGCCAAACGCG |
| | | KHVFKFEGDKVKEVKV | | | AGATTGTGAAATTTAAAGTTGATGGTAACATCG |
| | | KIVPL | | | CCTATGTGGAAGTGATTCTGAAAGCGAACGTGG |
| | | | | | ATGGCGAAGAGAAAGTGGTGAAACTGAAACATG |
| | | | | | TGTTTAAATTTGAAGGCGATAAAGTGAAAGAAG |
| | | | | | TTAAAGTCAAAATTGTGCCGCTGGG(TAA) |
| luxsiti_ | 170 | MSEESQREFWKRFYAA | 1116. | 370 | (ATG)AGCGAAGAAAGCCAGCGCGAATTTTGGA |
| 0.3_ | | LDAGDAETASALFPDG | 82792 | | AACGCTTTTATGCGGCGCTGGATGCGGGCGATG |
| 308 | | TEIHLWDGTVERTRAE | | | CGGAAACTGCAAGCGCATTGTTTCCGGATGGCA |
| | | FRAWFVDLHSKSDNAS | | | CCGAAATTCATCTGTGGGATGGTACCGTGTTTC |
| | | REITSFKVEGNKALVE | | | GCACCCGCGCGGAATTTCGCGCGTGGTTTGTGG |
| | | VVLHASFKGEERTVKL | | | ATCTGCATAGCAAAAGCGATAACGCGAGCCGCG |
| | | THVFEFEGDRLVRVEV | | | AAATTACCTCTTTTAAAGTGGAAGGCAACAAAG |
| | | EIKPL | | | CGCTGGTTGAAGTGGTGCTGCATGCGAGCTTTA |
| | | | | | AAGGTGAAGAACGCACCGTGAAACTGACCCACG |
| | | | | | TGTTTGAATTTGAAGGCGATCGCCTGGTGCGCG |
| | | | | | TCGAAGTTGAAATTAAACCGCTGGG(TAA) |
| luxsiti_ | 171 | MSEEAVREFVRRFYEA | 53. | 371 | (ATG)TCGGAAGAAGCGGTGCGTGAATTTGTGC |
| 0.3_ | | LDAGDAETASALEPDG | 31582585 | | GCCGCTTTTATGAAGCGCTGGATGCGGGCGATG |
| 309 | | TEIHLWDGTTERTRAE | | | CGGAAACTGCGAGCGCCTTGTTTCCTGATGGCA |
| | | FRAWERRLRATSEDAR | | | CCGAAATTCATCTATGGGATGGTACCACCTTTC |
| | | RRVVRLEVDGNVADVE | | | GCACCCGCGCGGAATTTCGTGCGTGGTTTCGTC |
| | | VVLHASYRGEERVVRL | | | GTCTGAGAGCAACTAGCGAAGATGCGCGCCGTC |
| | | RHRYEFEGDRLVRVDV | | | GTGTGGTGCGTCTGGAAGTGGATGGCAACGTTG |
| | | EIRPL | | | CGGATGTCGAAGTTGTGCTGCATGCGAGCTATC |
| | | | | | GCGGTGAAGAACGCGTTGTGCGGCTGCGCCATC |
| | | | | | GCTACGAATTTGAAGGCGATCGCCTGGTGCGCG |
| | | | | | TTGATGTTGAAATTCGCCCGCTGGG(TAA) |
| luxsiti_ | 172 | MSEEEQRQFVKRFYEA | 10. | 372 | (ATG)AGCGAAGAAGAACAGCGCCAGTTTGTGA |
| 0.3_ | | LDAGDSETASALFPDG | 17346234 | | AACGCTTTTACGAAGCGCTGGATGCGGGTGATA |
| 317 | | TVIHLWDGTTEHTQEE | | | GCGAAACCGCAAGCGCGCTGTTTCCGGATGGCA |
| | | FRAWEEKLRSTSDNAR | | | CCGTGATTCATCTGTGGGATGGTACCACCTTTC |
| | | REVVHFEVEGNVAYVE | | | ATACCCAGGAAGAATTTCGCGCGTGGTTTGAAA |
| | | VVLRASVGGEERVVRL | | | AACTGCGCAGCACCAGCGATAACGCGCGCCGTG |
| | | RHVFHFEGDHLVEVEV | | | AAGTGGTGCATTTTGAAGTTGAAGGCAACGTGG |
| | | EIEPL | | | CGTATGTGGAAGTTGTTCTGCGTGCGAGCGTGG |
| | | | | | GCGGTGAAGAACGCGTTGTGCGTCTGCGTCATG |
| | | | | | TGTTTCATTTCGAAGGCGATCATCTGGTTGAAG |
| | | | | | TCGAAGTGGAAATTGAACCGCTGGG(TAA) |
| luxsiti_ | 173 | MSEEEQRNEWKKEYEA | 5. | 373 | (ATG)AGCGAAGAAGAACAGCGCAACTTTTGGA |
| 0.3_ | | LDAGDAETASALFPDG | 974429855 | | AAAAGTTTTATGAAGCGCTGGATGCGGGTGATG |
| 323 | | TEIYLWDGTVEKTREE | | | CGGAAACCGCAAGCGCGCTGTTTCCTGATGGCA |
| | | FRAWFVELYSTSENAK | | | CCGAAATTTATCTGTGGGATGGTACCGTGTTTA |
| | | RHIVDFKVDGNESKVK | | | AAACCCGCGAAGAGTTCCGTGCGTGGTTTGTGG |
| | | VILKANIDGEEKVVEL | | | AACTGTATTCGACCTCGGAAAACGCGAAACGCC |
| | | EHYFLFEGDHLVEVKV | | | ATATTGTGGATTTTAAAGTGGATGGCAACGAAA |
| | | EIHPL | | | GCAAAGTGAAAGTGATTCTGAAAGCGAACATTG |
| | | | | | ATGGTGAAGAAAAGGTGGTTGAACTGGAACATT |
| | | | | | ATTTTCTGTTTGAAGGCGATCATCTGGTGGAAG |
| | | | | | TGAAGGTGGAAATTCATCCGCTGGG(TAA) |
| luxsiti_ | 174 | MSEEEIREFVERFYKA | 179. | 374 | (ATG)AGCGAGGAAGAAATTCGAGAATTTGTGG |
| 0.3_ | | LDAGDAETASSLEPDG | 7187284 | | AACGCTTTTATAAAGCGCTGGATGCGGGCGATG |
| 324 | | TEIHLWDGKTFHTQEE | | | CGGAAACTGCGAGCAGCCTGTTTCCAGATGGCA |
| | | FRSWFVELKSKSDNAK | | | CCGAAATTCATCTGTGGGATGGCAAAACATTTC |
| | | RKVVSFEVEGNKAKVK | | | ATACCCAAGAAGAATTTCGCAGCTGGTTTGTCG |
| | | VVLKASYKGEEKEVKL | | | AACTGAAAAGCAAAAGCGATAACGCCAAACGCA |
| | | EHEFEFEGDKLVEVNV | | | AAGTGGTGAGCTTTGAAGTGGAAGGCAACAAAG |
| | | KIEPL | | | CGAAAGTGAAAGTTGTGCTGAAAGCGAGCTATA |
| | | | | | AAGGGGAGGAAAAAGAAGTTAAACTGGAACATG |
| | | | | | AATTTGAATTCGAAGGCGATAAATTGGTTGAAG |
| | | | | | TCAACGTGAAAATTGAACCGCTGGG(TAA) |
| luxsiti_ | 175 | MSEKEQREFWKKEYEA | 560. | 375 | (ATG)AGCGAAAAAGAACAGCGCGAATTTTGGA |
| 0.3_ | | LDAGDAETASALEPDG | 5245335 | | AGAAATTTTATGAAGCGCTGGATGCGGGTGATG |
| 330 | | TEIHLWDGKTFHTREE | | | CGGAAACCGCCAGCGCGTTATTTCCCGATGGCA |
| | | FKEWFVKLYSRSENAK | | | CCGAAATTCATCTGTGGGATGGCAAAACCTTTC |
| | | RHIVKFEVDGDKAYVE | | | ATACCCGCGAAGAATTTAAAGAATGGTTTGTGA |
| | | VVLYAKIGGEEKVVKL | | | AACTGTATAGCCGTTCAGAAAACGCGAAACGTC |
| | | KHEFLFEGDKLVEVNV | | | ATATTGTGAAGTTTGAAGTGGATGGCGATAAAG |
| | | KIEPL | | | CGTATGTGGAAGTGGTGCTGTATGCGAAAATTG |
| | | | | | GCGGTGAAGAAAAAGTGGTTAAACTGAAACATG |
| | | | | | AATTTCTGTTTGAGGGCGACAAACTGGTTGAAG |
| | | | | | TTAACGTGAAAATCGAACCGCTGGG(TAA) |
| luxsiti_ | 176 | MSEEEIKEFVERFYKA | 53. | 376 | (ATG)AGCGAAGAAGAAATTAAAGAATTTGTGG |
| 0.3_ | | LDAGDADTASSLFPDG | 7512094 | | AACGCTTTTATAAAGCGCTGGATGCGGGTGATG |
| 338 | | TEIFLWDGKTEKTKEE | | | CGGATACCGCAAGCAGCCTGTTTCCAGATGGCA |
| | | FREWFVKLKSESDDAK | | | CCGAAATCTTCCTGTGGGATGGCAAAACCTTTA |
| | | REVVSLKVEGNKAYVE | | | AAACCAAAGAGGAATTTCGCGAATGGTTTGTGA |
| | | VVLHANYKGEKKEVKL | | | AACTGAAAAGCGAAAGCGATGATGCGAAACGCG |
| | | THIFEFEGDKVVKVEV | | | AAGTGGTGTCGCTGAAAGTGGAAGGCAACAAAG |
| | | TIVPL | | | CGTATGTGGAAGTAGTGCTGCATGCGAACTATA |
| | | | | | AAGGCGAAAAGAAAGAAGTTAAACTGACCCATA |
| | | | | | TTTTTGAATTTGAAGGTGATAAAGTCGTGAAAG |
| | | | | | TTGAAGTGACCATTGTGCCGCTGGG(TAA) |
| luxsiti_ | 177 | MSEEEQRQFVKEFYEA | 243. | 377 | (ATG)AGCGAAGAAGAGCAGCGCCAGTTTGTGA |
| 0.3_ | | LDAGDAETASALFPDG | 3538355 | | AAGAATTTTATGAAGCGCTGGATGCGGGTGATG |
| 366 | | TEIHLWDGKTFTTQAE | | | CGGAAACCGCGAGCGCGTTGTTTCCAGATGGCA |
| | | FRAWFERLYSTSENAR | | | CCGAAATTCATCTGTGGGATGGTAAAACCTTTA |
| | | RHVVDERVEGNVADVE | | | CCACCCAGGCGGAATTTCGCGCCTGGTTTGAAC |
| | | VVLHATKDGEEHTVRL | | | GCCTGTATAGCACCAGCGAAAATGCGCGCCGCC |
| | | RHKFEFEGDELVRVEV | | | ATGTCGTGGATTTTCGTGTGGAAGGCAACGTGG |
| | | VIEPL | | | CCGATGTTGAAGTGGTGCTGCATGCGACCAAAG |
| | | | | | ATGGTGAAGAACATACCGTGCGCCTGCGTCATA |
| | | | | | AATTTGAATTCGAAGGCGATGAACTGGTGCGCG |
| | | | | | TGGAAGTTGTGATTGAACCGCTGGG(TAA) |
| luxsiti_ | 178 | MSAEAQREFWARFYAA | 1. | 378 | (ATG)AGCGCGGAAGCGCAGCGCGAATTTTGGG |
| 0.3_ | | LDAGDADTASALFPDG | 97512094 | | CGCGCTTTTATGCGGCGTTGGATGCGGGTGATG |
| 381 | | TEIYLWDGKVERTREE | | | CGGATACCGCTAGCGCGCTGTTTCCTGATGGCA |
| | | FRAWEVELRSTSADAK | | | CCGAAATTTATCTGTGGGATGGCAAAGTGTTTC |
| | | REIVSFEVDGNRASVD | | | GCACCCGCGAAGAATTTCGCGCGTGGTTTGTGG |
| | | VVLHASVNGEEHTVHL | | | AACTGCGCAGCACCAGCGCCGATGCGAAACGTG |
| | | HHEFEFEGDRLVRVRV | | | AAATTGTGAGCTTTGAAGTGGATGGTAACCGTG |
| | | TIQPL | | | CGAGCGTGGATGTGGTGCTGCATGCCAGCGTGA |
| | | | | | ACGGTGAAGAACATACCGTGCATCTGCATCACG |
| | | | | | AATTTGAATTCGAAGGCGATCGCCTGGTGCGCG |
| | | | | | TGCGTGTGACCATTCAGCCGCTGGG(TAA) |
| luxsiti_ | 179 | MSAEQQREFVKRFYEA | 1119. | 379 | (ATG)AGCGCAGAACAGCAGCGCGAATTTGTGA |
| 0.3_ | | LDAGDADTASALFPDG | 172771 | | AACGCTTTTATGAAGCGCTGGATGCGGGTGATG |
| 383 | | TEIHLWDGTTERTRAE | | | CGGATACCGCAAGCGCACTGTTTCCGGATGGCA |
| | | FRAWFEELYSTSENAS | | | CCGAAATTCATCTGTGGGATGGTACCACCTTTC |
| | | REVTSFSVDGDVADVE | | | GCACCCGCGCGGAATTTCGCGCGTGGTTTGAAG |
| | | VVLRANLGGEDRTVSL | | | AACTGTATAGCACCTCGGAAAACGCGAGCCGCG |
| | | RHVFHFAGDRLVRVEV | | | AAGTGACCAGCTTTTCGGTGGATGGCGATGTGG |
| | | SIRPL | | | CAGATGTGGAAGTGGTGCTGCGCGCGAACCTGG |
| | | | | | GTGGTGAAGATCGCACCGTTAGCCTGAGACATG |
| | | | | | TGTTTCATTTTGCGGGCGATCGCCTGGTGCGCG |
| | | | | | TTGAAGTGAGCATTCGCCCGCTGGG(TAA) |
| luxsiti_ | 180 | MSEEEIKEFVRRFYEA | 347. | 380 | (ATG)AGCGAAGAAGAAATTAAAGAATTTGTGC |
| 0.3_ | | LDAGDADTASALFPDG | 1257775 | | GCCGCTTTTATGAAGCGCTGGATGCGGGTGATG |
| 385 | | TRIHLWDGTTETTRAE | | | CGGATACCGCAAGCGCACTGTTTCCGGATGGCA |
| | | FRAWFVDLYSKSDAAK | | | CCCGCATTCATCTGTGGGATGGTACCACCTTTA |
| | | REVTSLKVEGNVAKVK | | | CCACCCGCGCGGAATTTCGCGCGTGGTTTGTGG |
| | | VVLKANYKGEEKTVEL | | | ATCTGTATAGCAAAAGCGATGCGGCGAAACGTG |
| | | EHKFEFEGDRLVRVDV | | | AAGTGACCAGCCTGAAAGTGGAAGGTAATGTGG |
| | | TIKPM | | | CGAAAGTGAAAGTAGTGCTGAAAGCGAACTATA |
| | | | | | AAGGTGAAGAAAAGACCGTGGAACTGGAACATA |
| | | | | | AATTTGAATTCGAAGGCGATCGCCTGGTGCGCG |
| | | | | | TTGATGTTACCATTAAACCGATGGG(TAA) |

In another aspect, the disclosure provides protein having luciferase activity, comprising an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO:1, wherein:
Residue 14 is Y, D, or E and residue 98 is H or N;
Residue 18 is D or E and residue 65 is R.

The proteins of this aspect are non-naturally occurring. In one embodiment, the percent identity relative to the reference sequence is carried out by sequence alignment with the Needleman-Wunsch algorithm, a common sequence alignment tool for those of skill in the art, which allows for insertions and deletions.

In one embodiment, the protein comprises one or both of A96M and M110V substitutions relative to SEQ ID NO:1. In another embodiment, the protein comprises comprising an R60S substitution relative to SEQ ID NO:1. In a further embodiment, the protein comprises R60S, A96M, and M110V substitutions relative to SEQ ID NO:1. In another embodiment, any substitutions relative to SEQ ID NO:1 at residues F12, 135, W38, F49, V81, L83, V94, A 97, W100, M110, V112 are conservative amino acid substitutions. In one embodiment, the protein comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:1-3. In another embodiment, the protein comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO: 1-181.

In another embodiment, the proteins comprise the formula X1-Z1-X2-Z2-X3-Z3-X4-Z4-X5-Z5-X6-Z6-X7-Z7-X8-Z8, wherein:
X1 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of MSEEQIRQFLRRFYEALD (SEQ ID NO: 182), wherein residue 14 is Y, D, or E and residue 18 is D or E;
X2 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of ADTAASLF (SEQ ID NO: 183);
X3 has an amino acid sequence at least 50%, 75%, or 100% identical to the amino acid sequence of TIHL (SEQ ID NO: 184);
X4 has an amino acid sequence at least 33%, 66%, or 100% identical to the amino acid sequence of VTF;
X5 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of EEFREWFERLFST (SEQ ID NO: 185);
X6 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of QREIKSLEVR (SEQ ID NO: 186), wherein residue 2 is R;
X7 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of VEVHVQLHATH (SEQ ID NO: 187);
X8 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of KHTVDATHHWHFR (SEQ ID NO: 188), wherein residue 8 is H or N,
X9 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of VTEMRVHINPTG (SEQ ID NO: 189); and
wherein Z1, Z2, Z3, Z4, Z5, Z6, Z7, and Z8 are independently present or absent, and when present may comprise any amino acid sequence.

In one embodiment, 1, 2, 3, 4, 5, 6, 7, or all 8 of the following are true
Z1 comprises SGD;
Z2 comprises HPGV (SEQ ID NO: 190);
Z3 comprises WDG;
Z4 comprises TSR;
Z5 comprises RKDA (SEQ ID NO: 191);
Z6 comprises GDT;
Z7 comprises NGQ;
Z8 comprises GNR; and
wherein 0, 1, 2, 3, 4, 5, 6, 7, or all 8 of Z1, Z2, Z3, Z4, Z5, Z6, Z7, and Z8 further comprising an additional polypeptide domain.

In one embodiment of all of the proteins of the disclosure, amino acid substitutions relative to the reference protein are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Proteins comprising conservative amino acid substitutions can be tested in any one of the assays described herein to confirm that a desired activity, is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G). Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met. Ala. Val, Leu, Ile; (2) neutral hydrophilic: Cys, Ser. Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; lie into Leu or into Val; Leu into Ile or into Val; Lys into Arg, into Gln or into Glu; Met into Leu, into Tyr or into lie; Phe into Met, into Leu or into Tyr; Scr into Thr; Thr into Ser; Trp into Tyr; Tyr into Trp; and/or Phe into Val, into Ile or into Leu.

In another embodiment, the disclosure provides self-complementing multipartite protein having luciferase activity, comprising at least a first polypeptide component and a second polypeptide component, wherein the at least first polypeptide component and the second polypeptide component are not covalently linked, wherein in total the at least first polypeptide component and the second polypeptide component comprise domains X1-Z1-X2-Z2-X3-Z3-X4-Z4-X5-Z5-X6-Z6-X7-Z7-X8-Z8-X9, wherein each domain is as defined above; and
wherein (a) each X domain is fully present within one polypeptide component of the at least first polypeptide component and the second polypeptide component, and (b) none of the at least first polypeptide component and the second polypeptide component include each of X1, X2, X3, X4, X5, X6, X7, X8, and X9.

The split proteins are non-naturally occurring. The split proteins comprise at least a first polypeptide component and a second polypeptide component in which X domains are preserved while split points are taken only in the Z domains. In other words, each X strand or (X1, X2, X3, X4, X5, X6, X7, X8, and X9) is fully present within one polypeptide component of the at least first polypeptide component and the second polypeptide component, while the protein is split into separate components at a Z domain (of Z1, Z2, Z3, Z4, Z5, Z6, Z7, and Z8), wherein the Z domain that the split occurs at may be absent, or may be partially present in one or both of the first and second polypeptide components. By way of non-limiting example, in various embodiments of a split luciferase protein, the first polypeptide component and the second polypeptide component may comprise components as exemplified in Table 2.

TABLE 2

| | First polypeptide component | Second polypeptide |
|---|---|---|
| Example | comprises | component comprises |
| 1: Split at Z2 | X1-ZI-X2-(Z2) | (Z2)-X3-Z3-X4-Z4-X5-Z5- |
| | | X6-Z6-X7-Z7-X8-Z8-X9 |
| 2: Split at Z4 | X1-Z2-X2-Z2-X3-Z3-X4-(Z4) | (Z4)-X5-Z5-X6-Z6-X7-Z7- |
| | | X8-Z8-X9 |
| 3: Split at Z6 | X1-Z2-X2-Z2-X3-Z3-X4-Z4- | (Z6)-X7-Z7-X8-Z8-X9 |
| | X5-Z5-X6-(Z6) | |
| 4: Split at Z8 | X1-Z2-X2-Z2-X3-Z3-X4-Z4- | (Z8)-X9 |
| | X5-Z5-X6-Z6-X7-Z7-X8-(Z8) | |

In various embodiments, the split may occur at Z4, Z5, Z6, or Z7.

In another embodiment, the disclosure provides self-complementing multipartite protein having luciferase activity, comprising at least a first polypeptide component and a second polypeptide component, wherein the at least first polypeptide component and the second polypeptide component are not covalently linked, wherein in total the at least first polypeptide component and the second polypeptide component comprise the secondary structure arrangement H1-L1-H2-L2-E1-L3-E2-L4-H3-L5-E3-L6-E4-L7-E5-L8-E6, wherein each domain is as defined above;
wherein (a) each H and E domain is fully present within one polypeptide component of the at least first polypeptide component and the second polypeptide component, and (b) none of the at least first polypeptide component and the second polypeptide component include all of the H and E domains.

In this embodiment, the split proteins comprise at least a first polypeptide component and a second polypeptide component in which H and E domains are preserved while split points are taken only in the L domains. In various embodiments, the split occurs at L4. L5, L6, L7, or L8.

The split proteins of these embodiments are only active when they are brought together, and thus are conditionally active.

In another embodiment the disclosure provides fusion proteins comprising:
(a) the protein or polypeptide component of any embodiment or combination of embodiments herein; and
(b) one or more additional functional domains.

As used herein, a “functional domain” is any polypeptide that can be usefully fused to the luciferase protein or split protein component of the disclosure. By way of non-limiting examples, the one or more additional functional domains may comprise a diagnostic polypeptide, any protein that one might want to localize within a cell, tissue, or organism; etc.

In another aspect the disclosure provides nucleic acids encoding the protein, protein component, or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure. In various non-limiting embodiments, the nucleic acid may comprise the nucleotide sequence of any one of SEQ ID NO:200-380, wherein residues in parentheses are optional and may be present or absent.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), non-naturally occurring polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

The disclosure also provide kits, comprising:
(a) the protein, polypeptide component, fusion protein, nucleic acid, expression vector, and/or host cell of any embodiment or combination of embodiments herein; and
(b) instructions for their use.

In one embodiment, the kits further comprise diphenylterazine (DTZ).

In another aspect, the disclosure provides methods for use of the protein, polypeptide component, fusion protein, nucleic acid, expression vector, host cell, and/or kit of any preceding claim for any suitable purpose, including but not limited to use luminescent reporting assays, diagnostic assays, cellular localization of targets of interest, cellular imaging, gene editing, live animal imaging, cancer labeling, CART-cells reporting, secreted assay, gene delivery, tissue engineering, etc. Additional details can be found in the examples.

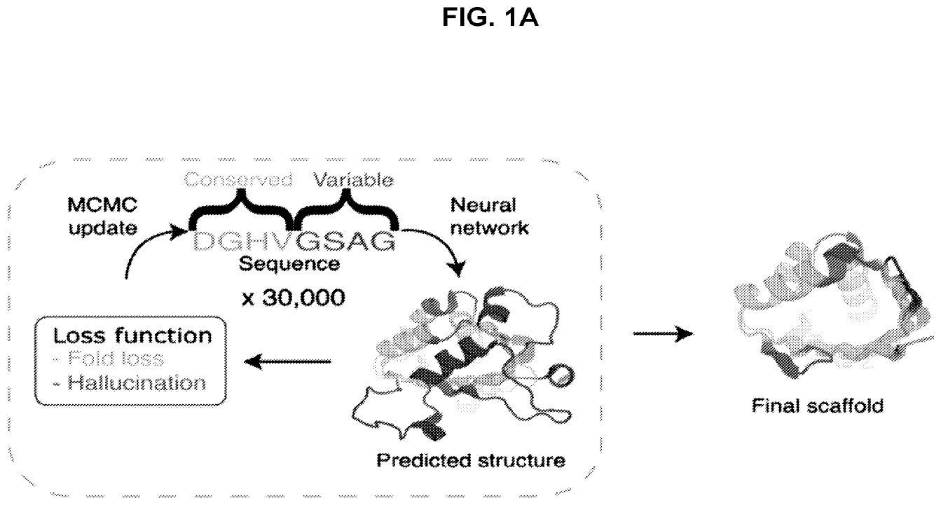

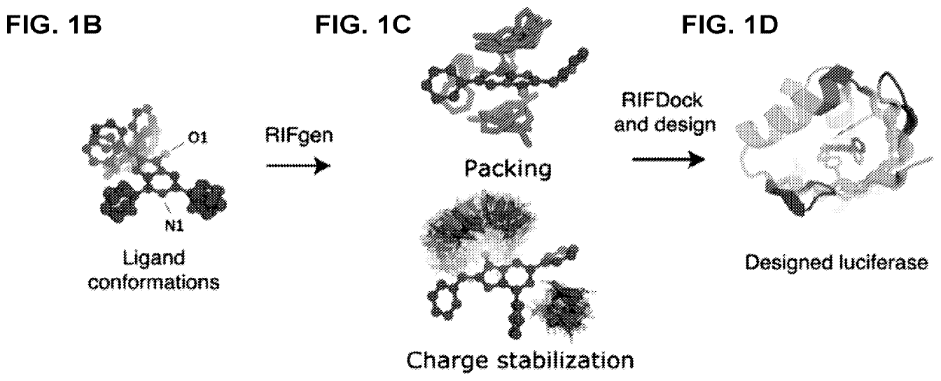

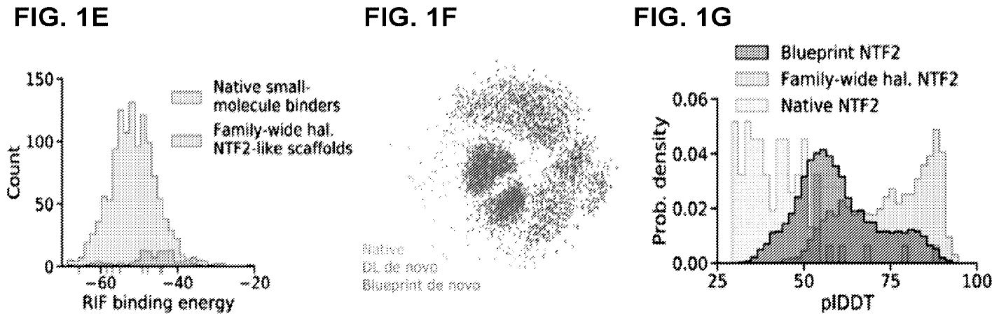

In another aspect, the disclosure provides methods for making a luciferase, comprising de novo design using the methods of any embodiment disclosed herein, starting with the protein comprising the amino acid sequence of SEQ ID NO:381. The examples provide detailed methods for de novo design of luciferases for DTZ. As described in the examples, the methods involve designing a shape complementary catalytic site that stabilizes the anionic state of DTZ and lowers the SET energy barrier, assuming that the downstream dioxetane light emitter thermolysis steps are spontaneous. To stabilize the anionic species of DTZ, we focused on the placement of the positively charged guanidinium group of an arginine residue to interact with the anionic imidazopyrazinone core. To computationally design such active sites, the inventors first used AIMNet to generate an ensemble of anionic DTZ conformers (FIG. 1b). Next, around each conformer, the inventors used the RIFgen method to enumerate Rotamer Interaction Fields (RIFs) on 3D grids consisting of millions of placements of amino acid sidechains making hydrogen bonding and nonpolar interactions with DTZ (FIG. 1c). Additionally, the inventors included an arginine guanidinium group near the deprotonation site at the nitrogen of imidazopyrazinone (N1 atom) in the RIF. RIFdock was then used to dock each DTZ conformer and associated RIF in the central cavity of each scaffold to maximize protein-DTZ interactions. An average of eight sidechain rotamers including an arginine to stabilize the anionic imidazopyrazinone core were positioned in each pocket (FIG. 1d). For the top 50,000 docks with the most favorable sidechain-DTZ interactions, the inventors optimized the remainder of the sequence (FIG. 1d) for high-affinity binding to DTZ with a bias towards the naturally observed sequence variation to ensure foldability. During the design process, pre-defined hydrogen bond networks (HBNets) in the scaffolds were kept intact for structural specificity and stability, and interactions of these HBNet side chains with DTZ were explicitly required in the RIFdock step to ensure preorganization of residues essential for catalysis. In the first sequence design phase, the identities of all RIF and HBNet residues were kept fixed, and the surrounding residues were optimized to hold the sidechain-DTZ interactions in place and maintain structural specificity. In the second sequence design step, the RIF residue identities (except the arginine) were also allowed to vary, to identify apolar and aromatic packing interactions missed in the RIF due to binning effects. During sequence design, the scaffold backbone, sidechains, and DTZ substrate were allowed to relax in Cartesian space.

### EXAMPLES

### Summary

De novo enzyme design has sought to introduce active sites and substrate binding pockets predicted to catalyze a reaction of interest into geometrically compatible native scaffolds^{1,2}, but has been limited by a lack of suitable protein structures and the complexity of native protein sequence-structure relationships. Here we describe a deep-learning based “family-wide hallucination” approach that generates large numbers of idealized protein structures containing diverse pocket shapes and designed sequences that encode them. We use these scaffolds to design artificial luciferases that selectively catalyze the oxidative chemiluminescence of the synthetic luciferin substrates, diphenylterazine (DTZ); through the placement of an arginine guanidinium group adjacent to an anion species that develops during the reaction in a high shape complementarity binding pocket. For both luciferin substrates, we obtain designed luciferases with high selectivity; the most active of these is a small (13.9 kDa) and thermostable (T_{M}>95° C.) enzyme with a catalytic efficiency on DTZ (k_{cat}/K_{M}=10⁶ M⁻¹s⁻¹) comparable to native luciferases but with much higher substrate specificity. The design of highly active and specific biocatalysts from scratch with broad applications in biomedicine is an important milestone for computational enzyme design, and our approach should enable the design of a wide range of new and useful luciferases and other enzymes.

### Study

Bioluminescent light produced by the enzymatic oxidation of a luciferin substrate is widely used for bioassays and imaging in biomedical research. Because no excitation light source is needed, luminescent photons are produced in the dark which results in higher sensitivity than fluorescence imaging in live animal models and in biological samples where autofluorescence or phototoxicity is a concern^{4,5}. However, the development of luciferases as molecular probes has lagged behind that of well-developed fluorescent protein toolkits for a number of reasons: (i) very few native luciferases have been identified; (ii) many of those that have been identified require multiple disulfide bonds to stabilize the structure and are therefore prone to misfolding in mammalian cells; (iii) most native luciferases do not recognize synthetic luciferins with more desirable photophysical properties; and (iv) multiplexed imaging to follow multiple processes in parallel using mutually orthogonal luciferase-luciferin pairs has been limited by the low substrate specificity of native luciferases.

We sought to use de novo protein design to create new luciferases that are small, highly stable, well-expressed in cells, specific for one substrate, and need no cofactors to function. As we are not constrained to natural luciferase substrates, we chose a synthetic luciferin, Diphenylterazine (DTZ) as the target substrate duo to its good quantum yield, red-shifted emission³, favorable in vivo pharmacokinetics^{14,15}, and lack of required cofactors for emission. Previous computational enzyme design studies have primarily repurposed native protein scaffolds in the PDB, but there are few native structures with binding pockets appropriate for DTZ, and the effects of sequence changes on native proteins can be unpredictable. To circumvent these limitations, we set out to generate large numbers of ideal protein scaffolds with pockets of the appropriate size and shape for DTZ, and with clear sequence-structure relationships to facilitate subsequent active site incorporation. To identify protein folds capable of hosting such pockets, we first docked DTZ into 4000 native small molecule binding proteins. We found that many NTF2 (nuclear transport factor 2)-like folds have binding pockets with appropriate shape-complementary and size for DTZ placement (FIG. 1e), and hence selected the NTF2-like superfamily as the target topology.

### Family-Wide Hallucination

Native NTF2 structures have a range of pocket sizes and shapes but also contain non-ideal features such as long loops which compromise stability. To create large numbers of ideal NTF2-like structures, we developed a deep-learning based “family-wide hallucination” approach that integrates unconstrained de novo design^{19,21} and fixed backbone sequence design approaches²¹ to enable the generation of an essentially unlimited number of proteins having a desired fold (FIG. 1a). The family-wide hallucination approach utilizes the de novo sequence and structure discovery capability of unconstrained protein hallucination^{19,20} for loop and variable regions, and structure-guided sequence optimization for core regions. We employed the trRosetta™ structure prediction neural network²², which is effective in identifying experimentally successful de novo designed proteins and hallucinating new globular proteins of diverse topologies. Starting from sequences and predicted structures of 2,000 naturally occurring NTF2s, we used trRosetta™ to optimize the amino sequence of conserved core and variable loop regions. Protein core idealization was carried out with a topology-specific loss function over core residue pair geometries (see Methods) and variable loop optimization, by optimizing sequence length and identity to maximize the confidence of the neural network in the predicted structure. To further encode structural specificity, we incorporated buried, long-range hydrogen-bonding networks. The resulting 1615 family-wide hallucinated NTF2 scaffolds provided more shape complementary binding pockets for DTZ than native small-molecule protein binding proteins (FIG. 1e). This approach samples protein backbones closer to native NTF2-like proteins (FIG. 1f) and with better scaffold quality metrics than a previous non deep-learning approach²³ (FIG. 1g).

We chose the NFT2 scaffold of SEQ ID NO:381 from which to design luciferases for DTZ, as described in detail below.

>2692_0_0.35_5_19_1806_2_0.45_5_
Y14_H98_W100dlo7nb_clean_0001_
D18_R65.xd1.pdb
(SEQ ID NO: 381)
MSEEEIRQFLRRFYEAFDKGDVDTFASLFHPGVTIHVWQGIT
FTSREELREWVERFLRNEKDMQREILSLEVRGDTVEVHVQVH
TTHNGQKYTFDVTHHWHFRGHRVTEIRVHVNPT

### De Novo Design of Luciferases for DTZ

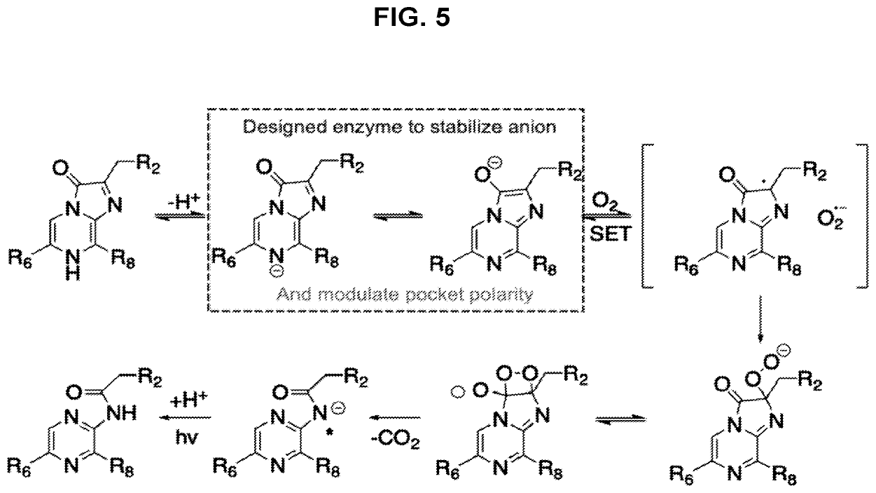

Standard computational enzyme design generally starts from an ideal active site or theozyme consisting of protein functional groups surrounding the reaction transition state that is then extrapolated into a set of existing scaffolds^{1,2}. However, the detailed mechanism of native marine luciferases is not well defined as only a handful of apo-structures and no holo-structures have been solved^{24,25} (excluding calcium-regulated photoproteins). Both quantum chemistry calculations^{26,27} and experimental data^{28,29} suggest that the chemiluminescent reaction proceeds through an anionic species and that the polarity of the surroundings can substantially alter the free energy of the subsequent single electron transfer (SET) process with triplet molecular oxygen (³O₂). Guided by these data (FIG. 5), we sought to design a shape complementary catalytic site that stabilizes the anionic state of DTZ and lowers the SET energy barrier, assuming that the downstream dioxetane light emitter thermolysis steps are spontaneous. To stabilize the anionic species of DTZ, we focused on the placement of the positively charged guanidinium group of an arginine residue to interact with the anionic imidazopyrazinone core.

To computationally design such active sites into large numbers of hallucinated NTF2 scaffolds, we first used AIMNet³⁰ to generate an ensemble of anionic DTZ conformers (FIG. 1b). Next, around each conformer, we used the RIFgen method^{31,32} to enumerate Rotamer Interaction Fields (RIFs) on 3D grids consisting of millions of placements of amino acid sidechains making hydrogen bonding and nonpolar interactions with DTZ (FIG. 1c). Additionally, we included an arginine guanidinium group near the deprotonation site at the nitrogen of imidazopyrazinone (N1 atom) in the RIF. RIFdock was then used to dock each DTZ conformer and associated RIF in the central cavity of each scaffold to maximize protein-DTZ interactions. An average of eight sidechain rotamers including an arginine to stabilize the anionic imidazopyrazinone core were positioned in each pocket (FIG. 1d). For the top 50,000 docks with the most favorable sidechain-DTZ interactions, we optimized the remainder of the sequence using RosettaDesign™ (FIG. 1d) for high-affinity binding to DTZ with a bias towards the naturally observed sequence variation to ensure foldability. During the design process, pre-defined hydrogen bond networks (HBNets) in the scaffolds were kept intact for structural specificity and stability, and interactions of these HBNet side chains with DTZ were explicitly required in the RIFdock step to ensure preorganization of residues essential for catalysis. In the first sequence design phase, the identities of all RIF and HBNet residues were kept fixed, and the surrounding residues were optimized to hold the sidechain-DTZ interactions in place and maintain structural specificity. In the second sequence design step, the RIF residue identities (except the arginine) were also allowed to vary, to identify apolar and aromatic packing interactions missed in the RIF due to binning effects. During sequence design, the scaffold backbone, sidechains, and DTZ substrate were allowed to relax in Cartesian space. Following sequence optimization, the designs were filtered based on ligand-binding energy, protein-ligand hydrogen bonds, shape complementarity, and contact molecular surface, and 7982 designs were selected and ordered as pooled oligos for experimental screening.

### Screening and Characterization of DTZ Specific Luciferases

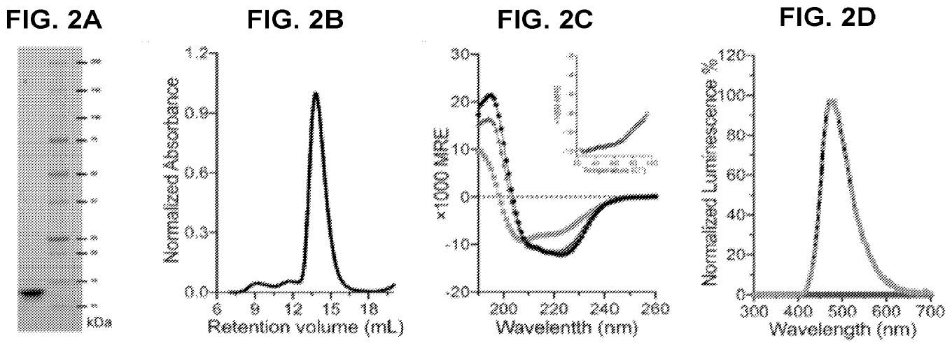

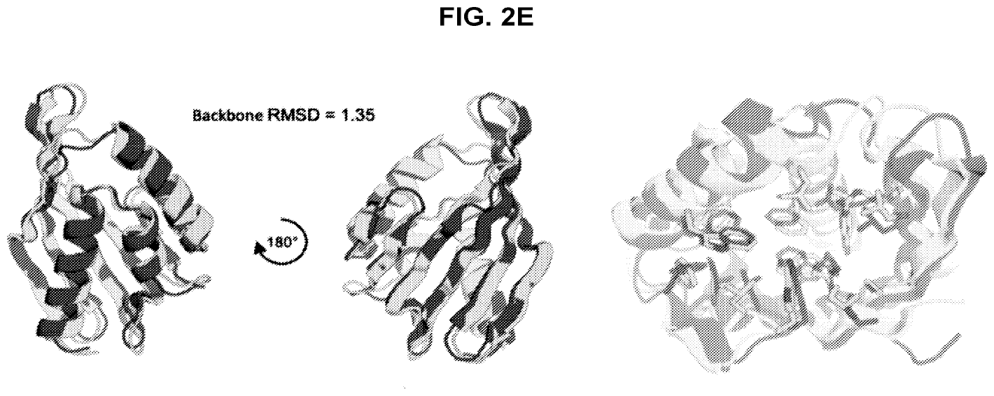

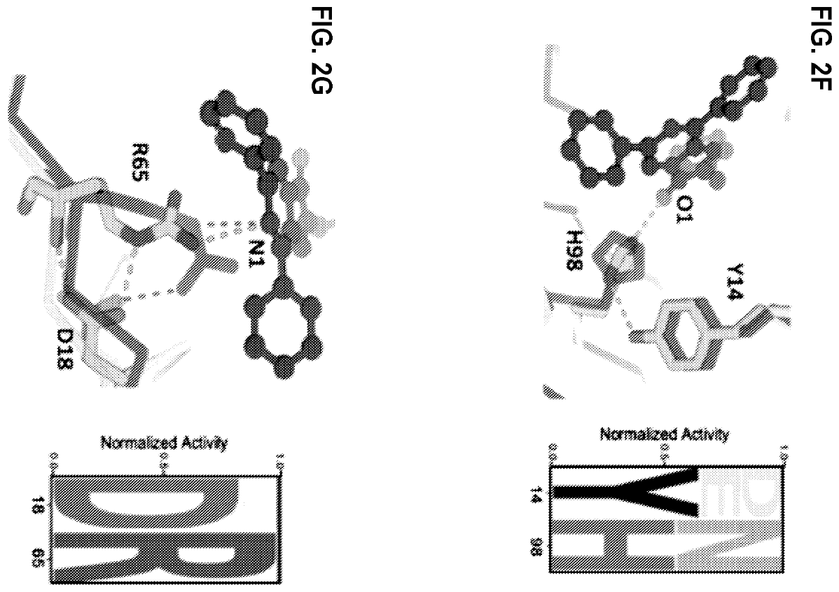

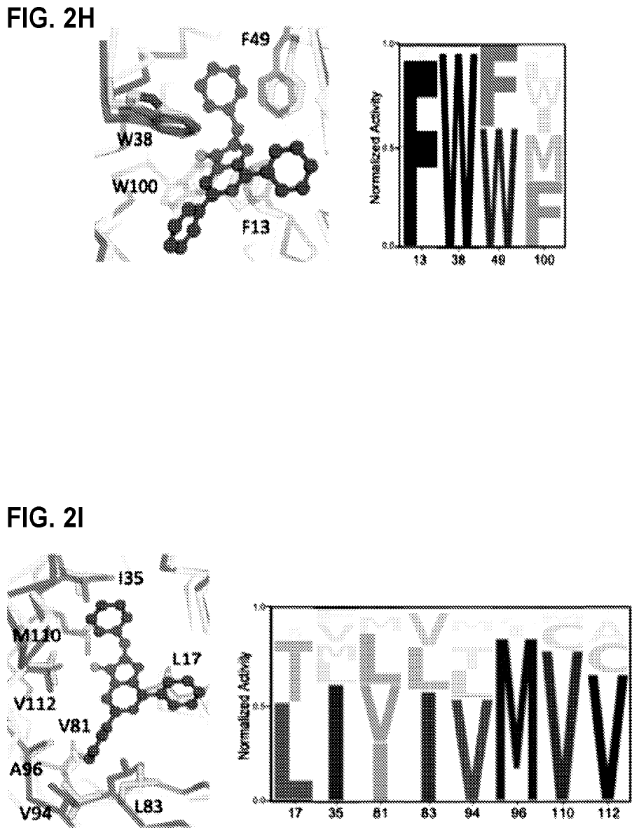

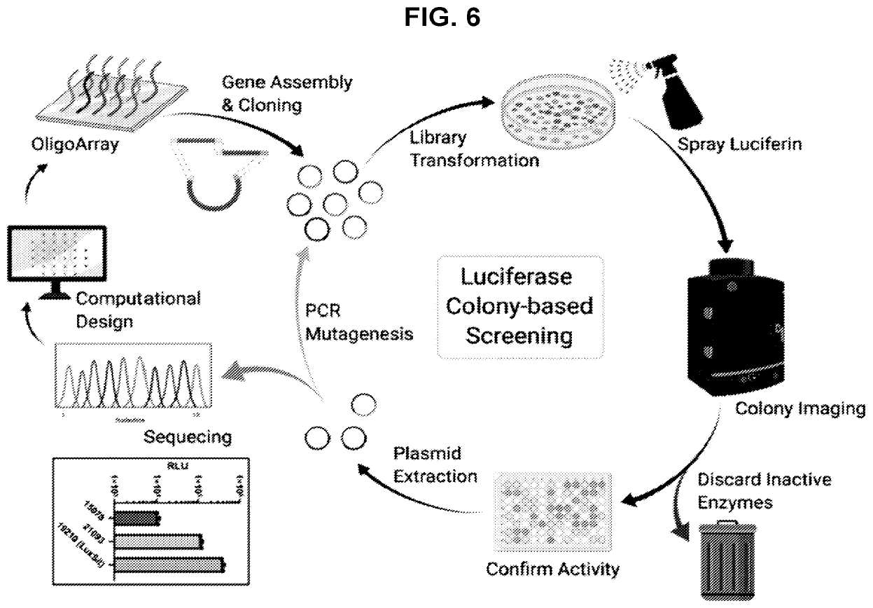

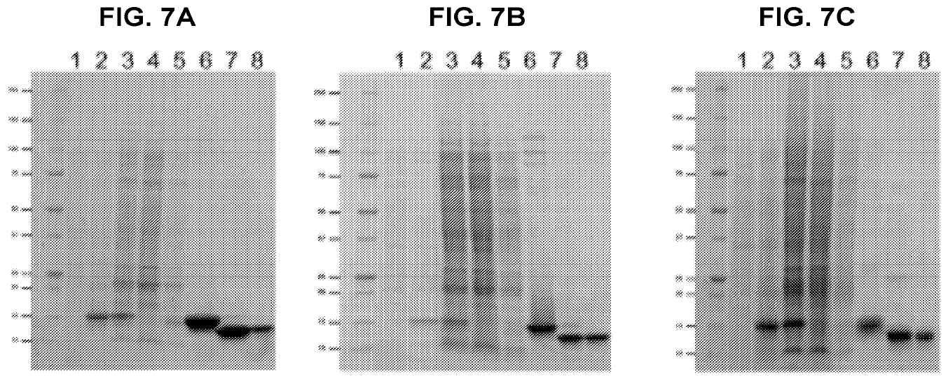

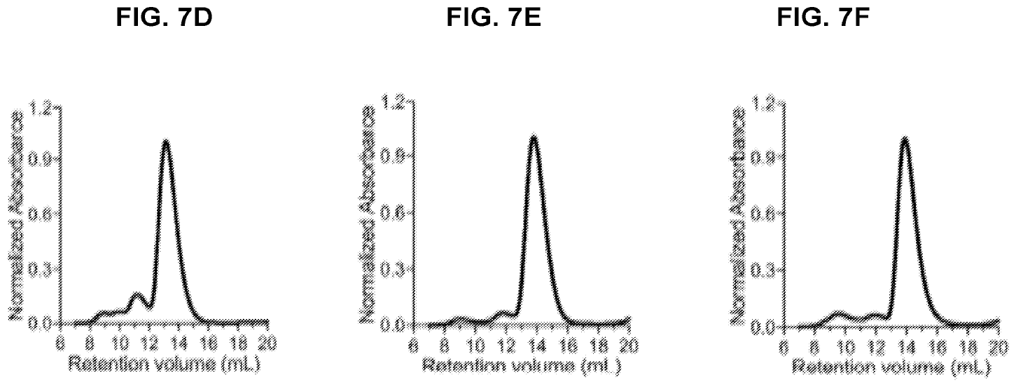

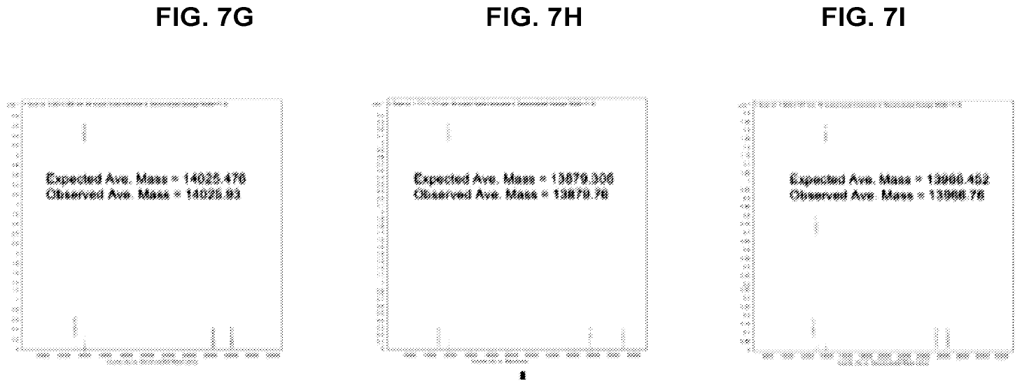

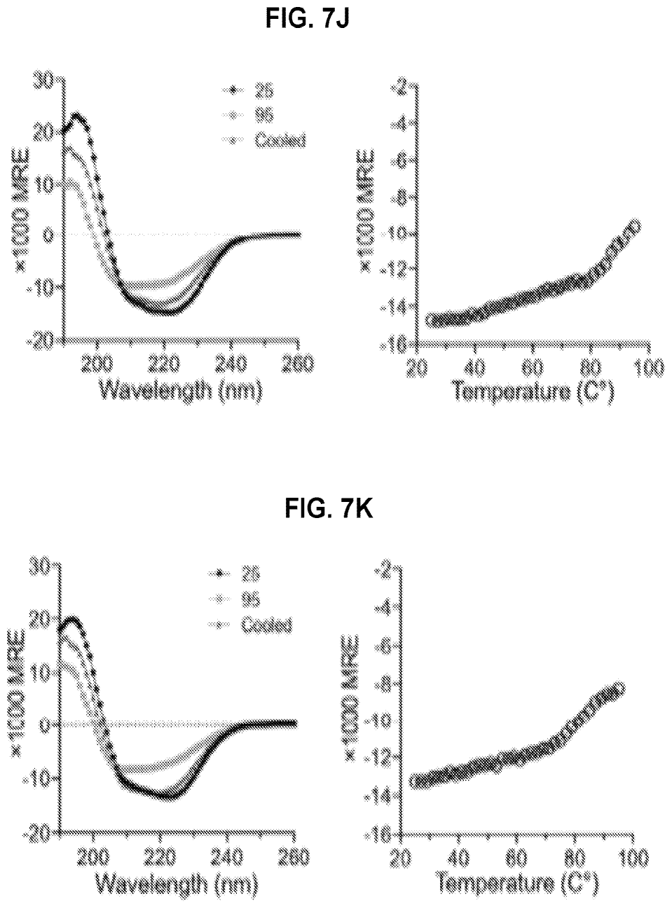

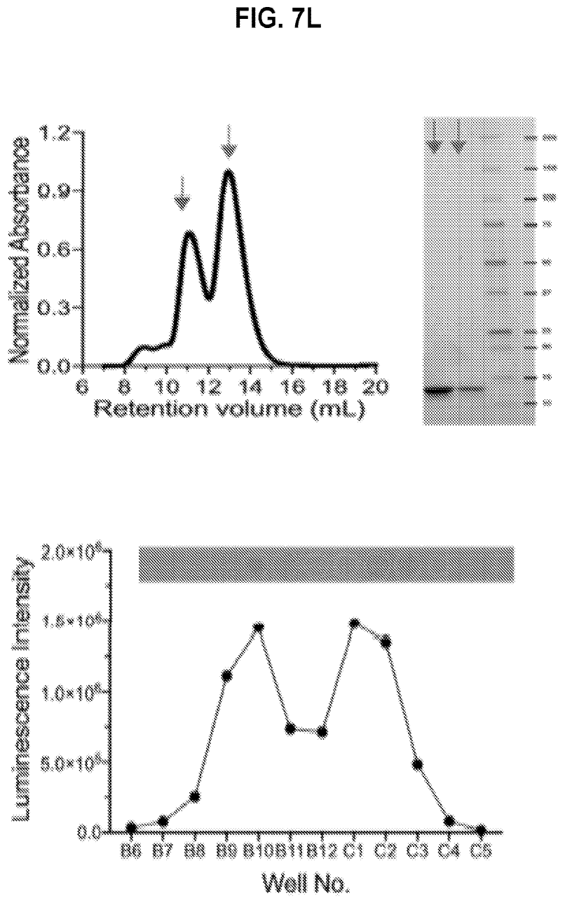

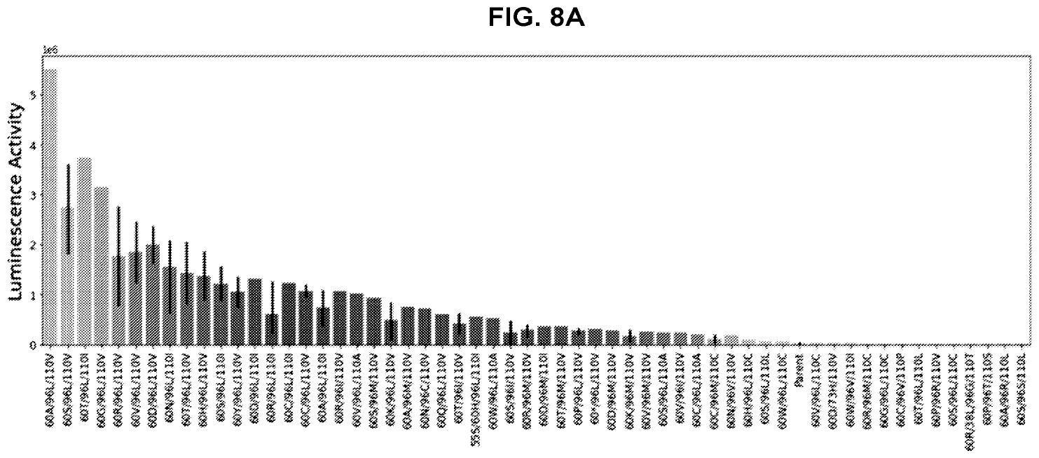

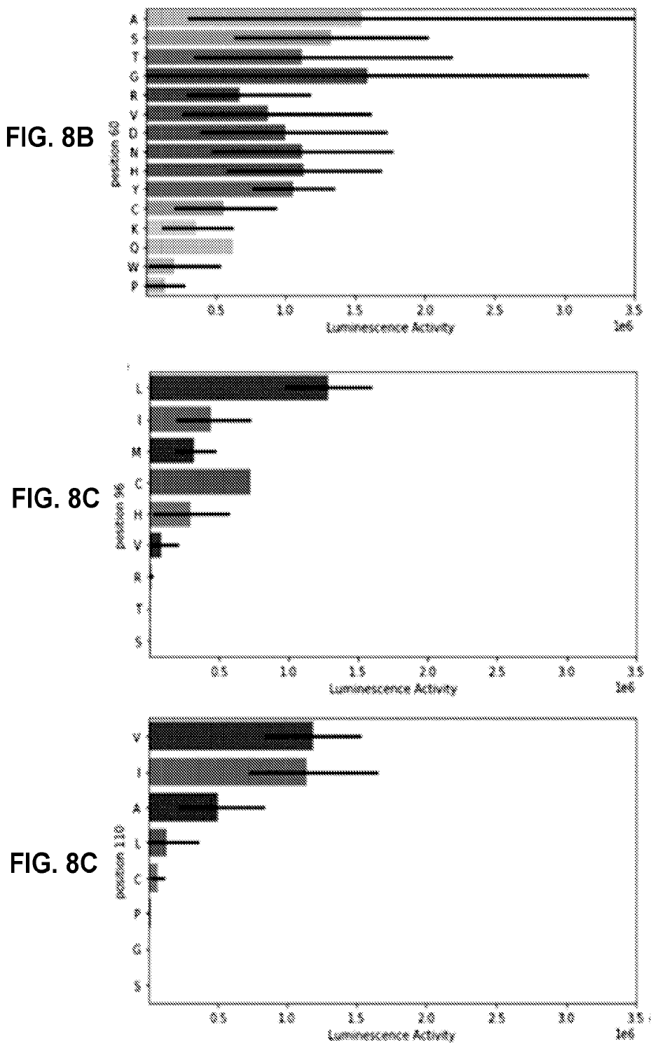

Oligonucleotides encoding the two halves of each design were assembled into full-length genes and cloned into an E. coli expression vector (see Methods). A colony-based screening method was used to directly image active luciferase colonies from the library and the activities of selected clones were confirmed using a 96-well plate expression (FIG. 6). Three active designs were identified; we refer to the most active of these as LuxSit (Latin: let light exist); LuxSit is the smallest known luciferase with 117 residues (13.9 kDa). Biochemical analysis, including SDS-PAGE and size exclusion chromatography (FIG. 2ab and FIG. 7), indicated that LuxSit is highly expressed, soluble, and monomeric from E. coli expression. Circular dichroism (CD) spectroscopy showed a strong far UV CD signature, suggesting an organized α-β structure. CD melting experiments showed that the protein is not fully unfolded at 95° C., and the full structure is regained when the temperature is dropped (FIG. 2c). Incubation of LuxSit with DTZ resulted in luminescence with an emission peak at ˜480 nm (FIG. 2d), consistent with the DTZ chemiluminescence spectrum. While we were not able to determine the crystal structure of LuxSit, the AlphaFold2³³ predicted structure is very close to the design model at the backbone level (RMSD=1.3 Å) and over the side chains interacting with the substrate (FIG. 2e). The designed LuxSit active site contains Tyr14-His98 and Asp18-Arg65 dyads; with the imidazole nitrogen atoms of His98 making hydrogen bond interactions with Tyr14 and the O1 atom of DTZ (FIG. 2f). The center of the Arg65 guanidinium cation is 4.2 Å from the N1 atom of DTZ and Asp18 forms a bidentate hydrogen bond to the guanidinium group and backbone N—H of Arg65 (FIG. 2g).

### Activity Optimization

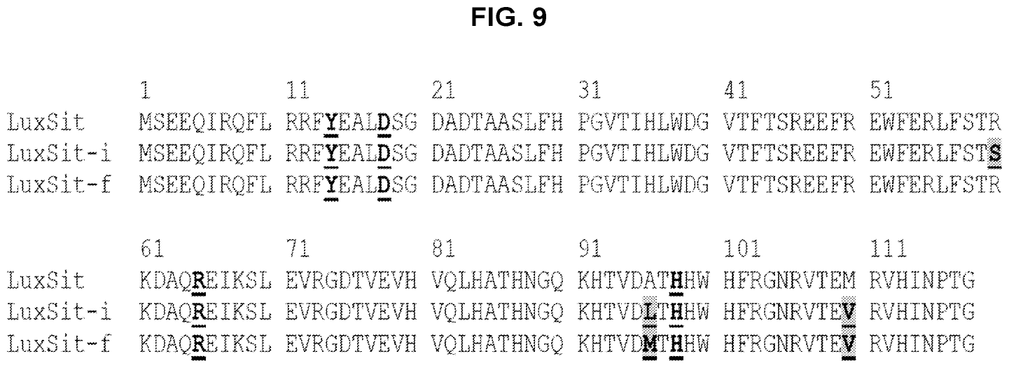

To better understand the contributions to catalysis of LuxSit, the most active of our luciferase designs, we constructed a site saturation mutagenesis (SSM) library in which every mutation was made at every pocket residue one at a time (see Methods). FIG. 2f-i illustrate the amino-acid preferences at key positions. Arg65 is highly conserved, and its dyad partner Asp18 can only be mutated to Glu (which reduces activity), suggesting the carboxylate-Arg65 hydrogen bond is important for luciferase activity. In the Tyr14-His98 dyad, Tyr14 can be substituted with Asp and Glu, while His98 can be replaced with Asn. As all active variants had hydrogen bond donors and acceptors at these positions, the dyad may help mediate the electron and proton transfer required for luminescence. Hydrophobic (FIG. 2h) and π-stacking (FIG. 2i) residues at the binding interface tolerate other aromatic or aliphatic substitutions and generally prefer the amino acid in the original design consistent with model-based affinity predictions of mutational effects. The A96M and M110V mutants increase activity by 16-fold and 19-fold over LuxSit respectively (Table 4). Optimization guided by these results yielded LuxSit-f (A96M/M110V) with strong initial flash emission and LuxSit-i (R60S/A96L/M110V) with more than 100-fold higher photon flux over LuxSit (FIG. 9). Overall, the active site saturation mutagenesis results support the design model, with the Tyr14-His98 and Asp18-Arg65 dyads playing key roles in catalysis and the substrate-binding pocket largely conserved.

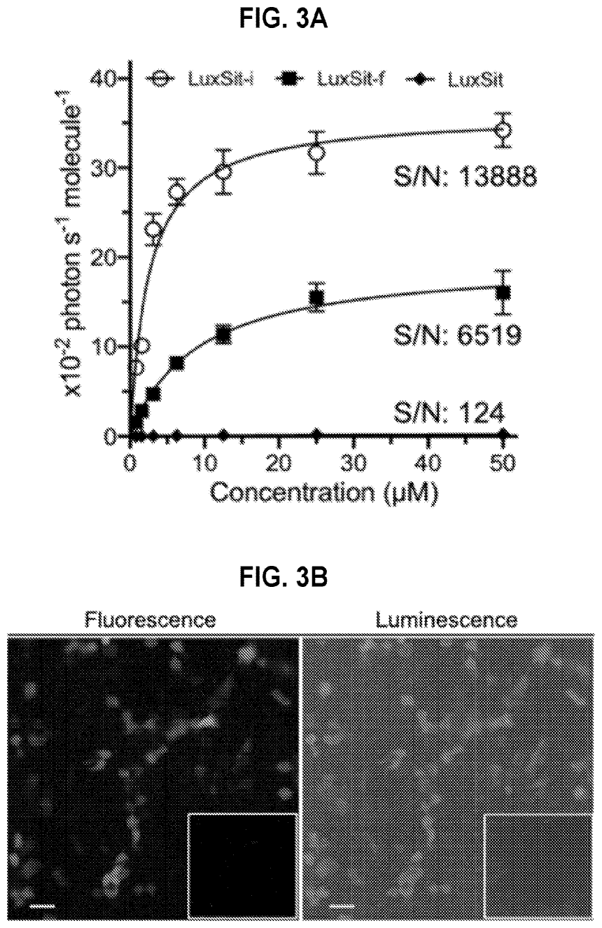

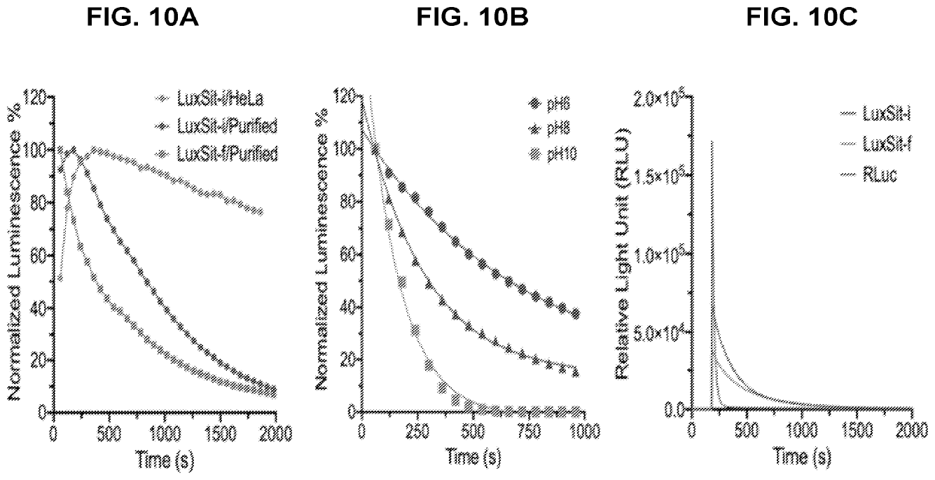

The most active catalysts, LuxSit-f and LuxSit-i were both expressed solubly in E. coli at high levels and are monomeric (some dimerization was observed at the high protein concentration, FIG. 7l) and thermostable (FIG. 7j-k). Similar to native CTZ-utilizing luciferases, the apparent Michaelis constants K % of both LuxSit-f and LuxSit-i are in the low μM range (FIG. 3a) and the luminescent signal decays over time due to fast catalytic turnover (FIG. 10a). LuxSit-i is a very efficient enzyme with a k_{cat}/K_{M} of 10⁶ M⁻¹s⁻¹. The luminescence signal is readily visible to the naked eye, and the photon flux (photon s⁻¹) is 38% greater than the native Renilla reniformis luciferase (RLuc) (Table 3). The DTZ luminescent reaction catalyzed by LuxSit-i is pH-dependent (FIG. 10b), consistent with the proposed mechanism.

### Cellular Imaging and Multiplexed Bioassay

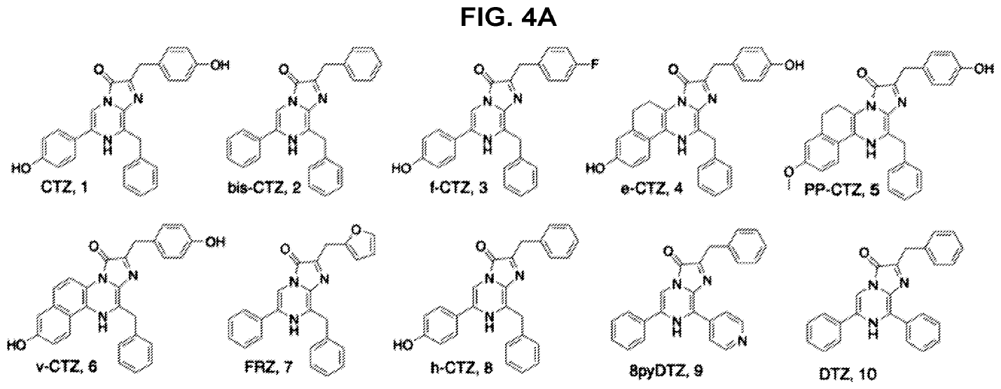

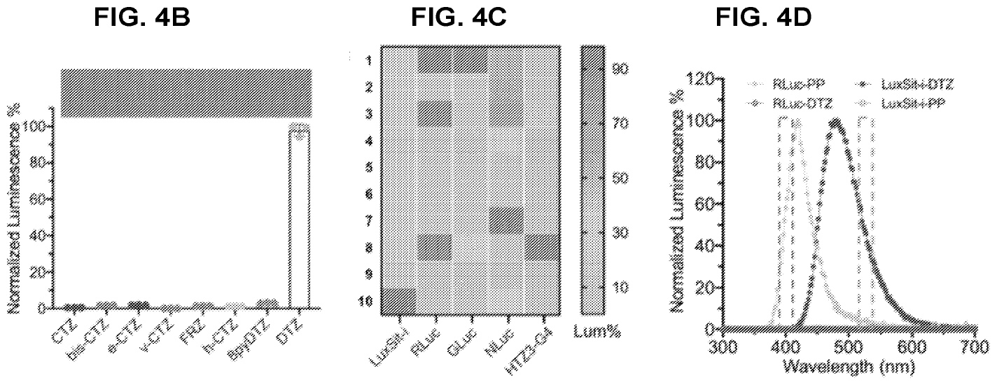

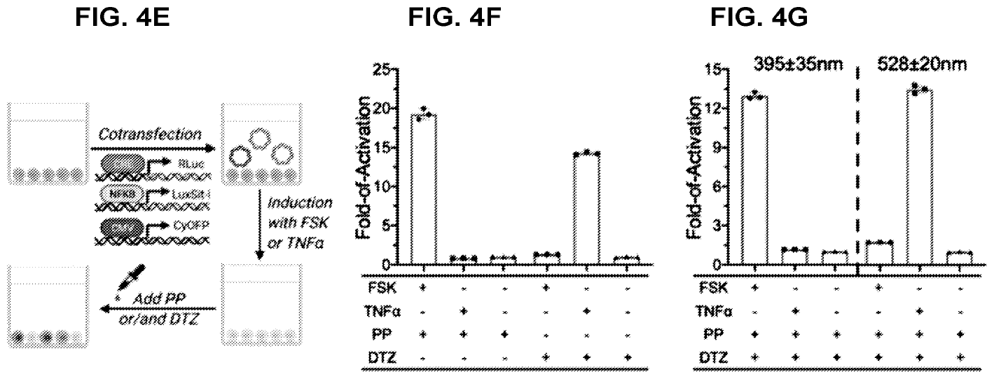

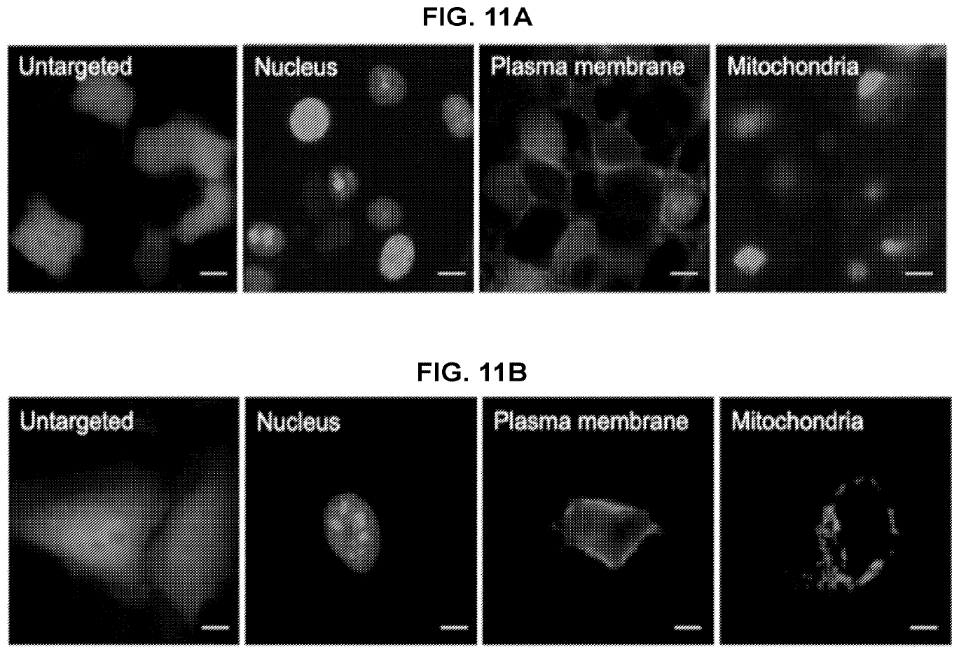

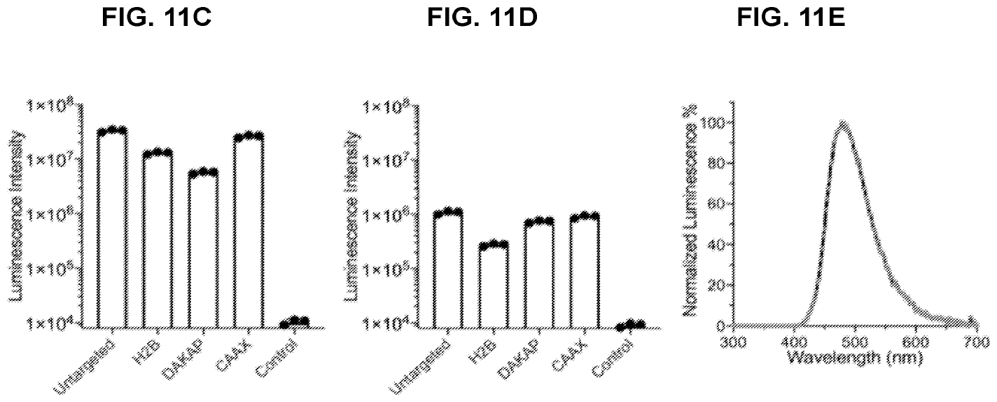

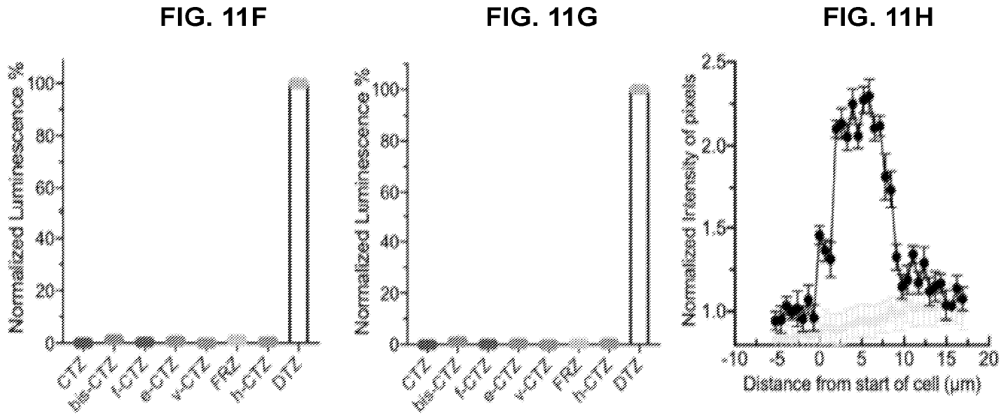

As luciferases are commonly used genetic tags and reporters for the study of cellular functions, we evaluated the expression and function of LuxSit-i in live mammalian cells. LuxSit-i-mTagBFP2-expressing HEK293T cells had DTZ specific luminescence (FIG. 3b), which was maintained following targeting of LuxSit-i to the nucleus, membrane, and mitochondria (FIG. 11). Native and previously engineered luciferases are quite promiscuous with activity on many luciferin substrates (FIG. 4ac), possibly due to their large and open pockets (a luciferase with high specificity to one luciferin substrate has been difficult to control even with extensive directed evolution³⁵). In contrast, LuxSit-i exhibited exquisite specificity to its target luciferin with 50-fold selectivity for DTZ over bis-CTZ (which differ only in a benzylic carbon. Overall, the specificity of our designed luciferases is much greater than native luciferases^{36,37} or previously engineered luciferases³⁸.

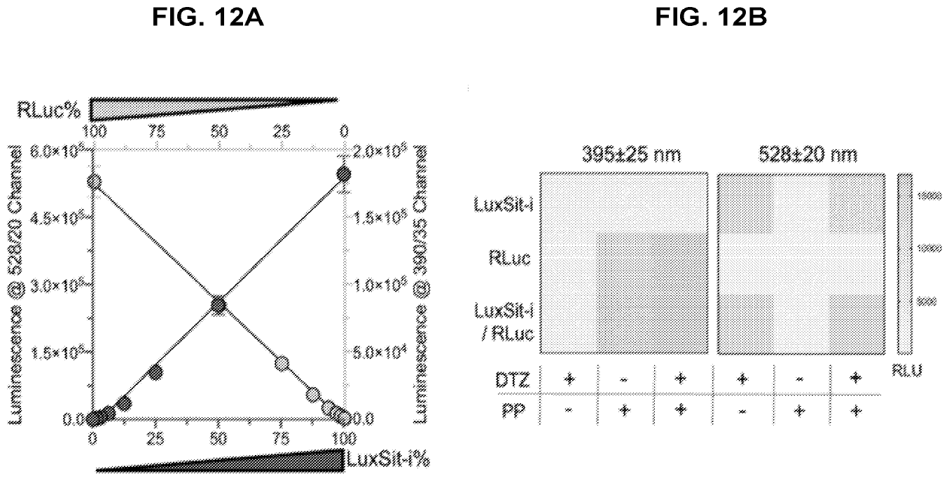

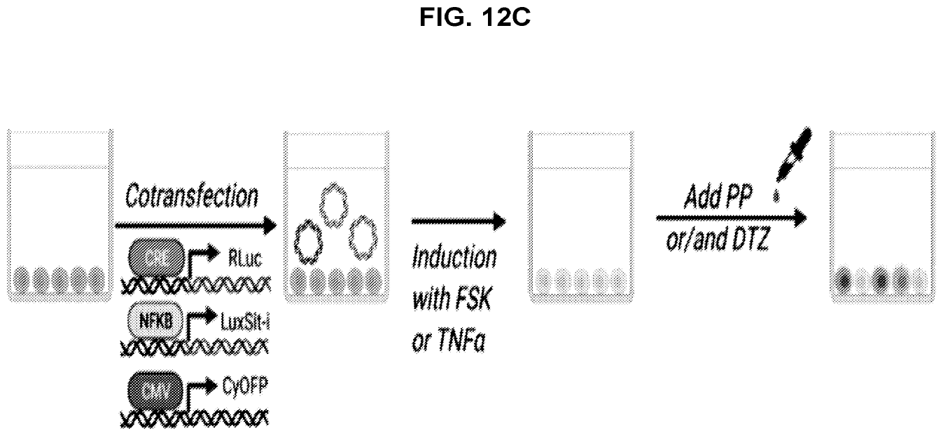

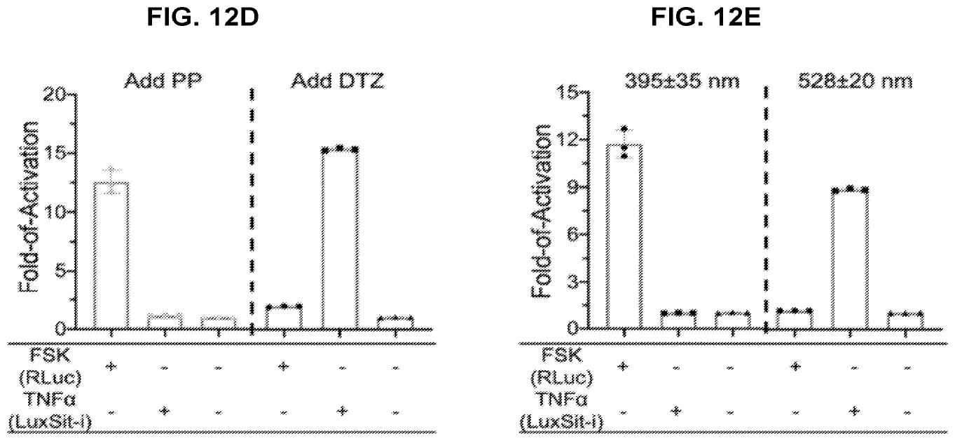

The high substrate specificity of LuxSit-i might allow multiplexing of luminescent reporters through substrate-specific or spectrally resolved luminescent signals (FIG. 4d and FIG. 12ab). To explore this possibility, we tracked two independent signaling pathways (cAMP/PKA and NF-κB) by placing the expression of either RLuc or LuxSit-i downstream of the NF-κB or cAMP response element promoters, respectively (FIG. 4e). Imaging in the presence of the substrates for the two luciferases (PP-CTZ for RLuc and DTZ for LuxSit-i) one at a time (FIG. 4f) can clearly distinguish known activators of the two pathways. Because the luminescence of the two reactions occurs at different wavelengths, we were also able to (FIG. 4g) simultaneously assess the activation of the two signaling pathways in the same sample in either intact HEK293T cells or cell lysates (FIG. 12c-e) by providing both substrates together and monitoring luminescence at different wavelengths.

### CONCLUSION

Computational enzyme design to date has been constrained by the number of available scaffolds, which limits the extent to which catalytic configurations and enzyme-substrate shape complementarity can be achieved¹⁶⁻¹⁸. The use of deep-learning to generate large numbers of de novo designed scaffolds here eliminates this restriction; moving forward, the more accurate RoseTTAfold™³⁹ and AlphaFold2™³³ should enable still more effective protein scaffold generation by leveraging family-wide hallucination abilities. The diversity of scaffold pocket shapes and sizes enabled the exploration of a range of catalytic geometries and the maximization of substrate-enzyme shape complementarity; to our knowledge, no native luciferases have folds similar to LuxSit, and the two enzymes have high specificity for fully synthetic luciferin substrates that do not exist in nature. With the incorporation of 2-3 substitutions that provide a more complementary pocket to stabilize the transition state, LuxSit-i has higher activity than any previously de novo designed enzyme; the k_{cat}/K_{M} of 10⁶ M⁻¹s⁻¹ is in the range of native luciferases. This is a notable advance for computational enzyme design, as tens of rounds of directed evolution were required to obtain catalytic proficiencies in this range for a designed retroaldolase, and the structure was remodeled considerably⁴⁰; in contrast, the predicted differences in ligand-sidechain interactions between LuxSit and LuxSit-i are very subtle. Achieving such high activities directly from the computer remains an outstanding goal for computational enzyme design. The small size of LuxSit makes it well suited as a genetic tag for capacity-limited viral vectors, biosensor development, and fusions to proteins of interest. On the basic science side, the small size, simplicity, and high activity make LuxSit-i an excellent model system for computational and experimental studies aimed at improving understanding of the luciferase catalytic mechanism. Extension of the approach used here to create similarly specific new luciferases for synthetic luciferin substrates beyond DTZ and h-CTZ would considerably extend the multiplexing opportunities illustrated in FIG. 4 or with the microscopy phasor⁴¹, leading to widely useful multiplexed luminescent toolkits. More generally, our family-wide hallucination method opens up an almost unlimited number of new scaffold possibilities for substrate binding and catalytic residue placement, which is particularly important in cases where the reaction mechanism, and how to promote it, are not completely understood: many structural and catalytic hypotheses can be readily enumerated with different catalytic residue placements in shape and chemically complementary binding pockets. While luciferases are unique in catalyzing the emission of light, the chemical transformation of substrates into products is common to all enzymes, and the approach developed here should be readily extendable to a wide variety of chemical reactions.

TABLE 3
Photoluminescence properties of selected LuxSit variants

| | | | | Vₘₐₓ | |
|---|---|---|---|---|---|
| | LQY^{a} | k_{cat} | K_{M} | (photon s⁻¹ | K_{cat}/K_{M} |
| | (%) | (1/s) | (μM) | molecule⁻¹) | (×10⁶ M⁻¹s⁻¹) |
| LuxSit-i | 14.5 ± 1.8 | 2.5 | 2.5 | 0.36 | 1.0 |
| LuxSit-f | 10.9 ± 1.5 | 1.8 | 8.9 | 0.20 | 0.2 |
| RLuc | 5.3^{b} | 4.9 | 1.5 | 0.26 | 3.3 |

^{a}mean ± s.d., n = 3 (technical triplicates). LQY (luminescent quantum yield) measurements were performed by consuming 125 pmol of DTZ (w/LuxSit-i, and LuxSit-f) or CTZ (w/RLuc) in 50 AL PBS with 50 nM corresponding recombinant luciferases.
^{b}All LQY values were estimated relative to the reported quantum yield of RLuc⁴². All values were calculated by the assumptions of the simplest Michaelis-Menten kinetics model, excluding potential substrate/product inhibition or enzyme modification during the reaction^{43,44}.

### Methods

### 1. Materials and General Methods

Synthetic genes and oligonucleotides were purchased from Integrated DNA Technologies or GenScript. The synthetic gene was inserted between NdeI and XhoI sites of a pET29b+ vector, containing an N-terminal hexahistidine tag followed by a TEV protease cleavage site and a C-terminal stop codon. Restriction endonucleases, Q5 PCR polymerase, and T4 ligase were purchased from NEB. Plasmid DNA, PCR products, or digested fragments were purified by Qiagen DNA purification kits. DNA sequences were analyzed by Genewiz. Coelenterazine (CTZ) was purchased from Gold Biotechnology. Diphenylterazine (DTZ), pyridyl diphenylterazine (8pyDTZ), and Furimazine (FRZ) were purchased from MedChemExpress. All other coelenterazine analogs (bis-CTZ: bisdeoxycoelenterazine; f-CTZ: f-Coelenterazine; e-CTZ: e-Coelenterazine-F; PP-CTZ: methoxy e-Coelenterazine; v-CTZ: v-Coelenterazine. All other chemicals were purchased from Sigma-Aldrich or Fisher Scientific and used without further purification. To identify the molecular mass of each protein, intact mass spectra were obtained via reverse-phase LC/MS on an Agilent 6230B TOF on an AdvanceBio RP-Desalting column and subsequently deconvoluted by Bioconfirm software using a total entropy algorithm. AKTA pure M with UNICORN 6.3.2 Workstation control (GE Healthcare) coupled with a Superdex™ 75 Increase 10/300 GL column was used for size exclusion chromatography. DNA and protein concentrations were determined by a NanoDrop™ small-volume 8 channel UV/vis spectrometer. CD spectra and CD melting experiments were performed by the default setting on a J-1500 Circular Dichroism Spectropolarimeter (Jasco). All luminescence measurements were acquired by a Biotek Synergy Neo2T™ Multi-Mode Plate Reader. To convert relative arbitrary unit (RLU) to the number of photons, Neo2 plate reader was calibrated by determining the chemiluminescence of luminol with known quantum yield in the presence of horseradish peroxidase and hydrogen peroxide in K₂CO₃ aqueous solution as previously described⁴⁵. SDS PAGE and luminescence images were captured by a Bio-Rad ChemiDocT™ XRS+. Images were analyzed using the Fiji image analysis software.

### 2. General Procedures for Protein Production and Purification

Lemo21(DE3) strain was used for transformation with the pET29b+ plasmid encoding the gene of interest. Transformed cells were grown for 12 h in TB medium supplemented with kanamycin. Cells were inoculated at 1:50 ratio in 100 mL fresh TB medium, grown at 37° C. for 4 h, and then induced by IPTG for an additional 18 h at 16° C. Cells were harvested by centrifugation at 4,000 g for 10 min and resuspended in 30 mL lysis buffer (20 mM Tris-HCl pH 8.0, 300 mM NaCl, 30 mM imidazole, and Pierce™ Protease Inhibitor Tablets). Cell resuspensions were lysed by sonication for 5 min (10 s per cycle). Lysates were clarified by centrifugation at 24,000 g at 12° C. for 40 min and pre-equilibrated with 1 mL of Ni-NTA nickel agarose at 4° C. for 1 h. The resin was washed twice with 10 mL wash buffer and then eluted in 1 mL elution buffer (20 mM Tris-HCl pH 8.0, 300 mM NaCl, 300 mM imidazole). The eluted proteins were purified by size exclusion chromatography in PBS. Fractions were collected based on A280 trace, snap-frozen in liquid nitrogen, and stored at −80° C.,

### 3. Computational Design of Idealized Scaffolds

Our generation of idealized NTF2-scaffolds can be divided into four parts: (3.1) Generation of seed-structures, (3.2) optimization of backbone geometries using trRosetta™-based hallucination, (3.3) generation of structure-conditioned sequence models to bias design, (3.4) design and filtering.

### 3.1 Generation of Seed Structures

We thought to increase the set of NTF2 structures by complementing experimentally resolved structures from the PDB with highly accurate models generated by trRosetta™²². To achieve this, we first collected 85 NTF2-like protein structures from the PDB based on SCOPe annotation (d. 17.4 SCOPe v2.05). Corresponding sequences were then used as queries to collect sequence homologs from UniProt™ by performing 8 iterations of hhblits at 1e-20 e-value cutoff against uniclust30_2018_08 database; default filtering cutoffs were relieved (−maxfilt 100000000−neffinax 20−nodiff-realign_max 10000000) to maximize the number of the output hits. All the hits were redundancy reduced using cd-hit⁴⁶ with a sequence identity cutoff of 60% yielding a set of 7,573 candidates for modeling.

To generate inputs for structure modeling with trRosetta™, we built multiple sequence alignments (MSAs) for each of the 7,573 selected sequences with hhblits using a more conservative e-value cutoff of 1e-50; the resulting MSAs were also complemented by hits from hmmsearch against uniref100 (release-2019_11) with the bit-score threshold of 115 (i.e. ˜1 bit per position). After joining the above two sets of alignments and filtering them at 90% sequence identity and 75% coverage cutoffs, only sequences with more than 50 homologs in the corresponding MSAs were retained for modeling (2,005 sequences). The filtered MSAs along with information on the top 25 putative structural homologs as identified by hhsearch against the PDB100 database of templates were used as inputs to the template-aware version of trRosetta™⁴⁷ to predict residue pair distances and orientations. Network predictions were then used to reconstruct full atom 3D structure models using a Rosetta™-based folding protocol described previously²².

### 3.2 Hallucination of Idealized NTF2s

Seeking to idealize the native structure seeds, we reasoned that trRosetta™, a convolutional residual neural network, which predicts residue-residue orientations and distances from sequence, could serve as a key component in a protein idealizer. Previously, this network has been used to generate diverse proteins that resemble the “ideal” structures of de novo designed proteins by changing the protein sequence to optimize the contrast (KL-divergence) between the predicted geometry and that of randomly generated sequences¹⁹.

For our purpose, the desired fold-space is not diverse but instead focused on the NTF2-like topology. To guarantee generation of ideal structures within this fold-space, we implemented a new fold-specific loss-function, which biased hallucinations based on observed geometries in native crystal structures. As many experimentally characterized NTF2s contain non-ideal regions, we began by creating a set (χ) of trimmed but ideal NTF2s by manually removing non-ideal structural elements such as kinked helices, and long or rarely observed loops. For each seed structure, we then used a structure-based sequence alignment method (see 3.3) to find equivalent positions between the seed structure and χ. Residue pairs were considered to be in a conserved tertiary motif (TERM) if there were 5 or more equivalent positions in χ. The smooth probability distributions based on observed geometries in χ were then computed. For distances we used a Gaussian distribution with mean equal to the true distance denoted by D and standard deviation denoted by a equal to 0.5 Å. The probability density function for distances d is given by:

$$f ( d ; D , σ ) = \frac{1}{\sqrt{2 {πσ}^{2}} exp ( - \frac{{( d - D )}^{2}}{2 {σ}^{2}} )}$$

Using this density function one can construct a categorical distribution for binned distances by evaluating this function at the centers of the bins and then normalizing by a sum of all values in different bins. Similarly, a von Mises distribution was used for omega angle smoothing with probability density function given by ƒ(ω; Ω, κ)=N(κ) exp[κ cos(ω−Ω)] where N(κ) is a normalizing constant, Ω is the crystal value, κ is the inverse variance chosen to be 100, and ω is the smoothed angle. For phi and theta angles a von Mises-Fisher blur is given by ƒ(x; y, K)=N(κ) exp[κ μ^{T}x] where N(κ) is a normalizing constant, μ is a unit vector on a 3D sphere corresponding to the phi and theta angles from the crystal structure, x is a smoothed unit vector, and K is the inverse variance chosen to be 100.

Next, we converted those probability distributions to energy landscapes (ie—negative log likelihoods) and sought to minimize the expected energy. This soft restraint encouraged the network to seek out the consensus structure, while still allowing deviations where needed. Specifically, we formulated the fold-specific loss as:

$${L}_{fold} = {\sum }_{x ϵ \{ d , ω , θ , Φ \vert \}} [ {\sum }_{i , j = 1}^{L} {\sum }_{k = 1}^{{N}_{x}} - {m}_{ij} {p}_{x , ijk} ln ( {s}_{x , ijk} ) ] / {\sum }_{i , j = 1}^{L} {m}_{ij}$$

$${m}_{ij} = \{ 1 if 1 and j are in a TERM ; else 0$$

where p is the network prediction and s is the smoothed probability distribution of the conserved residue pairs. For the second part of the loss function and similar to previous work¹⁹, we sought to maximize the Kullback-Leibler (KL) divergence between the predicted probability distribution and a background distribution for all i,j residue pairs not in a TERM.

$${L}_{hall} = - {\sum }_{x ϵ \{ d , ω , θ , Φ \vert \}} [ {\sum }_{i , j = 1}^{L} {\sum }_{k = 1}^{{N}_{x}} ( 1 - {m}_{ij} ) {p}_{x , ijk} ln ( {p}_{x , ijk} / {b}_{x , ijk} ) ] / {\sum }_{i , j = 1}^{L} ( 1 - {m}_{ij} )$$

where b is the background distribution and Nₓ is the number of bins in each probability distribution (N_{d}=37, N_{ω,θ}, =25, N_{φ}=13). Briefly, b is calculated by a network of similar architecture to trRosetta™ trained on the same training data, except it is never given sequence information as an input. The final loss is given by:

$$L = {L}_{fold} + {L}_{hall}$$

We used a Markov Chain Monte Carlo (MCMC) procedure to search for sequences that trRosetta™ predicted to fold into structures that minimize this loss function. We allowed four types of moves with different sampling probabilities: mutations (p=0.55), insertions (p=0.15), deletions (p=0.15), and moving segments (p=0.15). Mutations randomly changed one amino acid to another, with an equal transition probability for all 20 amino acids. Insertions inserted a new amino acid (all equally likely) into a random location subject to the KL-divergence loss. Deletions deleted a random residue from the same locations. Finally, we also allowed “segments” to move, cutting and pasting themselves from one part of the sequence to another, while maintaining the same overall segment order. Here, a “segment” is a continuous stretch of amino acids all subject to fold specific loss, often composed of a single strand or helix. Starting from a random sequence of an initial length (typically 120 amino acids), we used the standard Metropolis criteria to accept or reject moves:

$${A}_{i} = min [ 1 , exp ( - ( {L}_{i} - {L}_{i - 1} ) / T ) ]$$

where Aᵢ is the chance of accepting the move at step i, Lᵢ is the loss at the current step, Lᵢ₋₁ is the loss at the previous step and T is the temperature. The temperature started at 0.2 and was reduced by half every 5 k steps. Generally, it took 30 k steps to converge.

### 3.3 Structure-Conditioned Multiple Sequence Alignment

Given the complexity of the NTF2-like protein fold, we hypothesized that it was necessary to impose sequence design rules to disfavor alternative states (negative design). Towards this end, we computed a structure-conditioned multiple sequence alignment based on native NTF2-like proteins. Specifically, we used TMalign⁴⁸ to superimpose each of the 2005 predicted native structures (from 3.1) onto each hallucinated backbone (from 3.2). Next, to find structurally corresponding positions, we implemented a structure-based dynamic programming algorithm, similar to the Needleman-Wunsch algorithm⁴⁹. However, instead of using the amino acid similarity as the scoring metric, we used a tunable structure-based score function. After aligning the two structures, we scored the structural similarity of any two residues by empirically weighting several metrics: (1) Distance between Ca atoms, (2) differences between backbone torsion angles (phi and psi) backbone torsion angles and (3) the angle (degrees) between the vectors pointing from Cα to Cβ in each residue. To calculate the unweighted score for each component, we normalized each by a maximum possible value (180 degrees for angles and 10 Å for distances) and included a “set point” that approximately delineated when we judged a metric to indicate two residues to be more similar than not. Values above this setpoint are positive, indicating two residues are similar and values below the set point indicated two residues are dissimilar.

$${Score}_{unweighted} = ( set\_point - value ) / max\_value$$

Each value was scaled by its normalized weight and summed to give an overall similarity score between any two amino acids.

These similarity scores were used as the similarity metric in our dynamic programming algorithm, in place of the typical BLOSUM62 similarity metric. We used a gap penalty of 0.1 and an extension penalty of 0.0. Finally, after concatenating all the structure-conditioned aligned sequences, we used PSI-BLAST-exB^{50,51} to compute sequence redundancy weighted log-odds scores for each amino acid at each position (position-specific scoring matrices, PSSMs).

### 3.4 Design

To design the resulting backbones, we sought, in addition to the sequence patterns captured in the PSSM (3.3), to further specify the backbone conformation and functionalize the pocket, by installing entire hydrogen bonding networks from native NTF2-like proteins. We compiled two sets of hydrogen bonding networks: a set for the cavity containing 85 networks and another set of networks connecting the C-terminal region of the first helix with the third beta-strand containing 25 networks. In 20 independent attempts for each backbone, we randomly grafted a network from each set, fixed the identities of hydrogen bonding residues, and designed the sequences for all other positions under PSSM constraints. The resulting models were filtered for various backbone quality metrics and for maintenance of hydrogen bonding networks in the absence of constraints, resulting in a total of 1615 idealized scaffolds.

### 4 RIFdock Tuning Files

The hierarchical search framework of RifDock is a powerful way to search through 6-dimensional rigid body orientations. While originally designed to work with physics-based forcefields, the scoring machinery can easily be modified to do other things. A system was added called “Tuning Files” that allows one to tune the energetics of rifdock by “requiring” specific interactions. Specified interactions can range from specific hydrogen bonds, to specific bidentates, and even to specific hydrophobic interactions. The specifics are that during the RifGen stage, each stored rotamer is compared against a list of definitions in the Tuning File. If the rotamer satisfies a definition, it is stored into the RIF with a “Requirement Number”. Later during RifDock, these Requirement Numbers are available during scoring and the presence or absence of certain rotameric interactions may be used to penalize or even completely discard dock solutions. In this work, the Tuning Files were used to require the specific hydrogen bond interactions between the arginine and the secondary amine in the pyrazine ring of the colenterazine-like substrate.

5 Designing Theozyme Architectures into De Novo NTF2 Scaffolds

De novo design of luciferases can be divided into three main steps—scaffold construction, substrate placement with required interactions, and sequence design. With the idealized NTF2-like scaffolds in hand, we selected 5 diverse rotamers from AIMNet and used the Rotamer Interaction Field (RIF) docking method³¹ to exhaustively search a large space of interacting side chains to the anionic form of DTZ. Chemically, deprotonation of N1 hydrogen is the first step to forming an anionic species (FIG. 5). We first generated RIF using RifGen³¹ to guide placement in the protein scaffolds. We required the placement of a positively charged Arginine sidechain by a tuning file (see below) to stabilize the formation of negative charge N1 atom where the deprotonation occurs initially and enumerated large numbers of possible sidechain interactions with the rest of DTZ.

HBOND_DEFINITION
N1 1 ARG
END_HBOND_DEFINITION
REQUIREMENT_DEFINITION
1 HBOND N1 1
END_REQUIREMENT_DEFINITION

Rifdock was then used to hierarchically search for the best combination of RIF to place on the input backbone. Although the negative charge can move to another electronegative atom O1 via resonance of the imidazopyrazinone core, it is unclear which anionic species is more critical for the luciferase-catalyzed luminescence emission. Thus, we let RIFdock place the polar rotamers on the basis of hydrogen-bond geometry to O1 and apolar rotamers to DTZ without specific requirements. In the next docking step, we parsed the -scaffold_res argument with a list of residue numbers as scaffold backbone positions that were annotated as pocket residues to allow a hierarchical search of RIF placement. We only allowed the RIF placements in the pocket residues and left pre-defined hydrogen bond networks (HBNets) intact. After RIFdock, we continued for Rosetta™ sequence design where the score function was reweighted for higher buried_unsat_penalty⁵² and the amino acid selection was biased by giving a pre-generated PSSM file via SeqprofConsensus task operation. This would minimize buried unsatisfied residues and increase pre-organized architectures in the core that are known to be beneficial for a catalytic pocket⁵³. Two rounds of FastDesign calculation were included: we restricted the RIF rotamers and core HBNets to repacking in the first round while we allowed other residues for re-design based on PSSM during the Monte Carlo simulated annealing procedure. After the surrounding residues were optimized to retain the RIF interactions, we allowed the re-design of RIF rotamers, to find efficient aromatic and hydrophobic packing around DTZ while catalytic residues (the N1 requirement) were still limited to only repacking. The final set of designs was obtained after filtering by ligand-binding interface energy, shape complementarity, contact molecular surface, number of HbondsToResidue, and the presence of N1_hbond.

6. Structure Prediction of LuxSit with AlphaFold2 and Comparison to Design Model

To computationally assess the accuracy of the LuxSit design model, we performed single sequence structure prediction using AlphaFold2. All models were run with 12 recycles and generated models were relaxed using AMBER⁵⁴. The model with the highest pLDDT was used for comparison to the Rosetta™ design model and structural superpositions were performed using the Theseus alignment tool to determine backbone RMSD between the design model and AlphaFold2 model³³.

### 8. Computational SSM Experiment to Estimate Mutation Binding Free Energy

Rosetta™ cartesian_ddg application^{55,56} was used to computationally estimate enzyme and substrate binding free energy. The LuxSit design model was relaxed beforehand in cartesian space with the substrate-bound. For the 21 positions that were experimentally screened for single mutation effects on luciferase activity, each residue was computationally mutated into other amino acid types and packing and cartesian relaxation was performed to evaluate the final score in REU. This procedure was applied three times in parallel for both substrate-bound and apo-states. The average of the three calculation results was used to calculate the relative binding free energy (ddG_{bind}) by subtracting the total score of the apo-state from the complex state.

### 9. Construction and Screening of Designed Luciferase Libraries

The construction of assembled gene libraries was described previously in detail⁵⁷. In brief, the amino acid sequences of all designed luciferases were first reverse-translated into E. coli codon-optimized DNA sequences. All DNA sequences were categorized into multiple sub-pools by the gene length (˜500 designs per sub-pool). Each gene was subsequently split into two fragments (fragment A and fragment B) and added outer and inner primer sequences to the 5′ and 3′ end (e.g., Outer_oligoA_5primer+design_half A+Inner_oligoA_3primer and Inner_oligoB_5primer+design_half B+Outer_oligoB_3primer). All oligos were ordered in one Twist 250 nt Oligo Pool. To construct the library of each sub-pool, polymerase chain reaction (PCR) with oligoA_5primer/oligoA_3primer or oligoB_5primer/oligoB_3primer oligonucleotide pairs was used to amplify the individual fragment A or fragment B from each sub-pool. The pool-specific sequences were removed with Uracil Specific Excision Reagent (USER) followed by NEB End Repair kit. Outer primers (oligoA_5primer and oligoB_3primer) were then used for fragment A and fragment B assembly and amplification. The assembled full-length fragment was digested with XhoI/HindIII and ligated into a predigested pBAD/His B vector. All ligation products were used to transform ElectroMAX™ DH10B Cells, which were next plated on 150 mm×15 mm LB agar plates supplemented with carbenicillin and L-arabinose. We sequenced 30 random colonies and 11 of the sequences were in our designed library. The plates (˜2000 colonies per plate) were incubated at 37° C. overnight to form bacterial colonies and left at 4° C. for another 24 h. To directly image luminescence activity from bacterial colonies, we sprayed the PBS solution containing 30 μM DTZ to each agar plate, waited for 2 min, and the luminescence images were acquired and processed with Bio-Rad ChemiDoc XRS+. After screening 15 plates, active colonies were collected for sequencing, protein expression, and other downstream characterization where LuxSit was selected from three active designs shown catalytic signal above background.

### 10. Construction and Evaluation of LuxSit Site Saturation Mutagenesis Libraries

To create libraries of each single amino acid substitution at residues 13, 14, 17, 18, 35, 37, 38, 49, 52, 53, 56, 60, 65, 81, 83, 94, 96, 98, 100, 110, and 112, forward oligos mixture with degenerate codons (NDT, VHG, and TGG=1:1:0.1 ratio) and an overlapped reverse oligo were used to amplify the plasmid of LuxSit. The resulting PCR products were circularized by Gibson Assembly protocol and were subsequently used to transform ElectroMAX™ DH10B Cells. The cells were plated on 150 mm×15 mm LB agar plates supplemented with carbenicillin and L-arabinose, incubated at 37° C. overnight, and left at 4° C. for another 24 h. As described in the screening of luciferase libraries, colony-based screening by spraying DTZ solution was used to identify active colonies. Inactive colonies were also randomly picked. As a result, a total of 32 colonies were picked for each residue library. 32×21 individual colonies were grown in 1 mL of TB supplemented with carbenicillin and L-arabinose in 96-well deep-well culture plates. The plates were shaken at 37 C overnight (˜16-18 h) on 96-well plate shakers at 1,100 rpm. Cells were pelleted by centrifugation at 4,000 g for 15 min in a tabletop centrifuge. Media was discarded and the cell pellets were resuspended in 0.2 mL BugBuster HT Protein Extraction buffer. The plates were transferred back to 96-well plate shakers and incubated at 1,100 rpm for an additional 30 min. Cellular debris was pelleted again by centrifugation at 4,000 g for 15 min, soluble lysates were transferred to a new semi-deep 96-well plate, and incubated with 10 μL of magnetic Ni-NTA beads for 30 min to allow binding. The magnetic extractor was used to first transfer the beads from the binding plates to wash plates with 200 μL IMAC wash buffer in each well, and then transfer the beads to elusion plates containing 30 μL IMAC elution buffer in each well. The concentrations of all proteins in each well were determined by the Bradford assay directly. The elution solution in each well was used to make a 25 μL protein solution at indicated concentration and mixed with 25 μL of 50 μM DTZ PBS solution. The luminescence signals were acquired over a course of 15 min while the actual point mutation was identified by sequencing. Thus, the mutation-to-activity relationship can be mapped. To evaluate whether these beneficial mutations are synergistic, we ordered individual mutants with combinatorial mutations at residue 14, 60, 96, 98, and 110 (see Table 4), expressed, and purified these LuxSit variants for kinetic, emission spectra, and luminescence intensity. We identified four mutants that can produce 47 to 77-fold more photons than the parent LuxSit. We assigned one of which, LuxSit-f (A96M/M110V), for its strong initial flash emission. Since the mutations at residue 96 and 110 are robust and mutations at residue 60 are versatile, we generated a fully randomized library at 60, 96, and 110 positions to exhaustively screen all possible combinations. After the colony-based screening, we identified many colonies with strong luciferase activities with DTZ (FIG. 9). Among all selected mutants, Arg60 is confirmed to be mutable, Ala96 prefers larger hydrophobic sidechains (Leu, Ile, Met, and Cys), and Met110 favors hydrophobic residues (Val, Ile, and Ala). A newly discovered mutant R60S/A96L/M110V with more than 100-fold higher photon flux over LuxSit was assigned LuxSit-i for its high brightness.

### 11. In Vitro Characterization of Photoluminescence Properties

For Michaelis-Menten kinetics measurements, 25 μL of serial diluted DTZ substrate in Tris pH 8.0 buffer was added into the wells of a white 96-well half-area microplate containing 25 μL of purified luciferases (final enzyme concentration: 100 nM; substrate concentration: 0.78 to 50 μM). Measurements were taken every 1 min (0.1 s integration and 10 s shaking between each interval) for a total of 20 min. Initial velocities were estimated as the average of the light intensities from the first three data points to fit the Michaelis-Menten equation. All relative arbitrary unit (RLU) per second values were converted to photon/s by the luminol-H₂O₂—HRP calibration method⁴⁵. Following the equation: Iₘₐₓ=LQY×k_{cat}×[E], Iₘₐₓ is the maximal photon flux (photon s⁻¹), [E] is the total enzyme concentration, and V ax is the maximum photon flux per molecule (photon s⁻¹ molecule⁻¹) from the fitting of the Michaelis-Menten equation. To determine the luminescent quantum yields, 25 μL of 5 μM individual substrate in PBS was injected into 25 μL PBS containing 100 nM corresponding luciferase. DTZ was used for all LuxSit variants while CTZ was used as the substrate of native RLuc. The luminescence signals were monitored until the reactions were completed (0.1 s integration and measurements were taken every 5 s for a total of 40 min). The sum of luminescence photon counts was normalized to the total photon counts of RLuc/CTZ pair (LQY=5.3±0.1%)⁵⁸ to derive relative luminescent quantum yields of LuxSit variants (FIG. 10c). k_{cat} values for each individual enzyme were calculated using the equation: k_{cat}=Vₘₐₓ/LQY. To record emission spectra, 25 μL of 50 μM DTZ in PBS were injected into 25 μL of 200 nM pure luciferases and the emission spectra were collected with 0.1 s integration and 2 nm increments from 300 to 700 num. In vitro luminescence activity measurements of LuxSit-i expressing HEK293T or HeLa cells were done similarly as 15,000 intact cells or lysates were used in the assay instead of purified luciferases. To evaluate the substrate specificity, 25 μL of 50 μM substrate analogs in PBS were added to 25 μL of 200 nM indicated luciferases, and the signals were recorded over 20 min. Data were shown as the total luminescence signal over the first 10 min. We normalized the data by setting the highest emission substrate at 100%.

### 12. Circular Dichroism (CD)

Purified protein samples were prepared at 15 μM in pH 7.4 10 mM phosphate buffer. Spectra from 190 nm to 260 nm were recorded at 25° C., 50° C., 75° C., 95° C., and after cooling back to 25° C. Thermal denaturation was monitored at 220 nm from 25° C. to 95° C. (1° C. per min increments). Tm values were not reported because no obvious inflection points of the melting curves.

### 13. Mammalian Cell Culture and Transfection

HEK293T and HeLa cell lines were maintained at 37° C. with humidified 5% CO₂ atmosphere and cultured in Dulbecco's Modified Eagle's Medium (DMEM, GIBDO) supplemented with 10% fetal bovine serum (FBS, Sigma). Cells were transfected with Turbofectin™ 8.0 (Origene) with 500 μg of plasmid DNA. After 24 h at 37° C. in a CO₂ incubator, the medium was removed, and cells were collected and resuspended in Dulbecco's phosphate-buffered saline (DPBS).

### 14. Fluorescence Microscopy and Image Analysis

Cells were washed twice with HBSS and subsequently imaged in HBSS in the dark at 37° C. Right before imaging, cells were incubated with 25 μM DTZ. Epifluorescence imaging was conducted on a Yokogawa CSU-X1 microscope equipped with a Hamamatsu ORCA-Fusion scientific CMOS camera and Lumencor Celesta light engine. Objectives used were: 10×, NA 0.45. WD 4.0 mm, 20×, NA 1.4, WD 0.13 mm, and 40×, NA 0.95. WD 0.17-0.25 mm with correction collar for cover glass thickness (0.11 mm to 0.23 mm) (Plan Apochromat Lambda). Imaging for BFP utilized a 408 nm laser, 432/36 nm dichroic, and a 440/40 nm emission filter (Semrock). Exposure times were 200 ms for BFP and 10 s for luminescence. All epifluorescence experiments were subsequently analyzed using NIS Elements software.

15. Multiplex Dual-Luciferase Reporter Assay for the cAMP/PKA and NF-κB Pathways

HEK293T cells were grown in a tissue culture-grade white 96-well plate and transfected with indicated CRE-RLuc, NFκB-LuxSit-i, and CMV-CyOFP plasmids. 24 h after transfection, the medium was replaced by 2 μM of Forskolin (FSK) or 300 ng/mL human tumor necrosis factor alpha (TNFα) in regular cell media. 23 h after stimulation, the cells were resuspended in DPBS by pipette mixing. 25 μL of DPBS containing 30,000 intact cells was mixed with 25 μL of CelLytic M for 15 min to make cell lysates. For intact cell assay. 25 μL of DPBS containing 15.000 intact cells was mixed with 25 μL of PP-CTZ (2 μM) or/and DTZ (10 μM) in DPBS. For cell lysate assay, 25 μL of cell lysate was added to 25 μL of PP-CTZ (2 μM) or/and DTZ (10 μM) to initiate luminescence reactions. The signals were recorded every 1 min for a total of 10 min. The light signals were collected in the substrate-resolved mode without filters and with 528/20 and 390/35 filters under the spectrally resolved mode. Area scanning the fluorescence intensity of CyOFP at 480 nm (excitation wavelength) and 580 nm (emission wavelength) was used to estimate the total cell numbers and transfection efficiency. The reported unit was the average of the first 10 min luminescence (RLU) over the relative fluorescence units (a.u.). To derive fold-of-activation, all values were normalized to the corresponding non-stimulated control.

### Statistical Analysis

No statistical methods were used to pre-determine the sample size. No sample was excluded from data analysis. Results were reproduced using different batches of pure proteins on different days. Unless otherwise indicated, data are shown as mean±s.d., and error bars in figures represent s.d. of technical triplicate. Data were analyzed and plotted using GraphPad Prism 8, seaborn, and matplotlib.

### Supplementary Information

TABLE 4
Enzymatic and photoluminescence properties of LuxSit and its mutants

| | | | Vₘₐₓ | Relative |
|---|---|---|---|---|
| | λₘₐₓ | K_{M} | (×10⁻² photon s⁻¹ | emission |
| | (nm) | (μM)^{a} | molecule⁻¹)^{a} | intensity^{b} |
| LuxSit | 478 | 18.3(14.1-21) | 0.32(0.29-0.37) | 1 |
| (60R/96A/98H/110M) | | | | |
| Y14E | 478 | 12.6(9.9-16.1) | 0.19(0.17-0.21) | 0.95 |
| R60C | 476 | 45.2(28.7-78.9) | 0.57(0.44-0.8) | 1.55 |
| A96I | 476 | 17.5(12.1-25.9) | 1.3(1.1-1.5) | 4.32 |
| A96M | 476 | 4.8(3.1-7.3) | 6.0(5.3-6.9) | 16.4 |
| H98N | 476 | 29.3(22.1-39.8) | 0.17(0.15-0.2) | 0.48 |
| M110C | 478 | 67.5(43-121) | 0.4(0.3-0.6) | 0.64 |
| M110V | 480 | 30.3(22.7-41.5) | 7.1(6.2-8.4) | 19.3 |
| 60C/96I/98H/110C | 476 | 11.3(6.5-20.2) | 0.12(0.1-0.16) | 0.39 |
| 60R/96I/98N/110C | 476 | 8.7(3-7.4) | 0.02(0.017-0.022) | 0.12 |
| 60R/96I/98H/110C | 480 | 4.3(2,7-6.8) | 0.05(0.045-0.059) | 0.27 |
| 60C/96I/98H/110V | 478 | 6.3(4.8-8.5) | 14.7(13.4-16.2) | 77.1 |
| 60C/96M/98N/110V | 474 | 9.7(5.6-17.4) | 0.13(0.1-0.16) | 0.5 |
| 60C/96M/98H/110C | 478 | 30.9(18.3-57.2) | 2.44(1.9-3.4) | 4.26 |
| 60R/96I/98N/110V | 476 | 7.2(5.4-9.5) | 0.32(0.29-0.35) | 1.39 |
| 60R/96I/98H/110V | 482 | 10.8(8-14.6) | 12.9(11.7-14.5) | 47.5 |
| 60C/96M/98H/110V | 476 | 7.0(4.8-10.2) | 15.9(14-18.1) | 66.3 |
| 14E/60C/96M/98H/110V | 478 | 8.0(4.0-15.8) | 0.16(0.12-0.2) | 1.0 |
| 60R/96M/98N/110V | 478 | 8.0(5.3-12.2) | 0.044(0.04-0.05) | 0.21 |
| 60C/96M/98N/110C | 476 | 17.4(13.4-22.8) | 0.016(0.015-0.018) | 0.08 |
| 60C/961/98N/110C | 476 | 24.7(18.7-33.4) | 0.03(0.026-0.035) | 0.11 |
| 60R/96M/98H/110C | 474 | 23.4(15,9-35.8) | 0.28(0.24-0.35) | 0.58 |
| 60C/96I/98N/110V | 480 | 80.9(43.3-222) | 0.83(0.55-1.8) | 1.81 |
| 60R/96M/98H/110V | 480 | 8.9(6.7-11.9) | 19.7(17.9-21.9) | 60.9 |
| (LuxSit-f) | | | | |
| 60R/96M/98N/110C | 476 | 9.3(7-12.2) | 0.019(0.017-0.021) | 0.96 |
| 60S/96L/98H/110V | 482 | 2.5(1.9-3.3) | 36.1(33.7-38.6) | 153 |
| (LuxSit-i) | | | | |

^{a}mean (95% CI)., n = 3 (technical triplicates);
^{b}Integrated luminescence intensity over the first 20 min. All values were normalized to the signal of LuxSit with 25 μM DTZ in Tris pH 8.0 buffer.

TABLE 5
Amino acid and DNA sequences of de novo luciferases in this work
The sequences (underlined) below contain a PolyHis-TEV or PolyHis
tag for protein purification (which are optional
and may be present or deleted)

>LuxSit
MGSHHHHHHGSGSENLYFQGSMSEEQIRQFLRRFYEALDSGDADTAASLFHPGVTIHLWDGVTFTS
REEFREWFERLFSTRKDAQREIKSLEVRGDTVEVHVQLHATHNGQKHTVDATHHWHERGNRVTEMR
VHINPTG (SEQ ID NO: 192)
>LuxSit (codon optimized for E. coli expression)
ATGGGTAGCCATCACCACCATCACCATGGTAGCGGTAGCGAGAACTTGTACTTCCAAGGAAGCATG
AGCGAAGAACAGATTCGTCAGTTTCTGCGTCGTTTTTATGAAGCGCTGGATAGCGGCGATGCGGAT
ACCGCTGCGAGCCTGTTTCATCCGGGCGTGACAATTCATCTGTGGGATGGCGTTACCTTTACCAGC
CGTGAAGAATTTCGTGAATGGTTTGAACGTCTGTTTAGCACCCGTAAAGATGCGCAGCGTGAAATT
AAGAGCCTGGAAGTACGTGGCGATACCGTGGAAGTGCATGTGCAGTTGCACGCGACCCATAATGGC
CAGAAACATACCGTAGATGCAACCCATCATTGGCATTTTCGTGGCAATCGTGTGACCGAAATGCGT
GTGCATATCAATCCGACCGGCTAA (SEQ ID NO: 193)
>LuxSit-f
MGSHHHHHHGSGSENLYFOGMSEEQIROFLRRFYEALDSGDADTAASLFHPGVTIHLWDGVTFTSR
EEFREWFERLFSTRKDAQREIKSLEVRGDTVEVHVQLHATHNGQKHTVDMTHHWHERGNRVTEVRV
HINPTG (SEQ ID NO: 194)
> LuxSit-f (codon optimized for E. coli expression)
ATGGGTAGCCATCACCACCATCACCATGGTAGCGGTAGCGAGAACTTGTACTTCCAAGGAATGAGC
GAAGAACAGATTCGTCAGTTTCTGCGTCGTTTTTATGAAGCGCTGGATAGCGGCGATGCGGATACC
GCTGCGAGCCTGTTTCATCCGGGCGTGACAATTCATCTGTGGGATGGCGTTACCTTTACCAGCCGT
GAAGAATTTCGTGAATGGTTTGAACGTCTGTTTAGCACCCGCAAAGATGCGCAGCGTGAAATTAAG
AGCCTGGAAGTACGTGGCGATACCGTGGAAGTGCATGTGCAGTTGCACGCGACCCATAATGGCCAG
AAACATACCGTAGATATGACCCATCATTGGCATTTTCGTGGCAATCGTGTGACCGAAGTGCGTGTG
CATATCAATCCGACCGGCTAA (SEQ ID NO: 195)
>LuxSit-i
MGSHHHHHHGSGSENLYFQGMSEEQIRQFLRRFYEALDSGDADTAASLFHPGVTIHLWDGVTFTSR
EEFREWFERLFSTSKDAQREIKSLEVRGDTVEVHVQLHATHNGQKHTVDLTHHWHERGNRVTEVRV
HINPTG (SEQ ID NO: 196)
>LuxSit-i (codon optimized for E. coli expression)
ATGGGTAGCCATCACCACCATCACCATGGTAGCGGTAGCGAGAACTTGTACTTCCAAGGAATGAGC
GAAGAACAGATTCGTCAGTTTCTGCGTCGTTTTTATGAAGCGCTGGATAGCGGCGATGCGGATACC
GCTGCGAGCCTGTTTCATCCGGGCGTGACAATTCATCTGTGGGATGGCGTTACCTTTACCAGCCGT
GAAGAATTTCGTGAATGGTTTGAACGTCTGTTTAGCACCAGTAAAGATGCGCAGCGTGAAATTAAG
AGCCTGGAAGTACGTGGCGATACCGTGGAAGTGCATGTGCAGTTGCACGCGACCCATAATGGCCAG
AAACATACCGTAGATTTGACCCATCATTGGCATTTTCGTGGCAATCGTGTGACCGAAGTTCGTGTG
CATATCAATCCGACCGGCTAA (SEQ ID NO: 197)
>LuxSit-i (codon optimized for human cell expression)
ATGAGCGAGGAGCAGATCAGACAGTTCCTGAGGAGATTCTACGAGGCCCTGGATAGCGGAGACGCC
GACACAGCTGCCAGCCTTTTCCATCCTGGCGTGACCATCCACCTGTGGGACGGCGTCACCTTCACT
AGCAGGGAGGAGTTCAGGGAGTGGTTCGAGAGACTGTTCAGCACCAGCAAGGACGCCCAGAGAGAG
ATCAAGAGCCTGGAAGTTAGAGGCGACACCGTGGAAGTGCACGTGCAGCTGCACGCCACACACAAC
GGACAGAAGCACACCGTCGACCTGACCCACCACTGGCACTTCAGAGGCAACAGAGTGACCGAGGTG
AGAGTGCACATCAATCCCACCGGCTAA (SEQ ID NO: 198)

### REFERENCES

1. Jiang, L. et al. De novo computational design of retro-aldol enzymes. Science 319, 1387-1391 (2008).
2. Rothlisberger, D. et al. Kemp elimination catalysts by computational enzyme design. Nature 453, 190-195 (2008).
3. Yeh, H. W. et al. Red-shifted luciferase-luciferin pairs for enhanced bioluminescence imaging. Nat. Methods 14, 971-974 (2017).
4. Love, A. C. & Prescher, J. A. Seeing (and Using) the Light: Recent Developments in Bioluminescence Technology. Cell Chemical Biology 27, 904-920 (2020).
5. Syed, A. J. & Anderson, J. C. Applications of bioluminescence in biotechnology and beyond. Chem. Soc. Rev. 50, 5668-5705 (2021).
6. Yeh, H.-W. & Ai, H.-W. Development and Applications of Bioluminescent and Chemiluminescent Reporters and Biosensors. Annu. Rev. Anal. Chem. 12, 129-150 (2019).
7. Zambito, G., Chawda, C. & Mezzanotte, L. Emerging tools for bioluminescence imaging. Curr. Opin. Chem. Biol. 63, 86-94 (2021).
8. Markova, S. V., Larionova, M. D. & Vysotski, E. S. Shining Light on the Secreted Luciferases of Marine Copepods: Current Knowledge and Applications. Photochem. Photobiol. 95, 705-721 (2019).
9. Wu, N. et al. Solution structure of Gaussia Luciferase with five disulfide bonds and identification of a putative coelenterazine binding cavity by heteronuclear NMR. Sci. Rep. 10, (2020).
10. Jiang, T. Y., Du, L. P. & Li, M. Y. Lighting up bioluminescence with coelenterazine: strategies and applications. Photochem. Photobiol. Sci. 15, 466-480 (2016).
11. Shakhmin, A. et al. Coelenterazine analogues emit red-shifted bioluminescence with NanoLuc. Org. Biomol. Chem. 15, 8559-8567 (2017).
12. Michelini, E. et al. Spectral-resolved gene technology for multiplexed bioluminescence and high-content screening. Anal. Chem. 80, 260-267 (2008).
13. Rathbun, C. M. et al. Parallel screening for rapid identification of orthogonal bioluminescent tools. ACS Cent. Si. 3, 1254-1261 (2017).
14. Yeh, H.-W., Wu, T., Chen, M. & Ai, H.-W. Identification of Factors Complicating Bioluminescence Imaging. Biochemistry 58, 1689-1697 (2019).
15. Su, Y. C. et al. Novel NanoLuc substrates enable bright two-population bioluminescence imaging in animals. Nat. Methods 17, 852-860 (2020).
16. Lombardi. A., Pirro, F., Maglio, O., Chino, M. & DeGrado, W. F. De Novo design of four-helix bundle metalloproteins: One scaffold, diverse reactivities. Acc. Chem. Res. 52, 1148-1159 (2019).
17. Chino, M. et al. Artificial diiron enzymes with a DE Novo designed four-helix bundle structure. Eur. J. Inorg. Chem. 2015, 3352-3352 (2015).
18. Basler, S. et al. Efficient Lewis acid catalysis of an abiological reaction in a de novo protein scaffold. Nat. Chem. 13, 231-235 (2021).
19. Anishchenko, I. et al. De novo protein design by deep network hallucination. Nature (2021) doi:10.1038/s41586-021-04184-w.
20. Wang, J. et al. Scaffolding protein functional sites using deep learning. Science 377, 387-394 (2022).
21. Nom, C. et al. Protein sequence design by conformational landscape optimization. Proc. Natl. Acad Sci. U.S.A 118, (2021).
22. Yang, J. Y. et al. Improved protein structure prediction using predicted interresidue orientations. Proc. Natl. Acad. Sci. U.S.A 117, 1496-1503 (2020).
23. Basanta, B. et al. An enumerative algorithm for de novo design of proteins with diverse pocket structures. Proc. Natl. Acad. Si. U.S.A 117, 22135-22145 (2020).
24. Loening, A. M., Fenn, T. D. & Gambhir, S. S. Crystal structures of the luciferase and green fluorescent protein from Renilla reniformis. J. Mol. Biol. 374, 1017-1028 (2007).
25. Tomabechi, Y. et al. Crystal structure of nanoKAZ: The mutated 19 kDa component of Oplophorus luciferase catalyzing the bioluminescent reaction with coelenterazine. Biochem. Biophyr. Res. Commun. 470, 88-93 (2016).
26. Ding, B. W. & Liu, Y. J. Bioluminescence of Firefly Squid via Mechanism of Single Electron-Transfer Oxygenation and Charge-Transfer-Induced Luminescence. J Am. Chem. Soc. 139, 1106-1119 (2017).
27. Isobe, H., Yamanaka, S., Kuramitsu, S. & Yamaguchi, K. Regulation mechanism of spin-orbit coupling in charge-transfer-induced luminescence of imidazopyrazinone derivatives. J. Am. Chem. Soc. 130, 132-149 (2008).
28. Kondo, H. et al. Substituent effects on the kinetics for the chemiluminescence reaction of 6-arylimidazo[1,2-a]pyrazin-3(7H)-ones (Cypridina luciferin analogues): support for the single electron transfer (SET)-oxygenation mechanism with triplet molecular oxygen. Tetrahedron Lett. 46, 7701-7704 (2005).
29. Branchini, B. R. et al. Experimental Support for a Single Electron-Transfer Oxidation Mechanism in Firefly Bioluminescence. J. Am. Chem. Soc. 137, 7592-7595 (2015).
30. Zubatvuk, R., Smith, J. S., Leszczynski, J. & Isayev, O. Accurate and transferable multitask prediction of chemical properties with an atoms-in-molecules neural network. Science Advances 5, (2019).
31. Dou, J. Y. et al. De novo design of a fluorescence-activating beta-barrel. Nature 561, 485-491 (2018).
32. Cao, L. et al. Design of protein-binding proteins from the target structure alone. Nature 605, 551-560 (2022).
33. Jumper, J. et al. Highly accurate protein structure prediction with AlphaFold. Nature 596, 583-+(2021).
34. Dauparas, J. et al. Robust deep learning based protein sequence design using ProteinMPNN. bioRxiv (2022) doi:10.1101/2022.06.03.494563.
35. Yeh, H.-W. et al. ATP-Independent Bioluminescent Reporter Variants To Improve in Vivo Imaging. ACS Chem. Biol. 14, 959-965 (2019).
36. Bhaumik, S. & Gambhir, S. S. Optical imaging of Renilla luciferase reporter gene expression in living mice. Proc. Natl. Acad. Sci. U.S.A 99, 377-382 (2002).
37. Szent-Gyorgyi, C., Ballou, B. T., Dagnal, E. & Bryan, B. Cloning and characterization of new bioluminescent proteins. in Biomedical Imaging: Reporters, Dyes, and Instrumentation (eds. Bomhop, D. J., Contag, C. H. & Sevick-Muraca. E. M.) (SPIE, 1999). doi:10.1117/12.351015.
38. Hall, M. P. et al. Engineered luciferase reporter from a deep sea shrimp utilizing a novel imidazopyrazinone substrate. ACS Chem. Biol. 7, 1848-1857 (2012).
39. Back, M. et al. Accurate prediction of protein structures and interactions using a three-track neural network. Science 373, 871-+(2021).
40. Giger, L. et al. Evolution of a designed retro-aldolase leads to complete active site remodeling. Nat. Chem. Biol. 9, 494-498 (2013).
41. Yao, Z. et al. Multiplexed bioluminescence microscopy via phasor analysis. Nat. Methods 19, 893-898 (2022).
42. Loening, A. M., Dragulescu-Andrasi, A. & Gambhir, S. S. A red-shifted Renilla luciferase for transient reporter-gene expression. Nat. Methods 7, 5-6 (2010).
43. Dijkema, F. M. et al. Flash properties of Gaussia luciferase are the result of covalent inhibition after a limited number of cycles. Protein Sci. 30, 638-649 (2021).
44. Schenkmayerova, A. et 71. Engineering the protein dynamics of an ancestral luciferase. Nat. Commun. 12, (2021).
45. Ando, Y. et al. Development of a quantitative bio/chemiluminescence spectrometer determining quantum yields: Re-examination of the aqueous luminol chemiluminescence standard. Photochem. Photobiol. 83, 1205-1210 (2007).
46. Li, W. & Godzik, A. Cd-hit: a fast program for clustering and comparing large sets of protein or nucleotide sequences. Bioinformatics 22, 1658-1659 (2006).
47. Farrell, D. P. et 71. Deep learning enables the atomic structure determination of the Fanconi Anemia core complex from cryoEM. IUCrJ7, 881-892 (2020).
48. Zhang, Y. & Skolnick, J. TM-align: a protein structure alignment algorithm based on the TM-score. Nucleic Acids Res. 33, 2302-2309 (2005).
49. Needleman, S. B. & Wunsch, C. D. A general method applicable to the search for similarities in the amino acid sequence of two proteins J. Mol. Biol 48, 443-453 (1970).
50. Oda, T., Lim, K. & Tomii, K. Simple adjustment of the sequence weight algorithm remarkably enhances PSI-BLAST performance. BMC Bioinformatics 18, 288 (2017).
51. Altschul, S. F. et al. Gapped BLAST and PSI-BLAST: a new generation of protein database search programs. Nucleic Acids Res. 25, 3389-3402 (1997).
52. Coventry, B. & Baker, D. Protein sequence optimization with a pairwise decomposable penalty for buried unsatisfied hydrogen bonds. PLoS Comput. Biol. 17, (2021).
53. Smith, A. J. T. et al. Structural Reorganization and Preorganization in Enzyme Active Sites: Comparisons of Experimental and Theoretically Ideal Active Site Geometries in the Multistep Serine Esterase Reaction Cycle. J. Am. Chem. Soc. 130, 15361-15373 (2008).
54. Salomon-Ferrer, R., Case, D. A. & Walker, R. C. An overview of the Amber biomolecular simulation package. Wiley Interdiscip. Rev. Comput. Mol. Sci. 3, 198-210 (2013).
55. Kellogg, E. H., Leaver-Fay, A. & Baker, D. Role of conformational sampling in computing mutation-induced changes in protein structure and stability. Proteins 79, 830-838 (2011).
56. Park, H. et al. Simultaneous optimization of biomolecular energy functions on features from small molecules and macromolecules. J. Chem. Theory Comput. 12, 6201-6212 (2016).
57. Klein, J. C. et al. Multiplex pairwise assembly of array-derived DNA oligonucleotides. Nucleic Acids Res. 44, (2016).
58. Loening, A. M., Wu, A. M. & Gambhir, S. S. Red-shifted Renilla reniformis luciferase variants for imaging in living subjects. Nat. Methods 4, 641-643 (2007).
59. Liang, J., Feng, X., Hait, D. & Head-Gordon, M. Revisiting the performance of time-dependent density functional theory for electronic excitations: Assessment of 43 popular and recently developed functionals from rungs one to four. J. Chem. Theory Comput. 18, 3460-3473 (2022).
60. Chai, J.-D. & Head-Gordon, M. Long-range corrected hybrid density functionals with damped atom-atom dispersion corrections. Phys. Chem. Chem. Phys. 10, 6615-6620 (2008).
61. Ditchfield, R., Hehre, W. J. & Pople, J. A. Self-consistent molecular-orbital methods. IX. An extended Gaussian-type basis for molecular-orbital studies of organic molecules. J. Chem. Phys. 54, 724-728 (1971).
62. Grimme. S. Exploration of chemical compound, conformer, and reaction space with meta-dynamics simulations based on tight-binding quantum chemical calculations. J. Chem. Theory Comput. 15, 2847-2862 (2019).
63. Pracht, P., Bohle, F. & Grimme, S. Automated exploration of the low-energy chemical space with fast quantum chemical methods. Phys. Chem. Chem. Phys. 22, 7169-7192 (2020).
64. Luchini, G., Alegre-Requena, J. V., Funes-Ardoiz, I. & Paton, R. S. GoodVibes: automated thermochemistry for heterogeneous computational chemistry data. F1000Res. 9, 291 (2020).
65. Li, Y.-P., Gomes, J., Mallikarjun Sharada, S., Bell, A. T. & Head-Gordon, M. Improved force-field parameters for QM/MM simulations of the energies of adsorption for molecules in zeolites and a free rotor correction to the rigid rotor harmonic oscillator model for adsorption enthalpies. J. Phys. Chem. C Nanomater. Interfaces 119, 1840-1850 (2015).
66. Götz, A. W. et al. Routine microsecond molecular dynamics simulations with AMBER on GPUs. 1. Generalized Bom. J Chem. Theory Comput 8, 1542-1555 (2012).
67. Becke, A. D. Density-functional thermochemistry. 111. The role of exact exchange. J. Chem. Phys. 98, 5648-5652 (1993).
68. Grimme. S., Antony, J., Ehrlich, S. & Krieg, H. A consistent and accurate ab initio parametrization of density functional dispersion correction (DFT-D) for the 94 elements H-Pu. J. Chem. Phys. 132, 154104 (2010).
69. Grimme, S., Ehrlich, S. & Goerigk, L. Effect of the damping function in dispersion corrected density functional theory. J Comput. Chem. 32, 1456-1465 (2011).
70. Meiler, J. & Baker, D. ROSETTALIGAND: Protein-small molecule docking with full side-chain flexibility. Proteins 65, 538-548 (2006).
71. Davis, I. W. & Baker, D. RosettaLigand docking with full ligand and receptor flexibility. J. Mol. Biol. 385, 381-392 (2009).
72. Davis, I. W., Raha, K., Head, M. S. & Baker, D. Blind docking of pharmaceutically relevant compounds using RosettaLigand. Protein Sci. 18, 1998-2002 (2009).
73. Wang, J., Wolf, R. M., Caldwell, J. W., Kollman, P. A. & Case, D. A. Development and testing of a general amber force field. J. Comput. Chem. 25, 1157-1174 (2004).
74. Bayly, C. I., Cieplak, P., Cornell, W. & Kollman, P. A. A well-behaved electrostatic potential based method using charge restraints for deriving atomic charges: the RESP model. J Phys. Chem. 97, 10269-10280 (1993).
75. Besler, B. H., Merz, K. M. & Kollman, P. A. Atomic charges derived from semiempirical methods. J. Comput. Chem. 11, 431-439 (1990).
76. Singh, U. C. & Kollman, P. A. An approach to computing electrostatic charges for molecules. J Comput. Chem. 5, 129-145 (1984).
77. Jorgensen, W. L., Chandrasekhar, J., Madura, J. D., Impey, R. W. & Klein, M. L. Comparison of simple potential functions for simulating liquid water. J. Chem. Phys. 79, 926-935 (1983).
78. Maier, J. A. et al. Ff14SB: Improving the accuracy of protein side chain and backbone parameters from ff99SB. J Chem. Theory Comput 11, 3696-3713 (2015).
79. Darden, T., York, D. & Pedersen, L. Particle mesh Ewald: AnN·log(N) method for Ewald sums in large systems. J Chem. Phys. 98, 10089-10092 (1993).
80. Roe, D. R. & Cheatham, T. E., III. PTRAJ and CPPTRAJ: Software for processing and analysis of molecular dynamics trajectory data. J. Chem. Theory Comput. 9, 3084-3095 (2013).

We claim:

1. A protein having luciferase activity, comprising the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain; wherein:
(a) the H1 domain is at least 18 or 19 amino acids in length; residue 14 of the H1 domain is Y, D, or E, and residue 18 of the H1 domain is D or E;
(b) the E3 domain is at least 6, 7, 8, 9, or 10 amino acids in length and residue 2 of the E3 domain is R; and
(c) the E5 domain is at least 10, 11, 12, 13, or 14 amino acids in length and residue 9 of the E5 domain is H or N.

2. The protein of claim 1, wherein residue 7 of the E5 domain is M.

3. The protein of claim 1 or 2 wherein the E6 domain is at least 9, 10, 11, 12, or 13 amino acids in length and wherein residue 5 of the E6 domain is V.

4. The protein of any one of claims 1-3, wherein residue 1 of the L5 domain is S.

5. The protein of any one of claims 3-4, wherein residue 7 of the E5 domain is M and residue 5 of the E6 domain is V.

6. The protein of any one of claims 3-4, wherein residue 7 of the E5 domain is M, residue 5 of the E6 domain is V, and residue 1 of the L5 domain is S.

7. The protein of any one of claims 1-6, wherein the H2 domain is at least 5, 6, or 7 amino acids in length, the H3 domain is at least 9, 10, 11, 12, 13, or 14 amino acids in length, the E1 domain is at least 3 or 4 amino acids in length, the E2 domain is at least 3 or 4 amino acids in length, and/or the E4 domain is at least 8, 9, 10, 11, or 12 amino acids in length.

8. The protein of any one of claims 1-7, wherein:
the H1 domain is 19 amino acids in length;
the H2 domain is 7 amino acids in length;
the E1 domain is 4 amino acids in length;
the E2 domain is 4 amino acids in length;
the H3 domain is 14 amino acids in length;
the E3 domain is 10 amino acids in length;
the E4 domain is 12 amino acids in length;
the E5 domain is 14 amino acids in length; and
the E6 domain is 12 or 13 amino acids in length.

9. The protein of any one of claims 1-8, wherein 1, 2, 3, 4, or all 5 of the following is true:
(a) residue 13 of domain H1 is F;
(b) residue 1 of domain L3 is W;
(c) residue 5 of domain E5 is V or another hydrophobic residue;
(d) residue 8 of domain E5 is A or L or another hydrophobic residue; and/or
(c) residue 11 of domain E5 is W.

10. The protein of any one of claim 8 or 9, wherein 1, 2, 3, 4, 5, or all 6 of the following are true:
(a) residue 2 of domain E1 is I or another hydrophobic residue;
(b) residue 4 of domain H3 is F;
(c) residue 6 of domain E4 is V or another hydrophobic residue;
(d) residue 8 of domain E4 is L or another hydrophobic residue;
(e) residue 5 of domain E6 is M or V or another hydrophobic residue; and/or
(f) residue 7 of domain E6 is V or another hydrophobic residue.

11. The protein of any one of claims 1-10, comprising an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-181, or SEQ ID NO:1-3.

12. The protein of claim 11, comprising the amino acid sequence of SEQ ID NO:4.

13. A protein having luciferase activity, comprising an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1, wherein:
Residue 14 is Y, D, or E and residue 98 is H or N; and
Residue 18 is D or E and residue 65 is R.

14. The protein of claim 13, comprising one or both of A96M and M110V substitutions relative to SEQ ID NO:1.

15. The protein of claim 13, comprising both of A96M and M110V substitutions relative to SEQ ID NO:1.

16. The protein of any one of claims 13-15, comprising an R60S substitution relative to SEQ ID NO:1.

17. The protein of claim 16, comprising R60S, A96M, and M110V substitutions relative to SEQ ID NO:1.

18. The protein of any one of claims 10-14 and 16, wherein any substitutions relative to SEQ ID NO:1 at residues F12, 135, W38, F49, V81, L83, V94, A97, W100, M110, and/or V112 are conservative amino acid substitutions.

19. The protein of any one of claims 13-18, comprising an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-3, or 1-181.

20. The protein of any one of claims 11 and 13-19, wherein any substitutions relative to the reference sequence are conservative amino acid substitutions.

21. A protein comprising the formula X1-Z1-X2-Z2-X3-Z3-X4-Z4-X5-Z5-X6-Z6-X7-Z7-X8-Z8, wherein:
X1 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of MSEEQIRQFL RRFYEALD (SEQ ID NO: 182), wherein residue 14 is Y, D, or E and residue 18 is D or E;
X2 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of ADTAASLF (SEQ ID NO: 183);
X3 has an amino acid sequence at least 50%, 75%, or 100% identical to the amino acid sequence of TIHL (SEQ ID NO: 184);
X4 has an amino acid sequence at least 33%, 66%, or 100% identical to the amino acid sequence of VTF;
X5 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of EEFREWFERLFST (SEQ ID NO: 185);
X6 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of QREIKSLEVR (SEQ ID NO: 186), wherein residue 2 is R;
X7 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of VEVHVQLHATH (SEQ ID NO: 187);
X8 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of KHTVDATHHWHFR (SEQ ID NO: 188), wherein residue 8 is H or N;
X9 has an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of VTEMRVHINPTG (SEQ ID NO: 189); and
wherein Z1, Z2, Z3, Z4, Z5, Z6, Z7, and Z8 are independently present or absent, and when present may comprise any amino acid sequence.

22. The protein of claim 21, wherein 1, 2, 3, 4, 5, 6, 7, or all 8 of the following are true
Z1 comprises SGD;
Z2 comprises HPGV (SEQ ID NO: 190);
Z3 comprises WDG;
Z4 comprises TSR;
Z5 comprises RKDA (SEQ ID NO: 191);
Z6 comprises GDT;
Z7 comprises NGQ; and/or
Z8 comprises GNR; and
wherein 0, 1, 2, 3, 4, 5, 6, 7, or all 8 of Z1, Z2, Z3, Z4, Z5, Z6, Z7, and Z8 further comprise an additional polypeptide domain.

23. A self-complementing multipartite protein having luciferase activity, comprising at least a first polypeptide component and a second polypeptide component, wherein the at least first polypeptide component and the second polypeptide component are not covalently linked, wherein in total the at least first polypeptide component and the second polypeptide component comprise domains X1-Z1-X2-Z2-X3-Z3-X4-Z4-X5-Z5-X6-Z6-X7-Z7-X8-Z8-X9, wherein each domain is as defined in claims 21-22;
wherein (a) each X domain is fully present within one polypeptide component of the at least first polypeptide component and the second polypeptide component, and (b) none of the at least first polypeptide component and the second polypeptide component include each of X1, X2, X3, X4, X5, X6, X7, X8, and X9.

24. The self-complementing multipartite protein of claim 23, wherein the split occurs at Z4, Z5, Z6, or Z7.

25. A self-complementing multipartite protein having luciferase activity, comprising at least a first polypeptide component and a second polypeptide component, wherein the at least first polypeptide component and the second polypeptide component are not covalently linked, wherein in total the at least first polypeptide component and the second polypeptide component comprise the secondary structure arrangement H1-L1-H2-L2-E1-L3-E2-L4-H3-L5-E3-L6-E4-L7-E5-L8-E6, wherein each domain is as defined in any one of claims 1-9;
wherein (a) each H and E domain is fully present within one polypeptide component of the at least first polypeptide component and the second polypeptide component, and (b) none of the at least first polypeptide component and the second polypeptide component include all of the H and E domains.

26. The self-complementing multipartite protein of claim 25, wherein the split occurs at L4, L5, L6, L7, or L8.

27. A fusion protein comprising:
(a) the protein or polypeptide component of any preceding claims; and
(b) one or more additional functional domains.

28. A nucleic acid encoding the protein, polypeptide component, or fusion protein of any preceding claim.

29. The nucleic acid of claim 28, comprising the nucleotide sequence of any one of SEQ ID NO:200-380.

30. An expression vector comprising the nucleic acid of claim 28 or 29 operatively linked to a suitable control element.

31. A recombinant host cell comprising the protein, polypeptide component, fusion protein, nucleic acid, and/or expression vector of any preceding claim.

32. A kit comprising:
(a) the protein, polypeptide component, fusion protein, nucleic acid, expression vector, and/or host cell of any preceding claim; and
(b) instructions for their use.

33. The kit of claim 32, further comprising diphenylterazine (DTZ).

34. A method for using the protein, polypeptide component, fusion protein, nucleic acid, expression vector, host cell, and/or kit of any preceding claim for any suitable purpose, including but not limited to luminescent reporting assays, diagnostic assays, cellular localization of targets of interest, cellular imaging, gene editing, live animal imaging, cancer labeling, CART-cells reporting, secreted assay, gene delivery, and tissue engineering.

35. A method for making a luciferase, comprising de novo design using the methods of any embodiment disclosed herein, starting with the protein comprising the amino acid sequence of SEQ ID NO:381.


SEQUENCE LISTING (381 sequences)

SEQ ID NO: 1 - protein, 118 aa, synthetic construct
  VARIANT 1: note = optionally absent
  VARIANT 118: note = optionally absent
MSEEQIRQFLRRFYEALDSGDADTAASLFHPGVTIHLWDGVTFTSREEFREWFERLFSTRKDAQREIKSLEVRGDTVEVHVQLHATHNGQKHTVDATHHWHFRGNRVTEMRVHINPTG

SEQ ID NO: 2 - protein, 118 aa, synthetic construct
  VARIANT 1: note = optionally absent
  VARIANT 118: note = optionally absent
MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG

SEQ ID NO: 3 - protein, 118 aa, synthetic construct
  VARIANT 1: note = optionally absent
  VARIANT 118: note = optionally absent
MSEEQIRQFLRRFYEALDSGDADTAASLFHPGVTIHLWDGVTFTSREEFREWFERLFSTRKDAQREIKSLEVRGDTVEVHVQLHATHNGQKHTVDMTHHWHFRGNRVTEVRVHINPTG

SEQ ID NO: 4 - protein, 118 aa, synthetic construct
  VARIANT 1: note = optionally absent
  VARIANT 2: note = X is S or T
  VARIANT 3: note = X is A, E, K, or S
  VARIANT 4: note = X is A, D, E, K, Q, R, S, or T
  VARIANT 5: note = X is A, D, E, or Q
  VARIANT 6: note = X is I or Q
  VARIANT 7: note = X is E, K, or R
  VARIANT 8: note = X is A, D, E, K, N, Q, or R
  VARIANT 10: note = X is C, N, V, W, Y, or L
  VARIANT 11: note = X is A, D, E, K, Q, or R
  VARIANT 12: note = X is K, Q, R, or F
  VARIANT 15: note = X is A, D, E, K, L, N, Q, R, or S
  VARIANT 19: note = X is A, D, or S
  VARIANT 23: note = X is D, E, N, or V
  VARIANT 26..27: note = X is A or S
  VARIANT 30: note = X is K, P, R, or H
  VARIANT 31: note = X is D or P
  VARIANT 33: note = X is T or V
  VARIANT 34: note = X is E, I, K, L, N, Q, R, V, or T
  VARIANT 36: note = X is D, E, H, K, or Y
  VARIANT 41: note = X is I, K, R, T, or V
  VARIANT 42: note = X is E, I, T, or V
  VARIANT 44: note = X is E, F, H, K, N, R, S, T, or Y
  VARIANT 45: note = X is K, T, or S
  VARIANT 46: note = X is K, Q, or R
  VARIANT 47: note = X is A or E
  VARIANT 48: note = X is Q or E
  VARIANT 50: note = X is K, Q, or R
  VARIANT 51: note = X is A, D, E, K, Q, or S
  VARIANT 54: note = X is E, K, R, or V
  VARIANT 55: note = X is A, D, E, K, Q, R, or T
  VARIANT 57: note = X is F, H, K, R, or Y
  VARIANT 58: note = X is A or S
  VARIANT 59: note = X is E, K, L, Q, R, or T
  VARIANT 60: note = X is S or R
  VARIANT 61: note = X is A, D, E, K, N, Q, S, or T
  VARIANT 62: note = X is A, D, E, G, N, or Q
  VARIANT 64: note = X is A, K, R, S, or Q
  VARIANT 66: note = X is D, E, H, K, R, or S
  VARIANT 67: note = X is I or V
  VARIANT 68: note = X is E, I, T, V, or K
  VARIANT 69: note = X is A, D, E, K, N, Q, R, or S
  VARIANT 70: note = X is F, L, or M
  VARIANT 71: note = X is D, E, K, Q, R, S, T, or V
  VARIANT 73: note = X is A, D, E, H, I, N, or R
  VARIANT 75: note = X is D or N
  VARIANT 76: note = X is D, E, F, I, K, R, T, or V
  VARIANT 77: note = X is A, S, or V
  VARIANT 78: note = X is D, E, F, H, K, L, N, R, T, or Y
  VARIANT 79: note = X is I or V
  VARIANT 80: note = X is E, I, K, N, R, S, T, V, or H
  VARIANT 82: note = X is I, V, or Q
  VARIANT 84: note = X is D, E, H, K, R, or V
  VARIANT 86: note = X is D, E, F, I, K, N, R, S, T, V, or Y
  VARIANT 87: note = X is E, F, H, I, K, V, or Y
  VARIANT 88: note = X is A, D, E, K, N, Q, or R
  VARIANT 90: note = X is E, K, Q, R, or T
  VARIANT 91: note = X is A, D, E, H, K, P, or Q
  VARIANT 92: note = X is E, H, K, L, R, or V
  VARIANT 93: note = X is E, I, K, R, T, or V
  VARIANT 95: note = X is A, E, F, G, H, K, L, N, R, S, or D
  VARIANT 96: note = X is L, A, or M
  VARIANT 97: note = X is E, H, K, N, R, T, V, A, or L
  VARIANT 99: note = X is E, F, I, K, L, N, Q, R, T, V, W, Y, or H
  VARIANT 100: note = X is A, F, T, Y, or W
  VARIANT 102: note = X is F or W
  VARIANT 103: note = X is D, E, K, or R
  VARIANT 105: note = X is D, S, or N
  VARIANT 106: note = X is E, H, K, Q, or R
  VARIANT 107: note = X is L or V
  VARIANT 108: note = X is K, R, T, or V
  VARIANT 109: note = X is E, K, or R
  VARIANT 110: note = X is V or M
  VARIANT 111: note = X is D, E, F, H, K, N, R, S, T, W, or Y
  VARIANT 113: note = X is A, D, E, H, K, R, S, T, or V
  VARIANT 115: note = X is D, E, F, H, I, K, N, Q, R, S, T, V, or Y
  VARIANT 117: note = X is D, L, M, V, or T
  VARIANT 118: note = optionally absent
MXXXXXXXFXXXFYXALDXGDAXTAXXLFXXGXXIXLWDGXXFXXXXXFXXWFXXLXXXXXXAXRXXXXXXVXGXXXXXXVXLXAXXXGXXXXVXXXHXXXXXGXXXXXXXVXIXPXG

SEQ ID NO: 5 - protein, 117 aa, synthetic construct
  VARIANT 1: note = optionally absent
MSEEQIREFCKRFYEALDAGDAVTASALFPNGTRIHLWDGITFTTQEEFREWFERLYSQSEDAKREIVSLEVEGNVAYVEVILHASFRGEEKTVRLRHVFQFEGDKLVEVTVEIEPL

SEQ ID NO: 6 - protein, 117 aa, synthetic construct
  VARIANT 1: note = optionally absent
MSEEAQREFWKRFYEALDAGDAETAAALFPDGTEIHLWDGTTFRTQAEFRAWFEELYSTSQNAKREIVEFRVDGNKSTVTVVLHAIYEGEEKKVLLEHFTEWEGDRLVRVEVSIVPL

SEQ ID NO: 7 - protein, 117 aa, synthetic construct
  VARIANT 1: note = optionally absent
MSEEEQREFVRRFYEALDAGDADTASALFPDGTVIELWDGTTFRTRAEFKAWFEALHSKSENAVRHVVEFEVEGNKAKVRVVLHADYKGKKRVVELEHEFEFEGDRLVKVSVDIHPI

SEQ ID NO: 8 - protein, 117 aa, synthetic construct
  VARIANT 1: note = optionally absent
MSEEEIRKFCERFYAALDAGDAETASSLFPDGTEIHLWDGKTFRTQAEFREWFVRLRSTSDDARRHITKLKVDGNVAEVEVVLRASYDGEEKVVALKHVFKFEGDKLVEVKVEITPL

SEQ ID NO: 9 - protein, 117 aa, synthetic construct
  VARIANT 1: note = optionally absent
MSEEAQREFVRRFYEALDAGDAETASALFPDGTQIYLWDGKTFTTREEFRSWFVKLHSTSDNAKRRVVEFRVEGNKAKVKVVLKASINGKERTVLLEHFFEFEGDKLVKVEVRIRPL

SEQ ID NO: 10 - protein, 117 aa, synthetic construct
  VARIANT 1: note = optionally absent
MSEEEQREFWRRFYEALDAGDAETASALFPDGTEIYLWDGRVFRTQAEFRAWFVELRSKSDDARREVTSFRVDGNKADVRVVLHANYKGEKKVVELRHVYEFEGDRLVRVEVTINPL

SEQ ID NO: 11 - protein, 117 aa, synthetic construct
  VARIANT 1: note = optionally absent
MSEEEIREFVKRFYEALDAGDAETASALFPDGTKIHLWDGTVFTTREEFKSWFVELRSTSDDAKREVVDLKVEGNKAYVEVVLRAIVNGEDKVVKLRHVFKFEGDKLVEVHVEIDPL

SEQ ID NO: 12 - protein, 117 aa, synthetic construct
  VARIANT 1: note = optionally absent
MSKEEQRKFVERFYKALDAGDAETASALFPDGTKIHLWDGTTFHTRAEFRAWFVDLRSKSENAKREVVAFEVDGNKAKVVVVLKANYKGEERTVKLEHEFYFEGDHLVEVNVKIEPV

SEQ ID NO: 13 - protein, 117 aa, synthetic construct
  VARIANT 1: note = optionally absent
MSEEEQREFWKRFYEALDAGDAETASALFPDGTEIHLWDGKTFHTREEFRAWFVDLYSKSKDASREITKFEVEGNRAFVEVVLRASYDGEDRTVKLRHIFEFEGDHLVEVYVEIEPL

SEQ ID NO: 14 - protein, 117 aa, synthetic construct
  VARIANT 1: note = optionally absent
MSAEQIREFVARFYAALDAGDADTASALFPDGTEIHLWDGRTFRTQAEFRAWFETLRAQSADARRHVVALEVTGNTADVEVVLHASVDGEERTVALRHRFQFEGDRLVRVTVEITPL

SEQ ID NO: 15 - protein, 117 aa, synthetic construct
  VARIANT 1: note = optionally absent
MSEEEQKEFVKRFYEALDAGDAETASALFKDGTEIYLWDGTTFRTRAEFRAWFRRLKSTSEEARRSVVSFKVDGNVADVEVVLRARWKGEERVVKLRHRFVFEGDEVVRVEVEIRPL

SEQ ID NO: 16 - protein, 117 aa, synthetic construct
  VARIANT 1: note = optionally absent
MSAEEQREFVKRFYEALDAGDAETASALFPDGTKIHLWDGTVFNTQEEFKAWFVKLRSTSENAKREVTHFEVDGDKAVVEVVLHANIKGKEKTVRLRHEFLFEGDKIVEVNVEIEPL

SEQ ID NO: 17 - protein, 117 aa, synthetic construct
  VARIANT 1: note = optionally absent
MSAEAQREFVKKFYDALDAGDAETASALFPDGTEIYLWDGKVFKTQAEFRAWFVELYSKSDNAQRSVVRFEVDGNVAYVEVVLRANFDGEEKTVRLRHIFYFEGDSLVKVEVTIEPL

SEQ ID NO: 18 - protein, 117 aa, synthetic construct
  VARIANT 1: note = optionally absent
MSEEEIKEFWRRFYQALDAGDAETASALFPDGTEIYLWDGTTFRTRAEFRAWFEALRATSDAASRHIVKLEVKGNRAEVEVVLRARVDGEERVVRLRHIFEFEGDRLKRVEVEIEPL

SEQ ID NO: 19 - protein, 117 aa, synthetic construct
  VARIANT 1: note = optionally absent
MSEEEIREFWRRFYEALDAGDAETASALFPDGTKIYLWDGIVFSTKEEFRSWFVELKSKSDNAKRHVVSLRVDGNVANVKVVLEAEINGEKKVVELTHTTKFEGDKLVEVNVDINPL

SEQ ID NO: 20 - protein, 117 aa, synthetic construct
  VARIANT 1: note = optionally absent
MSEEEQRKFWEKFYEALDAGDAETASALFKDGTKIYLWDGTVFETQEEFRAWFEKLYSTSDNAQRHIVSFKVDGNISYVEVVLHANVNGEEKTVKLTHLFKFEGDHVVEVKVKIEPL

SEQ ID NO: 21 - protein, 117 aa, synthetic construct
  VARIANT 1: note = optionally absent
MSEEEQKEFWKKFYDALDAGDAETASALFPDGTKIHLWDGKTFTTQAEFKAWFVDLKSKSDDAKRYITKFKVDGNKAYIEVVLKASVKGKKKTVKLKHTAQFEGDKLVEVDVTINPL

SEQ ID NO: 22 - protein, 117 aa, synthetic construct
  VARIANT 1: note = optionally absent
MSEEAIREFVRRFYEALDAGDAETASALFPDGTRIHLWDGTTFRTRAEFRAWFVELHSKSDNAQREVVRLEVEGNRARVEVVLKANYKGKEKTVRLRHEFLFEGDALVEVRVEIEPL

SEQ ID NO: 23 - protein, 117 aa, synthetic construct
  VARIANT 1: note = optionally absent
MSAEAIREFWRRFYEALDAGDAETASALFPDGTEIHLWDGTTFRTREEFRAWFVELYSTSDNAKRKIVSLRVDGDRALVEVVLRASVGGEEKTVKLKHEALFEGDRLVEVRVQIEPL

SEQ ID NO: 24 - protein, 117 aa, synthetic construct
  VARIANT 1: note = optionally absent
MSEEAQRKFVERFYKALDAGDADTASALFPDGTKIYLWDGKVFTTQEEFRAWFVELYSKSENAKREVVSFNVDGDVAEVEVVLHANVRGELKTVKLKHTFKFEGDKLVEVNVTIKPL

SEQ ID NO: 25 - protein, 117 aa, synthetic construct
  VARIANT 1: note = optionally absent
MSAEAQREFWRRFYAALDAGDAETASALFPDGTKIHLWDGKTFTTRAEFREWFEKLYSTSDNAKREITELKVDGDKSYVKVVLKANINGEEKTVNLTHEFQFEGDKLVEVNVTINPL

SEQ ID NO: 26 - protein, 117 aa, synthetic construct
  VARIANT 1: note = optionally absent
MSEQAIREFWKRFYNALDAGDADTASALFPDGTVIHLWDGKTFHTQAEFRKWFVDLYSRSDNAKREIVSLEVDGNHAKIVVVLNAIINGEKKTVLLTHETLFEGDHLVEVFVEIKPL

SEQ ID NO: 27 - protein, 117 aa, synthetic construct
  VARIANT 1: note = optionally absent
MSEEEQREFWKRFYEALDAGDAETASALFPDGTKIHLWDGTTFTTQAEFRAWFVRLHSTSENASRSIVSFKVEGNKALVEVVLRASVKGEEKTVKLTHEFQFEGDKLVEVNVDIKPL

SEQ ID NO: 28 - protein, 117 aa, synthetic construct
  VARIANT 1: note = optionally absent
MSEEDIRRFVERFYAALDAGDAETASALFPDGTRIHLWDGTTFTTREEFRAWFEKLHATSENARRHVVKLKVEGNKAYVEVILHASFKGKEKTVKLTHVFLFEDDRLVEVYVKIEPL

SEQ ID NO: 29 - protein, 117 aa, synthetic construct
  VARIANT 1: note = optionally absent
MSEEEQREFWEKFYNALDAGDAETASSLFPDGTEIYLWDGTVFKTREEFRAWFEKLHSTSENAKRHIVSFEVDGNKSKVKVVLHAKINGEEVTVELEHVYEFEGDKLKKVEVEIKPL

SEQ ID NO: 30 - protein, 117 aa, synthetic construct
  VARIANT 1: note = optionally absent
MSEIAQREFWRRFYAALDAGDAETASALFPDGTEIYLWDGRTFHTREEFEAWFRELYSKSENARREITSFTVTGDIAEIRVVLRATIDGEKRTVSLNHVTHFEGDQLVRVEVSIFPL

SEQ ID NO: 31 - protein, 117 aa, synthetic construct
  VARIANT 1: note = optionally absent
MSEEEQKEFWKRFYEALDSGDADTASALFPDGTKIYLWDGTVFETKAQFKAWFVELYSRSDNAQRSITKFEVDGNTSRVVVVLKATVRGKEKVVELEHVFEFEGDELKEVKVEIHPL

SEQ ID NO: 32 - protein, 117 aa, synthetic construct
  VARIANT 1: note = optionally absent
MSEEAIREFVKRFYDALDAGDADTASALFPDGTLIHLWDGKTFRTQAEFRAWFEELYSKSENAKRHVVKLQVDGDRAQVEVVLHANIDGQEVVVRLRHEFLFEGDRLVEVYVQIQPL

SEQ ID NO: 33 - protein, 117 aa, synthetic construct
  VARIANT 1: note = optionally absent
MTAEAQRAFWDRFYRALDAGDAETASSLFPDGTEIHLWDGKTFHTREEFRQWFENLRSKSENARRHIVDFRVDGDRADVRVVLRANYQGEERVVQLHHEFLFEGDQLVRVEVRIIPE

SEQ ID NO: 34 - protein, 117 aa, synthetic construct
  VARIANT 1: note = optionally absent
MSEKEQREFCARFYAALDAGDAETASALFPDGTRIHLWDGRTFTTQAEFGAWFRRLRSTSDNARRHIVDFRVDGDVAKVRVVLHASVGGRERTVQLEHVFIFEGDRLVEVHVSIQPE

SEQ ID NO: 35 - protein, 117 aa, synthetic construct
  VARIANT 1: note = optionally absent
MSEEEQRKFWEKFYNALDAGDAETASALFPDGTEIYLWDGKVFRTRAEFREWFERLYSKSDNAQRSITSFEVNGNVAEIKVILKANVNGEEKEVSLNHKTEFEGDKLVKVEVDISPL

SEQ ID NO: 36 - protein, 117 aa, synthetic construct
  VARIANT 1: note = optionally absent
MSEKEQREFWKKFYEALDAGDAETAAALFPDGTVIHLWDGKTFHTQEEFKEWFIKLRSTSENAKRKIISFKVDGNISYVEVELKAKIKGESKVVKLRHIFKFEGDKLVEVDVTIKPI

SEQ ID NO: 37 - protein, 117 aa, synthetic construct
  VARIANT 1: note = optionally absent
MSAEEIKEFCRRFYEALDAGDAETASALFPDGTIIHLWDGRTFHTQAEFRAWFRELRSTSEDAKREIERLEVDGNQAKVEVILHANRDGEKKVVRLTHEFLFEGDRLVEVTVAIRPL

SEQ ID NO: 38 - protein, 117 aa, synthetic construct
  VARIANT 1: note = optionally absent
MSKQEIKDFCEKFYNALDAGDADTASALFKDGTKIYLWDGKTFETQAEFKAWFTKLHSTSDNAKRYITSLEVDGDKAFVKVVLKANVNGEEKTVELTHIFEFEGDELVKVQVKIKPL

SEQ ID NO: 39 - protein, 117 aa, synthetic construct
  VARIANT 1: note = optionally absent
MSEEEIRQFIKRFYDALDAGDAETASALFPDGTEIDLWDGTTFHTQEEFRSWFVRLKSTSDNAKRHVVALEVQGNRAKVEVVLEASIDGKKITVQLTHEFYFEGDKLVKVNVTIKPL

SEQ ID NO: 40 - protein, 117 aa, synthetic construct
  VARIANT 1: note = optionally absent
MSEEEIREFVRRFYAALDAGDAETASALFPDGTVIHLWDGRTFHTQAEFRAWFEELYSRSENAKREVTQLEVDGDHAFVKVVLRANIHGEEKVVGLEHYFHFEGDQLKEVEVVIRPE

SEQ ID NO: 41 - protein, 117 aa, synthetic construct
  VARIANT 1: note = optionally absent
MSKEEIEEFWKKFYQALDAGDAETASALFPDGTVIHLWDGKVFHTKAEFREWFVKLKSESEDAKREIVSLRVDGNKAEVEVVLKAKYKGEEKEVRLKHESLFEGDRLVEVDVDIFPL

SEQ ID NO: 42 - protein, 117 aa, synthetic construct
  VARIANT 1: note = optionally absent
MSEEEIREFWKRFYEALDAGDAETASALFKDGTKIYLWDGITFFTRDEFRAWFEKLYSTSEDAKREIVKFNVDGNKAKVEVVLHAIVDGQKKTVKLTHTSYFEGDELKKVEVSIEPM

SEQ ID NO: 43 - protein, 117 aa, synthetic construct
  VARIANT 1: note = optionally absent
MSEEEIREFIKRFYEALDAGDAETASALFPDGTKIYLWDGTVFSTQAEFRDWFVRLRSTSQDARRKVVDLKVDGDVADVEVVLRANVEGEERVVRLRHVFHFEGDKVVEVEVEIEPE

SEQ ID NO: 44 - protein, 117 aa, synthetic construct
  VARIANT 1: note = optionally absent
MSEEEQREFWRRFYAALDAGDAETASALFPDGTKIYLWDGKTFTTQAEFRAWFRKLRSQSEGASRSVEYFEVDGNKSHVEVVLRASVNGEQHVVKLRHEFLFEGDKLVEVEVKIEPL

SEQ ID NO: 45 - protein, 117 aa, synthetic construct
  VARIANT 1: note = optionally absent
MSEKAQREFWKKFYEALDAGDADTASALFKDGTEIYLWDGKVFKKQEEFKEWFEKLHSTSENAKRKIVKFEVKGNVSYVEVVLEASLAGEKKTVKLKHMFQFEGDKLVKVTVDIYPL

SEQ ID NO: 46 - protein, 117 aa, synthetic construct
  VARIANT 1: note = optionally absent
MSEKAQREFVKRFYDALDAGDADTASALFPDGTIIYLWDGKTFRTQTEFRRWFVDLKSTSENAKRKVTDFRVDGNVANVTVVLEANINGKKETVKLNHIFHWEGDRLVEVEVTIEPL

SEQ ID NO: 47 - protein, 117 aa, synthetic construct
  VARIANT 1: note = optionally absent
MSAEAIREFVRDFYEALDAGDADTASALFPDGTEIHLWDGTVFRTRAEFKAWFTKLYSTSEDARRKVVRLEVEGDVARVEVVLHASHAGKESTVKLQHEFSFEGDRLVEVRVVIEPL

SEQ ID NO: 48 - protein, 117 aa, synthetic construct
  VARIANT 1: note = optionally absent
MSEEEIKKFCEKFYEALDAGDAETASALFPDGTEIYLWDGRVFKTREEFRAWFVELYSKSDNAQRKIVDLKVDGNKAKVKVVLHANHNGEQHKVALTHEFLFEGDKLVEVNVEIKPL

SEQ ID NO: 49 - protein, 117 aa, synthetic construct
  VARIANT 1: note = optionally absent
MSEEAIREFWKKFYEALDAGDAETASALFPDGTIIHLWDGTTFTTQDEFKKWFVELFSKSENASRKIVKLEVKGNVAYVEVVLKAKIDGKEKTVRLKHVTKFEGDKLVEVEVDIDPL

SEQ ID NO: 50 - protein, 117 aa, synthetic construct
  VARIANT 1: note = optionally absent
MSADDQKEFWKRFYEALDAGNADEASALFPDGTEIYLWDGTTFKTKAQFKAWFCQLRSTSENAKREIVKFEVDGNFSYVEVVLEATVNGKKFIVSLDHYFEFEGEQLVEVYVKIRPL

SEQ ID NO: 51 - protein, 117 aa, synthetic construct
  VARIANT 1: note = optionally absent
MSAKAQREFWKKFYEALDAGDADTAAALFPDGTRIHLWDGRTFTTQAEFRAWFQTLHSRSDNAKRSIVAFNVSGDVSDVVVVLRASYEGEERTVRLRHTFRFEGDHLVEVDVAIDPL

SEQ ID NO: 52 - protein, 117 aa, synthetic construct
  VARIANT 1: note = optionally absent
MSEAEQREFWRRFYEALDAGDAETASALFPDGTEIHLWDGKTFHTQAEFRAWFVELRSTSEAARRHVVAFNVDGDRARVEVVLHAVRDGEARTVQLTHETVFAGDQVVRVRVQIKPL

SEQ ID NO: 53 - protein, 117 aa, synthetic construct
  VARIANT 1: note = optionally absent
MSEEAVREFVARFYEALDAGDADTASALFPDGTVIHLWSGQTFHTRAEFRAWFERLYAESAAARRRVVEMRVDGDVAFVEVVLHASHQGQPQVVRLRHIFRFEGDRLREVEVSIDPL

SEQ ID NO: 54 - protein, 117 aa, synthetic construct
  VARIANT 1: note = optionally absent
MSAEQQREFWDRFYAALDAGDADTASALFNDGTEIYLWDGKVFHTQAEFKAWFVDLRSRSTGASREIVAFKVTGNRAEIEVVLHADVDGQPKTVRLTHYTEFEGDRLVRVEVKINPL

SEQ ID NO: 55 - protein, 117 aa, synthetic construct
  VARIANT 1: note = optionally absent
MSEQAIREFVRRFYDALDAGDAETAAALFPDGTVIHLWDGRTFHTRAEFRAWFVRLRSTSDDARRRVVELRVVGDRARVRVVLQANVDGEPRVVELEHEFEFEGDRLREVRVDIRPL

SEQ ID NO: 56 - protein, 117 aa, synthetic construct
  VARIANT 1: note = optionally absent
MSAEEQRKFVEKFYTALDSGDAETASSLFPDGTLIYLWDGRVFHTQAEFRAWFEKLYSQSANAKRSVVRFEVEGNEAYVEVVLHAVVDGKETVVRLKHNYLFEGDRLVRVEVEIKPM

SEQ ID NO: 57 - protein, 117 aa, synthetic construct
  VARIANT 1: note = optionally absent
MSEEAQREFAKRFYAALDAGDADTAAALFPDGTEIYLWDGKTFTTRAEFRAWFEELRSTSDRAKRRVDRFEVNGDTAHVEVTLDAYVNGENRTVRLRHVFHFEGDRVKRVEVEIKPL

SEQ ID NO: 58 - protein, 117 aa, synthetic construct
  VARIANT 1: note = optionally absent
MSEEEIREFVRRFYEALDAGDAATASALFPDGTEIHLWDGTTFRTQAQFRAWFERLRAQSANARREIVDLKVEGDRAKVEVILRASFDGEEKVVNLTHEFLFEGDRLVRVSVTITPL

SEQ ID NO: 59 - protein, 117 aa, synthetic construct
  VARIANT 1: note = optionally absent
MSEEEIKEFWKRFYEALDAGDAETASALFPDGTEIHLWSGKTFRTREEFRAWFEELYSQSENAKRHIVSLEVDGDEAYVRVVLHAFVKGESRTVELEHFFRFEGDHLVEVKVDIIPL

SEQ ID NO: 60 - protein, 117 aa, synthetic construct
  VARIANT 1: note = optionally absent
MSEAAIREFVDKFYAALDAGDAETAASLFPDGTKIYLWDGRTFTTQAEFKAWFEELHATSDNARREVVSMEVDGDVADVEVVLHASFRGEQREVRLRHVFHFEGDELREVEVEILPL

SEQ ID NO: 61 - protein, 117 aa, synthetic construct
  VARIANT 1: note = optionally absent
MSEEQQREFWARFYAALDAGDADTASALFPDGTKIHLWDGKTFTSRAEFRDWFVRLHSRSENAKRRITSFKVEGDISYVEVVLHASRDGQEHVVKLKHVAKFEGDELVEVKVEITPL

SEQ ID NO: 62 - protein, 117 aa, synthetic construct
  VARIANT 1: note = optionally absent
MSKEEQKNFCKKFYEALDAGDAETASSLFPDGTKIYLWDGKTFSTQEEFRAWFEKLRSESANASRRIVSFNVDGNVAKVKVVLKANYRGKKSTVKLEHKFEFEGDKLVKVEVTIEPL

SEQ ID NO: 63 - protein, 117 aa, synthetic construct
  VARIANT 1: note = optionally absent
MSEEAIREFVKRFYEALDAGDADTASALFPDGTRIYLWDGTTFRTQAEFRAWFRELYSKSEGARREVINLKVDGDVAYVEVVLHAVYKGEEKTVKLVHVFKFEGDKVVEVHVEIVPL

SEQ ID NO: 64 - protein, 117 aa, synthetic construct
  VARIANT 1: note = optionally absent
MSEEEIREFVKRFYTALDAGDAETASALFPDGTKIYLWDGTTFETREGFAAWFRKLKSQSEEAKRHVVKLKVEGNVAYIEVVLHAKHKGEEKTVRLTHIYQFEGDKLVEVRVEIKPL

SEQ ID NO: 65 - protein, 117 aa, synthetic construct
  VARIANT 1: note = optionally absent
MSEAEIKNFVKRFYEALDAGDAETASALFPDGTEIHLWDGTTFRTRAEFRAWFEELYSRSEDAKREVTKLEVKGNVAFVEVVLKANLNGKERTVKLKHIFHFEGDSLVRVEVSIVPL

SEQ ID NO: 66 - protein, 117 aa, synthetic construct
  VARIANT 1: note = optionally absent
MSEQEQKEFWEKFYQALDAGDAETASALFKDGTEIYLWDGKVFKTQEEFKAWFVELYSKSLNAKRKIVEHEVKGNESYVKVVLRASVDGEERTVLLEHKFLFEGDKLVKVSVEITPL

SEQ ID NO: 67 - protein, 117 aa, synthetic construct
  VARIANT 1: note = optionally absent
MTAEEQREFVKRFYEALDAGDAETASALFPDGTLIHLWDGKTFHTREEFRAWFVELRSTSDDAKREVTAFEVDGDVARVEVVLHANIQGNKKTVALTHEFLFEGDKLVEVRVDIKPL

SEQ ID NO: 68 - protein, 117 aa, synthetic construct
  VARIANT 1: note = optionally absent
MSEEEQKNFVRKFYDALDAGDSETASALFKDGTKIYLWDGQVFETQEEFRAWFEKLYSTSTDAKREIVSFKVDGNKAVVEVVLKAIKDGEEKVVNLKHVFLFEGDEVVEVEVDIVPS

SEQ ID NO: 69 - protein, 117 aa, synthetic construct
  VARIANT 1: note = optionally absent
MTAEQQRNFWARFYAALDAGDAETAAALFPDGTVIHLWDGRTFHTQAEFRAWFEALRSTSENARREIVFFQVDGNTADVEVVLHASVDGDARTVRLRHIFQFEGDHLVEVHVDIRPL

SEQ ID NO: 70 - protein, 117 aa, synthetic construct
  VARIANT 1: note = optionally absent
MSEEEQKNFVAAFYKALDAGDAETASALFPDGTEIHLWDGKTFKTQAEFKAWFEKLFSTSDNAKRKVVSFKVNGNIADVQVVLEANINGEPVKVKLNHTFKFKGDKLVEVNVQIEPL

SEQ ID NO: 71 - protein, 117 aa, synthetic construct
  VARIANT 1: note = optionally absent
MSEEEIREFVRKFYAALDAGDADTASALFPDGTEIHLWDGVTFKTQAEFKAWFTELKSKSDNAKREVVKLKVDGNKADVEVVLHATIDGKEVTVHLRHIFEFEGDKLVKVEVVIEPL

SEQ ID NO: 72 - protein, 117 aa, synthetic construct
  VARIANT 1: note = optionally absent
MSAEAQREFVKRFYDALDAGDADTASALFADGTRIYLWDGREFRTRAEFRAWFERLRSRSDAAKRHVTSFKVEGNVAQVVVVLKANFRGKEHVVELLHEFVFEGDRLVEVHVEIRPR

SEQ ID NO: 73 - protein, 117 aa, synthetic construct
  VARIANT 1: note = optionally absent
MSEEEIREFVKRFYEALDAGDAETASALFPDGTKIHLWDGITFNTQEEFQKWFVELRSQSDNAKREVVSLKVDGNHAKVEVVLKANIGGEDRVVKLTHHFLFEGDKLIEVRVEIEPL

SEQ ID NO: 74 - protein, 117 aa, synthetic construct
  VARIANT 1: note = optionally absent
MSAEAIREFVERFYAALDAGDAETASALFPDGTIIHLWDGRVFHTREEFRRWFVDLFSTSDNASRSVVDLHVDGNVAHVVVRLKASWRGKKRTVLLTHVFEFEGDHLVKVTVSIQPV

SEQ ID NO: 75 - protein, 117 aa, synthetic construct
  VARIANT 1: note = optionally absent
MSAEEIREFVARFYAALDAGDADTASALFPNGTKIYLWDGTVFTTQAEFRAWFVKLRSTSENAKREVVFLEVEGNEAFVEVVLHAEIKGQKKTVRLRHVFKFEGDHLVEVSVDIEPL

SEQ ID NO: 76 - protein, 117 aa, synthetic construct
  VARIANT 1: note = optionally absent
MSKEDIEEFCKKFYDALDAGDAETASALFPDGTEINLWDGKVFTTKSEFRAWFRELYARSDHAKREITHLEVDGNFATIEVVLNASVDGEQKTVSLKHFFQFEGDRLVRVDVKINPL

SEQ ID NO: 77 - protein, 117 aa, synthetic construct
  VARIANT 1: note = optionally absent
MSEEEIREFWQRFYEALDAGDAETASALFPDGTEIYLWDGKTFRTRAEFRAWFEELHSTSENAKRHVTALEVDGNVARVQVVLHASINGEEKTVKLDHETHFEGDRLVRVTVDIKPL

SEQ ID NO: 78 - protein, 117 aa, synthetic construct
  VARIANT 1: note = optionally absent
MSEEEQRNFVKRFYEALDAGDAETASALFPDGTVIHLWDGTTFHTQAEFRAWFTELRSTSENAKREVTKFAVDGNVAHVEVVLHANHNGEPRVVRLTHEFHFEGDHLVEVTVRIDPL

SEQ ID NO: 79 - protein, 117 aa, synthetic construct
  VARIANT 1: note = optionally absent
MSEEEQREFVRRFYEALDAGDAETASALFPDGTVIDLWDGKTFHTRAEFREWFVKLKSTSDNAKREVTNFKVDGDVAHVEVVLKASINGEEKVVKLTHTFQFEGDRLVRVSVKIEPL

SEQ ID NO: 80 - protein, 117 aa, synthetic construct
  VARIANT 1: note = optionally absent
MSEEEQKEFCKKFYEALDAGDADTASALFPDGTKIHLWDGKTFTTQAQFKAWFVELYSKSENAKREITKFEVDGNVADVEVVLHAIVNGEKKTVKLKHVFKFEGDKLVEVNVEIKPL

SEQ ID NO: 81 - protein, 117 aa, synthetic construct
  VARIANT 1: note = optionally absent
MSAEEIKNFCDKFYKALDAGDADTASALFPDGTEIYLWDGKTFKTQAEFKAWFEKLYSTSKDAKRKITSLEVNGNVAKVKVVLEAIIDGEKKTVNLEHIFYFEGDKLVKVEVSIEPL

SEQ ID NO: 82 - protein, 117 aa, synthetic construct
  VARIANT 1: note = optionally absent
MSEEEQREFVARFYAALDAGDAETASALFPDGTEIHLWDGKTFTTRAEFRAWFEKLHSLSDNASRHVTSFKVDGNVAEVEVVLHADFKGKKLTVKLRHRYQFEGDRLVRVDVEIFPL

SEQ ID NO: 83 - protein, 117 aa, synthetic construct
  VARIANT 1: note = optionally absent
MSADAQREFVRRFYEALDAGDAETAAALFPDGTEIHLWDGKTFRTQAEFRAWFEKLRAQSENAKRHVVNFQVDGNDADVEVVLHATYNGEQKTVRLKHKFHFEGDQLVRVDVTIEPL

SEQ ID NO: 84 - protein, 117 aa, synthetic construct
  VARIANT 1: note = optionally absent
MSEEQIRQFLRRFYEALDSGDADTAASLFHPGVTIHLWDGVTFTSREEFREWFERLFSTRKDAQAEIKSLEVRGDTVEVHVQLHATHNGQKHTVDATHHWHFRGNRVTEMRVHINPT

SEQ ID NO: 85 - protein, 117 aa, synthetic construct
  VARIANT 1: note = optionally absent
MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPT

SEQ ID NO: 86 - protein, 117 aa, synthetic construct; identical to SEQ ID NO: 80
  VARIANT 1: note = optionally absent

SEQ ID NO: 87 - protein, 117 aa, synthetic construct; identical to SEQ ID NO: 81
  VARIANT 1: note = optionally absent

SEQ ID NO: 88 - protein, 117 aa, synthetic construct; identical to SEQ ID NO: 82
  VARIANT 1: note = optionally absent

SEQ ID NO: 89 - protein, 117 aa, synthetic construct
  VARIANT 1: note = optionally absent
MSEEEIRQFVKKFYEALDAGDAETASALFPDGTKIYLWDGTVFNTQAEFKAWFVELYSTSENAKREVVKFEVDGNKAKVEVVLHASIDGEEKTVKLKHEFYFEGDKVKEVKVEIEPL

SEQ ID NO: 90 - protein, 117 aa, synthetic construct
  VARIANT 1: note = optionally absent
MSEEEQREFWRRFYEALDAGDAETASALFPDGTEIYLWDGRTFRTQAEFRAWFERLRATSEDARREIVSFRVEGNRSEVEVVLHANVNGEKKTVRLRHYFEFEGDRLVRVEVEIVPL

SEQ ID NO: 91 - protein, 117 aa, synthetic construct
  VARIANT 1: note = optionally absent
MSEEEIKEFCKKFYEALDAGDADTASALFKDGTEIYLWDGRTFKTRAEFKAWFTRLRSTSDNAKREIVSLSVNGNVAKVKVVLRASINGEERTVLLTHEFLFEGDELVEVRVSIKPL

SEQ ID NO: 92 - protein, 117 aa, synthetic construct
  VARIANT 1: note = optionally absent
MSKEEQEEFVKQFYEALDAGDAETASALFPDGTVIHLWDGKTFHTQAEFRAWFEELKSTSENAKREVTKFEVDGDVADVEVVLKANINGEEKVVNLKHKFKFEGDKVVEVWVEIEPL

SEQ ID NO: 93 - protein, 117 aa, synthetic construct
  VARIANT 1: note = optionally absent
MSEDAIREFVKRFYEALDAGDADTASALFPDGTEIHLWDGRVFRTRAEFRAWFVELRSQSENAKREVTDLEVEGNVARVEVVLHANYKGEERTVRLRHEFQFEGDKVVEVRVEIEPL

SEQ ID NO: 94 - protein, 117 aa, synthetic construct
  VARIANT 1: note = optionally absent
MSEEEQKEFCRRFYLALDAGDADTASALFKDGTKIYLWDGKVFNTQEEFRKWFVELRSTSDQAKRSIEDFKVEGNTAKVKVVLEASKNGEKRTVGLEHIFEFEGDEVKEVYVKIKPL

SEQ ID NO: 95 - protein, 117 aa, synthetic construct
  VARIANT 1: note = optionally absent
MSATEQREFNKRFYEALDAGDADTASALFPDGTEIYLWDGTTFHTQEEFRAWFVELRSTSENARRHIVSFTVDGNKADVEVVLEATVKGEPKRVRLTHNYQWEGDKLVKVTVKIVPL

SEQ ID NO: 96 - protein, 117 aa, synthetic construct
  VARIANT 1: note = optionally absent
MSEEEIREFVKRFYQALDAGDADTASALFPDGTKIYLWDGVIFTKREEFRKWFVELKSKSDNAKREVVDLRVEGDTADVSVVLKAKYKGEPKVVKLNHKFYFEGDKLVEVEVEIVPM

SEQ ID NO: 97 - protein, 117 aa, synthetic construct
  VARIANT 1: note = optionally absent
MTEEEQREFWERFYKALDAGDAETASALFPDGTEIYLWDGTVFKTQEEFRSWFVDLYSKSNNAKRHIVEFRVEGDVSYVEVVLHASENGTEKTVRLTHITEFEGDKVVKVEVSITPL

SEQ ID NO: 98 - protein, 117 aa, synthetic construct
  VARIANT 1: note = optionally absent
MSEDEIREFWKKFYEALDAGDADTASALFPDGTKIYLWDGKVFHTQAEFREWFVKLYSTSENAKREITRFEVDGNVANIEVVLEANENGEQKKVKLRHIAEFEGDKLVEVNVEIEPL

SEQ ID NO: 99 - protein, 117 aa, synthetic construct
  VARIANT 1: note = optionally absent
MSEKEIREFVERFYKALDDGDAETASSLFPDGTRIYLWDGTEFSTQAEFRAWFEELHSRSEAARRRVVRLEVDGDEADVEVVLDADFEGEHHRVRLRHRFFFEGDRLVEVEVTIEPL

SEQ ID NO: 100 - protein, 117 aa, synthetic construct
  VARIANT 1: note = optionally absent
MSEKEIREFVDKFYKALDSGDAETASALFPDGTNIYLWDGTVFKTKEEFRKWFVELKSTSDNAKRKVTKLEVHGNTAHVEVVLKASVNGEEKVVKLKHIFLFEGDKLREVYVEIEPL

SEQ ID NO: 101 - protein, 117 aa, synthetic construct
  VARIANT 1: note = optionally absent
MSEEEQREFWRKFYEALDAGDAETASALFPDGTEIYLWDGTVFRTRAEFRAWFRQLYSQSDNAKRRITSFVVEGNRAYVEVVLVANIKGKKVEVSLKHLFEFEGSRLVRVYVEIKPL

SEQ ID NO: 102 - protein, 117 aa, synthetic construct
  VARIANT 1: note = optionally absent
MSEQEQRDFVARFYAALDAGDAETASALFRDGTKIYLWDGKTFRTQAEFRSWFVELHSQSKDAKRRVVSFRVDGNVADVNVILHASVEGEERTVRLNHVFKFEGDHLVEVRVDIEPL

SEQ ID NO: 103 - protein, 117 aa, synthetic construct
  VARIANT 1: note = optionally absent
MSEEEIRAFWERFYSALDAGDAETASSLFPDGTEIYLWDGKTFRTQEEFRAWFEELRSTSADASRHITSLKVEGNRADIEVVLRANFKGEEKTVRLRHEAEFEGDRLVRVEVRIDPL

SEQ ID NO: 104 - protein, 117 aa, synthetic construct
  VARIANT 1: note = optionally absent
MSAAEQREFVDRFYKALDAGDAETASALFPDGTVIDLWDGRTFRTRAEFRAWFERLHATSTDAKREVTQMKVDGNTVDVEVVLRATVNGEEKVVKLRHQFHFEGDQLVRVRVDITPL

SEQ ID NO: 105 - protein, 117 aa, synthetic construct
  VARIANT 1: note = optionally absent
MSESEIKEFVRKFYEALDAGDADTAAALFPDGTVIKLWDGTTFYTQAEFRAWFVKLYSTSDEASREVTSLKVEGDKAEVEVVLKAKINGEEKTVKLKHIFEFKGDKLVRVEVSISPL

SEQ ID NO: 106 - protein, 117 aa, synthetic construct
  VARIANT 1: note = optionally absent
MSEEEQREFWKRFYEALDAGDAETAAALFPDGTEIHLWDGKTFRTQAEFRAWFVQLRSTSEAAKRKIIKFEVIGNKSFVEVVLKASINGEEKEVFLRHYFEFEGDKLVRVYVTIKPL

SEQ ID NO: 107 - protein, 117 aa, synthetic construct
  VARIANT 1: note = optionally absent
MSEREIRQFVDRFYAALDAGDAETAAALFPDGTEIHLWDGRTFTTQAEFRAWFEKLRARSDNARREVVDLQVDGDEADVRVVLDATFRGEARRVRLTHRFLFEGDQVTRVEVEIRPD

SEQ ID NO: 108 - protein, 117 aa, synthetic construct
  VARIANT 1: note = optionally absent
MSEAAQREFWDRFYRALDAGDAETASALFPDGTEIHLWDGTTFRTRAEFRAWFRDLHARSDAARRSIVSFEVDGDDARVEVVLHANVDGQERTVRLRHEAHFEGDRLVRVHVVIQPL

SEQ ID NO: 109 - protein, 117 aa, synthetic construct
  VARIANT 1: note = optionally absent
MSETAIRQFVERFYEALDAGDAETAAALFPDGTRIYLWDGTTFHTQAEFRAWFEALHATSSGAKRHVVALQVDGDVADVEVVLHADVDGEKRTVHLKHTFKFRGDRLVEVEVHIQPL

SEQ ID NO: 110 - protein, 117 aa, synthetic construct
  VARIANT 1: note = optionally absent
MSSDAQRAFVDRFYRALDAGDAETASALFPDGTRIHLWDGTTFTTREEFRAWFVDLRSRSENAAREVVSFDVDGDVAHVEVVLKAVIEGEEVVVRLRHVFEWEGDRLVEVYVEIDPL

SEQ ID NO: 111 - protein, 117 aa, synthetic construct
  VARIANT 1: note = optionally absent
MSEKDQKEFVKKFYEALDAGDAETASSLFPDGTEIHLWDGKVFHTQAEFKAWFEELYSRSDDAKRSIISFKVDGNIAKVIVVLKAFVDGEKLEVLLEHEFKFEGDKLVEVKVKIYPL

SEQ ID NO: 112 - protein, 117 aa, synthetic construct
  VARIANT 1: note = optionally absent
MSEEEQREFVRRFYDALDAGDAETASALFPDGTQIELWDGKTFHTREEFRAWFVKLHSTSDDARREVVSFSVAGDVANVEVVLRANIRGEKKVVRLRHIFEFEGDRLVRVRVDIEPL

SEQ ID NO: 113 - protein, 117 aa, synthetic construct
  VARIANT 1: note = optionally absent
MSEDEIREFVARFYAALDAGDANTASALFPDGTVIHLWDGITFHTQAEFRAWFEKLHSTSENASREVVSLKVDGNVAHVEVVLRASVDGEERTVRLRHVFRFEGDKLVEVSVSITPL

SEQ ID NO: 114 - protein, 117 aa, synthetic construct
  VARIANT 1: note = optionally absent
MSEEEIREFWKRFYEALDAGDAETASALFPDGTRIYLWDGKEFTTQAEFRAWFEELYSTSEDASREIVKLEVEGNVAYVEVVLKANINGEEKVVKLKHVFHFEGDRLVEVEVEIEPL

SEQ ID NO: 115 - protein, 117 aa, synthetic construct
  VARIANT 1: note = optionally absent
MSAEAQRRFVDRFYAALDAGDADTASALFPDGTEIHLWDGRTFRTRAEFRAWFRELRARSDNARREVVAFEVDGDTAHVEVVLRASIDGEERVVRLRHTFYFEGDRLVRVEVEIEPL

SEQ ID NO: 116 - protein, 117 aa, synthetic construct
  VARIANT 1: note = optionally absent
MSEEEQREFVDRFYAALDAGDAETASALFPDGTKIYLWDGKVFTTREEFRAWFEKLYSTSENAKRHVVSFKVDGNKADVEVVLHANINGEKKTVRLRHVFYFEGDKLVEVKVEIKPL

SEQ ID NO: 117 - protein, 117 aa, synthetic construct
  VARIANT 1: note = optionally absent
MSEEEQREFVKRFYEALDAGDAETASALFPDGTEIHLWDGKTFYTREEFRAWFEKLYSTSDNASRSVTEFKVDGNKAKVKVVLKANINGEKKTVKLEHYFEFEGDKLVRVNVTIKPL

SEQ ID NO: 118 - protein, 117 aa, synthetic construct
  VARIANT 1: note = optionally absent
MSEEEQRRFVERFYAALDAGDADTASALFPDGTEIHLWDGTVFRTRAEFRAWFERLYSTSENAKRHVVRFEVEGNVARVEVVLHANIDGEERTVRLTHVFEFEGDRLVRVNVTINPL

SEQ ID NO: 119 - protein, 117 aa, synthetic construct
  VARIANT 1: note = optionally absent
MSEEEIKEFVKRFYEALDAGDAETASALFPDGTRIYLWDGRVFRTRAEFRAWFVELHSTSEDAKREVIELKVEGNVAKVKVVLHANINGEKKTVLLEHYFEFEGDRLVEVRVEIKPL

SEQ ID NO: 120 - protein, 117 aa, synthetic construct
  VARIANT 1: note = optionally absent
MSEEAQKEFVKKFYEALDAGDADTASALFPDGTEIYLWDGKTFHTKAEFKAWFVKLKSTSDNAKRSVVKFEVDGNVAYVEVVLHANINGEEKVVKLTHIFEFEGDKLVKVNVTIKPL

SEQ ID NO: 121 - protein, 117 aa, synthetic construct
  VARIANT 1: note = optionally absent
MSEEEIREFVRRFYEALDAGDAETASALFPDGTVIYLWDGKTFHTREEFRAWFVELRSKSENAKRHVVSLRVDGNVADVEVVLDADINGEKKTVKLRHEFRFEGDRLVEVRVEIEPL

SEQ ID NO: 122 - protein, 117 aa, synthetic construct
  VARIANT 1: note = optionally absent
MSEEEIREFVKRFYEALDAGDADTASALFPDGTKIYLWDGKTFSTQAEFKAWFVKLKSTSENAKRKIVKLKVDGDVAEVEVVLHANVNGEEKVVKLKHKFKFEGDKLVEVEVEIKPL

SEQ ID NO: 123 - protein, 117 aa, synthetic construct
  VARIANT 1: note = optionally absent
MSEEEQREFWKRFYEALDAGDADTASALFPDGTKIHLWDGKTFTTQAEFRAWFVELHSTSDDAKREIVKFEVDGNKSYVEVVLRANINGEEKVVRLTHETQFEGDKLVEVKVTIEPL

SEQ ID NO: 124 - protein, 117 aa, synthetic construct
  VARIANT 1: note = optionally absent
MSEEAQREFVRRFYAALDAGDAETAAALFPDGTVIHLWDGRTFRTQAEFRAWFVELYSKSEDAKREVVRFEVDGDRARVEVVLKANFKGKKEVVKLVHYFLFEGDRLVEVRVEIKPL

SEQ ID NO: 125 - protein, 117 aa, synthetic construct
  VARIANT 1: note = optionally absent
MSAEAQREFVRRFYAALDAGDAETASALFPDGTRIHLWDGRVFRTREEFRAWFVKLYSTSEDARREVTAFRVDGDRADVEVVLRASINGEEKTVRLRHVFQFEGDRLREVRVAIEPL

SEQ ID NO: 126 - protein, 117 aa, synthetic construct
  VARIANT 1: note = optionally absent
MSEEEIREFCKRFYEALDAGDADTASALFPDGTEIYLWDGRTFRTREEFRAWFVELHSTSDDARRSIVKLEVEGNVAYVEVVLRANVDGEEKVVRLVHIFYFEGDKLVKVYVSIRPL

SEQ ID NO: 127 - protein, 117 aa, synthetic construct
  VARIANT 1: note = optionally absent
MSEEEIREFWKKFYEALDAGDAETASALFPDGTKIHLWDGKTFNTQAEFKAWFVKLYSESDDAKREIVELEVDGNVAYVKVVLHANYKGEQKTVELKHIFKFEGDKLVEVNVEIKPL

SEQ ID NO: 128 - protein, 117 aa, synthetic construct
  VARIANT 1: note = optionally absent
MSEEEQREFWKKFYEALDAGDAETASALFPDGTKIYLWDGKVFYTQEEFRAWFVELRSQSDDAKRKIVDFKVEGDVAYVEVVLEANINGEKKTVKLKHIYKFEGDKLVEVKVKIEPL

SEQ ID NO: 129 - protein, 117 aa, synthetic construct
  VARIANT 1: note = optionally absent
MSEEEQREFWRRFYEALDAGDAETASALFPDGTKIHLWDGRTFTTQEEFRAWFVDLHSRSDDAKREIVSFKVEGNKARVEVVLRAREDGEERVVALRHETEFEGDRLVEVNVDIRPL

SEQ ID NO: 130 - protein, 117 aa, synthetic construct
  VARIANT 1: note = optionally absent
MSEEAQREFVRRFYAALDAGDADTASALFPDGTRIYLWDGRTFTTRAEFRAWFERLHATSADAKREVVDFEVEGNKAKVKVVLHANVNGEEKTVLLEHWFEFEGDRLVRVEVTIKPL

SEQ ID NO: 131 - protein, 117 aa, synthetic construct
  VARIANT 1: note = optionally absent
MSEKEQREFWKKFYEALDAGDAETASALFPDGTKIYLWDGKTFTTQAEFRAWFVELRSTSENAKRKIVSFKVDGNKSEVEVVLEANINGEKKVVKLKHIFKFEGDKLVEVKVEIIPL

SEQ ID NO: 132 - protein, 117 aa, synthetic construct
  VARIANT 1: note = optionally absent
MSEEEQREFWRKFYEALDAGDADTASALFPDGTEIHLWDGKTFRTREEFRAWFEELYSTSEDAKREIVKFEVEGNKSFVEVVLKANVNGKKVVVKLRHVFEFEGDKVVKVEVEIEPL

SEQ ID NO: 133 - protein, 117 aa, synthetic construct
  VARIANT 1: note = optionally absent
MSEEEIKKFWEKFYNALDAGDADTASALFPDGTEIHLWDGKTFKTQAEFREWFVKLYSTSDDAKREIVKLEVDGNVAYVEVVLRANVDGEEKTVKLRHVAVFEGDKLKEVKVTIKPL

SEQ ID NO: 134 - protein, 117 aa, synthetic construct
  VARIANT 1: note = optionally absent
MSEEEQREFVRRFYEALDAGDAETASALFPDGTKIHLWDGKTFSTRAEFRAWFVELRSTSENAKRRVTSFRVEGNEADVEVVLEATVNGEKKRVRLRHRFLFEGDRLVEVYVEIKPL

SEQ ID NO: 135 - protein, 117 aa, synthetic construct
  VARIANT 1: note = optionally absent
MSEEEQREFVKRFYEALDAGDAETASALFPDGTLIYLWDGKTFTTQAEFRAWFEKLRSTSEDAKREVVEFKVDGNVADVVVVLEANINGEKKVVKLRHRFHFEGDKLVKVEVEIEPL

SEQ ID NO: 136 - protein, 117 aa, synthetic construct
  VARIANT 1: note = optionally absent
MSEEEQREFVKRFYEALDAGDADTASALFPDGTKIHLWDGTTFTTQAEFKAWFEKLYSKSDNAKRHVVSFKVEGNVAYVEVVLHANFKGEEKTVSLKHIFKFEGDKLVEVEVKIKPL

SEQ ID NO: 137 - protein, 117 aa, synthetic construct
  VARIANT 1: note = optionally absent
MSEEEQREFVRRFYEALDAGDAETASALFPDGTVIHLWDGKTFRTQAEFRAWFVELKSTSEDAKRRVVSFRVDGDEAEVVVVLHANIDGEERVVRLTHRFLFEGDRVVEVWVEIEPL

SEQ ID NO: 138 - protein, 117 aa, synthetic construct
  VARIANT 1: note = optionally absent
MSEQEQREFVDRFYRALDAGDADTASALFPDGTEIYLWDGKTFRTQAEFREWFVKLHSTSEDAKRRVVSFSVDGNVADVEVVLEANVNGEKKTVKLKHRFHFEGDKVVRVEVSIEPL

SEQ ID NO: 139 - protein, 117 aa, synthetic construct
  VARIANT 1: note = optionally absent
MSEEEIREFVRRFYAALDAGDAETASALFPDGTRIHLWDGRTFTTQAEFREWFVKLYSESDDARREVVSLEVDGDRALVRVVLRASYKGEERVVELRHEFLFEGDELVEVRVEIRPL

SEQ ID NO: 140 - protein, 117 aa, synthetic construct
  VARIANT 1: note = optionally absent
MSEEEQREFWKRFYEALDAGDAETASALFPDGTEIHLWDGTVFRTRAEFRAWFEQLYAESDDAKREIVSFKVEGNVSDVEVVLHASYKGEKKTVKLRHRFFFEGDRLVEVEVEIEPL

SEQ ID NO: 141 - protein, 117 aa, synthetic construct
  VARIANT 1: note = optionally absent
MSEEEQREFVKRFYEALDAGDAETASALFPDGTEIHLWDGITFHTQAEFREWFVKLRSTSENAKREVVKFEVDGNKAKVEVVLKANINGEEKEVKLTHTFEFEGDKLVKVNVDIKPL

SEQ ID NO: 142 - protein, 117 aa, synthetic construct
  VARIANT 1: note = optionally absent
MSAEEIREFVRRFYEALDAGDADTASALFPDGTEIHLWDGRTFRTQAEFRAWFVRLRATSDDASREVVSLEVDGNVADVEVVLHANVNGEKKVVRLRHRFYFEGDRLVRVEVTIVPL

SEQ ID NO: 143 - protein, 117 aa, synthetic construct
  VARIANT 1: note = optionally absent
MSKEEIKEFVKRFYQALDAGDAETASSLFPDGTKIYLWDGKVFKTQEEFRAWFEKLHSTSENAKREVTKLEVEGNVAYIEVVLKANVNGEEKIVNLTHKFYFEGDKLVEVEVKIVPL

SEQ ID NO: 144 - protein, 117 aa, synthetic construct
  VARIANT 1: note = optionally absent
MSEEEIREFVKRFYEALDAGDAETASALFPDGTKIHLWDGTVFTTQAEFRAWFEKLYSTSDNASRSVVSFKVDGNVAEVEVVLKANIKGREKVVKLKHIFHFEGDKLVEVEVSIEPL

SEQ ID NO: 145 - protein, 117 aa, synthetic construct
  VARIANT 1: note = optionally absent
MSEEEIREFVKRFYEALDAGDAETASALFPDGTVIHLWDGITFHTQAEFRAWFEALYSTSENARREVVALRVEGNRARVEVVLHANVDGEERTVRLRHEFLFEGDRLVEVRVEIEPL

SEQ ID NO: 146 - protein, 117 aa, synthetic construct
  VARIANT 1: note = optionally absent
MSEEAQREFVKRFYEALDAGDAETASALFPDGTRIHLWDGRTFTTREEFRAWFEELRSKSEDAKREVTSFRVDGNVAEVEVVLHANINGEEKTVLLKHRFVFEGDRLVEVHVEIKPL

SEQ ID NO: 147 - protein, 117 aa, synthetic construct
  VARIANT 1: note = optionally absent
MSEEAQRQFVRRFYEALDAGDADTASALFPDGTEIHLWDGRTFRTRAEFRAWFERLRATSADARRSVTSFEVDGDFARVEVVLRASFAGEERVVRLRHYFQFEGDRLVRVEVEIRPL

SEQ ID NO: 148 - protein, 117 aa, synthetic construct
  VARIANT 1: note = optionally absent
MSKKAQEEFWKRFYEALDAGDADTASALFPDGTKIYLWDGKTFTTQAEFRAWFVELHSKSDNAKRRITKFEVDGNKSYVEVVLEANVNGKKEVVKLVHETLFEGDKLVEVRVDIKPL

SEQ ID NO: 149 - protein, 117 aa, synthetic construct
  VARIANT 1: note = optionally absent
MSEEEQREFWRRFYEALDAGDAETASALFPDGTEIHLWDGKVFRTQAEFRAWFERLRATSADAKREIVSFRVDGDRADVEVVLRAVVDGEEKEVKLNHRFFFEGDRLVRVEVEIKPL

SEQ ID NO: 150 - protein, 117 aa, synthetic construct
  VARIANT 1: note = optionally absent
MSEEAIREFWKRFYEALDAGDAETASALFPDGTEIHLWDGKTFRTRAEFKAWFEELYSTSENAKREITSLKVEGNRAEVEVVLHANIEGKEKTVNLKHWFEFEGDHLVRVRVDIKPL

SEQ ID NO: 151 - protein, 117 aa, synthetic construct
  VARIANT 1: note = optionally absent
MSEEEQREFVRRFYEALDAGDAETASALFPDGTVIHLWDGRTFRTQAEFRAWFEELYSTSEDASRHVVSFKVDGNKARVEVVLHANINGEEHTVNLTHVFHFEGDKLVEVEVDIRPL

SEQ ID NO: 152 - protein, 117 aa, synthetic construct
  VARIANT 1: note = optionally absent
MSEEEQREFWKRFYEALDAGDAETASALFPDGTVIHLWDGKTFHTREEFRAWFVELKSKSEDAKREIVDFEVDGNVSRVKVVLHANYKGEEKVVELEHVFHFEGDRLVEVEVKIKPL

SEQ ID NO: 153 - protein, 117 aa, synthetic construct
  VARIANT 1: note = optionally absent
MSEEEIREFVKRFYAALDAGDAETASALFPDGTEIYLWDGTTFRTQAEFRAWFVELYSRSDDASREVVDLKVDGNKAEVEVVLKASVDGEERVVKLKHFFEFEGDKLVKVEVTIEPL

SEQ ID NO: 154 - protein, 117 aa, synthetic construct
  VARIANT 1: note = optionally absent
MSEEEQREFVKKFYEALDAGDAETASALFPDGTEIHLWDGKTFRTQAEFRAWFEKLKSTSENAKRKIVSFKVDGNKADVEVVLKANINGEEKTVKLKHTFEFEGDKLKKVNVEIVPL

SEQ ID NO: 155 - protein, 117 aa, synthetic construct
  VARIANT 1: note = optionally absent
MSEEAQREFVRRFYEALDAGDAETASALFPDGTKIYLWDGRVFETQEEFRAWFVELRSTSENARRRVVDFEVDGNVARVEVVLHANIDGEEKTVRLRHIFKFEGDKLVEVEVTIEPL

SEQ ID NO: 156 - protein, 117 aa, synthetic construct
  VARIANT 1: note = optionally absent
MSEEEIREFVRRFYEALDAGDAETASALFPDGTKIYLWDGITFTTQAEFRAWFVELRSTSDAARREVVRLEVDGNVAFVEVVLHASHRGEERTVLLRHVFQFEGDKLVKVEVSIDPL

SEQ ID NO: 157 - protein, 117 aa, synthetic construct
  VARIANT 1: note = optionally absent
MSEKEQKEFWKKFYDALDAGDAETASALFPDGTIIYLWDGIVFRTREEFKEWFVKLKSTSENAKRKIVSFKVDGNKSDVEVVLEANVNGEKKKVKLKHVFHFEGDKLVEVEVKIDPL

SEQ ID NO: 158 - protein, 117 aa, synthetic construct
  VARIANT 1: note = optionally absent
MSEEEIRNFVKRFYEALDAGDAETASALFPDGTEIYLWDGKTFRTQAEFRAWFEELRSTSDDAKREVVKFEVEGNVAYVEVVLKANHKGKKEVVKLKHEFEFEGDKVVKVRVEIKPL

SEQ ID NO: 159 - protein, 117 aa, synthetic construct
  VARIANT 1: note = optionally absent
MSEEEQREFWKRFYEALDAGDAETASALFPDGTEIHLWDGRTFRTRAEFRAWFEELYSQSENARREITRFEVDGNVSHVEVVLEAEYEGEKRVVRLRHVFEFEGDRLVRVEVEIEPL

SEQ ID NO: 160 - protein, 117 aa, synthetic construct
  VARIANT 1: note = optionally absent
MSEEEIREFVKRFYEALDAGDAETASALFPDGTEIHLWDGTVFRTREEFRAWFVELRSKSDDAKREVVKFEVDGNKAYVEVVLHANVDGEEKTVRLVHEFLFEGDKLVEVKVDIEPL

SEQ ID NO: 161 - protein, 117 aa, synthetic construct
  VARIANT 1: note = optionally absent
MSEEDIREFVRRFYEALDAGDADTASALFKDGTKIHLWDGKTFTTREEFREWFVRLHSTSDDARREVVRLEVEGNRAHVEVVLRASYQGEDRVVRLTHEFLFEGDEVVEVHVRIEPL

SEQ ID NO: 162 - protein, 117 aa, synthetic construct
  VARIANT 1: note = optionally absent
MSEEAQREFWRRFYEALDAGDAETASALFPDGTEIHLWDGRTFRTQAEFRDWFVKLHSTSADARREITRFRVEGDVAHVEVVLHASIDGEKKTVKLRHVAHFEGDKLVEVHVDIEPL

SEQ ID NO: 163 - protein, 117 aa, synthetic construct
  VARIANT 1: note = optionally absent
MSEEEIREFVRRFYEALDAGDAETASALFKDGTKIYLWDGTVFETREEFRAWFVELYSKSENARRRVVSFKVDGNVAEVEVVLHASFQGEDKVVRLKHRFKFEGDEVVEVEVDIEPL

SEQ ID NO: 164 - protein, 117 aa, synthetic construct
  VARIANT 1: note = optionally absent
MSEEAQREFWKRFYEALDAGDAETASALFPDGTRIYLWDGRVFTTQAEFRAWFVELRSTSADAKREIVKFEVDGDVSYVEVVLKANVDGEEKIVRLRHVFHFEGDRLVEVEVEIEPL

SEQ ID NO: 165 - protein, 117 aa, synthetic construct
  VARIANT 1: note = optionally absent
MSEEEQREFWKRFYEALDAGDAETASALFPDGTRIYLWDGRTFTTRAEFRAWFEELHSTSENARREIVSFRVDGDVADVEVVLRANVNGEERTVRLRHVFHFEGDRLVEVHVEIEPL

SEQ ID NO: 166 - protein, 117 aa, synthetic construct
  VARIANT 1: note = optionally absent
MSEEAQREFWRRFYDALDAGDAETASALFPDGTRIHLWDGRTFHTRAEFRAWFVELRSTSDDAKREIISFEVDGNRSRVEVVLHANIDGEEKKVLLRHEFLFEGDRLVEVHVEIKPL

SEQ ID NO: 167 - protein, 117 aa, synthetic construct
  VARIANT 1: note = optionally absent
MSEEEIREFVDRFYKALDAGDAETASALFPDGTEIHLWDGTTFHTQAEFRAWFVKLKSTSDNAKREIVKLEVDGNKAKVEVVLKANVNGEEKVVKLTHEFEFEGDRLVKVTVKIEPL

SEQ ID NO: 168 - protein, 117 aa, synthetic construct
  VARIANT 1: note = optionally absent
MSAEAIRQFVERFYAALDAGDADTASALFPDGTEIHLWDGRTFRTQAEFRAWFEELYSTSDGASREVVALEVDGNRARVEVVLHASVGGEEKTVRLRHEFEFEGDQLVRVTVEIEPL

SEQ ID NO: 169 - protein, 117 aa, synthetic construct
  VARIANT 1: note = optionally absent
MSEKEQKEFWKKFYDALDAGDADTASALFPDGTKIYLWDGKVFRTQEEFREWFEKLYSKSDNAKREIVKFKVDGNIAYVEVILKANVDGEEKVVKLKHVFKFEGDKVKEVKVKIVPL

SEQ ID NO: 170 - protein, 117 aa, synthetic construct
  VARIANT 1: note = optionally absent
MSEESQREFWKRFYAALDAGDAETASALFPDGTEIHLWDGTVFRTRAEFRAWFVDLHSKSDNASREITSFKVEGNKALVEVVLHASFKGEERTVKLTHVFEFEGDRLVRVEVEIKPL

SEQ ID NO: 171 - protein, 117 aa, synthetic construct
  VARIANT 1: note = optionally absent
MSEEAVREFVRRFYEALDAGDAETASALFPDGTEIHLWDGTTFRTRAEFRAWFRRLRATSEDARRRVVRLEVDGNVADVEVVLHASYRGEERVVRLRHRYEFEGDRLVRVDVEIRPL

SEQ ID NO: 172 - protein, 117 aa, synthetic construct
  VARIANT 1: note = optionally absent
MSEEEQRQFVKRFYEALDAGDSETASALFPDGTVIHLWDGTTFHTQEEFRAWFEKLRSTSDNARREVVHFEVEGNVAYVEVVLRASVGGEERVVRLRHVFHFEGDHLVEVEVEIEPL

SEQ ID NO: 173 - protein, 117 aa, synthetic construct
  VARIANT 1: note = optionally absent
MSEEEQRNFWKKFYEALDAGDAETASALFPDGTEIYLWDGTVFKTREEFRAWFVELYSTSENAKRHIVDFKVDGNESKVKVILKANIDGEEKVVELEHYFLFEGDHLVEVKVEIHPL

SEQ ID NO: 174 - protein, 117 aa, synthetic construct
  VARIANT 1: note = optionally absent
MSEEEIREFVERFYKALDAGDAETASSLFPDGTEIHLWDGKTFHTQEEFRSWFVELKSKSDNAKRKVVSFEVEGNKAKVKVVLKASYKGEEKEVKLEHEFEFEGDKLVEVNVKIEPL

SEQ ID NO: 175 - protein, 117 aa, synthetic construct
  VARIANT 1: note = optionally absent
MSEKEQREFWKKFYEALDAGDAETASALFPDGTEIHLWDGKTFHTREEFKEWFVKLYSRSENAKRHIVKFEVDGDKAYVEVVLYAKIGGEEKVVKLKHEFLFEGDKLVEVNVKIEPL

SEQ ID NO: 176 - protein, 117 aa, synthetic construct
  VARIANT 1: note = optionally absent
MSEEEIKEFVERFYKALDAGDADTASSLFPDGTEIFLWDGKTFKTKEEFREWFVKLKSESDDAKREVVSLKVEGNKAYVEVVLHANYKGEKKEVKLTHIFEFEGDKVVKVEVTIVPL

SEQ ID NO: 177 - protein, 117 aa, synthetic construct
  VARIANT 1: note = optionally absent
MSEEEQRQFVKEFYEALDAGDAETASALFPDGTEIHLWDGKTFTTQAEFRAWFERLYSTSENARRHVVDFRVEGNVADVEVVLHATKDGEEHTVRLRHKFEFEGDELVRVEVVIEPL

SEQ ID NO: 178 - protein, 117 aa, synthetic construct
  VARIANT 1: note = optionally absent
MSAEAQREFWARFYAALDAGDADTASALFPDGTEIYLWDGKVFRTREEFRAWFVELRSTSADAKREIVSFEVDGNRASVDVVLHASVNGEEHTVHLHHEFEFEGDRLVRVRVTIQPL

SEQ ID NO: 179 - protein, 117 aa, synthetic construct
  VARIANT 1: note = optionally absent
MSAEQQREFVKRFYEALDAGDADTASALFPDGTEIHLWDGTTFRTRAEFRAWFEELYSTSENASREVTSFSVDGDVADVEVVLRANLGGEDRTVSLRHVFHFAGDRLVRVEVSIRPL

SEQ ID NO: 180 - protein, 117 aa, synthetic construct
  VARIANT 1: note = optionally absent
MSEEEIKEFVRRFYEALDAGDADTASALFPDGTRIHLWDGTTFTTRAEFRAWFVDLYSKSDAAKREVTSLKVEGNVAKVKVVLKANYKGEEKTVELEHKFEFEGDRLVRVDVTIKPM

SEQ ID NO: 181 - protein, 117 aa, synthetic construct
  VARIANT 1: note = optionally absent
MSEQAQRDFVKKFYEALDAGDAETASALFPDGTQIYLWDGQTFTTQEQFRAWFVELRSTSKDAKREVVSFKVDGNNAEVEVVLHAVIDGEERTVKLKHYFQWEGDQLVKVTVSIEPL

SEQ ID NO: 182 - protein, 18 aa, synthetic construct
MSEEQIRQFLRRFYEALD

SEQ ID NO: 183 - protein, 8 aa, synthetic construct
ADTAASLF

SEQ ID NO: 184 - protein, 4 aa, synthetic construct
TIHL

SEQ ID NO: 185 - protein, 13 aa, synthetic construct
EEFREWFERLFST

SEQ ID NO: 186 - protein, 10 aa, synthetic construct
QREIKSLEVR

SEQ ID NO: 187 - protein, 11 aa, synthetic construct
VEVHVQLHATH

SEQ ID NO: 188 - protein, 13 aa, synthetic construct
KHTVDATHHWHFR

SEQ ID NO: 189 - protein, 12 aa, synthetic construct
VTEMRVHINPTG

SEQ ID NO: 190 - protein, 4 aa, synthetic construct
HPGV

SEQ ID NO: 191 - protein, 4 aa, synthetic construct
RKDA

SEQ ID NO: 192 - protein, 139 aa, synthetic construct
MGSHHHHHHGSGSENLYFQGSMSEEQIRQFLRRFYEALDSGDADTAASLFHPGVTIHLWDGVTFTSREEFREWFERLFSTRKDAQREIKSLEVRGDTVEVHVQLHATHNGQKHTVDATHHWHFRGNRVTEMRVHINPTG

SEQ ID NO: 193 - DNA, 420 bp, synthetic construct
  source 1..420: mol_type = other DNA
atgggtagccatcaccaccatcaccatggtagcggtagcgagaacttgtacttccaaggaagcatgagcgaagaacagattcgtcagtttctgcgtcgtttttatgaagcgctggatagcggcgatgcggataccgctgcgagcctgtttcatccgggcgtgacaattcatctgtgggatggcgttacctttaccagccgtgaagaatttcgtgaatggtttgaacgtctgtttagcacccgtaaagatgcgcagcgtgaaattaagagcctggaagtacgtggcgataccgtggaagtgcatgtgcagttgcacgcgacccataatggccagaaacataccgtagatgcaacccatcattggcattttcgtggcaatcgtgtgaccgaaatgcgtgtgcatatcaatccgaccggctaa

SEQ ID NO: 194 - protein, 138 aa, synthetic construct
MGSHHHHHHGSGSENLYFQGMSEEQIRQFLRRFYEALDSGDADTAASLFHPGVTIHLWDGVTFTSREEFREWFERLFSTRKDAQREIKSLEVRGDTVEVHVQLHATHNGQKHTVDMTHHWHFRGNRVTEVRVHINPTG

SEQ ID NO: 195 - DNA, 417 bp, synthetic construct
  source 1..417: mol_type = other DNA
atgggtagccatcaccaccatcaccatggtagcggtagcgagaacttgtacttccaaggaatgagcgaagaacagattcgtcagtttctgcgtcgtttttatgaagcgctggatagcggcgatgcggataccgctgcgagcctgtttcatccgggcgtgacaattcatctgtgggatggcgttacctttaccagccgtgaagaatttcgtgaatggtttgaacgtctgtttagcacccgcaaagatgcgcagcgtgaaattaagagcctggaagtacgtggcgataccgtggaagtgcatgtgcagttgcacgcgacccataatggccagaaacataccgtagatatgacccatcattggcattttcgtggcaatcgtgtgaccgaagtgcgtgtgcatatcaatccgaccggctaa

SEQ ID NO: 196 - protein, 138 aa, synthetic construct
MGSHHHHHHGSGSENLYFQGMSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG

SEQ ID NO: 197 - DNA, 417 bp, synthetic construct
  source 1..417: mol_type = other DNA
atgggtagccatcaccaccatcaccatggtagcggtagcgagaacttgtacttccaaggaatgagcgaagaacagattcgtcagtttctgcgtcgtttttatgaagcgctggatagcggcgatgcggataccgctgcgagcctgtttcatccgggcgtgacaattcatctgtgggatggcgttacctttaccagccgtgaagaatttcgtgaatggtttgaacgtctgtttagcaccagtaaagatgcgcagcgtgaaattaagagcctggaagtacgtggcgataccgtggaagtgcatgtgcagttgcacgcgacccataatggccagaaacataccgtagatttgacccatcattggcattttcgtggcaatcgtgtgaccgaagttcgtgtgcatatcaatccgaccggctaa

SEQ ID NO: 198 - DNA, 357 bp, synthetic construct
  source 1..357: mol_type = other DNA
atgagcgaggagcagatcagacagttcctgaggagattctacgaggccctggatagcggagacgccgacacagctgccagccttttccatcctggcgtgaccatccacctgtgggacggcgtcaccttcactagcagggaggagttcagggagtggttcgagagactgttcagcaccagcaaggacgcccagagagagatcaagagcctggaagttagaggcgacaccgtggaagtgcacgtgcagctgcacgccacacacaacggacagaagcacaccgtcgacctgacccaccactggcacttcagaggcaacagagtgaccgaggtgagagtgcacatcaatcccaccggctaa

SEQ ID NO: 199 - skipped

SEQ ID NO: 200 - DNA, 357 bp, synthetic construct
  source 1..357: mol_type = other DNA
  variation 1..3: note = optionally absent
  variation 352..357: note = optionally absent
atgagcgaagaacagattcgtcagtttctgcgtcgtttttatgaagcgctggatagcggcgatgcggataccgctgcgagcctgtttcatccgggcgtgacaattcatctgtgggatggcgttacctttaccagccgtgaagaatttcgtgaatggtttgaacgtctgtttagcacccgtaaagatgcgcagcgtgaaattaagagcctggaagtacgtggcgataccgtggaagtgcatgtgcagttgcacgcgacccataatggccagaaacataccgtagatgcaacccatcattggcattttcgtggcaatcgtgtgaccgaaatgcgtgtgcatatcaatccgaccggctaa

SEQ ID NO: 201 - DNA, 357 bp, synthetic construct
  source 1..357: mol_type = other DNA
  variation 1..3: note = optionally absent
  variation 352..357: note = optionally absent
atgagcgaagaacagattcgtcagtttctgcgtcgtttttatgaagcgctggatagcggcgatgcggataccgctgcgagcctgtttcatccgggcgtgacaattcatctgtgggatggcgttacctttaccagccgtgaagaatttcgtgaatggtttgaacgtctgtttagcaccagtaaagatgcgcagcgtgaaattaagagcctggaagtacgtggcgataccgtggaagtgcatgtgcagttgcacgcgacccataatggccagaaacataccgtagatttgacccatcattggcattttcgtggcaatcgtgtgaccgaagttcgtgtgcatatcaatccgaccggctaa

SEQ ID NO: 202 - DNA, 357 bp, synthetic construct; identical to SEQ ID NO: 198
  source 1..357: mol_type = other DNA
  variation 1..3: note = optionally absent
  variation 352..357: note = optionally absent

SEQ ID NO: 203 - DNA, 357 bp, synthetic construct
  source 1..357: mol_type = other DNA
  variation 1..3: note = optionally absent
  variation 352..357: note = optionally absent
atgagcgaagaacagattcgtcagtttctgcgtcgtttttatgaagcgctggatagcggcgatgcggataccgctgcgagcctgtttcatccgggcgtgacaattcatctgtgggatggcgttacctttaccagccgtgaagaatttcgtgaatggtttgaacgtctgtttagcacccgcaaagatgcgcagcgtgaaattaagagcctggaagtacgtggcgataccgtggaagtgcatgtgcagttgcacgcgacccataatggccagaaacataccgtagatatgacccatcattggcattttcgtggcaatcgtgtgaccgaagtgcgtgtgcatatcaatccgaccggctaa

SEQ ID NO: 204 - DNA, 356 bp, synthetic construct
  source 1..356: mol_type = other DNA
  variation 1..3: note = optionally absent
  variation 354..356: note = optionally absent
atgagcgaacaggcgcagcgcgattttgtgaagaaattttatgaagccctggatgcgggcgatgccgaaactgcaagcgcactgtttccggatggcacccagatttatctgtgggatggccagacctttaccacccaggaacagtttcgcgcgtggtttgtggaactgcgcagcaccagcaaagatgcgaaacgcgaagtggtgagctttaaagtggatggtaacaacgcggaagtggaagttgtgctgcatgcggtgattgatggcgaagaacgcaccgtgaaactgaaacattattttcagtgggaaggcgatcagctggtgaaagtgaccgtgagcattgaaccgctgggtaa

SEQ ID NO: 205 - DNA, 356 bp, synthetic construct
  source 1..356: mol_type = other DNA
  variation 1..3: note = optionally absent
  variation 354..356: note = optionally absent
atgtctgaagaacaaattcgcgaattttgcaaacgcttttatgaagcgctggatgcgggcgatgcggtgactgctagcgcgttgtttccgaatggcacccgcattcatctgtgggatggcattacctttaccacccaggaagaatttcgtgaatggtttgaacgcctgtatagccagagcgaagatgcgaaacgcgaaattgtgagcctggaagtggaaggcaacgtggcgtatgtggaagttattctgcatgcgagctttcgcggtgaagagaaaaccgtgcgcctgcgccatgtttttcagtttgaaggcgataaactggtcgaagtgaccgtggaaattgaaccgctgggtaa

SEQ ID NO: 206 - DNA, 356 bp, synthetic construct
  source 1..356: mol_type = other DNA
  variation 1..3: note = optionally absent
  variation 354..356: note = optionally absent
atgagcgaagaagcgcagcgcgaattttggaaacgcttttatgaagcgctggatgcgggcgatgccgaaactgcagcggcgttgtttcctgatggcaccgaaatccatctgtgggatggtaccacctttcgcacccaggccgaatttcgcgcgtggtttgaagaactgtattcgaccagccagaacgcgaaacgtgaaattgtggaattccgcgtggatggcaacaaaagcaccgtgaccgtggtgctgcatgcgatttacgaaggtgaagaaaagaaagtgctgctggaacattttaccgaatgggaaggcgatcgcctggtgcgcgttgaagtgagcatcgtgccgctgggtaa

SEQ ID NO: 207 - DNA, 356 bp, synthetic construct
  source 1..356: mol_type = other DNA
  variation 1..3: note = optionally absent
  variation 354..356: note = optionally absent
atgtccgaagaagaacagcgtgaatttgtgcgccgcttttatgaagcgctggatgcgggtgatgcggataccgcgagcgcactgtttccagatggcaccgtgattgaactgtgggatggtaccacctttcgcacccgcgcggaatttaaagcgtggtttgaagccctgcatagcaaaagcgaaaacgcggtgcgccatgtggtggaatttgaagtggaaggcaacaaagccaaagtgcgcgtggtgctgcatgctgattataaaggcaagaaacgcgttgtggaactggaacatgaattcgagttcgaaggcgatcgcctggtgaaagtgtctgtggatattcacccgattggtaa

SEQ ID NO: 208 - DNA, 356 bp, synthetic construct
  source 1..356: mol_type = other DNA
  variation 1..3: note = optionally absent
  variation 354..356: note = optionally absent
atgagcgaagaagaaattcgcaaattttgcgaacgcttttatgcggcgctggatgcgggcgatgcggaaactgcaagcagcctgtttcctgatggcaccgaaattcatctgtgggatggcaaaacctttcgcacccaggcggaatttcgcgaatggtttgttcgtctgcgcagcaccagcgatgatgcgcgccgtcatattaccaaactgaaagtggatggtaacgtggcggaagtggaagttgtgctgcgcgcgagctatgatggtgaagaaaaagtggtggcgctgaaacatgtgtttaaatttgaaggcgataaactggttgaagtgaaagttgaaattaccccgctgggtaa

SEQ ID NO: 209 - DNA, 356 bp, synthetic construct
  source 1..356: mol_type = other DNA
  variation 1..3: note = optionally absent
  variation 354..356: note = optionally absent
atgagcgaagaagcccagcgtgaatttgtgcgccgcttttatgaagcgctggatgcgggcgatgcggaaaccgcaagcgcactgtttccagatggcacccagatttatctgtgggatggcaaaacctttaccacccgtgaagaatttcgcagctggtttgtgaaattgcatagcaccagcgataacgcgaaacgccgcgtggttgaatttagagtggaaggcaacaaagcgaaagtaaaagtggtgctgaaagcgagcattaacggtaaagaacgcaccgtgctgctggaacatttctttgaattcgaaggcgataaactggttaaagtagaagtgcgcattcgcccgctgggtaa

SEQ ID NO: 210 - DNA, 356 bp, synthetic construct
  source 1..356: mol_type = other DNA
  variation 1..3: note = optionally absent
  variation 354..356: note = optionally absent
atgtctgaagaagaacagcgcgaattttggcgtcgcttttatgaagcgctggatgcgggcgatgccgaaaccgcaagcgcgttgtttcctgatggcaccgaaatttatctgtgggatggccgcgtgtttcgcacccaggcggaatttcgcgcatggtttgtggaactgcgcagcaaaagcgatgatgcgcgccgcgaagtgaccagctttcgcgtggatggcaacaaagcggatgtgcgcgtggtgctgcatgcgaactataaaggcgaaaagaaagtggtcgaactgagacatgtgtatgaatttgaaggcgatcgcctggtgcgtgtggaagttaccattaacccgctgggtaa

SEQ ID NO: 211 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgaaacgcttttatgaagcgctggatgcaggcgatgcggaaaccgcgagcgcattgtttccggatggcaccaaaattcatctgtgggatggtaccgtgtttaccacccgtgaagaatttaaaagctggtttgtggaactgcgcagcaccagcgatgatgcgaaacgcgaagtggtggatctgaaagtggaaggcaacaaagcgtatgtggaagttgtgctgcgcgcgatcgtgaacggcgaagataaagtggttaaactgcgtcatgtgtttaaatttgaaggcgataaactggtcgaagtgcatgttgaaattgatccgctgggtaa

SEQ ID NO: 212 - DNA, 356 bp, synthetic construct
  source 1..356: mol_type = other DNA
  variation 1..3: note = optionally absent
  variation 354..356: note = optionally absent
atgagcaaagaagaacagcgcaaatttgtggaacgcttttataaagcgctggatgcgggcgatgcggaaactgcgagcgcattgtttccggatggcaccaaaattcatctgtgggatggtaccacctttcatacccgcgcggaatttcgcgcgtggttcgtggatctgcgcagcaaaagcgaaaacgcgaaacgtgaagtggtggcgtttgaagttgatggcaataaagccaaagtggttgtggtgctgaaagcaaactataaaggcgaagaacgcaccgtgaaactggaacatgaattttattttgaaggcgatcatctggtggaagtgaacgttaaaattgaaccagtgggtaa

SEQ ID NO: 213 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaacgcttttatgaagcgctggatgcgggcgatgccgaaactgccagcgcactgtttccagatggcaccgaaattcatctgtgggatggcaaaacctttcatacccgtgaagaatttcgcgcgtggtttgtggatctgtattcgaaaagcaaagatgcgagccgcgaaattaccaaatttgaagtggaaggcaaccgcgcgtttgttgaagttgtgctgcgcgcgagctatgatggcgaagatcgcaccgtgaaactgagacatatttttgaattcgaaggcgatcatctggtggaagtgtatgtggaaattgaaccgctgggtaa

SEQ ID NO: 214 - DNA, 356 bp, synthetic construct
  source 1..356: mol_type = other DNA
  variation 1..3: note = optionally absent
  variation 354..356: note = optionally absent
atgagcgcggaacaaattcgcgaatttgtggcgcgcttttatgcggccttggatgccggtgatgcggatacggcaagcgcgttgtttccggatggcaccgaaattcatctgtgggatggccgcacctttcgcacccaggcggaatttcgcgcatggtttgaaaccctgcgcgcgcagagcgcagatgcgcgtagacatgtggtggcattggaagtgaccggcaacaccgcggatgtggaagttgtgctgcatgcgagcgtggatggcgaagaacgcaccgtggcgctgagacatcgctttcagtttgaaggcgatcgcctggtgcgcgtgaccgttgaaattaccccgctgggtaa

SEQ ID NO: 215 - DNA, 356 bp, synthetic construct
  source 1..356: mol_type = other DNA
  variation 1..3: note = optionally absent
  variation 354..356: note = optionally absent
atgtcggaagaagaacagaaagaatttgtgaaacgcttttatgaagcgctggatgcgggcgatgcggaaaccgcgagcgcattgtttaaagatggcaccgaaatttatctgtgggatggtaccacctttcgtacccgcgcggaatttcgcgcgtggtttcgccgtctgaaaagcaccagcgaagaagcccgccgtagcgtggtgagctttaaagtggatggcaatgtggcggatgtggaagtggtgctgcgtgcgcgttggaaaggtgaagaacgcgtagtgaaactgcgccatcgctttgtgtttgaaggcgatgaagttgtacgcgttgaagtggaaattcgcccgctgggtaa

SEQ ID NO: 216 - DNA, 356 bp, synthetic construct
  source 1..356: mol_type = other DNA
  variation 1..3: note = optionally absent
  variation 354..356: note = optionally absent
atgagcgcggaagaacagcgcgaatttgtgaaacgtttttatgaagcgctggatgccggcgatgcggaaactgcaagcgcactgtttccggatggcaccaaaattcatctgtgggatggtaccgtgtttaacacccaggaagaatttaaagcgtggtttgttaaactgcgcagcaccagcgaaaacgcgaaacgtgaagtgacccattttgaagtggatggcgataaagcggtggtggaagtggtgctgcatgcgaacattaaaggcaaagaaaagaccgtgcgcctgcgccatgaatttctgtttgaaggcgacaaaattgttgaagttaatgtggaaattgaaccgctgggtaa

SEQ ID NO: 217 - DNA, 356 bp, synthetic construct
  source 1..356: mol_type = other DNA
  variation 1..3: note = optionally absent
  variation 354..356: note = optionally absent
atgagcgcggaagcgcagcgcgaatttgtgaagaaattttatgatgcgctggatgcgggcgatgcggaaactgcaagcgcgttgtttccggatggaaccgaaatttatctgtgggatggcaaagtgtttaaaacccaggcggaatttcgcgcgtggtttgtggaactgtatagcaaaagcgataacgcgcagagaagcgtggtgcgttttgaagtggatggtaacgtggcgtatgtggaagtggtgctgcgcgcgaactttgatggcgaagaaaagaccgtgcgcctgcgccatattttctactttgaaggtgatagcctggtgaaagttgaagttaccattgaaccgctgggtaa

SEQ ID NO: 218 - DNA, 356 bp, synthetic construct
  source 1..356: mol_type = other DNA
  variation 1..3: note = optionally absent
  variation 354..356: note = optionally absent
atgagcgaagaagaaattaaagaattttggcgccgcttttatcaggcgctggatgcgggtgatgccgaaaccgcaagcgcattgtttccggatggcaccgaaatttatctgtgggatggtaccacctttcgcacccgcgcggaatttcgtgcatggtttgaagcgctgcgtgcaaccagcgatgcggcgtctagacatattgtgaaactggaagtgaaaggcaaccgcgccgaagtggaagttgtgctgcgcgcgagagttgatggtgaagaacgcgttgtgcgcctgcgccatatttttgaatttgaaggcgatcgtctgaaacgcgtcgaagttgaaattgaaccgctgggtaa

SEQ ID NO: 219 - DNA, 356 bp, synthetic construct
  source 1..356: mol_type = other DNA
  variation 1..3: note = optionally absent
  variation 354..356: note = optionally absent
atgagcgaagaagaaattcgtgaattttggcgccgcttttatgaagcgctggatgcgggcgatgcggaaaccgcaagcgcactgtttccagatggtaccaaaatttatctgtgggatggcattgtgtttagcaccaaagaagagtttcgcagctggtttgtggaactgaaaagcaaaagcgataatgcgaaacgccatgtggtgagcctgcgcgtggatggtaacgtggccaacgtgaaagtggtgctggaagcggaaatcaacggcgaaaagaaagttgtggagctgacccataccaccaaatttgaaggcgataaactggtggaagtgaacgtggatattaacccgctgggtaa

SEQ ID NO: 220 - DNA, 356 bp, synthetic construct
  source 1..356: mol_type = other DNA
  variation 1..3: note = optionally absent
  variation 354..356: note = optionally absent
atgagcgaagaagaacagcgcaaattttgggaaaaattttatgaagcgctggatgcgggcgatgccgaaaccgcaagcgcattgtttaaagatggcaccaaaatttatctgtgggatggtaccgtttttgaaacccaggaagaatttcgcgcgtggtttgaaaaactgtatagcaccagcgataacgcgcagcgccatattgtgagctttaaagtggatggcaacattagctatgtggaagtggtgctgcatgcgaacgtgaacggtgaagaaaagaccgtgaaactgacccacctgttcaaatttgaaggcgatcatgtggttgaggtgaaagtgaaaattgaaccgctgggtaa

SEQ ID NO: 221 - DNA, 356 bp, synthetic construct
  source 1..356: mol_type = other DNA
  variation 1..3: note = optionally absent
  variation 354..356: note = optionally absent
atgagcgaagaagaacagaaagaattttggaaaaagttttacgatgcgctggatgcgggcgatgcagaaactgcgagcgcactgtttccggatggcaccaaaattcatctgtgggatggtaaaacctttaccacccaggccgaatttaaagcgtggtttgtggatctgaaaagcaaaagcgatgatgcgaaacgctatattaccaaattcaaagtggatggaaacaaagcgtatattgaagtggtgctgaaagcgagcgtgaaaggcaagaaaaagaccgtgaaactgaaacataccgcgcagtttgaaggcgataaactggtggaagtggacgtgaccattaacccgctgggtaa

SEQ ID NO: 222 - DNA, 356 bp, synthetic construct
  source 1..356: mol_type = other DNA
  variation 1..3: note = optionally absent
  variation 354..356: note = optionally absent
atgagcgaagaagcgattcgcgaatttgtgcgccgcttttatgaagcgctggatgcaggcgatgcggaaactgcgagcgcactgtttccggatggcacccgcattcatctgtgggatggtaccacctttcgtacccgtgcggaatttcgcgcgtggtttgtggaactgcatagcaaaagcgataacgcgcaacgcgaagtggtgcgcctggaagtggaaggcaaccgtgcaagagttgaagttgtgctgaaagcgaactataaaggcaaagaaaagaccgtgcgtctgcgccatgaatttctgtttgaaggcgacgcgctggtcgaagtgcgcgttgaaattgaaccgctgggtaa

SEQ ID NO: 223 - DNA, 356 bp, synthetic construct
  source 1..356: mol_type = other DNA
  variation 1..3: note = optionally absent
  variation 354..356: note = optionally absent
atgagcgcggaagcgatccgcgaattttggcgccgcttttatgaagcgctggatgcaggcgatgcggaaaccgcaagcgctctgtttccggatggcaccgaaattcatctgtgggatggtaccacctttcgtacgcgcgaagaatttcgcgcgtggtttgtggaactgtatagcaccagcgataacgcgaaacgcaaaattgtgagcctgcgcgttgatggcgatcgcgcgctggttgaagtggtgctgagagcaagcgtgggtggtgaagaaaagaccgtgaaactgaaacatgaagccctgtttgaaggagatcgcctggtggaagtgcgcgtgcagattgaaccgctgggtaa

SEQ ID NO: 224 - DNA, 356 bp, synthetic construct
  source 1..356: mol_type = other DNA
  variation 1..3: note = optionally absent
  variation 354..356: note = optionally absent
atgagcgaagaagcgcagcgtaaatttgtggaacgcttttacaaagcgctggatgcgggtgatgcggataccgcaagcgcgctgtttcctgatggcaccaaaatttatctgtgggatggcaaagtgtttaccacccaggaagaatttcgcgcgtggtttgttgaactgtatagcaaaagcgaaaacgcgaaacgtgaagtggtgtcctttaacgtggatggcgatgtggcggaagtggaagttgtgctgcatgcgaacgtgcgcggcgaactgaaaaccgtgaaattgaaacatacctttaaattcgaaggcgataaactggttgaagtgaacgtgaccattaaaccgctgggtaa

SEQ ID NO: 225 - DNA, 356 bp, synthetic construct
  source 1..356: mol_type = other DNA
  variation 1..3: note = optionally absent
  variation 354..356: note = optionally absent
atgagcgcggaagcgcagcgcgaattttggcgccgcttttatgcggcgttggatgcgggcgatgcggaaactgcaagcgcgctgtttccagatggcaccaaaattcatctgtgggatggcaaaacctttaccacccgcgccgaatttcgcgaatggtttgaaaaactgtatagcacctctgataacgcgaaacgtgaaattaccgaactgaaagtggatggcgataaaagctatgtgaaagttgtgctgaaagcgaacattaacggcgaagaaaagaccgtgaacctgacccatgaatttcagtttgaaggcgacaaactggtggaagtgaacgtgaccattaacccgctgggtaa

SEQ ID NO: 226 - DNA, 356 bp, synthetic construct
  source 1..356: mol_type = other DNA
  variation 1..3: note = optionally absent
  variation 354..356: note = optionally absent
atgagcgaacaggcgattcgcgaattttggaaacgcttttataacgcgctggatgctggtgatgcggataccgcgagcgcgctgtttcctgatggtaccgtgattcatctgtgggatggcaaaacctttcatacccaggcggaatttcgcaaatggtttgtggatctgtattcccgcagcgataacgccaaacgcgaaattgtgagcctggaagtggatggtaaccatgcgaaaattgttgtggtgctgaatgccattattaacggcgaaaagaaaaccgtgctgctgacccatgaaaccctgtttgaaggcgatcatctggtggaagtttttgttgaaattaaaccgctgggtaa

SEQ ID NO: 227 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaacgcttttatgaagcgctggatgcgggcgatgcggaaactgcaagcgcactgtttccggatggcaccaaaattcatctgtgggatggtaccacctttaccacccaggcggaatttcgcgcgtggtttgtgcgcctgcatagcaccagcgaaaacgccagccgtagtattgtgagctttaaagtggaaggcaacaaagcgttggtggaagtggtgctgcgcgcgagcgtgaaaggtgaagaaaagaccgtgaaactgacccatgaatttcagtttgaaggcgataaactggttgaagtgaacgtggatattaaaccgctgggtaa

SEQ ID NO: 228 - DNA, 356 bp, synthetic construct
  source 1..356: mol_type = other DNA
  variation 1..3: note = optionally absent
  variation 354..356: note = optionally absent
atgagcgaagaagatattcgccgctttgtggaacgcttttatgcagcgctggatgcgggcgatgcggaaactgcgagcgcattgtttccggatggcacccgcattcatctgtgggatggaaccacctttaccacccgtgaagaatttcgcgcgtggtttgaaaaactgcacgcgaccagcgaaaacgcgcgccgccatgtggtgaaactgaaagtggaaggtaacaaagcgtatgtggaagtgattctgcatgccagctttaaaggcaaagaaaagaccgttaaactgacccatgtgtttctgtttgaagatgatcgcctggttgaagtgtatgtgaaaattgaaccgctgggtaa

SEQ ID NO: 229 - DNA, 356 bp, synthetic construct
  source 1..356: mol_type = other DNA
  variation 1..3: note = optionally absent
  variation 354..356: note = optionally absent
atgagcgaagaagaacagcgcgaattttgggaaaaattttataacgcgctggatgcgggtgatgccgaaaccgcaagtagcctgtttccggatggcaccgaaatttatctgtgggatggtaccgtgtttaaaacccgtgaagaatttcgcgcgtggtttgaaaaactgcatagcaccagcgaaaacgcgaaacgccatattgtgagctttgaagtggacggcaacaaaagcaaagtgaaagtggtgctgcatgcgaaaattaacggagaagaagtgaccgtggaactggaacatgtgtatgaatttgaaggcgataaactgaaaaaggtggaagttgaaattaaaccgctgggtaa

SEQ ID NO: 230 - DNA, 356 bp, synthetic construct
  source 1..356: mol_type = other DNA
  variation 1..3: note = optionally absent
  variation 354..356: note = optionally absent
atgagcgaaattgcgcagcgcgaattttggcgccgcttttatgcggcgctggatgcgggtgatgcggaaactgcaagcgcgctgtttccagatggcacggaaatttatctgtgggatggccgcacctttcatacccgcgaagaatttgaagcgtggtttcgcgaactgtatagcaaaagcgaaaacgcgcgtcgcgaaatcacctcttttaccgtgaccggcgatattgccgaaattcgcgtggtgctgcgcgcgaccattgatggcgaaaaacgcaccgtgagcctgaaccatgtgacccattttgaaggcgatcagctggtgcgcgtggaagtgagcatttttccgctgggtaa

SEQ ID NO: 231 - DNA, 356 bp, synthetic construct
  source 1..356: mol_type = other DNA
  variation 1..3: note = optionally absent
  variation 354..356: note = optionally absent
atgagcgaagaagaacagaaagaattttggaaacgcttttatgaagcgctggatagcggcgatgcggatacagcgtccgctctgtttccggatggcaccaaaatttatctgtgggacggcaccgtgtttgaaaccaaagcgcagtttaaagcgtggtttgtggaactgtatagccgcagcgataacgcgcagcgcagcattaccaaatttgaagtggatggtaacaccagccgcgtggtggttgtgctgaaagcgaccgtgcgcggcaaagaaaaagtggttgaactggaacatgtgttcgaattcgaaggcgatgaactgaaagaagtgaaagttgaaattcatccgctgggtaa

SEQ ID NO: 232 - DNA, 356 bp, synthetic construct
  source 1..356: mol_type = other DNA
  variation 1..3: note = optionally absent
  variation 354..356: note = optionally absent
atgagcgaagaagcgattcgcgaatttgtgaaacgcttttatgatgccctggatgcgggcgatgcggataccgcctctgccttgtttccagatggcaccctgattcatctgtgggatggcaaaacctttcgtacccaggcggaatttcgcgcctggtttgaagaactgtatagcaaaagcgaaaacgcgaaacgccatgtggtgaaactgcaggtggatggcgatcgcgcgcaggtcgaagtggtgctgcatgcgaacattgatggccaggaagttgtggtgcgcctgcgccatgaatttctgttcgaaggcgatagactggtggaagtgtatgtgcagattcagccgctgggtaa

SEQ ID NO: 233 - DNA, 356 bp, synthetic construct
  source 1..356: mol_type = other DNA
  variation 1..3: note = optionally absent
  variation 354..356: note = optionally absent
atgaccgccgaagcgcagcgcgcgttttgggatcgcttttatcgcgcgcttgatgcgggcgatgcggaaaccgcaagcagcctgtttccagatggcaccgaaattcatctgtgggatggtaaaacctttcatacccgcgaagaatttcgccagtggtttgaaaacctgcgcagcaaaagcgaaaacgcgcgccgccatattgtggattttcgcgtggatggcgatcgcgcagatgtgcgcgtggtgctgcgtgcaaactatcagggtgaagaacgcgttgtgcagctgcatcatgaatttctgtttgaaggcgatcagctggtgcgtgtggaagtgcgcattattccggaaggtaa

SEQ ID NO: 234 - DNA, 356 bp, synthetic construct
  source 1..356: mol_type = other DNA
  variation 1..3: note = optionally absent
  variation 354..356: note = optionally absent
atgagcgaaaaagaacagcgcgaattttgcgcgcgcttttatgcggccctggatgcgggtgatgcggaaactgcaagcgcgttgtttccggatggcacccgcattcatctgtgggatggccgcacctttaccacccaggcggaatttggcgcgtggtttcgtcgtctgcgtagcacaagcgataacgcgcgccgtcatattgtggattttcgcgtggatggcgatgtggcgaaagtgcgcgtagtgttgcatgcgagcgtgggtggtagagaacgcaccgtgcagctggaacatgtgtttatttttgaaggcgatcgcctggtggaagtgcatgtgagcattcagccggaaggtaa

SEQ ID NO: 235 - DNA, 356 bp, synthetic construct
  source 1..356: mol_type = other DNA
  variation 1..3: note = optionally absent
  variation 354..356: note = optionally absent
atgagcgaagaagaacagcgcaaattttgggaaaaattttataacgcgcttgatgcgggcgatgcggaaaccgcaagcgcactgtttccggatggtaccgaaatttatctgtgggatggcaaagtgtttcgcacccgcgcggaatttcgcgaatggtttgaacgcctgtattcgaaaagcgataacgcccagcgcagcattaccagctttgaagtgaacggcaacgtggcggaaattaaagtgattctgaaagcgaacgttaacggtgaagaaaaagaagtgagcctgaaccataaaaccgaatttgaaggcgataaactggtgaaagtggaagtggatattagcccgctgggtaa

SEQ ID NO: 236 - DNA, 356 bp, synthetic construct
  source 1..356: mol_type = other DNA
  variation 1..3: note = optionally absent
  variation 354..356: note = optionally absent
atgagcgagaaagaacagcgcgaattttggaaaaagttttatgaagcgctggatgcgggcgatgcggaaaccgcagcagccttgtttccagatggtaccgtgattcatctgtgggatggcaaaacctttcatacccaggaagaatttaaagaatggtttattaaactgcgcagcacctctgaaaacgcgaaacgcaaaattattagttttaaagtggatggtaacattagctatgttgaagtggaactgaaagcgaaaattaaaggtgaaagcaaagtggtgaaactgagacatatttttaaatttgaaggtgataaactggtggaagtcgatgtgaccattaaaccgattggtaa

SEQ ID NO: 237 - DNA, 356 bp, synthetic construct
  source 1..356: mol_type = other DNA
  variation 1..3: note = optionally absent
  variation 354..356: note = optionally absent
atgagcgcggaagaaattaaagaattttgccgccgcttttatgaagcgctggatgcgggcgatgcggaaaccgcaagcgcgttgtttcctgatggcaccattattcatctgtgggatggccgtacctttcatacccaggcggaatttcgcgcgtggtttcgcgaactgcgcagcaccagcgaagatgcgaaacgcgaaattgaacgcctggaagtggatggcaaccaggccaaagtggaagttattctgcatgcgaaccgcgatggcgaaaagaaagtggtgcgcctgacccatgaatttctgtttgaaggcgatcgcctggttgaagtgaccgtggcgatccgcccgctgggtaa

SEQ ID NO: 238 - DNA, 356 bp, synthetic construct
  source 1..356: mol_type = other DNA
  variation 1..3: note = optionally absent
  variation 354..356: note = optionally absent
atgagcaaacaggaaattaaagatttttgcgaaaaattttataacgcgctggatgcgggcgatgcggataccgcgagcgcactgttcaaagatggcaccaaaatttatctgtgggatggcaaaacctttgaaacccaggcggaatttaaagcgtggtttaccaaactgcatagcaccagcgataatgcgaaacgctatattaccagcctggaagtggatggcgataaagcctttgtgaaagtggtgctgaaagccaacgtgaatggtgaagaaaagaccgtggaactgactcatatttttgaatttgaaggcgatgaactggttaaagtgcaggttaaaattaagccgctgggtaa

SEQ ID NO: 239 - DNA, 356 bp, synthetic construct
  source 1..356: mol_type = other DNA
  variation 1..3: note = optionally absent
  variation 354..356: note = optionally absent
atgagcgaagaagaaattcgccagtttattaaacgcttttatgatgcgctggatgcgggcgatgccgaaactgcgagcgcattgtttccggatggcaccgaaattgatctgtgggatggtaccacctttcatacccaggaagaatttcgctcgtggtttgtgcgtctgaaaagcaccagcgataacgcgaaacgccatgtggtggcgttagaagtgcagggcaaccgtgcgaaagtggaagtggtgctggaagccagcattgatggcaagaaaattaccgtgcagctgacccatgaattttattttgaaggcgataaactggtgaaagtgaacgtgaccattaaaccgctgggtaa

SEQ ID NO: 240 - DNA, 356 bp, synthetic construct
  source 1..356: mol_type = other DNA
  variation 1..3: note = optionally absent
  variation 354..356: note = optionally absent
atgagcgaagaagaaatccgcgaatttgtgcgccgcttttatgcggcgctggatgcgggtgatgcggaaactgcaagcgcgctgtttccagatggcaccgtgattcatctgtgggatggccgcacctttcatacccaggcggaatttcgtgcgtggtttgaagaactgtatagccgcagcgaaaacgcgaaacgtgaagtgacccagctggaagtggatggcgatcatgcgtttgtgaaagtggtgctgcgcgcgaacattcatggtgaagaaaaagttgtgggcctggaacattattttcattttgaaggtgatcagctgaaagaagttgaagtggttattcgtccggaaggtaa

SEQ ID NO: 241 - DNA, 356 bp, synthetic construct
  source 1..356: mol_type = other DNA
  variation 1..3: note = optionally absent
  variation 354..356: note = optionally absent
atgagcaaagaagaaattgaagaattttggaaaaagttttatcaggccctggatgcgggcgatgcggaaaccgcaagcgccttgtttcctgatggcaccgtgattcatctgtgggatggcaaagtgtttcataccaaagcggaatttcgcgaatggtttgtgaaactgaaaagcgagagcgaagatgcgaaacgcgaaattgtgagcctgcgcgtggatggtaacaaagccgaggtggaagtggtgctgaaagcgaaatataaaggcgaagaaaaagaagtgcgcctgaaacatgaatccctgtttgaaggcgatcgcctggtcgaagtggatgtggatatttttccgctgggtaa

SEQ ID NO: 242 - DNA, 356 bp, synthetic construct
  source 1..356: mol_type = other DNA
  variation 1..3: note = optionally absent
  variation 354..356: note = optionally absent
atgagcgaagaagaaatccgcgaattttggaaacgcttttatgaagcgctggatgcgggcgatgccgaaaccgcgagcgcactgtttaaagatggcaccaaaatttatctgtgggatggcattaccttctttacccgcgatgaatttcgcgcgtggtttgaaaaactgtatagcacctcagaagatgcgaaacgcgaaattgtgaaatttaacgtggatggtaacaaagcgaaagtggaagtggtgctgcatgcgattgttgatggccaaaagaaaaccgtgaaactgacccataccagctattttgaaggtgatgaactgaaaaaggtcgaagtgagcattgaaccgatgggtaa

SEQ ID NO: 243 - DNA, 356 bp, synthetic construct
  source 1..356: mol_type = other DNA
  variation 1..3: note = optionally absent
  variation 354..356: note = optionally absent
atgagcgaagaagaaattcgcgaatttattaaacgcttttatgaagcgctggatgcgggcgatgcggaaaccgcaagcgcattgtttccggatggcaccaaaatttatctgtgggatggtaccgtgttttcgacccaggcggaatttcgcgattggtttgtgcgtctgcgtagcaccagccaggatgcccgccgtaaagtggttgatctgaaagtggatggtgatgtggcggatgtggaagtggtgctgcgcgcgaacgtggaaggtgaagaacgcgtggtgcgactgagacatgtgtttcattttgaaggcgataaagttgttgaagtcgaagtggaaattgaaccggaaggtaa

SEQ ID NO: 244 - DNA, 356 bp, synthetic construct
  source 1..356: mol_type = other DNA
  variation 1..3: note = optionally absent
  variation 354..356: note = optionally absent
atgagcgaagaagaacagcgcgaattttggcgccgcttttatgcggcgctggatgcgggtgatgcggaaaccgcatcagcgctgtttcctgatggtaccaaaatttatctgtgggatggcaaaacctttaccacccaggcggaatttcgcgcgtggtttcgcaaactgcgcagccaaagcgaaggcgcgtctagaagcgtggaatattttgaagtggatggtaacaaaagccatgtggaagtggtgctgcgcgccagcgtgaacggcgaacagcatgtggtgaaactgagacatgaatttctgtttgaaggcgataaactggttgaagtcgaagtgaaaattgaaccgctgggtaa

SEQ ID NO: 245 - DNA, 356 bp, synthetic construct
  source 1..356: mol_type = other DNA
  variation 1..3: note = optionally absent
  variation 354..356: note = optionally absent
atgagcgaaaaagcgcagcgcgaattttggaaaaagttttatgaagcgctggatgcgggcgatgccgataccgcgagcgcgctgtttaaagatggcaccgaaatttatctgtgggatggcaaagtgttcaaaaagcaggaagaattcaaagaatggtttgaaaaactgcatagcaccagcgagaacgcgaaacgtaaaattgtgaaatttgaagtgaaaggcaacgtgagctatgtggaagtggtgctggaagcgagcctggcgggtgaaaagaaaaccgtgaaactgaaacacatgtttcagtttgaaggcgataaactggtgaaagtgaccgtggatatttatccgctgggtaa

SEQ ID NO: 246 - DNA, 356 bp, synthetic construct
  source 1..356: mol_type = other DNA
  variation 1..3: note = optionally absent
  variation 354..356: note = optionally absent
atgagcgagaaagcgcagcgcgaatttgtgaaacgcttttatgatgcgttggatgcaggcgatgcggataccgcgagcgcactgtttccggatggcaccattatttatctgtgggatggtaaaacctttcgcacccagaccgaatttcgccgctggtttgtggatctgaaaagcaccagcgaaaacgcgaaacgcaaagttaccgattttcgcgtggatggaaacgtggcgaacgtgaccgtggttctggaagcgaacattaacggcaagaaagaaaccgtgaaactgaaccatatttttcattgggaaggcgatcgcctggtggaagttgaagtgaccattgaaccgctgggtaa

SEQ ID NO: 247 - DNA, 356 bp, synthetic construct
  source 1..356: mol_type = other DNA
  variation 1..3: note = optionally absent
  variation 354..356: note = optionally absent
atgtctgcggaagcgattcgcgaatttgtgcgcgatttttatgaagcgctggatgcgggtgatgcggataccgccagcgcgttatttccggatggcacggaaattcacctgtgggatggtaccgtgtttcgtacccgcgcggaatttaaagcgtggtttaccaaactgtatagcaccagcgaagatgcgcgtcgcaaagtggtgcgtctggaagtggaaggcgatgtggcgcgtgttgaagtggttctgcatgcgagccatgcgggcaaagaaagcaccgtgaaactgcagcatgaatttagctttgaaggtgatcgcctggtggaagttcgtgtggtgattgaaccgctgggtaa

SEQ ID NO: 248 - DNA, 356 bp, synthetic construct
  source 1..356: mol_type = other DNA
  variation 1..3: note = optionally absent
  variation 354..356: note = optionally absent
atgtctgaagaagaaatcaagaaattttgtgaaaaattttatgaagcgctggatgcgggcgatgccgaaactgcgagcgcgttgtttcctgatggcaccgaaatttatctgtgggatggccgcgtgtttaaaacccgcgaagaatttcgcgcgtggtttgtggaactgtatagcaaaagcgataatgcgcagcgcaaaattgtggatctgaaagtggatggtaacaaagcgaaagtgaaagttgtgctgcatgcgaaccataacggcgaacagcataaagtggcgctgacccatgaatttctgtttgaaggcgataaactggtggaagtgaacgtggaaattaaaccgctgggtaa

SEQ ID NO: 249 - DNA, 356 bp, synthetic construct
  source 1..356: mol_type = other DNA
  variation 1..3: note = optionally absent
  variation 354..356: note = optionally absent
atgagcgaagaagcgattcgcgaattttggaaaaagttctatgaagcactggatgcgggcgatgcggaaaccgcaagcgcactgtttccggatggcaccattattcatctgtgggatggtaccacctttaccacccaggatgaatttaaaaagtggtttgtggaactgtttagtaaaagcgaaaacgcgagccgcaaaattgtgaaactggaagtgaaaggcaacgtggcgtatgtggaagttgtgctgaaagccaaaatcgatggcaaagaaaagaccgtgcgcctgaaacatgtgaccaaatttgaaggcgataaactggttgaagtcgaagtggatattgatccgctgggtaa

SEQ ID NO: 250 - DNA, 356 bp, synthetic construct
  source 1..356: mol_type = other DNA
  variation 1..3: note = optionally absent
  variation 354..356: note = optionally absent
atgagcgcggatgatcagaaagaattttggaaacgtttttacgaagcgctggatgcgggcaatgcagatgaagcgagcgcgctgtttccggatggcaccgaaatctatctgtgggatggtaccacctttaaaaccaaagcgcagtttaaagcgtggttctgccagctgcgcagcaccagcgaaaacgcgaaacgtgaaattgttaaatttgaagtggatgggaactttagctatgtggaagtggtgctggaagcgaccgtgaacggcaagaaatttattgtgagcctggatcattattttgaatttgagggcgaacagctggttgaagtctatgtgaaaattcgcccgctgggtaa

SEQ ID NO: 251 - DNA, 356 bp, synthetic construct
  source 1..356: mol_type = other DNA
  variation 1..3: note = optionally absent
  variation 354..356: note = optionally absent
atgtctgcgaaagcgcagcgtgaattttggaaaaagttttatgaagcgctggatgcgggtgatgcggataccgcagcagcactgtttccggatggcacccgcattcatctgtgggatggccgtacctttaccacccaggcggaatttcgcgcgtggtttcagaccctgcatagccgcagcgataacgcgaaacgctctatcgtggcgtttaacgtgagcggtgatgtgagcgatgtggtggttgtgctgcgcgcgagctatgaaggcgaagaacgcaccgtgcgtctgcgtcatacctttcgctttgaaggtgatcatctggtggaagttgatgtggcgattgatccgctgggtaa

SEQ ID NO: 252 - DNA, 356 bp, synthetic construct
  source 1..356: mol_type = other DNA
  variation 1..3: note = optionally absent
  variation 354..356: note = optionally absent
atgagcgaagcggaacagcgcgaattttggcgccgcttttatgaagcgctggatgcgggcgatgcggaaactgcaagcgcgttgtttccagatggcaccgaaattcatctgtgggatggcaaaacctttcatacccaggcggaatttcgcgcgtggtttgtggaattacgcagcacctctgaagcggcgcgccgtcatgtggttgcgtttaacgtggatggcgatcgcgctagagtggaagtcgtgctgcatgcggtgcgtgatggtgaagcgagaaccgtgcagctgacccatgaaaccgtgtttgcgggtgatcaggtggtgcgcgtgcgtgtgcagattaaaccgctgggtaa

SEQ ID NO: 253 - DNA, 356 bp, synthetic construct
  source 1..356: mol_type = other DNA
  variation 1..3: note = optionally absent
  variation 354..356: note = optionally absent
atgagcgaagaagcggtgcgcgaatttgttgcgcgcttttatgaagcgctggatgcgggtgatgcggataccgcgagcgcattgtttccggatggcaccgtgattcatctgtggagcggccagacctttcatacccgcgcggaatttcgcgcgtggtttgaacgcctgtatgcggaaagcgcggcggcgagaagaagagtggtggaaatgcgcgttgatggcgatgtggcgtttgtggaagtggttctgcatgcgagccatcagggccagccgcaggtggttcgtctgagacatatttttcgctttgaaggcgatcgtctgcgtgaagtcgaagtgagcatcgatccgctgggtaa

SEQ ID NO: 254 - DNA, 356 bp, synthetic construct
  source 1..356: mol_type = other DNA
  variation 1..3: note = optionally absent
  variation 354..356: note = optionally absent
atgagcgcggaacagcagcgcgaattttgggatcgcttttatgcggcgctggatgcgggcgatgcggataccgcaagcgcattgtttaacgatggcaccgaaatttatctgtgggatggcaaagtgtttcatacccaggcggaatttaaagcgtggtttgtggatctgcgcagccgcagcaccggcgcgagcagagaaattgtggcgtttaaagtgaccggcaaccgcgccgaaatcgaagtggtgctgcatgcagatgttgatggccagccgaaaaccgtgcgcctgacccattataccgaatttgaaggcgatcgcctggtgcgcgtagaagtgaaaattaatccgctgggtaa

SEQ ID NO: 255 - DNA, 356 bp, synthetic construct
  source 1..356: mol_type = other DNA
  variation 1..3: note = optionally absent
  variation 354..356: note = optionally absent
atgagcgaacaggcgattcgtgaatttgtgcgccgcttttatgatgcgctggatgcgggtgatgccgaaaccgcagctgcactgtttccggatggcaccgtgattcatctgtgggatggccgcacctttcatacccgcgcggaatttcgcgcgtggtttgtgaggctgcgtagcactagcgatgatgcccgccgtagggtggtggaactgcgtgtggttggtgatcgtgctagagtgcgcgttgtgctgcaggccaacgtggatggcgaaccgagagtggttgaactggaacacgaatttgaattcgaaggcgatcgtctgcgcgaagtgcgtgttgatattcgcccgctgggtaa

SEQ ID NO: 256 - DNA, 356 bp, synthetic construct
  source 1..356: mol_type = other DNA
  variation 1..3: note = optionally absent
  variation 354..356: note = optionally absent
atgagcgcggaagaacagcgtaaatttgtggaaaaattttataccgcgctggatagcggcgatgccgaaaccgcgagcagcctgtttcctgatggcaccctgatttatctgtgggatggccgcgtgtttcatacccaggcggaatttcgcgcgtggttcgaaaaactgtatagccagagcgcgaacgcgaaacgcagcgtggtgcgctttgaagtggaaggcaatgaagcgtacgtggaagtggtgctgcatgcggtggtggatggcaaagaaaccgtggtgagactgaaacataactatctatttgaaggcgatcgcctggttcgcgtcgaagttgaaattaaaccgatgggtaa

SEQ ID NO: 257 - DNA, 356 bp, synthetic construct
  source 1..356: mol_type = other DNA
  variation 1..3: note = optionally absent
  variation 354..356: note = optionally absent
atgagcgaagaagcgcagcgcgaatttgcgaaacgcttttatgcggcgctggatgcgggtgatgcggataccgcagcagcactgtttccagatggcaccgaaatttatctgtgggatggcaaaacctttaccacccgcgcggaatttcgcgcgtggtttgaagaactgcgcagcaccagcgatcgcgcgaaaaggcgcgttgatcgttttgaagtgaacggcgatacggcgcatgtggaagtgaccctggacgcgtatgttaacggtgaaaaccgtaccgtgcgcctgcgccatgtgtttcatttcgaaggcgatcgtgtgaaacgcgtcgaagtggaaattaaaccgctgggtaa

SEQ ID NO: 258 - DNA, 356 bp, synthetic construct
  source 1..356: mol_type = other DNA
  variation 1..3: note = optionally absent
  variation 354..356: note = optionally absent
atgtccgaagaagaaattcgcgaatttgtgcgccgcttttatgaagcgctggatgcgggtgatgcggcaaccgcaagcgcattgtttccggatggcaccgaaattcatctgtgggatggtaccacctttcgcacccaggcgcagtttcgcgcgtggtttgaacgcctgcgtgcacaaagcgcaaacgcgcgcagagaaattgtcgatctgaaagtggaaggcgatcgcgcgaaagttgaagtgattctgcgcgcgagctttgatggtgaagaaaaagtggtgaacctgacccatgaatttctgtttgaaggtgatcgcctggtgcgcgtgagcgtgaccattaccccgctgggtaa

SEQ ID NO: 259 - DNA, 356 bp, synthetic construct
  source 1..356: mol_type = other DNA
  variation 1..3: note = optionally absent
  variation 354..356: note = optionally absent
atgagcgaagaagaaattaaagaattttggaaacgcttttatgaagcgctggatgcgggcgatgcggaaaccgcaagcgcattgtttccggatggcaccgaaattcatctgtggagcggcaaaacctttcgcacccgtgaagaattccgcgcgtggtttgaagaactgtatagccagagcgaaaacgcgaaacgccatattgtgagcctggaagtggatggcgatgaagcctatgtgcgcgttgtgctgcatgcgtttgtgaaaggcgaaagccgcaccgtggaactggaacatttctttcgctttgaaggtgatcatctggtggaagttaaagtggacattattccgctgggtaa

SEQ ID NO: 260 - DNA, 356 bp, synthetic construct
  source 1..356: mol_type = other DNA
  variation 1..3: note = optionally absent
  variation 354..356: note = optionally absent
atgagcgaagcggcgattcgcgaatttgttgataaattttatgcggcgctggatgcgggcgatgcggaaacagcagcaagcctgtttccagatggcaccaaaatttatctgtgggatggccgcacctttaccacccaggcggaatttaaagcgtggtttgaagaactgcatgcgaccagcgataacgcgcgccgcgaagtggtgagcatggaagttgatggcgatgtggcagatgtcgaagtcgtgctgcacgcgagctttcgcggcgaacagcgtgaagttcgtctgcgtcatgtgtttcattttgaaggtgatgaactgcgggaagtggaagtagaaattctgccgctgggtaa

SEQ ID NO: 261 - DNA, 356 bp, synthetic construct
  source 1..356: mol_type = other DNA
  variation 1..3: note = optionally absent
  variation 354..356: note = optionally absent
atgagcgaagaacagcagcgcgaattttgggcgcgcttttatgcggcgttagatgcgggtgatgcggataccgcgagcgcgctgtttccagatggcaccaaaatccatctgtgggatggcaaaacctttaccagccgcgcggaatttcgcgattggtttgtgcgcctgcatagccgctctgaaaacgccaaacgccgcattacgagctttaaagtggaaggcgatattagctatgtggaagtggtgctgcatgcgagccgcgatggccaggaacatgtagtgaaactgaaacatgtggcgaaatttgaaggtgatgaactggttgaagtgaaagttgaaattaccccgctgggtaa

SEQ ID NO: 262 - DNA, 356 bp, synthetic construct
  source 1..356: mol_type = other DNA
  variation 1..3: note = optionally absent
  variation 354..356: note = optionally absent
atgagcaaagaagaacaaaagaacttttgcaaaaagttttatgaagcgctggatgcgggtgatgcggaaaccgctagcagcctgtttccggatggcaccaaaatttatctgtgggatggtaaaacctttagcacccaggaagaatttcgcgcgtggtttgaaaaactgcgcagcgaatcggcgaatgcgagccgccgtattgtgagctttaacgtggatgggaacgtggcgaaagtgaaagtggtgctgaaagcgaactatcgcggcaagaaaagcaccgtgaaactggaacataaatttgaattcgaaggcgataaactggttaaagtagaagtgaccattgaaccgctgggtaa

SEQ ID NO: 263 - DNA, 356 bp, synthetic construct
  source 1..356: mol_type = other DNA
  variation 1..3: note = optionally absent
  variation 354..356: note = optionally absent
atgagcgaagaagcgattcgcgaatttgtgaaacgcttttatgaagcgctggatgcgggtgatgcggataccgcaagcgcgttgtttccggatggcacccgcatttatctgtgggatggtaccacctttcgcacccaggcggaatttcgcgcgtggtttcgtgaactgtatagcaaaagcgaaggcgcgcgccgcgaagttattaacctgaaagtggatggtgatgtggcgtatgtggaagtggtgctgcatgcggtgtataaaggtgaagaaaagaccgtgaaactggtgcatgtgtttaaatttgaaggcgataaagtggttgaagtgcacgtggaaattgtgccgctgggtaa

SEQ ID NO: 264 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgaaacgcttttataccgcgctggatgcgggcgatgcggaaactgcgagcgcactgtttccagatggcacgaaaatttatctgtgggatggtaccacctttgaaacccgtgaaggctttgcggcgtggtttcgcaaactgaaaagccagtcggaagaagcgaaacgccatgtggtgaaattgaaagtggaaggcaacgtggcctatattgaagtggtgctgcatgcgaaacataaaggtgaagaaaagaccgtgcgcctgacccatatctatcagtttgaaggcgataaactggttgaagttcgcgtggaaattaaaccgctgggtaa

SEQ ID NO: 265 - DNA, 356 bp, synthetic construct
  source 1..356: mol_type = other DNA
  variation 1..3: note = optionally absent
  variation 354..356: note = optionally absent
atgagcgaagcggaaattaaaaactttgtgaaacgcttttatgaagcgctggatgcaggcgatgcggaaaccgctagcgcactgtttccggatggcaccgaaattcatctgtgggatggtaccacctttcgcacccgcgccgaatttcgcgcgtggtttgaagaactgtatagccgcagcgaagatgcgaaacgcgaagtgaccaaactggaagtgaaaggcaacgtggcgtttgtggaagttgtgctgaaagcgaacctgaacggcaaagaacgcaccgtgaaactgaaacatatttttcattttgaaggcgatagcctggtgcgcgttgaagtgtcgattgtgccgctgggtaa

SEQ ID NO: 266 - DNA, 356 bp, synthetic construct
  source 1..356: mol_type = other DNA
  variation 1..3: note = optionally absent
  variation 354..356: note = optionally absent
atgagcgaacaggaacagaaagaattttgggaaaaattttatcaggcgctggatgcgggcgatgcggaaaccgcgagcgcgttgtttaaagatggcaccgaaatttatctgtgggatggcaaagtgttcaaaacccaggaagaattcaaagcgtggttcgtggaactgtatagcaaaagcctgaacgcgaaacgcaaaattgtggaacatgaagtgaaaggcaacgaatcttatgtgaaagtggtgctgcgcgccagcgtggatggcgaagaacgcaccgtgttgttggaacacaaatttctgtttgaaggcgataaactggttaaagtgagcgttgaaattaccccgctgggtaa

SEQ ID NO: 267 - DNA, 356 bp, synthetic construct
  source 1..356: mol_type = other DNA
  variation 1..3: note = optionally absent
  variation 354..356: note = optionally absent
atgaccgcggaagaacagcgcgaatttgtgaaacgcttttatgaagcgctggatgcgggcgatgcggaaactgcgagcgctctgtttccagatggcaccctgattcatctgtgggatggcaaaacctttcatacccgcgaagaatttcgcgcgtggtttgtggaactgcgcagcaccagcgatgatgcgaaacgcgaagtgaccgcctttgaagtggatggcgatgtggcgcgcgttgaagttgtgctgcatgcgaacattcagggcaacaaaaagaccgtcgcgctgacccacgaatttctgtttgaaggcgataaactggtggaagtgcgtgtggatattaaaccgctgggtaa

SEQ ID NO: 268 - DNA, 356 bp, synthetic construct
  source 1..356: mol_type = other DNA
  variation 1..3: note = optionally absent
  variation 354..356: note = optionally absent
atgagcgaagaagaacagaaaaactttgtgcgcaaattttatgatgcgctggatgcgggcgatagcgaaaccgcgagcgcgctgtttaaagatggcaccaaaatttatctgtgggatggccaggtttttgaaacccaggaagaatttcgcgcgtggtttgaaaaattgtatagcaccagcaccgatgccaaacgcgaaattgtgagctttaaagtggatggcaacaaagcggtggttgaggtggtgctgaaagcgattaaagacggtgaagaaaaagtggtgaacctgaaacatgtgtttctgtttgaaggtgatgaagtggtagaagtggaagttgatattgtgccgagcggtaa

SEQ ID NO: 269 - DNA, 356 bp, synthetic construct
  source 1..356: mol_type = other DNA
  variation 1..3: note = optionally absent
  variation 354..356: note = optionally absent
atgaccgcggaacagcagcgcaacttttgggcgcgcttttatgcggcgctggatgcgggtgatgcggaaactgcagcagccttatttcctgatggcaccgtgattcatctgtgggatggccgcacctttcatacccaggcggaatttcgcgcgtggtttgaagcgctgcgtagcaccagcgaaaacgcgcgtcgtgaaattgtgtttttccaggtggatggcaacaccgccgatgtggaagtggtgctgcatgcgagcgtggacggtgacgcaagaaccgtgcgtctgagacatatttttcagttcgaaggcgatcatctggttgaagtgcatgtggatattcgcccgctgggtaa

SEQ ID NO: 270 - DNA, 356 bp, synthetic construct
  source 1..356: mol_type = other DNA
  variation 1..3: note = optionally absent
  variation 354..356: note = optionally absent
atgagcgaagaagaacaaaagaactttgtggcggcgttttataaagcgctggatgcgggcgatgccgaaactgcgagcgcgttgtttccagatggcaccgaaattcatctgtgggatggcaaaacctttaaaacccaggccgaatttaaagcgtggtttgaaaaactgtttagcaccagcgataacgcgaaacgcaaagtggtgagctttaaagtgaacggcaacattgcggatgtgcaggtggtgctggaagcgaatattaacggcgaaccagtgaaagttaaactgaaccataccttcaaattcaaaggcgataaactggtggaagttaacgtgcagattgaaccgctgggtaa

SEQ ID NO: 271 - DNA, 356 bp, synthetic construct
  source 1..356: mol_type = other DNA
  variation 1..3: note = optionally absent
  variation 354..356: note = optionally absent
atgagcgaagaagaaattcgtgaatttgtgcgcaaattttatgcggcgctggatgcgggtgatgcggataccgcaagcgcattgtttccggatggcaccgaaattcatctgtgggatggcgtgacctttaaaacccaggcggaatttaaagcgtggtttaccgaactgaaaagcaaaagcgataacgcgaaacgcgaggtggtgaaattgaaagtggatggtaacaaagcggatgtggaagtggtgctgcatgcgaccattgatggcaaagaagtgaccgtgcatctgcgccatatttttgaattcgaaggcgataaactggttaaagttgaagttgtgattgaaccgctgggtaa

SEQ ID NO: 272 - DNA, 356 bp, synthetic construct
  source 1..356: mol_type = other DNA
  variation 1..3: note = optionally absent
  variation 354..356: note = optionally absent
atgagcgcggaagcgcagcgtgaatttgtgaaacgcttttatgatgccctggatgcgggtgatgcggataccgcgagtgcgttgtttgccgatggcacccgcatttatctgtgggatggccgcgaatttcgcacccgtgcggaatttagagcgtggtttgaacgtctgcgcagccgcagcgatgcggcgaaacgtcatgtgaccagctttaaagtggaaggcaacgtggcgcaggtggtggttgtgctgaaagcgaactttcgcggcaaagaacatgtggtggaactgctgcatgaattcgtgtttgaaggcgatcgcctggttgaagtgcatgtggaaattcgcccgcgcggtaa

SEQ ID NO: 273 - DNA, 356 bp, synthetic construct
  source 1..356: mol_type = other DNA
  variation 1..3: note = optionally absent
  variation 354..356: note = optionally absent
atgagcgaagaagaaatccgcgaatttgtgaaacgcttttatgaagcgctggatgcgggtgatgcggaaaccgcaagcgcactgtttccggatgggaccaaaattcatctgtgggatggcattacctttaacacccaggaagaatttcagaaatggtttgtggaactgcgcagccagagcgataacgcaaaacgtgaagtggtgagcctgaaagtggatggtaaccatgcgaaagttgaagttgtgctgaaagcgaacattggcggcgaagatcgcgtggtgaaactgacccatcattttctgtttgaaggcgataaactgattgaagtgcgcgtggaaattgaaccgctgggtaa

SEQ ID NO: 274 - DNA, 356 bp, synthetic construct
  source 1..356: mol_type = other DNA
  variation 1..3: note = optionally absent
  variation 354..356: note = optionally absent
atgagcgcggaagcgattcgtgaatttgttgaacgtttttatgcggcgctggatgcgggcgatgcggaaactgcaagcgcactgtttcctgatggcaccattattcatctgtgggatggccgcgtgtttcatacccgcgaagaatttcgccgctggtttgtggatttgtttagcaccagcgataacgcgagccgcagcgtggtggatctgcatgtggatggcaacgtggctcatgtggtggtgcgcctgaaagcgagctggcgtggcaagaaacgcaccgtgttgctgacccatgtgtttgaattcgaaggtgatcatctggtgaaagtgaccgtgagcattcagccggtgggtaa

SEQ ID NO: 275 - DNA, 356 bp, synthetic construct
  source 1..356: mol_type = other DNA
  variation 1..3: note = optionally absent
  variation 354..356: note = optionally absent
atgagcgcggaagaaattcgcgaatttgtggcgcgcttttatgcggcgttggatgcgggtgatgcggataccgcaagcgcgctgtttccgaacggtaccaaaatttatctgtgggatggcaccgtgtttaccacccaggcggaatttcgcgcgtggtttgtgaaactgcgcagcaccagcgaaaacgcgaaacgtgaagtggtgtttctggaagtggaaggcaacgaagcgtttgtggaagttgtgctgcatgcggaaattaaaggccagaagaaaaccgtgcgcctgcgccatgtgtttaaatttgaaggcgatcatctggttgaagtgtctgtggatattgaaccgctgggtaa

SEQ ID NO: 276 - DNA, 356 bp, synthetic construct
  source 1..356: mol_type = other DNA
  variation 1..3: note = optionally absent
  variation 354..356: phenotype = optionally absent
atgagcaaagaagatattgaagaattttgcaaaaagttttatgatgcgctggatgcgggcgatgccgaaacagcaagcgccctgtttccagatggcaccgaaattaacctgtgggatggcaaagtgtttaccaccaaaagcgaatttcgcgcgtggtttcgcgaactgtatgcccgcagcgatcatgccaaacgtgaaattacccatctggaagtggatggtaactttgcgaccattgaagtggtgctgaacgcgagcgtggacggcgaacagaaaaccgtgagcctgaaacatttctttcagtttgaaggcgatcgcctggtgcgcgttgatgtgaaaatcaacccgctgggtaa

SEQ ID NO: 277 - DNA, 356 bp, synthetic construct
  source 1..356: mol_type = other DNA
  variation 1..3: note = optionally absent
  variation 354..356: note = optionally absent
atgagcgaagaagaaattcgcgaattttggcagcgcttttatgaagcgctggatgcgggcgatgcggaaaccgcgagcgcattgtttcctgatggcaccgaaatttatctgtgggatggcaaaacctttcgcacccgcgcggaatttcgcgcgtggtttgaagaactgcatagcaccagcgaaaacgccaaacgtcatgtgaccgctctggaagtggatggtaacgtggcgcgcgttcaggtggtgctgcatgcgagcattaatggtgaagaaaagaccgtgaaactggatcatgaaacccattttgaaggtgatcgcctggtgcgcgtgaccgtggatattaaaccgctgggtaa

SEQ ID NO: 278 - DNA, 356 bp, synthetic construct
  source 1..356: mol_type = other DNA
  variation 1..3: note = optionally absent
  variation 354..356: note = optionally absent
atgagtgaagaagaacagcgcaactttgtgaaacgcttttatgaagcgctggatgcgggcgatgcggaaaccgcgagcgcgttatttcctgatggcaccgtgattcatctgtgggatggtaccacctttcatacccaggcggaatttcgcgcgtggtttaccgaactgcgcagcaccagcgaaaacgcgaaacgtgaagtgaccaaatttgcggtggatggcaacgtggcgcatgtggaagttgtgctgcatgcgaaccataacggtgaaccgcgcgttgtgcgcctgacccatgaatttcattttgaaggcgatcatctggttgaagttaccgtgcgcatcgatccgctgggtaa

SEQ ID NO: 279 - DNA, 356 bp, synthetic construct
  source 1..356: mol_type = other DNA
  variation 1..3: note = optionally absent
  variation 354..356: note = optionally absent
atgagcgaagaagaacagcgcgaatttgtgcgccgcttttatgaagcgctggatgcgggcgatgcggaaactgcaagcgcgctgtttccagatggcaccgtgatcgatctgtgggatggcaaaacctttcatacccgcgccgaatttcgcgaatggtttgtgaaactgaaaagcaccagcgataacgcgaaacgtgaagtgaccaactttaaagtggatggcgatgtggcgcatgtggaagtggtgctgaaagcgagcattaacggtgaagaaaaagtggttaaactgacccataccttccagtttgaaggcgatcgcctggtgcgcgtgagcgtgaaaattgaaccgctgggtaa

SEQ ID NO: 280 - DNA, 356 bp, synthetic construct
  source 1..356: mol_type = other DNA
  variation 1..3: note = optionally absent
  variation 354..356: note = optionally absent
atgtcggaagaagaacagaaagaattttgcaaaaagttttatgaagcattggatgcgggtgatgccgatacggcgagcgccctgtttccagatggcaccaaaatccatctgtgggatggcaaaacctttaccacccaggcgcagtttaaagcctggtttgtggaactgtatagcaaaagcgaaaacgccaaacgtgaaattacgaaatttgaagtggatggtaacgtggcggatgtggaagtggtgctgcatgcgatcgtgaacggcgaaaagaaaaccgtgaaactgaaacatgtttttaaattcgaaggtgataaactggttgaagtcaacgtcgaaatcaaaccgctgggtaa

SEQ ID NO: 281 - DNA, 356 bp, synthetic construct
  source 1..356: mol_type = other DNA
  variation 1..3: note = optionally absent
  variation 354..356: note = optionally absent
atgagcgcggaagaaattaaaaacttctgcgacaaattttataaagcgctggatgcgggcgatgcggataccgcaagcgccttgtttccagatggcaccgaaatttatctgtgggatggcaaaacctttaaaacccaggcggaatttaaagcgtggtttgaaaaactgtatagcaccagcaaagatgcgaaacgcaaaattaccagcctggaagtgaatggcaatgttgcgaaagtgaaagtggtgctggaagcgattattgatggcgaaaagaaaaccgtgaacctggaacatattttctattttgaaggcgataaactggtcaaagttgaagtgagcattgaaccgctgggtaa

SEQ ID NO: 282 - DNA, 356 bp, synthetic construct
  source 1..356: mol_type = other DNA
  variation 1..3: note = optionally absent
  variation 354..356: note = optionally absent
atgagcgaagaagaacagcgcgaatttgtggcgcgcttttatgcggcgctggatgcgggtgatgcggaaactgcaagcgcgttgtttccggatggcaccgaaattcatctgtgggacggcaaaacctttaccacccgcgcggaatttcgcgcgtggtttgaaaaattgcatagcctgagcgataatgcgagccgccatgtgaccagctttaaagtggatggtaacgtggcggaagtggaagttgtgctgcatgcggattttaaaggcaagaaactgaccgtgaaattgcgccatcgctatcagtttgaaggcgatcgcctggtgcgcgtcgatgtggaaatttttccgctgggtaa

SEQ ID NO: 283 - DNA, 356 bp, synthetic construct
  source 1..356: mol_type = other DNA
  variation 1..3: note = optionally absent
  variation 354..356: note = optionally absent
atgagcgcggatgcgcagcgcgaatttgtgcgccgcttttatgaagcgctggatgcgggtgatgcggaaaccgcagccgcgttgtttcctgatggcaccgaaattcatctgtgggatggcaaaacctttcgcacccaggcggaatttcgtgcgtggtttgaaaaactgcgcgcgcagagcgaaaacgcgaaacgccatgtggtgaactttcaggtggatggtaacgatgccgatgtggaagtggtgctgcatgcgacctataacggcgaacagaaaaccgtgcgcctgaaacataaatttcattttgaaggcgatcagctggtgcgcgttgatgtgaccattgaaccgctgggtaa

SEQ ID NO: 284 - DNA, 356 bp, synthetic construct
  source 1..356: mol_type = other DNA
  variation 1..3: note = optionally absent
  variation 354..356: note = optionally absent
atgagcgaagaacaaattcgccagtttctgcgccgcttttatgaagcgttagattctggcgatgcggataccgcggccagcctctttcatccgggcgtgaccattcatctgtgggatggcgttacctttaccagccgtgaagaatttcgcgaatggtttgaacgcctgtttagcacccgcaaagatgcgcaggcggaaattaaaagcctggaagtgcgcggcgataccgtggaagttcacgtgcagctgcatgcgacccataacggccagaaacataccgttgatgcgacgcatcattggcattttcgcggcaaccgcgtgacggaaatgcgcgtgcatattaacccgaccggtaa

SEQ ID NO: 285 - DNA, 356 bp, synthetic construct
  source 1..356: mol_type = other DNA
  variation 1..3: note = optionally absent
  variation 354..356: note = optionally absent
atgagcgaagaacaaatccgccagtttctgcgccgcttttatgaagcgctggatagcggcgacgcggataccgcagcgagcttatttcatccgggcgtgaccattcatctgtgggatggcgttacctttaccagccgtgaagaatttcgcgaatggtttgaacgcctgtttagcaccagcaaagatgcgcagcgcgaaattaaaagcctggaagtgcgcggcgataccgtggaagttcatgtgcagctgcatgcgacccataacggccagaaacataccgttgatctgacccatcattggcattttcgcggcaaccgcgtgacggaagtgagagtgcatattaacccgaccggtaa

SEQ ID NO: 286 - DNA, 356 bp, synthetic construct
  source 1..356: mol_type = other DNA
  variation 1..3: note = optionally absent
  variation 354..356: note = optionally absent
atgagcgaagaagaacagaaagaattttgcaagaaattttatgaagcgctggatgcgggtgatgcggataccgcaagcgcactgtttcccgatggtaccaaaatccatctgtgggatggcaaaacctttaccacccaggcgcagtttaaagcgtggtttgtggaactgtacagcaaaagcgaaaatgcgaaacgtgaaattaccaaatttgaagtggatggtaacgtggcggatgtggaagtggtgctgcatgcgattgtgaacggcgaaaagaaaaccgtgaaactgaaacatgtgtttaaattcgaaggcgataaactggttgaagttaatgttgaaatcaaaccgctgggtaa

SEQ ID NO: 287 - DNA, 356 bp, synthetic construct
  source 1..356: mol_type = other DNA
  variation 1..3: note = optionally absent
  variation 354..356: note = optionally absent
atgagcgcggaagaaattaaaaacttttgcgataaattttataaagcgctggatgcgggtgatgcggataccgcgagtgcactgtttcctgatggcaccgaaatttatctgtgggatggcaaaacctttaaaacccaggcggaatttaaagcgtggtttgaaaaactgtatagcaccagcaaagatgcgaaacgtaaaattaccagcctggaagtgaacggcaacgttgcgaaagtgaaagtggtgctggaagcgattattgatggcgaaaagaaaaccgttaacctggaacatattttctattttgaaggtgataaactggttaaagtggaagtatctattgaaccgctgggtaa

SEQ ID NO: 288 - DNA, 356 bp, synthetic construct
  source 1..356: mol_type = other DNA
  variation 1..3: note = optionally absent
  variation 354..356: note = optionally absent
atgagcgaagaagaacagcgtgaatttgtggcgcgcttttatgcggcgctggatgcgggtgatgcggaaactgcaagcgcgctgtttcctgatggcaccgaaattcatctgtgggatggcaaaacctttaccacccgcgcggaatttcgcgcgtggtttgaaaaattgcatagcctgagcgataacgcgagccgccatgtgaccagctttaaagtggatggtaacgtggcggaagtggaagttgtgctgcatgcggattttaaaggcaaaaagctgaccgtgaaactgagacatcgctatcagtttgaaggcgatcgcctggtgcgcgttgatgtggaaatttttccgctgggtaa

SEQ ID NO: 289 - DNA, 356 bp, synthetic construct
  source 1..356: mol_type = other DNA
  variation 1..3: note = optionally absent
  variation 354..356: note = optionally absent
atgagcgaagaagagattcgccagtttgtgaagaaattttatgaagcgctggatgcaggtgatgcggaaaccgcaagcgccctgtttccggatggcaccaaaatttatctgtgggatggtaccgtgtttaacacccaggcggaatttaaagcgtggtttgtggaactgtattcgaccagcgaaaacgcgaaacgtgaagtggtgaaatttgaagtcgatggtaacaaagcgaaagtggaagtcgtgctgcatgcgagcattgatggcgaggaaaagaccgtgaaactgaaacatgaattttactttgaaggcgataaagtgaaagaagttaaagttgaaattgaaccgctgggtaa

SEQ ID NO: 290 - DNA, 356 bp, synthetic construct
  source 1..356: mol_type = other DNA
  variation 1..3: note = optionally absent
  variation 354..356: note = optionally absent
atgtccgaagaagaacagcgcgaattttggcgccgcttttatgaagccctggatgcaggcgatgctgaaaccgcgagcgcgttatttccggatggcaccgaaatttatctgtgggatggccgtacctttcgcacccaggcggaatttcgcgcatggtttgaacgcctgcgcgccactagcgaagatgcgcgcagagaaattgtgagctttcgcgtggaaggcaatcgcagcgaagtggaagttgtgctgcatgcgaacgtgaacggcgaaaagaaaaccgtgcgcctgagacattattttgaatttgaaggcgatcgcctggtgcgcgttgaagttgaaatcgtgccgctgggtaa

SEQ ID NO: 291 - DNA, 356 bp, synthetic construct
  source 1..356: mol_type = other DNA
  variation 1..3: note = optionally absent
  variation 354..356: note = optionally absent
atgagcgaagaagaaattaaagaattttgcaaaaagttttatgaagcgctggatgcgggcgatgcggataccgcgagcgcgctgtttaaagatggcaccgaaatttatctgtgggatggtcgtacctttaaaacccgcgcggaatttaaagcgtggtttacccgtctgcgcagcacctctgataacgcgaaacgtgaaattgtgagcctgtcggtgaacggcaacgtggcgaaagtgaaagtggtgctgcgcgcgagcattaacggtgaagaacgcaccgtgctgctgacccatgaatttctgtttgaaggcgatgaactggtggaagtgcgcgtgagcatcaaaccgctgggtaa

SEQ ID NO: 292 - DNA, 356 bp, synthetic construct
  source 1..356: mol_type = other DNA
  variation 1..3: note = optionally absent
  variation 354..356: note = optionally absent
atgagcaaagaagaacaagaggaatttgtgaaacagttttatgaagcgctggatgcgggcgatgcggaaaccgcaagcgcactgtttccagatggcaccgtgatccatctgtgggatggcaaaacctttcatacccaggcggaatttcgcgcgtggtttgaagaactgaaaagcaccagcgaaaacgcgaaacgtgaagtgaccaaatttgaagtggatggcgatgtggcggatgtggaagtggtgctgaaagcgaacattaacggcgaagaaaaagtggttaacctgaaacataaatttaaattcgaaggcgataaagttgtcgaagtttgggtcgaaattgaaccgctgggtaa

SEQ ID NO: 293 - DNA, 356 bp, synthetic construct
  source 1..356: mol_type = other DNA
  variation 1..3: note = optionally absent
  variation 354..356: note = optionally absent
atgagcgaagatgccattcgcgaatttgtgaaacgtttttatgaagcgctggatgcgggcgatgcggataccgcgagcgcattatttccggatggcaccgaaattcatctgtgggatggccgcgtgtttcgcacccgcgcggaatttcgtgcctggtttgtggaactgcgcagccagagcgaaaacgcgaaacgtgaagtgaccgatctggaagtggaaggcaacgtggcgcgcgtggaagtcgtgctgcatgcgaactataaaggcgaagaacgcaccgtgcgcctgcgccatgaatttcagtttgaaggcgataaagtggttgaagtgcgcgttgaaattgaaccgctgggtaa

SEQ ID NO: 294 - DNA, 356 bp, synthetic construct
  source 1..356: mol_type = other DNA
  variation 1..3: note = optionally absent
  variation 354..356: note = optionally absent
atgtcagaagaagaacagaaagaattctgccgccgcttttatctggcgctggatgcgggtgatgcggataccgcaagcgcactgtttaaagatggcaccaaaatttatctttgggatggcaaagtgtttaacacccaggaagaatttcgcaaatggtttgtggaactgcgcagcaccagcgatcaggcgaaacgcagcattgaagattttaaagtggaaggcaacaccgcgaaagtgaaagtggtgctggaagcgagcaaaaacggcgaaaaacgcacggtgggcctggaacatatttttgaatttgaaggcgatgaggtgaaagaagtgtatgtgaaaattaaaccgctgggtaa

SEQ ID NO: 295 - DNA, 356 bp, synthetic construct
  source 1..356: mol_type = other DNA
  variation 1..3: note = optionally absent
  variation 354..356: note = optionally absent
atgagcgcgaccgaacagcgcgaatttaacaaacgcttttatgaagcgctggatgcgggtgatgcggataccgcaagcgcactgtttccggatggcaccgaaatttatctgtgggatggtaccacctttcatacccaggaagaatttcgcgcgtggtttgtggaactgcgcagcacctctgaaaacgcgcgcagacatattgtgagctttaccgtggacggcaacaaagcggatgtggaagtggtgctggaagcgaccgtgaaaggcgaaccgaaacgcgtgcgcctgactcataactatcagtgggaaggcgataaactggtgaaagtgaccgttaaaattgtgccgctgggtaa

SEQ ID NO: 296 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgaaacgcttttatcaggcgctggatgcgggtgatgcggataccgcaagtgcgctgtttccggatggtaccaaaatttatctgtgggatggcgttatttttaccaaacgcgaggaatttcgcaaatggttcgtggaactgaaaagcaaaagcgataacgcgaaacgagaagtggtggatctgcgcgtggaaggcgatacggcggatgtgagcgtggtgctgaaagcgaaatataaaggcgaaccgaaagtggttaaactgaaccataaattttattttgaaggtgataaactggttgaagtggaagttgaaattgtgccgatgggtaa

SEQ ID NO: 297 - DNA, 356 bp, synthetic construct
  source 1..356: mol_type = other DNA
  variation 1..3: note = optionally absent
  variation 354..356: note = optionally absent
atgaccgaagaagaacagcgcgaattttgggaacgtttttataaagcgctggatgcgggtgatgcggaaaccgcgagcgcattgtttccggatggcaccgaaatctatctgtgggatggtaccgtgtttaaaacccaggaagaatttcgcagctggtttgtggatctgtatagcaaaagcaacaacgccaaacgccatattgtggaatttagggtggaaggcgatgtgagctatgtggaagttgtgctgcatgcgagcgaaaacggcacggaaaagaccgtgcgcctgacccatattaccgaatttgaaggtgataaagtggttaaagttgaagtgagcattaccccgctgggtaa

SEQ ID NO: 298 - DNA, 356 bp, synthetic construct
  source 1..356: mol_type = other DNA
  variation 1..3: note = optionally absent
  variation 354..356: note = optionally absent
atgagcgaagatgaaattcgcgaattttggaaaaagttttatgaagcgctggatgcgggtgatgcggataccgcgagcgcgttgtttcctgatggcaccaaaatttatctgtgggatggcaaagtgtttcatacccaggcggaattccgcgaatggtttgtgaaactgtatagtaccagcgaaaatgcgaaacgtgaaatcacccgctttgaagtggatggtaacgtggcgaacattgaagttgtgctggaagcgaatgaaaacggtgaacagaagaaagttaaactgcgtcatattgccgaatttgaaggcgataaactggtggaagtgaacgtggaaattgaaccgctgggtaa

SEQ ID NO: 299 - DNA, 356 bp, synthetic construct
  source 1..356: mol_type = other DNA
  variation 1..3: note = optionally absent
  variation 354..356: note = optionally absent
atgagcgaaaaagaaattcgcgaatttgttgaacgtttttataaagcgctggatgatggcgatgcggaaaccgcgagcagcctgtttccggatggcacccgcatttatctgtgggatggtaccgaatttagcacccaggcggaatttcgcgcgtggtttgaagaactgcatagccgcagcgaagccgcgcgcagaagagtggtgcgtttggaagttgatggtgatgaagcggatgtggaagtggttctggatgctgattttgaaggcgaacatcatcgcgtgcgcctgcgccatcgctttttcttcgaaggtgatcgcctggttgaagtcgaagtgaccattgaaccgctgggtaa

SEQ ID NO: 300 - DNA, 356 bp, synthetic construct
  source 1..356: mol_type = other DNA
  variation 1..3: note = optionally absent
  variation 354..356: note = optionally absent
atgagcgaaaaagaaattcgcgaatttgtggataaattttataaagcgctggatagcggcgatgccgaaaccgctagcgcgttgtttccggatggcaccaacatttatctgtgggatggtaccgtgtttaaaaccaaagaagaatttcgcaaatggtttgttgaactgaaaagcaccagcgataacgcgaaacgcaaagtgaccaaactggaagtgcatggtaataccgcgcatgtggaagttgtgctgaaagcgagcgtgaacggtgaagaaaaagtggtgaaattgaaacatatttttctgtttgaaggcgataaactgcgtgaagtttatgttgaaatcgaaccgctgggtaa

SEQ ID NO: 301 - DNA, 356 bp, synthetic construct
  source 1..356: mol_type = other DNA
  variation 1..3: note = optionally absent
  variation 354..356: note = optionally absent
atgagcgaagaagaacagcgcgaattttggcgcaaattttatgaagcgctggatgcgggcgatgccgaaactgctagcgcactgtttccggatggcaccgaaatttatctgtgggatggtaccgtgtttcgcacccgcgcggaatttcgcgcgtggtttcgccagctgtatagccagagcgataacgcgaaacgccgcattaccagctttgtggtggaaggcaaccgcgcgtatgtggaagtggtgctggttgcgaacattaaaggcaagaaagttgaagtgagcctgaaacatttgtttgaatttgaaggcagccgcctggtgcgcgtgtatgttgaaattaaaccgctgggtaa

SEQ ID NO: 302 - DNA, 356 bp, synthetic construct
  source 1..356: mol_type = other DNA
  variation 1..3: note = optionally absent
  variation 354..356: note = optionally absent
atgagcgaacaggaacagcgcgattttgtggcgcgcttttatgcggcgctggatgcgggtgatgcggaaactgcaagcgcgctgtttcgtgatggcaccaaaatttacctgtgggatggcaaaacctttcgcacccaggcggaatttcgcagctggtttgtggaactgcatagccagagcaaagatgcgaaacgccgcgtggtgagctttcgcgtggatggtaacgtggcggatgtgaacgtgattctgcatgcgagcgtggaaggcgaagaacgcaccgttcgcctgaatcatgtgtttaaatttgaaggtgatcatctggtggaagtgcgcgttgatatcgaaccgctgggtaa

SEQ ID NO: 303 - DNA, 356 bp, synthetic construct
  source 1..356: mol_type = other DNA
  variation 1..3: note = optionally absent
  variation 354..356: note = optionally absent
atgagcgaagaagaaattcgcgcgttttgggaacgcttttatagcgcgctggatgcaggcgatgccgaaacggcgagcagcctgtttccagatggcaccgaaatttatctgtgggatggcaaaacctttcgcacccaggaagaatttcgcgcctggtttgaagaactgcgcagcaccagcgcggatgcaagcagacatattaccagcctgaaagtggaaggcaaccgcgcggacattgaagtggtgctgcgcgcgaactttaaaggtgaagaaaagaccgtgcgcctgcgccatgaagcggaatttgaaggcgatcgcttggtgcgcgtggaagtgcgcattgatccgctgggtaa

SEQ ID NO: 304 - DNA, 356 bp, synthetic construct
  source 1..356: mol_type = other DNA
  variation 1..3: note = optionally absent
  variation 354..356: note = optionally absent
atgagcgcggcggaacagcgcgaatttgttgatcgcttttataaagcgctggatgcgggcgatgctgaaaccgcaagcgcgttgttccctgatggcaccgtgattgatctgtgggatggccgcacctttcgcacccgcgcagaatttcgcgcgtggtttgaacgcctgcatgcgaccagcaccgatgcgaaacgcgaagtgacccagatgaaagtggatggcaacaccgtggatgttgaagtggtgctgcgcgcgaccgtgaacggtgaagaaaaagtggttaaactgcgccatcagtttcattttgaaggcgatcagctggtgcgcgtgcgtgtggatattaccccgctgggtaa

SEQ ID NO: 305 - DNA, 356 bp, synthetic construct
  source 1..356: mol_type = other DNA
  variation 1..3: note = optionally absent
  variation 354..356: note = optionally absent
atgagcgaaagcgaaattaaagaatttgtgcgcaaattttatgaagcgctggatgcaggtgatgccgataccgcagcggcactgtttccggatggcaccgtgattaaactgtgggatggtaccaccttttatacccaggcggaatttcgcgcgtggtttgtgaaactgtatagcaccagcgatgaagccagccgcgaagtgaccagcctgaaagtggaaggcgataaagcggaagttgaagtggtgctgaaagccaaaattaacggtgaagaaaagaccgtgaaattgaaacatatttttgaatttaaaggcgacaaactggtgcgcgtcgaagttagcattagcccgctgggtaa

SEQ ID NO: 306 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaacgcttttatgaagcgctggatgcgggcgatgcggaaaccgcagcagcgttgtttccagatggcaccgaaattcatctgtgggatggcaaaacctttcgcacccaggctgaatttcgcgcgtggtttgtgcagctgcgcagcaccagtgaagcggcgaaacgcaaaattattaaatttgaagtgattggcaacaaaagctttgtggaagtggtgctgaaagcgagcattaacggtgaagaaaaagaagtgtttctgcgccattattttgaattcgaaggcgataaactggtgcgcgtgtatgtgaccatcaaaccgctgggtaa

SEQ ID NO: 307 - DNA, 356 bp, synthetic construct
  source 1..356: mol_type = other DNA
  variation 1..3: note = optionally absent
  variation 354..356: note = optionally absent
atgagcgaacgcgaaattcgccagtttgtggatcgcttttatgcggcgctggatgcgggcgatgcggaaactgcagcagcgttgtttccagatggcaccgaaatccatctgtgggatggccgcacctttaccacccaggcggaatttcgcgcgtggtttgaaaaactgcgtgcgcgcagcgataacgcgcgtcgcgaagtggttgatctgcaggttgatggcgatgaagcggatgtgcgcgttgtgctggacgcgacctttcgcggtgaagcgagaagagtgcgtctgacccatcgcttcctgtttgaaggcgatcaggtgacccgcgtggaagtggaaattaggccggatggtaa

SEQ ID NO: 308 - DNA, 356 bp, synthetic construct
  source 1..356: mol_type = other DNA
  variation 1..3: note = optionally absent
  variation 354..356: note = optionally absent
atgagcgaagcggcgcagcgcgaattttgggatcgcttttatcgtgcgctggatgcgggtgatgcggaaaccgcaagcgcactgtttccggatggcaccgaaatccatctgtgggatggtaccacctttcgcacccgtgcggaatttcgcgcgtggtttcgcgatctgcatgcgagaagcgatgcggcccgtagaagcattgtgagctttgaagtggatggcgatgatgcccgtgtggaagtggtgctgcacgccaacgtggacggtcaggaacgtaccgtgcgtctgagacatgaagcgcattttgaaggcgaccgcctggtgcgcgtgcatgtggtgattcagccgctgggtaa

SEQ ID NO: 309 - DNA, 356 bp, synthetic construct
  source 1..356: mol_type = other DNA
  variation 1..3: note = optionally absent
  variation 354..356: note = optionally absent
atgagcgaaaccgcgatccgccagtttgtggaacgcttttatgaagcgctggatgcgggcgatgcggaaactgcagcagccctgtttccagatggcacccgcatttatctgtgggatggtaccacctttcatacccaggcggaatttcgcgcgtggtttgaagccctgcatgcgaccagcagcggtgcgaaacgtcatgtggtggcgttgcaggttgatggcgatgtggcggatgtggaagtggtgctgcacgccgatgtagatggtgaaaaacgcaccgttcatctgaaacatacctttaaatttcgtggcgatcgcctggttgaagtcgaagtgcatattcagccgctgggtaa

SEQ ID NO: 310 - DNA, 356 bp, synthetic construct
  source 1..356: mol_type = other DNA
  variation 1..3: note = optionally absent
  variation 354..356: note = optionally absent
atgagcagcgatgcgcagcgcgcctttgtggatcgcttttatcgtgcgctggatgcgggcgatgccgaaactgcgagcgcgttgtttccagatggcacccgcattcatctgtgggatggtaccacctttaccacccgcgaagaatttcgcgcgtggtttgttgatctgcgctctcgcagcgaaaacgcggcgcgtgaagtggtgagttttgatgtggatggcgatgtggcgcatgtggaagttgtgctgaaagcggtgattgaaggtgaagaagtcgtggttcgcctgcgccatgtgtttgaatgggaaggcgatcgcctggttgaagtgtatgtagaaattgatccgctgggtaa

SEQ ID NO: 311 - DNA, 356 bp, synthetic construct
  source 1..356: mol_type = other DNA
  variation 1..3: note = optionally absent
  variation 354..356: note = optionally absent
atgagcgaaaaagatcagaaagaatttgtgaagaaattttatgaagcgctggatgcgggcgatgcggaaaccgcaagcagcttgtttccggatggcaccgaaatccatctgtgggatggtaaagtgtttcatacccaggcggaatttaaagcgtggtttgaagaactgtatagccgcagcgatgatgcgaaacgcagcattattagcttcaaagtggacggcaacattgcgaaagtgattgtggtgctgaaagcgtttgttgatggtgaaaaactggaagtgctgctggaacatgaattcaaatttgaaggcgataaactggtggaagtaaaagtgaaaatttatccgctgggtaa

SEQ ID NO: 312 - DNA, 356 bp, synthetic construct
  source 1..356: mol_type = other DNA
  variation 1..3: note = optionally absent
  variation 354..356: note = optionally absent
atgagcgaagaagaacagcgtgaatttgtgcgccgcttttatgatgcgctggatgcgggcgatgcggaaactgcaagcgcgctgtttccagatggcacccagattgaactgtgggatggcaaaacctttcatacccgtgaagaatttcgcgcgtggtttgtgaaattgcatagcacctcggatgatgcccgccgcgaagtggtgagctttagcgtggcaggtgatgtggcgaacgtggaagttgtgctgcgtgcgaacattcgcggcgaaaagaaagtggttcgcctgcgccatatttttgaattcgaaggcgatcgcctggtgcgcgtgcgtgtggatattgaaccgctgggtaa

SEQ ID NO: 313 - DNA, 356 bp, synthetic construct
  source 1..356: mol_type = other DNA
  variation 1..3: note = optionally absent
  variation 354..356: note = optionally absent
atgagcgaagatgaaattcgcgaatttgtggcgcgcttttatgcggcgctggatgcgggtgatgccaacactgcaagcgcgctgtttcctgatggcaccgtgattcatctgtgggatggtattacctttcatacccaggcggaatttcgcgcgtggtttgaaaaactgcatagcaccagcgaaaacgcgagccgcgaagtggtgagcctgaaagtggatggcaacgtggcgcatgtggaagttgttctgcgcgcgagcgtggacggtgaagaacgcacggttcgtctgcgtcatgtgtttcgctttgaaggcgataaactggttgaagtgagcgtgagcattaccccgctgggtaa

SEQ ID NO: 314 - DNA, 356 bp, synthetic construct
  source 1..356: mol_type = other DNA
  variation 1..3: note = optionally absent
  variation 354..356: note = optionally absent
atgtcagaagaagaaattcgcgaattttggaaacgcttttatgaagcgttagatgcgggtgatgcggaaaccgctagtgccctgtttccggatggcacccgcatttatctgtgggatggtaaagaatttaccacccaggcggaatttcgcgcgtggtttgaagaactgtatagcaccagcgaagatgctagccgcgaaattgtgaaactggaagtggaaggcaacgtggcgtatgtggaagttgtgctgaaagcgaacattaacggcgaagaaaaagtggttaaactgaaacatgtgtttcattttgaaggcgatcgcctggttgaagtcgaagttgaaattgaaccgctgggtaa

SEQ ID NO: 315 - DNA, 356 bp, synthetic construct
  source 1..356: mol_type = other DNA
  variation 1..3: note = optionally absent
  variation 354..356: note = optionally absent
atgagcgcggaagcgcagcgccgctttgtggatcgcttttatgcggcgttggatgcgggcgatgcggataccgcaagtgcgctgtttccagatggcaccgaaattcatctgtgggatggccgcacctttcgcacccgcgccgaatttcgcgcatggtttcgtgaactgcgtgcgcgtagcgataacgcgcgccgtgaagtggtggcgtttgaagttgatggcgatacggcgcatgtggaagttgtgctgcgcgcgagcattgatggtgaagaacgcgtggtgcgcctgcgtcataccttttattttgaaggtgatcgcctggtgcgtgttgaagtcgaaattgaaccgctgggtaa

SEQ ID NO: 316 - DNA, 356 bp, synthetic construct
  source 1..356: mol_type = other DNA
  variation 1..3: note = optionally absent
  variation 354..356: note = optionally absent
atgagcgaagaagaacagcgcgaatttgttgatcgcttttatgcggcgctggatgcgggcgatgcagaaaccgcgagcgcattgtttcctgatggcaccaaaatttatctgtgggatggcaaagtgtttaccacccgtgaagaatttcgcgcgtggtttgaaaaactgtatagcaccagcgaaaacgcgaaacgccatgttgtgagctttaaagtggatggtaacaaagcggatgtggaagtggtgctgcatgcgaacattaacggcgaaaagaaaaccgtgcgcctgcgccacgtgttttattttgaaggcgataaactggttgaagtgaaagttgaaattaaaccgctgggtaa

SEQ ID NO: 317 - DNA, 356 bp, synthetic construct
  source 1..356: mol_type = other DNA
  variation 1..3: note = optionally absent
  variation 354..356: note = optionally absent
atgagcgaagaagaacagcgtgaatttgtgaaacgcttttatgaagcgttagatgcgggcgatgccgaaaccgcgagcgcattgtttccggatggcaccgaaattcatctgtgggatggtaaaaccttttatacccgcgaagagtttcgcgcgtggtttgaaaaactgtatagcaccagcgataacgcgagccgtagcgtgaccgaatttaaagtggatgggaacaaagcgaaagtgaaagtggtgctgaaagccaacattaacggcgaaaagaaaaccgtgaaactggaacattattttgaattcgaaggcgataaactggtgcgcgtgaacgtgaccattaaaccgctgggtaa

SEQ ID NO: 318 - DNA, 356 bp, synthetic construct
  source 1..356: mol_type = other DNA
  variation 1..3: note = optionally absent
  variation 354..356: note = optionally absent
atgagcgaagaagaacagcgccgctttgtggaacgcttttatgcggccctggatgcgggtgatgcggataccgcaagcgcattgtttccggatggcaccgaaattcatctgtgggatggtaccgtgtttcgcacccgcgcggaatttcgcgcgtggtttgaacgcctgtatagtaccagcgaaaacgcgaaacgccatgtggtgcgctttgaagtggaaggcaacgtggcgcgcgttgaagttgtgctgcatgcgaacattgatggtgaagaacgcaccgtgcgcctgacccatgtgtttgaatttgaaggcgaccgcctggtgcgtgtgaacgtgaccattaacccgctgggtaa

SEQ ID NO: 319 - DNA, 356 bp, synthetic construct
  source 1..356: mol_type = other DNA
  variation 1..3: note = optionally absent
  variation 354..356: note = optionally absent
atgagtgaagaagagattaaagaatttgtgaaacgcttttatgaagccctggatgcgggcgatgcggaaaccgcgagcgctttgtttccagatggcacccgcatttatctgtgggatggtcgcgtgtttcgcacccgtgcggaatttcgcgcatggtttgtggaactgcatagcaccagcgaagatgcgaaacgcgaagtgattgaactgaaagtggaaggcaacgtggcgaaagtgaaagttgtgctgcatgccaacatcaacggcgaaaagaaaaccgttctgctggaacattattttgaatttgaaggcgatcgcctggtggaagtgcgcgtggaaattaaaccgctgggtaa

SEQ ID NO: 320 - DNA, 356 bp, synthetic construct
  source 1..356: mol_type = other DNA
  variation 1..3: note = optionally absent
  variation 354..356: note = optionally absent
atgagcgaagaagcgcagaaagaatttgtgaagaaattttatgaagcgctggatgcgggcgatgcggataccgcatcggctttgtttccggatggcaccgaaatttatctgtgggatggtaaaacctttcataccaaagcggaatttaaagcgtggtttgttaaactgaaaagcaccagcgataacgccaaacgcagcgttgtgaaatttgaagtggatgggaacgtggcgtatgtggaagtggtgctgcatgcgaacattaacggtgaagagaaagtggtcaaactgacccatatttttgaattcgaaggcgataaattagtgaaagtgaacgtgaccattaaaccgctgggtaa

SEQ ID NO: 321 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgcgccgcttttatgaagcgctggatgcgggcgatgcggaaactgcgagcgcattgtttcctgatggcaccgtgatttatctgtgggatggcaaaacctttcatacccgtgaagaatttcgtgcgtggtttgtggaactgcgcagcaaaagcgaaaacgcgaaacgccatgtggtgagcctgcgcgtggatggtaatgtggcggatgtggaagtggtgctggacgcggatattaacggcgaaaagaaaaccgtgaaactgagacatgaatttagatttgaaggcgatcgcctggttgaagtgcgcgtcgaaattgaaccgctgggtaa

SEQ ID NO: 322 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgaaacgtttttatgaagcgctggatgcgggtgatgcggataccgcatctgcgctgtttccggatggcaccaaaatttacctgtgggatggtaaaacctttagcacccaggcggaatttaaagcctggtttgttaaactgaaaagcaccagcgaaaacgccaaacgcaaaattgtgaagctgaaagtggatggcgatgtggcggaagtggaagttgtgctgcatgcgaacgtgaacggtgaagaaaaagtggtgaaattgaaacataaattcaaatttgaaggcgataaactggttgaggttgaagttgaaattaaaccgctgggtaa

SEQ ID NO: 323 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaacgcttttatgaagcgctggatgcgggtgatgcggataccgcgagcgcgttgtttccagatggcaccaaaattcatctgtgggatggcaaaacctttaccacccaggcggaatttcgcgcgtggtttgttgaactgcatagcaccagcgatgatgcgaaacgcgaaattgtgaaatttgaagtggatggtaacaaaagctatgtggaagtggtgctgcgcgcgaacattaacggtgaagaaaaagtggttcgcctgacccatgaaacccagtttgaaggcgataaactggttgaagttaaagtgaccattgaaccgctgggtaa

SEQ ID NO: 324 - DNA, 356 bp, synthetic construct
  source 1..356: mol_type = other DNA
  variation 1..3: note = optionally absent
  variation 354..356: note = optionally absent
atgagtgaagaagcgcagcgcgaatttgtgcgccgcttttatgcggcgctggatgcgggtgatgcggaaacagcagcagcgttgtttccggatggcaccgtgattcatctgtgggatggccgcacctttcgcacccaggcggaatttcgcgcctggtttgtggaactgtatagcaaaagcgaagatgcgaaacgtgaagtggtgcgttttgaagttgatggcgatcgcgcgcgtgtggaagttgtgctgaaagccaattttaaaggcaagaaagaagtcgtgaaactggtgcattattttctgtttgaaggcgatagactggttgaagtgcgcgtggaaattaaaccgctgggtaa

SEQ ID NO: 325 - DNA, 356 bp, synthetic construct
  source 1..356: mol_type = other DNA
  variation 1..3: note = optionally absent
  variation 354..356: note = optionally absent
atgagcgcggaagcgcagcgcgaatttgtgcgccgcttttatgcggcgttggatgcgggtgatgcggaaaccgcaagcgcgctgtttccagatggcacccgcattcatctgtgggatggccgcgtgtttcgcacccgtgaagaatttcgcgcgtggtttgtgaaactgtatagcaccagtgaagatgcgcgccgcgaagtgacggcgtttcgtgttgatggcgatcgtgccgatgtggaagtggtgctgcgcgcgagcattaacggcgaagaaaagaccgtgcgcctgcgtcatgtgtttcagtttgaaggggatcgtctgcgtgaagtgcgcgtcgccattgaaccgctgggtaa

SEQ ID NO: 326 - DNA, 356 bp, synthetic construct
  source 1..356: mol_type = other DNA
  variation 1..3: note = optionally absent
  variation 354..356: note = optionally absent
atgagcgaagaagaaattcgcgaattttgcaaacgcttttatgaagcgctggatgcgggcgatgcggataccgcaagtgcgctgtttccagatggcaccgaaatttatctgtgggatggccgcacctttcgcacccgtgaagaatttcgcgcgtggtttgtggaactgcatagcaccagcgatgatgcccgccgcagcattgtgaaactggaagtggaaggcaacgtggcgtatgtggaagttgtgctgcgcgcgaacgtggatggggaagaaaaagtggtgcgcctggtgcatattttctattttgaaggcgataaactggtgaaagtgtatgtgagcattcgcccgctgggtaa

SEQ ID NO: 327 - DNA, 356 bp, synthetic construct
  source 1..356: mol_type = other DNA
  variation 1..3: note = optionally absent
  variation 354..356: note = optionally absent
atgagcgaagaagaaattcgcgaattttggaaaaagttttatgaagcgctggatgcgggtgatgcggaaaccgcgagcgcactgtttcctgatggtaccaaaattcatctgtgggatggcaaaacctttaacacccaggcggaatttaaagcgtggtttgtgaaactgtatagcgaaagcgatgatgcgaaacgcgaaattgtggaactggaagtggatggtaacgtggcgtatgtgaaagtggtgctgcatgcgaactataaaggcgaacagaaaaccgttgaactgaaacatatttttaaatttgaaggcgataaactggtggaagttaacgttgaaattaaaccgctgggtaa

SEQ ID NO: 328 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaaaagttttatgaagcgctggatgcgggcgatgcggaaaccgcaagcgccttgtttccagatggcaccaaaatttatctgtgggatggcaaagtgttctatacccaggaagaatttcgcgcgtggtttgtggaactgcgcagccagagcgatgatgcgaaacgcaaaattgtggattttaaagtggaaggcgatgtggcgtatgtggaagtggtgctggaagcgaacattaatggcgaaaagaaaaccgtgaaactgaaacatatttataaattcgaaggtgataaactggttgaagtgaaagttaaaatcgaaccgctgggtaa

SEQ ID NO: 329 - DNA, 356 bp, synthetic construct
  source 1..356: mol_type = other DNA
  variation 1..3: note = optionally absent
  variation 354..356: note = optionally absent
atgagcgaagaagaacagcgcgaattttggcgccgcttttatgaagcgctggatgcgggcgatgcggaaaccgcctctgcactgtttccagatggcaccaaaattcatctgtgggatggccgcacctttaccacccaggaagaatttcgcgcgtggtttgtggatctgcatagccgcagcgatgatgcgaaacgcgaaattgtgagctttaaagtggaaggcaacaaagcgcgcgttgaagtggtgctgcgcgcacgtgaagatggtgaagaacgcgttgtggcgctgcgtcatgaaaccgaatttgaaggcgatcgcctggtggaagtgaacgtggatattcgcccgctgggtaa

SEQ ID NO: 330 - DNA, 356 bp, synthetic construct
  source 1..356: mol_type = other DNA
  variation 1..3: note = optionally absent
  variation 354..356: note = optionally absent
atgagcgaagaagcgcagcgtgaatttgtgcgccgcttttatgcggcgttggatgcgggtgatgcggataccgctagcgccttatttccggatggcacccgcatttatctgtgggatggccgcacctttaccacgcgcgcggaatttcgcgcatggtttgaacgcctgcatgcgaccagcgcagatgcgaaacgggaagtggtggattttgaagtggaaggcaacaaagcgaaagtgaaagtggttctgcacgcgaacgtgaacggtgaagaaaagaccgtgctgctggaacattggttcgaatttgaaggcgatcgcctggtgcgcgtggaagtgaccattaaaccgctgggtaa

SEQ ID NO: 331 - DNA, 356 bp, synthetic construct
  source 1..356: mol_type = other DNA
  variation 1..3: note = optionally absent
  variation 354..356: note = optionally absent
atgagcgaaaaagaacagcgcgaattttggaaaaagttttatgaagcgttggatgcgggtgatgccgaaaccgcgagcgcgctgtttccagatggcaccaaaatttatctgtgggacggcaaaacctttaccacccaggccgaatttcgcgcgtggtttgtggaactgcgcagcaccagcgagaacgccaaacgcaaaattgtgagctttaaagtggatggcaacaaaagcgaagtggaagtggtcctggaagcgaacatcaacggcgaaaagaaagtggttaaactgaaacacatttttaaatttgaaggcgataaactggttgaagtgaaagttgaaattattccgctgggtaa

SEQ ID NO: 332 - DNA, 356 bp, synthetic construct
  source 1..356: mol_type = other DNA
  variation 1..3: note = optionally absent
  variation 354..356: note = optionally absent
atgtccgaagaagaacagcgcgaattttggcgcaaattttatgaagcgctggatgcgggtgatgcggataccgcgtctgcgctgtttccagatggcaccgaaattcatctgtgggatggcaaaacctttcgcacccgcgaagaatttcgcgcgtggtttgaagaactgtatagcaccagcgaagatgcgaaacgcgaaattgtgaaatttgaagtggaaggcaacaaaagctttgtggaagtggtgctgaaagcgaacgtcaacggcaagaaagtggttgtcaaactgcgccatgtgtttgaattcgaaggcgataaagttgtgaaggttgaagttgaaattgaaccgctgggtaa

SEQ ID NO: 333 - DNA, 356 bp, synthetic construct
  source 1..356: mol_type = other DNA
  variation 1..3: note = optionally absent
  variation 354..356: note = optionally absent
atgagcgaagaagaaattaaaaagttttgggaaaaattttataacgccctggatgcgggtgatgcggatactgcaagcgctctgtttccggacggtaccgaaattcatctgtgggatggcaaaacctttaaaacccaggcggaatttcgcgaatggtttgtgaaactgtatagcaccagcgatgatgcgaaacgcgaaattgttaaactggaagtggatggtaacgtggcgtatgtggaagttgtgctgcgcgccaacgtggacggtgaagaaaagaccgtaaaactgcgccatgtggcggtgtttgaaggcgataaactgaaagaagtgaaagtgaccattaaaccgctgggtaa

SEQ ID NO: 334 - DNA, 356 bp, synthetic construct
  source 1..356: mol_type = other DNA
  variation 1..3: note = optionally absent
  variation 354..356: note = optionally absent
atgagcgaagaagaacagcgcgaatttgtgcgccgcttttatgaagcgctggatgcgggcgatgcggaaactgcgagcgcgttgtttccagatggcaccaaaattcatctgtgggatggcaaaacctttagcacccgcgcggaatttcgtgcgtggtttgtggaactgcgtagcaccagcgaaaacgccaaacgccgcgtgaccagctttcgtgtggaaggcaacgaagcggatgttgaagtggtgctggaagcgaccgtgaacggcgaaaagaaacgcgtgcgcctgcgtcaccgctttctgtttgaaggcgatcgcctggtggaagtgtatgtggaaattaaaccgctgggtaa

SEQ ID NO: 335 - DNA, 356 bp, synthetic construct
  source 1..356: mol_type = other DNA
  variation 1..3: note = optionally absent
  variation 354..356: note = optionally absent
atgagcgaagaagaacagcgcgaatttgtgaaacgtttttatgaagcgctggatgcgggcgatgcggaaactgcaagcgcactgtttccggatggcaccctgatttatctgtgggatgggaaaacctttaccacccaggcggaatttcgcgcatggtttgaaaaactgcgcagcaccagcgaggatgcgaaacgcgaagtggtggaatttaaagttgatggtaacgtggcggatgtggtggttgtgctggaagcgaacattaacggcgaaaagaaagtggttaagctgcgtcatcgctttcattttgaaggcgataaactggtcaaagtggaagttgaaattgaaccgctgggtaa

SEQ ID NO: 336 - DNA, 356 bp, synthetic construct
  source 1..356: mol_type = other DNA
  variation 1..3: note = optionally absent
  variation 354..356: note = optionally absent
atgagcgaagaagaacagcgcgaatttgtaaaacgcttttatgaagcgttggatgccggtgatgcggataccgcgagcgcgttgtttccggatggcaccaaaattcatctgtgggatggtaccacctttaccacccaggcggaatttaaagcgtggtttgaaaaactgtatagcaaaagcgataacgccaaacgccatgtggtgagctttaaagtggaaggcaacgtggcgtatgtggaagtggtgctgcatgccaattttaaaggtgaagaaaagaccgtgagcctgaaacatatctttaaatttgaaggcgataaactggttgaagtcgaagtgaaaattaaaccgctgggtaa

SEQ ID NO: 337 - DNA, 356 bp, synthetic construct
  source 1..356: mol_type = other DNA
  variation 1..3: note = optionally absent
  variation 354..356: note = optionally absent
atgagcgaagaagaacagcgcgaatttgtgcgccgtttttatgaagcgctggatgcgggtgatgcggaaactgcgagcgcactgtttcctgatggcaccgtgattcatctgtgggatggcaaaacctttcgcacccaggcggaatttcgcgcgtggtttgtggaactgaaaagcaccagcgaggatgcgaaacgtcgcgttgtgagctttcgtgtggatggcgatgaagccgaagtggtggttgtgctgcatgcgaacattgatggtgaagaacgcgtcgtgcgcctgacccatcgctttctgtttgaaggtgatcgcgtagtggaagtgtgggtggaaattgaaccgctgggtaa

SEQ ID NO: 338 - DNA, 356 bp, synthetic construct
  source 1..356: mol_type = other DNA
  variation 1..3: note = optionally absent
  variation 354..356: note = optionally absent
atgagcgaacaggaacagcgcgaatttgtggatcgcttttatcgtgcgctggatgcgggtgatgcggataccgcgagcgcactgtttcctgatggtaccgaaatttatctgtgggatggcaaaacctttcgcacccaggcggaatttcgcgaatggtttgtgaaactgcatagcaccagcgaagatgccaaacgccgcgtggtgagctttagcgtggatggtaacgtggcggatgtggaagtggtgctggaagcgaatgtgaacggcgaaaagaaaaccgtcaaactgaaacatcgcttccattttgaaggcgataaagtggttcgcgttgaagtgagcattgaaccgctgggtaa

SEQ ID NO: 339 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgcgccgcttttatgcggcgctggatgcgggtgatgcggaaactgcaagcgcgttgtttccggatggcacccgcattcatctgtgggatggccgcacctttaccacccaggcggaatttcgtgaatggtttgtgaaactgtatagcgaaagcgatgatgcgcgccgcgaagtggtgagcctggaagtggatggcgatcgtgcattggtgcgcgtggtcctgcgtgcaagctataaaggtgaagaacgcgtcgtggaactgcgccatgaatttctgtttgaaggcgatgaactggtggaagttcgcgtggaaattagaccgctgggtaa

SEQ ID NO: 340 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaacgcttttatgaagcgctggatgcgggcgatgcggaaactgcaagcgcgctgtttccagatggcaccgaaattcatctgtgggatggtaccgtgtttcgcacccgcgcggaatttcgcgcgtggtttgaacagctgtatgccgaaagcgatgatgcgaaacgcgaaattgtgagctttaaagtggaaggcaacgttagcgatgtggaagtggtgctgcatgcgagctataaaggcgaaaagaaaaccgtgaaactgagacatcgctttttctttgaaggcgatcgtctggttgaagtcgaagttgaaattgaaccgctgggtaa

SEQ ID NO: 341 - DNA, 356 bp, synthetic construct
  source 1..356: mol_type = other DNA
  variation 1..3: note = optionally absent
  variation 354..356: note = optionally absent
atgagcgaagaagaacagcgtgaatttgtgaaacgcttttatgaagcgctggatgcgggtgatgcggaaaccgcaagcgcgctgtttccagatggcaccgaaattcatctgtgggatggcattacctttcatacccaggcggaatttcgcgaatggtttgttaaactgcgcagcaccagcgaaaacgcgaaacgtgaagtggtgaaatttgaagttgatggtaacaaagcgaaagtggaagttgtgctgaaagcaaacattaacggtgaagaaaaagaagtgaaactgacccatacctttgaattcgaaggcgataaattagtgaaagtgaacgtggatattaaaccgctgggtaa

SEQ ID NO: 342 - DNA, 356 bp, synthetic construct
  source 1..356: mol_type = other DNA
  variation 1..3: note = optionally absent
  variation 354..356: note = optionally absent
atgagcgcggaagaaattcgcgaatttgtgcgtcgcttttatgaagcgctggatgcgggtgatgccgataccgcgagtgcgctgtttcctgatggcaccgaaattcatctgtgggatggccgcacctttcgcacccaggcggaatttcgcgcatggtttgtgaggctgcgcgcgacaagcgatgatgcgagccgtgaagtggtgagcttggaagtggatggcaacgttgcggatgtggaagttgtgctgcatgcgaacgtgaacggcgaaaagaaagtggttcgcctgcgccatcgcttctattttgaaggcgatcgcctggtgcgcgttgaagtgaccattgtgccgctgggtaa

SEQ ID NO: 343 - DNA, 356 bp, synthetic construct
  source 1..356: mol_type = other DNA
  variation 1..3: note = optionally absent
  variation 354..356: note = optionally absent
atgagcaaagaagaaattaaagaatttgttaaacgcttttatcaggcgctggatgcgggcgatgcggaaacagcgagcagcctgtttccagatggcaccaaaatttatctgtgggatggcaaagtgtttaaaacccaggaagagtttcgcgcgtggtttgaaaaactgcatagcaccagcgaaaacgcgaaacgtgaagtgaccaaactggaagttgaaggcaacgtggcgtatattgaagtggtgctgaaagcgaacgttaacggcgaagaaaagattgtgaacctgacccataaattttatttcgaaggcgataaactggtcgaagtggaagtgaaaattgttccgctgggtaa

SEQ ID NO: 344 - DNA, 356 bp, synthetic construct
  source 1..356: mol_type = other DNA
  variation 1..3: note = optionally absent
  variation 354..356: note = optionally absent
atgagcgaagaagaaatccgcgaatttgtgaaacgcttttatgaagcgctggatgcgggcgatgcggaaacggcgagcgcattgtttccagatggcaccaaaattcatctgtgggatggtaccgtgtttaccacccaggcggaatttcgcgcgtggtttgaaaaactgtatagcaccagcgataacgcgagccgcagcgtggtgagctttaaagtggatggcaacgtggcggaagtggaagttgtgctgaaagcgaacattaaaggccgcgaaaaagtggtgaaactgaaacatatttttcattttgaaggcgataaactggttgaagtcgaagtgagcattgaaccgctgggtaa

SEQ ID NO: 345 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgaaacgcttttatgaagcgctggatgcgggtgatgccgaaacagcgtccgcgctgtttccagatggcaccgtgattcatctgtgggatggcattacctttcatacccaggcggaatttcgcgcgtggtttgaagccctgtatagcaccagcgaaaatgcgcgccgcgaagtggtggcgttgagagtggaaggtaaccgtgctcgtgtggaagttgtgctgcatgcgaacgtggatggtgaagaacgcaccgttcgcctgcgccatgaatttctgttcgaaggcgatcgcctggtcgaagtgcgcgttgaaattgaaccgctgggtaa

SEQ ID NO: 346 - DNA, 356 bp, synthetic construct
  source 1..356: mol_type = other DNA
  variation 1..3: note = optionally absent
  variation 354..356: note = optionally absent
atgtccgaagaagcgcagcgcgaatttgtgaaacgcttttatgaagcgctggatgcgggtgatgcggaaaccgccagcgcgttgtttccagatggcacccgcattcatctgtgggatggccgcacctttaccacccgtgaagaatttcgcgcgtggtttgaagaactgcgcagcaaaagcgaagatgcgaaacgcgaagtgaccagctttcgcgtggatggcaacgtggcggaagtggaagttgtgctgcatgccaacatcaacggcgaagaaaagaccgtgctgctgaaacatcgctttgtgttcgaaggcgatcgcctggttgaagtgcatgtggaaattaaaccgctgggtaa

SEQ ID NO: 347 - DNA, 356 bp, synthetic construct
  source 1..356: mol_type = other DNA
  variation 1..3: note = optionally absent
  variation 354..356: note = optionally absent
atgagcgaagaagcccagcgtcagtttgtgcgccgcttttatgaagccctggatgcgggcgatgcggataccgctagcgcactgtttccagatggcaccgaaattcatctgtgggatggccgcacctttcgcacccgcgcggaatttcgtgcatggtttgaacgcctgcgcgcgacaagcgcagatgcccgcagaagcgtgacttcttttgaagtggatggcgattttgcgcgtgtggaagtggtgctgcgtgcgagctttgccggagaagaacgtgtggtgcgtctgcgtcattattttcagttcgaaggcgatcgcctggtgcgcgttgaagttgaaattcgcccgctgggtaa

SEQ ID NO: 348 - DNA, 356 bp, synthetic construct
  source 1..356: mol_type = other DNA
  variation 1..3: note = optionally absent
  variation 354..356: note = optionally absent
atgagcaaaaaggcgcaggaagaattttggaaacgcttttacgaagcgctggatgcgggtgatgcggataccgcaagcgcgctgtttcctgatggcaccaaaatttatctgtgggatggcaaaacctttaccacccaggccgaatttcgcgcgtggtttgtggaactgcatagcaaaagcgataacgcgaaacgccgcattaccaaatttgaagtggatggtaacaaaagctatgtggaagtggtgctggaagcgaacgtgaacggcaagaaagaagttgtgaaactggtgcatgaaaccctgtttgaaggcgataaattggttgaagttcgcgtggatattaaaccgctgggtaa

SEQ ID NO: 349 - DNA, 356 bp, synthetic construct
  source 1..356: mol_type = other DNA
  variation 1..3: note = optionally absent
  variation 354..356: note = optionally absent
atgagcgaagaagaacagcgcgaattttggcgtcgcttttatgaagcgctggatgcgggcgatgccgaaactgctagcgcactgtttccagatggcaccgaaattcatctgtgggatggcaaagtgtttcgcacccaggcggaatttcgcgcgtggtttgaacgtctgcgcgcgacaagcgcagatgcgaaacgcgaaattgtgagctttcgcgtggatggcgatcgcgcggatgtggaagtggtgctgcgtgcagttgttgatggtgaagaaaaagaagtgaaactgaaccatcgcttctttttcgaaggcgatagactggtgcgcgttgaagtggaaattaaaccgctgggtaa

SEQ ID NO: 350 - DNA, 356 bp, synthetic construct
  source 1..356: mol_type = other DNA
  variation 1..3: note = optionally absent
  variation 354..356: note = optionally absent
atgtccgaagaagcgattcgcgaattttggaaacgcttttatgaagccctggatgcgggcgatgccgaaaccgcgagcgcattgtttccagatggcaccgaaattcatctgtgggatggcaaaacctttcgcacccgcgcggaatttaaagcgtggtttgaagaactgtatagcaccagcgaaaatgccaaacgtgaaattaccagcctgaaagtggaaggcaaccgcgccgaagttgaagtggtgctgcatgcgaacattgaaggtaaagaaaagaccgtgaacctgaaacattggttcgaatttgaaggcgatcatctggtgcgcgtgcgtgtggatattaaaccgctgggtaa

SEQ ID NO: 351 - DNA, 356 bp, synthetic construct
  source 1..356: mol_type = other DNA
  variation 1..3: note = optionally absent
  variation 354..356: note = optionally absent
atgtcggaagaagaacagcgcgaatttgtgcgccgtttttatgaagcgctggatgcgggcgatgccgaaactgcgagcgctctgtttccagatggcaccgtgattcatctgtgggatggccgcacctttcgcacccaggcggaatttcgcgcctggtttgaagaactgtatagcaccagcgaagatgcgtctcgccatgtggtgagctttaaagtggatggcaacaaagcgcgcgtggaagtggtgctgcatgccaacattaacggcgaagagcataccgttaacctgacccatgtgtttcattttgaaggcgataaactggttgaagtcgaagtggacattcgtccgctgggtaa

SEQ ID NO: 352 - DNA, 356 bp, synthetic construct
  source 1..356: mol_type = other DNA
  variation 1..3: note = optionally absent
  variation 354..356: note = optionally absent
atgagcgaggaagaacagcgcgaattttggaaacgcttttatgaagcgctggatgcgggcgatgcggaaactgcgagcgcgttgtttcctgatggcaccgtgattcatctgtgggatggcaaaaccttccatacccgtgaagaatttcgcgcgtggtttgtggaactgaaaagcaaaagcgaagatgcgaaacgcgaaattgtggattttgaagttgacggcaacgtgagccgcgtgaaagtggtgctgcatgccaactataaaggcgaagaaaaagttgttgaactggaacatgtgtttcatttcgaaggcgatcgcctggtggaagtcgaagttaaaattaaaccgctgggtaa

SEQ ID NO: 353 - DNA, 356 bp, synthetic construct
  source 1..356: mol_type = other DNA
  variation 1..3: note = optionally absent
  variation 354..356: note = optionally absent
atgagcgaagaagaaattcgtgaatttgtgaaacgcttttatgcggcgctggatgcgggcgatgcggaaaccgcaagcgcactttttcctgatggcaccgaaatttatctgtgggatggtaccacctttcgcacccaggcggaatttcgcgcgtggtttgtggaactgtatagccgcagcgatgatgcgagccgcgaagtggtggatctgaaagtggatggcaataaagcggaagtggaagttgtgctgaaagcgagcgtagatggtgaagaacgcgtagtgaaactgaaacatttctttgaattcgaaggcgataaactggtgaaagttgaagtgaccattgaaccgctgggtaa

SEQ ID NO: 354 - DNA, 356 bp, synthetic construct
  source 1..356: mol_type = other DNA
  variation 1..3: note = optionally absent
  variation 354..356: note = optionally absent
atgagcgaagaagaacagcgcgaatttgtgaagaaattttatgaagcgctggatgcgggcgatgcggaaaccgcaagcgcgttgtttccagatggcaccgaaattcatctgtgggatggtaaaacctttcgcacccaggcggaatttcgtgcgtggtttgaaaaactgaaaagcaccagcgaaaacgcgaaacgcaaaattgtgagctttaaagtggatggcaacaaagcggatgtggaagttgtgctgaaagcgaatattaacggggaagaaaagaccgtgaaattgaaacatacctttgaatttgaaggcgataagctgaaaaaggtgaacgtggaaattgttccgctgggtaa

SEQ ID NO: 355 - DNA, 356 bp, synthetic construct
  source 1..356: mol_type = other DNA
  variation 1..3: note = optionally absent
  variation 354..356: note = optionally absent
atgagcgaagaagcgcagcgcgaatttgtgcgccgcttttatgaagcgctggatgccggcgatgcggaaaccgcaagcgcactgtttcctgatggcaccaaaatttatctgtgggatggccgcgtgtttgaaacccaggaagaatttcgcgcgtggtttgtggaactgcgcagcaccagcgaaaacgcccgcagaagggtggtggattttgaagtggatggcaatgttgcgcgcgttgaagttgtgctgcatgcgaacattgatggtgaagaaaagaccgtgcgcctgcgccatatttttaaatttgaaggcgataaactggtggaagtcgaagtgaccattgaaccgctgggtaa

SEQ ID NO: 356 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgcgccgcttttatgaagcgctggatgcgggtgatgcggaaacagcaagcgcgctgtttcccgatggcaccaaaatttatctgtgggatggcattacctttaccacccaggcggaatttcgcgcgtggtttgtggaactgcgcagcaccagcgatgcggcgagaagagaagtggtgcgcttggaagtggatggtaacgtggcgtttgttgaagttgtgctgcatgcgagccatcgcggtgaagaacgcaccgtgctgctgcgtcatgtgtttcagtttgaaggcgataaactggtgaaagtggaagttagcatcgatccgctgggtaa

SEQ ID NO: 357 - DNA, 356 bp, synthetic construct
  source 1..356: mol_type = other DNA
  variation 1..3: note = optionally absent
  variation 354..356: note = optionally absent
atgagcgaaaaagaacagaaagaattttggaaaaagttttatgatgcgctggatgcgggcgatgcggaaactgcgagcgcactgtttccagatggcaccattatctatctgtgggatggcattgtgtttcgcacgcgcgaagaattcaaagaatggtttgtgaaactgaaaagcacctcggaaaacgccaaacgcaaaattgtgagctttaaagtggatggtaacaaatctgatgtggaagtggtgctggaagcgaacgtgaacggcgaaaagaaaaaggttaaattgaaacatgtgttccattttgaaggcgataaactggttgaagtcgaagtgaaaattgatccgctgggtaa

SEQ ID NO: 358 - DNA, 356 bp, synthetic construct
  source 1..356: mol_type = other DNA
  variation 1..3: note = optionally absent
  variation 354..356: note = optionally absent
atgagcgaagaagaaattcgtaactttgtgaaacgcttttatgaagcgctggatgcgggcgatgcggaaaccgccagtgcgttgtttccagatggcaccgaaatttatctgtgggatggcaaaacctttcgcacccaggcggaatttcgcgcgtggtttgaagaactgcgcagcaccagtgatgatgcgaaacgtgaagtggttaaatttgaagttgaaggcaacgtggcgtatgtggaagttgtgctgaaagcgaaccataaaggcaagaaagaagtcgtgaaactgaaacatgaatttgagttcgaaggcgataaagtggtgaaagtgcgcgttgaaatcaaaccgctgggtaa

SEQ ID NO: 359 - DNA, 356 bp, synthetic construct
  source 1..356: mol_type = other DNA
  variation 1..3: note = optionally absent
  variation 354..356: note = optionally absent
atgagcgaagaagaacagcgcgaattttggaaacgcttttatgaagcgctggatgcgggcgatgcggaaaccgcaagcgcgttgtttcctgatggcaccgaaattcatctgtgggatggccgcacctttcgcacccgcgccgaatttcgtgcgtggtttgaagaactgtatagccagagcgaaaacgcgcgccgcgaaattacccgctttgaagtggatggcaacgtgtcacatgtggaagtggtgctggaagcggaatatgaaggcgaaaaacgtgtggtgcgcctgcgccatgtgtttgaatttgaaggtgatcgcctggtgcgtgttgaagtcgaaattgaaccgctgggtaa

SEQ ID NO: 360 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgaaacgcttttatgaagcgctggatgcgggcgatgccgaaactgcgagcgcactatttccggatggcaccgaaattcatctgtgggatggtaccgtgtttcgcacccgtgaagaatttcgcgcgtggtttgtggaactgcgctcaaaaagcgatgatgcgaaacgtgaagtggtgaaatttgaagttgatggtaacaaagcgtatgtggaagttgtgctgcatgcgaacgtggatggggaagaaaagaccgtgcgcctggtgcatgaatttctgtttgaaggcgataaactggtcgaagtgaaagtggatattgaaccgctgggtaa

SEQ ID NO: 361 - DNA, 356 bp, synthetic construct
  source 1..356: mol_type = other DNA
  variation 1..3: note = optionally absent
  variation 354..356: note = optionally absent
atgagcgaagaagatattcgcgaatttgtgcgccgcttttatgaagcgctggatgcgggcgatgcggataccgcaagcgcgctgtttaaagatggcaccaaaattcatctgtgggatggcaaaacctttaccacccgtgaagaatttcgtgaatggtttgtgagactgcatagcaccagcgatgatgcgcgccgggaagtggtacgcctggaagttgaaggcaaccgtgcacatgtggaagtcgtgctgcgcgcgagctatcagggcgaagatcgcgtggttagactgacccatgaatttctgtttgaaggtgatgaagttgttgaagtgcatgtgcgcattgaaccgctgggtaa

SEQ ID NO: 362 - DNA, 356 bp, synthetic construct
  source 1..356: mol_type = other DNA
  variation 1..3: note = optionally absent
  variation 354..356: note = optionally absent
atgagcgaagaagcgcagcgcgaattctggcgccgcttttatgaagcgctggatgcgggtgatgcggaaaccgcctctgcactgtttccggatggtaccgaaattcatctgtgggatggccgcacctttcgcacccaggcagaatttcgcgattggtttgtgaaattgcatagcaccagcgcggatgcgcgccgcgaaattacccgctttagagtggaaggtgatgtggcgcatgttgaagtggtcctgcatgcgagcattgatggcgaaaagaaaaccgtgaaactgcgccatgtggcccattttgaaggcgataaactggtggaagtgcatgtggatattgaaccgctgggtaa

SEQ ID NO: 363 - DNA, 356 bp, synthetic construct
  source 1..356: mol_type = other DNA
  variation 1..3: note = optionally absent
  variation 354..356: note = optionally absent
atgagcgaagaagaaattcgcgaatttgtgcgccgcttttatgaagcgctggatgcgggcgatgcggaaaccgcaagcgcgctgtttaaagatggcaccaaaatttatctgtgggatggtaccgtgtttgaaacccgtgaagaatttcgcgcgtggtttgtggaactgtatagcaaaagcgaaaacgcgcgccgccgagtggtgagctttaaagtggatggcaacgtggctgaagtggaagtggtgctgcatgcgagctttcagggcgaagataaagttgtgcgtctgaaacatcgttttaaatttgaaggcgatgaagttgttgaagtagaagttgatattgaaccgctgggtaa

SEQ ID NO: 364 - DNA, 356 bp, synthetic construct
  source 1..356: mol_type = other DNA
  variation 1..3: note = optionally absent
  variation 354..356: note = optionally absent
atgagcgaagaagcgcagcgcgaattttggaaacgcttttatgaagcgctggatgcgggcgatgcggaaaccgcgagcgcattgtttccagatggcacccgcatttatctgtgggatggccgcgtttttaccacccaggcggaatttcgcgcgtggtttgtggaactgcgttcgaccagcgccgatgcgaaacgtgaaattgttaaatttgaagtggatggcgatgtgtcctatgtggaagtggtgctgaaagcgaacgtagatggtgaagaaaagattgtgcgcctgcgccatgtgtttcattttgaaggcgatcgcctggttgaagtcgaagttgaaatcgaaccgctgggtaa

SEQ ID NO: 365 - DNA, 356 bp, synthetic construct
  source 1..356: mol_type = other DNA
  variation 1..3: note = optionally absent
  variation 354..356: note = optionally absent
atgtctgaagaagaacagcgcgaattttggaaacgcttttatgaagcgctggatgcgggcgatgcggaaaccgcgagcgcactttttccagatggcacccgcatttatctgtgggatggccgcacctttaccacccgcgccgaatttcgcgcgtggtttgaagaactgcatagcaccagtgaaaacgcgcgccgcgaaattgtgagctttcgtgtggatggcgatgtggcggatgtggaagtggtgctgcgtgcgaatgtgaatggcgaagaacgtaccgtgcgcctgcgtcatgtgtttcattttgaaggcgatcgcctggttgaagtgcatgttgaaattgaaccgctgggtaa

SEQ ID NO: 366 - DNA, 356 bp, synthetic construct
  source 1..356: mol_type = other DNA
  variation 1..3: note = optionally absent
  variation 354..356: note = optionally absent
atgagcgaagaagcgcagcgcgaattttggcgccgcttttatgatgcgctggatgcgggcgatgcggaaaccgcaagcgcgttgtttcctgatggcacccgcattcatctgtgggatggccgcacctttcatacccgcgccgaatttcgcgcgtggtttgtggaactgcgtagcacgagcgatgatgccaaacgcgaaattattagctttgaagtggatggcaaccgcagccgcgtggaagtggtgctgcatgcgaacatcgatggtgaagaaaagaaagtgctgctgcgccatgaatttctgtttgaaggcgatcgcctggttgaagttcatgtggaaattaaaccgctgggtaa

SEQ ID NO: 367 - DNA, 356 bp, synthetic construct
  source 1..356: mol_type = other DNA
  variation 1..3: note = optionally absent
  variation 354..356: note = optionally absent
atgagcgaagaagaaatccgcgaatttgttgatcgcttttataaagcgctggatgcgggtgatgcggaaaccgcaagcgcgttatttcccgatggcaccgaaattcatctgtgggatggtaccacctttcatacccaggcggaatttcgcgcgtggtttgtgaaactgaaatcgaccagcgataacgcgaaacgtgaaattgttaaactggaagtggatggcaacaaagcgaaagtggaagttgtgctgaaagccaacgttaacggtgaagaaaaagtggtcaaactgacccatgaatttgaattcgaaggcgatcgcctggtgaaagtgaccgtgaaaattgaaccgctgggtaa

SEQ ID NO: 368 - DNA, 356 bp, synthetic construct
  source 1..356: mol_type = other DNA
  variation 1..3: note = optionally absent
  variation 354..356: note = optionally absent
atgagcgcggaagcaattcgccagtttgtggaacgcttttatgcggcgctggatgcgggtgatgcggatactgcatcagcgctgtttccggatggtaccgaaattcatctgtgggatggccgtacctttcgcacccaggcggaatttcgcgcgtggtttgaagaactgtatagcaccagcgatggcgcgagccgcgaagtggtggcacttgaagttgatggtaaccgcgcgagagtggaagttgtgctgcatgcgagcgttggcggcgaagaaaagaccgtgcgcctgcgtcatgaatttgaattcgaaggcgatcagctggtgcgcgtgaccgtggaaattgaaccgctgggtaa

SEQ ID NO: 369 - DNA, 356 bp, synthetic construct
  source 1..356: mol_type = other DNA
  variation 1..3: note = optionally absent
  variation 354..356: note = optionally absent
atgagcgaaaaagaacagaaagaattttggaaaaagttttatgatgcgctggatgcgggtgatgcggataccgcgagcgcactgtttccagatggcaccaaaatttatctgtgggatggcaaagtgtttcgcacccaggaagaattccgtgaatggtttgaaaaactgtatagcaaaagcgataacgccaaacgcgagattgtgaaatttaaagttgatggtaacatcgcctatgtggaagtgattctgaaagcgaacgtggatggcgaagagaaagtggtgaaactgaaacatgtgtttaaatttgaaggcgataaagtgaaagaagttaaagtcaaaattgtgccgctgggtaa

SEQ ID NO: 370 - DNA, 356 bp, synthetic construct
  source 1..356: mol_type = other DNA
  variation 1..3: note = optionally absent
  variation 354..356: note = optionally absent
atgagcgaagaaagccagcgcgaattttggaaacgcttttatgcggcgctggatgcgggcgatgcggaaactgcaagcgcattgtttccggatggcaccgaaattcatctgtgggatggtaccgtgtttcgcacccgcgcggaatttcgcgcgtggtttgtggatctgcatagcaaaagcgataacgcgagccgcgaaattacctcttttaaagtggaaggcaacaaagcgctggttgaagtggtgctgcatgcgagctttaaaggtgaagaacgcaccgtgaaactgacccacgtgtttgaatttgaaggcgatcgcctggtgcgcgtcgaagttgaaattaaaccgctgggtaa

SEQ ID NO: 371 - DNA, 356 bp, synthetic construct
  source 1..356: mol_type = other DNA
  variation 1..3: note = optionally absent
  variation 354..356: note = optionally absent
atgtcggaagaagcggtgcgtgaatttgtgcgccgcttttatgaagcgctggatgcgggcgatgcggaaactgcgagcgccttgtttcctgatggcaccgaaattcatctatgggatggtaccacctttcgcacccgcgcggaatttcgtgcgtggtttcgtcgtctgagagcaactagcgaagatgcgcgccgtcgtgtggtgcgtctggaagtggatggcaacgttgcggatgtcgaagttgtgctgcatgcgagctatcgcggtgaagaacgcgttgtgcggctgcgccatcgctacgaatttgaaggcgatcgcctggtgcgcgttgatgttgaaattcgcccgctgggtaa

SEQ ID NO: 372 - DNA, 356 bp, synthetic construct
  source 1..356: mol_type = other DNA
  variation 1..3: note = optionally absent
  variation 354..356: note = optionally absent
atgagcgaagaagaacagcgccagtttgtgaaacgcttttacgaagcgctggatgcgggtgatagcgaaaccgcaagcgcgctgtttccggatggcaccgtgattcatctgtgggatggtaccacctttcatacccaggaagaatttcgcgcgtggtttgaaaaactgcgcagcaccagcgataacgcgcgccgtgaagtggtgcattttgaagttgaaggcaacgtggcgtatgtggaagttgttctgcgtgcgagcgtgggcggtgaagaacgcgttgtgcgtctgcgtcatgtgtttcatttcgaaggcgatcatctggttgaagtcgaagtggaaattgaaccgctgggtaa

SEQ ID NO: 373 - DNA, 356 bp, synthetic construct
  source 1..356: mol_type = other DNA
  variation 1..3: note = optionally absent
  variation 354..356: note = optionally absent
atgagcgaagaagaacagcgcaacttttggaaaaagttttatgaagcgctggatgcgggtgatgcggaaaccgcaagcgcgctgtttcctgatggcaccgaaatttatctgtgggatggtaccgtgtttaaaacccgcgaagagttccgtgcgtggtttgtggaactgtattcgacctcggaaaacgcgaaacgccatattgtggattttaaagtggatggcaacgaaagcaaagtgaaagtgattctgaaagcgaacattgatggtgaagaaaaggtggttgaactggaacattattttctgtttgaaggcgatcatctggtggaagtgaaggtggaaattcatccgctgggtaa

SEQ ID NO: 374 - DNA, 356 bp, synthetic construct
  source 1..356: mol_type = other DNA
  variation 1..3: note = optionally absent
  variation 354..356: note = optionally absent
atgagcgaggaagaaattcgagaatttgtggaacgcttttataaagcgctggatgcgggcgatgcggaaactgcgagcagcctgtttccagatggcaccgaaattcatctgtgggatggcaaaacatttcatacccaagaagaatttcgcagctggtttgtcgaactgaaaagcaaaagcgataacgccaaacgcaaagtggtgagctttgaagtggaaggcaacaaagcgaaagtgaaagttgtgctgaaagcgagctataaaggggaggaaaaagaagttaaactggaacatgaatttgaattcgaaggcgataaattggttgaagtcaacgtgaaaattgaaccgctgggtaa

SEQ ID NO: 375 - DNA, 356 bp, synthetic construct
  source 1..356: mol_type = other DNA
  variation 1..3: note = optionally absent
  variation 354..356: note = optionally absent
atgagcgaaaaagaacagcgcgaattttggaagaaattttatgaagcgctggatgcgggtgatgcggaaaccgccagcgcgttatttcccgatggcaccgaaattcatctgtgggatggcaaaacctttcatacccgcgaagaatttaaagaatggtttgtgaaactgtatagccgttcagaaaacgcgaaacgtcatattgtgaagtttgaagtggatggcgataaagcgtatgtggaagtggtgctgtatgcgaaaattggcggtgaagaaaaagtggttaaactgaaacatgaatttctgtttgagggcgacaaactggttgaagttaacgtgaaaatcgaaccgctgggtaa

SEQ ID NO: 376 - DNA, 356 bp, synthetic construct
  source 1..356: mol_type = other DNA
  variation 1..3: note = optionally absent
  variation 354..356: note = optionally absent
atgagcgaagaagaaattaaagaatttgtggaacgcttttataaagcgctggatgcgggtgatgcggataccgcaagcagcctgtttccagatggcaccgaaatcttcctgtgggatggcaaaacctttaaaaccaaagaggaatttcgcgaatggtttgtgaaactgaaaagcgaaagcgatgatgcgaaacgcgaagtggtgtcgctgaaagtggaaggcaacaaagcgtatgtggaagtagtgctgcatgcgaactataaaggcgaaaagaaagaagttaaactgacccatatttttgaatttgaaggtgataaagtcgtgaaagttgaagtgaccattgtgccgctgggtaa

SEQ ID NO: 377 - DNA, 356 bp, synthetic construct
  source 1..356: mol_type = other DNA
  variation 1..3: note = optionally absent
  variation 354..356: note = optionally absent
atgagcgaagaagagcagcgccagtttgtgaaagaattttatgaagcgctggatgcgggtgatgcggaaaccgcgagcgcgttgtttccagatggcaccgaaattcatctgtgggatggtaaaacctttaccacccaggcggaatttcgcgcctggtttgaacgcctgtatagcaccagcgaaaatgcgcgccgccatgtcgtggattttcgtgtggaaggcaacgtggccgatgttgaagtggtgctgcatgcgaccaaagatggtgaagaacataccgtgcgcctgcgtcataaatttgaattcgaaggcgatgaactggtgcgcgtggaagttgtgattgaaccgctgggtaa

SEQ ID NO: 378 - DNA, 356 bp, synthetic construct
  source 1..356: mol_type = other DNA
  variation 1..3: note = optionally absent
  variation 354..356: note = optionally absent
atgagcgcggaagcgcagcgcgaattttgggcgcgcttttatgcggcgttggatgcgggtgatgcggataccgctagcgcgctgtttcctgatggcaccgaaatttatctgtgggatggcaaagtgtttcgcacccgcgaagaatttcgcgcgtggtttgtggaactgcgcagcaccagcgccgatgcgaaacgtgaaattgtgagctttgaagtggatggtaaccgtgcgagcgtggatgtggtgctgcatgccagcgtgaacggtgaagaacataccgtgcatctgcatcacgaatttgaattcgaaggcgatcgcctggtgcgcgtgcgtgtgaccattcagccgctgggtaa

SEQ ID NO: 379 - DNA, 356 bp, synthetic construct
  source 1..356: mol_type = other DNA
  variation 1..3: note = optionally absent
  variation 354..356: note = optionally absent
atgagcgcagaacagcagcgcgaatttgtgaaacgcttttatgaagcgctggatgcgggtgatgcggataccgcaagcgcactgtttccggatggcaccgaaattcatctgtgggatggtaccacctttcgcacccgcgcggaatttcgcgcgtggtttgaagaactgtatagcacctcggaaaacgcgagccgcgaagtgaccagcttttcggtggatggcgatgtggcagatgtggaagtggtgctgcgcgcgaacctgggtggtgaagatcgcaccgttagcctgagacatgtgtttcattttgcgggcgatcgcctggtgcgcgttgaagtgagcattcgcccgctgggtaa

SEQ ID NO: 380 - DNA, 356 bp, synthetic construct
  source 1..356: mol_type = other DNA
  variation 1..3: note = optionally absent
  variation 354..356: note = optionally absent
atgagcgaagaagaaattaaagaatttgtgcgccgcttttatgaagcgctggatgcgggtgatgcggataccgcaagcgcactgtttccggatggcacccgcattcatctgtgggatggtaccacctttaccacccgcgcggaatttcgcgcgtggtttgtggatctgtatagcaaaagcgatgcggcgaaacgtgaagtgaccagcctgaaagtggaaggtaatgtggcgaaagtgaaagtagtgctgaaagcgaactataaaggtgaagaaaagaccgtggaactggaacataaatttgaattcgaaggcgatcgcctggtgcgcgttgatgttaccattaaaccgatgggtaa

SEQ ID NO: 381 - protein, 117 aa, synthetic construct
MSEEEIRQFLRRFYEAFDKGDVDTFASLFHPGVTIHVWQGITFTSREELREWVERFLRNFKDMQREILSLEVRGDTVEVHVQVHTTHNGQKYTFDVTHHWHFRGHRVTEIRVHVNPT